(19) United States
(12) Patent Application Publication
(10) Pub. No.: US 2019/0144536 A1
(43) Pub. Date: May 16, 2019

(54) ASPROSIN, A FAST-INDUCED GLUCOGENIC PROTEIN HORMONE

(71) Applicant: Baylor College of Medicine, Houston, TX (US)

(72) Inventors: Atul Chopra, Houston, TX (US); David D. Moore, Bellaire, TX (US)

(21) Appl. No.: 16/092,653

(22) PCT Filed: Apr. 13, 2017
(86) PCT No.: PCT/US17/27467

§ 371 Date: Oct. 10, 2018

Related U.S. Application Data
(60) Provisional application No. 62/322,043, filed on Apr. 13, 2016.
(60) Provisional application No. 62/373,565, filed on Aug. 11, 2016.

(51) Int. Cl. C07K 16/26; A61P 3/04; G01N 33/74

(52) U.S. Cl. CPC C07K 16/26; A61P 3/04; G01N 33/74; C07K 2317/24; A61K 2039/505; C07K 2317/14; C07K 2317/622; G01N 2333/575; C07K 2317/31

(57) ABSTRACT

Embodiments of the disclosure concern methods and compositions that relate to increasing or decreasing the weight (including, for example, by increasing or decreasing the adipose mass) in individuals in need thereof. Such methods and compositions, in particular embodiments, concern providing an effective amount of the hormone asprosin to increase adipose mass in an individual with insufficient adipose mass and providing an antibody or inhibitor of asprosin in an individual with obesity or diabetes, for example, to reduce adipose mass.

This application claims priority to U.S. Provisional Patent Application No. 62/322,043, filed Apr. 13, 2016, and to U.S. Provisional Patent Application No. 62/373,565, filed Aug. 11, 2016, both of which applications are incorporated by reference herein in their entirety.

This invention was made with government support under 1K08DK102529 awarded by NIDDK. The government has certain rights in the invention.

### TECHNICAL FIELD

Embodiments of the disclosure include at least the fields of cell biology, molecular biology, endocrinology, and medicine.

### BACKGROUND

The American Medical Association classified obesity as a disease in 2013 (Morgen & Sorenson, 2014), and as a leading preventable cause of death worldwide a clearer understanding of its genetic and molecular underpinnings has never been more important (Morgen & Sorenson, 2014; Malik, et al., 2013). Obesity is caused by an imbalance between energy intake and output (Spiegelman, et al., 2001; Spiegelman, et al., 1996). Because of the number of organs that impact these two processes and the complexity of energy homeostasis, the study of obesity remains a significant scientific challenge (Spiegelman, et al., 2001). Historically, study of extreme human variation has been a powerful tool for solving complex biological problems and for developing therapeutic targets against disease (Goldstein, et al., 2009; Friedman, et al., 2009). The present disclosure describes the loss of a new circulating polypeptide hormone responsible for maintenance of fat mass and associated glycemic control as the molecular mechanism driving the phenotype of an extreme thinness disorder in humans known as Neonatal Progeroid Syndrome (NPS) (Hou, et al., 2009; O'Neill, et al., 2007).

### BRIEF SUMMARY

Embodiments of the disclosure concern methods and compositions that impact the weight of an individual, where certain compositions are useful to increase the weight of an individual and certain compositions are useful to decrease the weight of an individual. Although the loss or increase in weight may be by any suitable means, in specific embodiments the loss or increase in weight is because of the corresponding loss or increase of adipose mass. An individual that increases their weight may do so at least in part by increasing their appetite, although in certain embodiments their weight increases without increasing their appetite.

Embodiments of the disclosure include methods and compositions that encompass a C-terminal fragment of Fibrillin-1, referred to herein as asprosin, or functional fragments or functional derivatives thereof. The increase in asprosin, such as in circulating asprosin, is useful for increasing weight of an individual, whereas the decrease in asprosin is useful for decreasing weight of an individual, in particular embodiments.

In particular embodiments, asprosin or functional fragments or functional derivatives thereof are provided to an individual in need of gaining weight, including in need of gaining adipose mass. Such an individual may be in need of gaining weight because they have a medical condition that prevents them from gaining weight or retaining weight and/or because they cannot or do not gain or retain weight for other reasons, such as being naturally underweight or by external causes. In specific embodiments, the medical condition is because of one or more genetic defects in the individual. In certain embodiments, the medical condition comprises cachexia as a symptom.

In certain embodiments, an individual is in need of losing weight and is therefore provided an effective amount of an inhibitor of the native asprosin in the individual. The inhibitor may be of any kind, but in specific embodiments the inhibitor is an antibody or small molecule, including an antibody or small molecule that targets an epitope on the N-terminal end of asprosin, the C-terminal end of asprosin, or an internal region of asprosin, for example.

In embodiments of the disclosure, an individual is in need of an improvement of glucose control and is therefore provided an effective amount of an inhibitor of the native asprosin in the individual. The inhibitor may be of any kind, but in specific embodiments the inhibitor is an antibody or small molecule, including an antibody or small molecule that targets an epitope on the N-terminal end of asprosin, the C-terminal end of asprosin, or an internal region of asprosin, for example. Such an individual may be of any kind, but in specific embodiments, the individual is diabetic, pre-diabetic (either or which may be determined by the fasting plasma glucose test, the oral glucose tolerance test and/or the Hemoglobin A1C test), insulin-resistant, and so forth. In specific embodiments, hyperglycemics and insulin-resistant individuals are provided an effective amount of one or more asprosin inhibitors. In certain embodiments, an individual is provided an effective amount of an asprosin inhibitor when the individual is in need of an improvement in the control of blood sugar and the asprosin inhibitors is given to the individual specifically for such improvement.

Embodiments of the disclosure include an appetite stimulant that comprises asprosin or functional fragments or functional derivatives thereof. Embodiments of the disclosure also include an appetite suppressant that comprises one or more inhibitors of asprosin.

In one embodiment, there is a recombinant asprosin polypeptide or a functional derivative or functional fragment thereof. In a specific embodiment, the asprosin polypeptide comprises, consists essentially of, or consists of the sequence of SEQ ID NO:1. In particular embodiments, the polypeptide is comprised in a pharmaceutically acceptable carrier. In specific embodiments, the functional derivative or fragment thereof comprises 1, 2, 3, 4, 5, 6, 7, 8, 9, 10, 11, 12, 13, 14, 15, 16, 17, 18, 19, or 20 or more amino acid alterations compared to SEQ ID NO:1. The functional derivative or functional fragment thereof may comprise an N-terminal truncation of SEQ ID NO:1, in certain embodiments, and the truncation may be no more than 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids or wherein the truncation is at least 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids, in particular embodiments. In certain embodiments, the functional derivative or functional fragment thereof comprises a C-terminal truncation of SEQ ID NO:1, such as wherein the truncation is no more than 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids or is at least 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids, for example. In some embodiments, the functional derivative or functional fragment thereof comprises an internal deletion in SEQ ID NO:1, such as an internal deletion that is no more than 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids or is at least 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids, for example. In some cases, the asprosin functional derivative or fragment thereof may comprise sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO:1. In specific embodiments, the polypeptide is labeled.

In one embodiment, there is a method of modulating the weight of an individual, comprising the step of modulating the level of native asprosin in the individual. In a specific embodiment, when the individual has insufficient weight, the level of native asprosin is increased. In a specific embodiment, when the individual has excessive weight, the level of native asprosin is decreased. In particular cases, the level of native asprosin is modulated by modulating transcription of asprosin and/or is modulated by modulating translation of asprosin. In specific embodiments, the level of native asprosin is modulated by modulating secretion of asprosin from cells and/or is modulated by modulating the stability of asprosin.

In one embodiment, there is a method of increasing the weight of an individual, comprising the step of providing an effective amount of any polypeptide contemplated herein to the individual. In a specific embodiment, the appetite level of the individual is increased.

In one embodiment, there is a method of decreasing the weight of an individual, comprising the step of providing an effective amount of an inhibitor of asprosin to the individual. In a specific embodiment, the inhibitor is an antibody, although it may be a small molecule.

In one embodiment, there is a method of decreasing the level of glucose in the blood of an individual, comprising the step of providing an effective amount of an inhibitor of asprosin to the individual.

In a particular embodiment, there is a method of increasing the level of glucose in the blood of an individual, comprising the step of providing an effective amount of any polypeptide as contemplated herein to the individual.

In an embodiment, there is a kit comprising any polypeptide as contemplated herein, wherein the polypeptide is housed in a suitable container.

In one embodiment, there is a method of stimulating the appetite of an individual, comprising the step of providing an effective amount of any polypeptide contemplated herein to the individual.

In a certain embodiment, there is an inhibitor of any polypeptide as contemplated herein.

Embodiments of the disclosure provide certain isolated antibodies or antibody fragments that specifically bind a peptide comprising, consisting of, or consisting essentially of SEQ ID NO:4. In specific embodiments the antibody or antibody fragment specifically binds an epitope that is on the peptide of SEQ ID NO:4, and that epitope may be in any region of the peptide of SEQ ID NO:4. The epitope may comprise continuous amino acids of the peptide or it may not comprise continuous amino acids of the peptide, such as when the epitope is a particular three-dimensional configuration. In specific cases the antibody is a monoclonal antibody, and the monoclonal antibody may be human or mouse. In certain aspects an asprosin antibody or antibody fragment is specific for asprosin in that it does not substantially bind FBN-1, for example as determined by routine methods such as western blotting.

Embodiments of the disclosure encompass antibodies produced by a hybridoma cell line deposited with the American Type Culture Collection under accession number ATCC PTA-123085, as well as the hybridoma cell or cell line deposited with the American Type Culture Collection under accession number ATCC PTA-123085.

Methods of measuring the level of asprosin in a sample are encompassed in the disclosure, and such methods may be of any kind, including those that utilize antibodies that specifically bind asprosin. Such antibodies may be of any kind, including monoclonal, and including antibodies produced by a hybridoma cell line deposited with the American Type Culture Collection under accession number ATCC PTA-123085. The sample may be of any kind, but in specific cases the sample is from a mammal and the individual is in need of determining whether or not he or she has or is at risk of developing insulin resistance, type II diabetes, and/or metabolic syndrome. The individual may be overweight or obese or may be at risk of being overweight or obese (for example having a family history).

Methods of treatment are encompassed by the disclosure, including methods of treating insulin resistance, obesity, diabetes of any type (including at least type I diabetes, type II diabetes, and Maturity-Onset Diabetes of the Young), obesity, and/or metabolic syndrome. Such methods may utilize administration of one or more antibodies that specifically bind asprosin, and although the antibody may be of any kind, including an antibody fragment, in specific embodiments the antibody is a monoclonal antibody produced by a hybridoma cell line deposited with the American Type Culture Collection under accession number ATCC PTA-123085.

One embodiment of the present disclosure comprises an isolated antibody or antibody fragment that specifically binds a peptide consisting of SEQ ID NO:4. In specific embodiments, the antibody is produced by hybridoma cell line deposited with the American Type Culture Collection under accession number ATCC PTA-123085. In some embodiments, the antibody is a humanized antibody, a single chain antibody, a nanobody, a humanized single chain antibody, a nanobody, a bispecific antibody, or a humanized bispecific antibody. In some cases, the antibody or antibody fragment is conjugated to a biologically active effector domain. Embodiments of the disclosure also encompass a composition comprising any antibody or antibody fragment encompassed by the disclosure. Any antibody or antibody fragment of the disclosure may be immobilized on a support and/or may be coupled to a detectable label.

In one embodiment, there is a hybridoma cell as deposited with the American Type Culture Collection under accession number ATCC PTA-123085. In some embodiments, there is provided a monoclonal antibody produced by a hybridoma deposited with the American Type Culture Collection under accession number ATCC PTA-123085. Other embodiments include hybridoma cell line ATCC PTA-123085 and an antibody produced by the cell line.

In a certain embodiment, there is a method of measuring the level of asprosin in a sample from an individual, comprising the steps of a) contacting an antibody or antibody fragment that specifically binds a peptide consisting of SEQ ID NO:4 with a sample; b) forming a complex between the antibody and asprosin from the sample; and c) detecting the antibody/asprosin complex and determining the level of asprosin in the sample. In specific embodiments, an individual is suspected of having or is known to have insulin resistance, type II diabetes, or metabolic syndrome or is obese or overweight. Samples may be of any kind, including biological samples such as plasma, blood, biopsy, saliva, semen, urine, hair, cerebrospinal fluid, cheek scrapings, nipple aspirate, or a combination thereof. In embodiments of the method, the antibody or antibody fragment is immobilized on a support or the antibody/asprosin complex is immobilized on a support. The antibody or antibody fragment may be coupled to a detectable label. In particular embodiments, the individual is identified as having or is at risk of developing insulin resistance, type II diabetes, or metabolic syndrome if the level of asprosin is greater than a reference level.

In some embodiments, there is a method of treating insulin resistance, obesity, type II diabetes, and/or metabolic syndrome in an individual, comprising the step of providing an effective amount of an antibody or antibody fragment or composition of the disclosure to the individual.

In an embodiment, there is provided a method of inhibiting asprosin in an individual, comprising the step of providing an effective amount of an antibody or antibody fragment or composition encompassed by the disclosure to the individual. In specific embodiments, the individual has or is suspected of having insulin resistance, obesity, type II diabetes, and/or metabolic syndrome. In a specific embodiment, an individual has a body mass index (BMI) of 30 or greater. In a certain embodiments, an individual has a BMI of between 25 and 29.9.

In some embodiments, there is use of an antibody or antibody fragment composition of the disclosure for the manufacture of a medicament for reducing asprosin levels in an individual. In certain cases, the individual has or is suspected of having insulin resistance, obesity, type II diabetes, and/or metabolic syndrome, for example. There is also use of an antibody or antibody fragment or composition of the disclosure for the manufacture of a medicament for treating insulin resistance, obesity, type II diabetes, and/or metabolic syndrome in an individual.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIGS. 1A-1D: Neonatal progeroid syndrome results from de novo, heterozygous, truncating mutations at the 3′ end of FBN1—FIG. 1A, Representative images of two NPS patients showing the associated lipodystrophy, which predominantly affects the face and extremities while sparing the gluteal area. FIG. 1B, FBN1 mutations, body mass indices (BMI) and family pedigrees of two NPS patients. FIG. 1C, 3′ FBN1 mutations in the two NPS patients of the disclosure and five NPS patients from published case reports. Patient #2 also has a heterozygous missense mutation (c.8222T>C) in FBN1 that is predicted to be benign and is not indicated in the figure for clarity. FIG. 1D, All seven NPS mutations (SEQ ID NO:8-14) are clustered around the Furin cleavage site (RGRKRR [SEQ ID NO:6] motif shown in red) and are predicted to result in heterozygous ablation of all of, or the majority of, the C-terminal polypeptide, which is shown in black following the RGRKRR (SEQ ID NO:6) motif. Non-native amino acids added on due to frame-shift are shown in blue. A wild type (WT) sequence is presented for reference (SEQ ID NO:7).

FIGS. 2A-2C: FBN1 is highly and dynamically expressed in white adipose tissue—FIG. 2A, FBN1 expression was measured by quantitative polymerase chain reaction in mouse white adipose tissue, brown adipose tissue and skeletal muscle (n=5 in each group). FIG. 2B, FBN1 expression was measured by quantitative polymerase chain reaction in human pre-adipocytes that were subjected to adipogenic differentiation for 7 days. CEBPa expression is shown as a marker of adipogenic differentiation. FIG. 2C, FBN1 expression was measured by quantitative polymerase chain reaction in inguinal white adipose tissue from male, WT mice subjected to normal chow or 10 weeks of high fat diet (n=5 in each group). Data are represented as the mean±SEM. Unpaired student's t test was used for evaluation of statistical significance. *P<0.05, **P<0.01, and ***P<0.001.

FIGS. 3A-3D: Asprosin is a highly conserved, circulating, C-terminal cleavage product of Fibrillin-1—FIG. 3A, Human FBN1 gene and its evolutionary conservation are depicted using the UCSC genome browser. The Asprosin coding region is boxed. FIG. 3B, A zoomed in view of exons 65 and 66, which contribute to the Asprosin coding region, is depicted using the UCSC genome browser. FIG. 3C, Western blot analysis targeted against Asprosin was performed on plasma from 14 week old WT mice subjected to normal chow or 8 weeks of high fat diet, or from 8 week old male mice either heterozygous or homozygous for the spontaneous Leptin mutation known as ob. FIG. 3D, Western blot analysis targeted against Asprosin was performed on plasma from obese humans or normal weight control subjects.

FIGS. 4A-4H: Asprosin rescues the NPS associated adipogenic differentiation defect in vitro—FIG. A4, Expression of several early and late markers of adipogenesis was measured by quantitative polymerase chain reaction in human dermal fibroblasts from NPS patients (mutant) or unaffected control subjects (WT) that were subjected to adipogenic differentiation for 7 days. FIG. 4B, Animated depictions of expression constructs expressing WT fibrillin-1 (WT FBN1), Asprosin without a signal peptide (FBN1 CT), and Asprosin with an attached signal peptide (FBN1 CTSP), all under control of the CMV promoter. The 27 amino acid native fibrillin-1 signal peptide is shown in red. FIG. 4C, Western blot analysis targeted against Asprosin was performed on cell culture media from WT human dermal fibroblasts exposed to adipogenic induction for 7 days and concurrently exposed to expression constructs driving WT fibrillin-1 (WT FBN1), Asprosin without a signal peptide (FBN1 CT), and Asprosin with an attached signal peptide (FBN1 CTSP), or Green Fluorescent Protein (GFP) as a control. FIG. 4D, Expression of an early (CEBPa) and a late (AP2) marker of adipogenesis was measured by quantitative polymerase chain reaction in human dermal fibroblasts from NPS patients (mutant) or unaffected control subjects (WT) that were subjected to adipogenic differentiation for 7 days, while concurrently exposed to expression constructs driving WT fibrillin-1 (WT FBN1) or GFP. Statistical comparison is shown between the Mutant+GFP group and the Mutant+WT FBN1 group. FIG. 4E, Expression of an early (CEBPa) and a late (AP2) marker of adipogenesis was measured by quantitative polymerase chain reaction in human dermal fibroblasts from NPS patients (mutant) or unaffected control subjects (WT) that were subjected to adipogenic differentiation for 7 days, while concurrently exposed to expression constructs driving Asprosin without a signal peptide (FBN1 CT) or GFP. Statistical comparison is shown between the Mutant+GFP group and the Mutant+FBN1 CT group. FIG. 4F, Expression of an early (CEBPa) and a late (AP2) marker of adipogenesis was measured by quantitative polymerase chain reaction in human dermal fibroblasts from NPS patients (mutant) or unaffected control subjects (WT) that were subjected to adipogenic differentiation for 7 days, while concurrently exposed to expression constructs driving Asprosin with an attached signal peptide (FBN1 CTSP) or GFP. Statistical comparison is shown between the Mutant+GFP group and the Mutant+FBN1 CTSP group. FIG. 4G, Expression of several early and late markers of adipogenesis was measured by quantitative polymerase chain reaction in human dermal fibroblasts from unaffected control subjects (WT) that were subjected to adipogenic differentiation for 7 days, while concurrently exposed to 60 nanomolar recombinant Asprosin or GFP. Induction of CEBPa expression was observed with a range of Asprosin doses from 30 nanomolar to 625 nanomolar. FIG. 4H, Expression of an early (CEBPa) and a late (AP2) marker of adipogenesis was measured by quantitative polymerase chain reaction in human dermal fibroblasts from NPS patients (mutant) or unaffected control subjects (WT) that were subjected to adipogenic differentiation for 7 days, while concurrently exposed to 60 nanomolar recombinant Asprosin or GFP. Data are represented as the mean±SEM. Unpaired student's t test was used for evaluation of statistical significance. *P<0.05, **P<0.01, and ***P<0.001.

FIGS. 5A-5J: High circulating Asprosin is obesogenic and diabetogenic—FIG. 5A, FIG. 5B, FIG. 5C, Fat mass and lean mass using Magnetic Resonance Imaging (MM), and total body weight were measured in WT mice subjected to a one-time tail vein injection of 10¹¹ viral particles of adenovirus carrying cDNA (under control of the CMV promoter) for FBN1 or GFP (n=6 in each group). Measurements were conducted on the indicated days. FIG. 5D, FIG. 5E, FIG. 5F, Fat mass and lean mass using Magnetic Resonance Imaging (MM), and total body weight were measured in WT mice subjected to daily subcutaneous injection of 2.6 micro molar recombinant Asprosin or GFP for 10 days (n=6 in each group). Measurements were conducted on the indicated days. FIG. 5G, FIG. 5I, Glucose tolerance test and insulin tolerance test were performed on fasted WT mice subjected to a one-time tail vein injection of 10¹¹ viral particles of adenovirus carrying cDNA (under control of the CMV promoter) for FBN1 or GFP (n=6 in each group). Measurements were conducted 10 days after the adenoviral injection. FIG. 5H, FIG. 5J, Glucose tolerance test and insulin tolerance test were performed on fasted WT mice subjected to daily subcutaneous injection of 2.6 micro molar recombinant Asprosin or GFP for 10 days (n=6 in each group). Measurements were conducted 10 days after the initial injection. Of note, insulin tolerance test on the GFP mice (both adenovirus and peptide mediated delivery) was complicated by severe hypoglycemia at the 60 minute mark that manifested as a “too low to measure” value on the glucometer. Those mice had to be injected with exogenous glucose to prevent fatal hypoglycemia. FBN1 adenovirus and Asprosin injected mice however maintained their blood glucose levels as indicated in the figure. Data are represented as the mean±SEM. For evaluation of statistical significance, unpaired student's t test was used when comparing two groups, or ANOVA was used when comparing more than two groups. *P<0.05, **P<0.01, and ***P<0.001.

FIGS. 6A-6D: Dominant negative effect of truncated profibrillin—FIG. 6A, Western blot analysis targeted against Asprosin was performed on plasma from NPS patients and unaffected control subjects (WT). FIG. 6B, Western blot analysis targeted against Asprosin was performed on cell culture media from human dermal fibroblasts from NPS patients (NPS) or unaffected control subjects (WT) exposed to adipogenic induction for 7 days, and concurrently exposed to vehicle or Monensin to block the secretory pathway. FIG. 6C, Animated depiction of expression constructs expressing WT fibrillin-1 (WT FBN1) or mutant profibrillin carrying the c.8207_8208Ins1bp mutation that induces a frame-shift and C-terminal truncation (FBN1 NTΔ). FIG. 6D, Western blot analysis targeted against Asprosin was performed on cell culture media from human dermal fibroblasts from unaffected control subjects (WT) exposed to adipogenic induction for 7 days, and concurrently exposed to expression constructs driving GFP or mutant, truncated profibrillin (FBN1 NTΔ), along with vehicle or Monensin to block the secretory pathway.

FIGS. 7A-7B: FBN1 Adenovirus or Asprosin injection increase the amount of circulating Asprosin—FIG. 7A, Western blot analysis targeted against Asprosin was performed on plasma from WT mice subjected to a one-time tail vein injection of 10¹¹ viral particles of adenovirus carrying cDNA (under control of the CMV promoter) for FBN1 or GFP. Measurements were conducted 10 days after the adenoviral injection. FIG. 7B, Western blot analysis targeted against Asprosin was performed on plasma from WT mice subjected to daily subcutaneous injection of 2.6 micro molar recombinant Asprosin or GFP for 10 days. Measurements were conducted 10 days after the initial injection.

FIGS. 8A-8B: Higher circulating Asprosin results in increased fat cell size—FIG. 8A, Formalin-fixed inguinal white adipose tissue sections were stained with hematoxylin and eosin from 4-hour fasted WT mice subjected to a one-time tail vein injection of 10¹¹ viral particles of adenovirus carrying cDNA (under control of the CMV promoter) for FBN1 or GFP. Sections were taken 10 days after the adenoviral injection. FIG. 8B, Formalin-fixed inguinal white adipose tissue sections were stained with hematoxylin and eosin from 4-hour fasted WT mice subjected to daily subcutaneous injection of 2.6 micro molar recombinant Asprosin or GFP for 10 days. Sections were taken 10 days after the adenoviral injection.

FIGS. 9A-9D: Increased circulating Asprosin results in higher plasma levels of adipose derived hormones—FIG. 9A, FIG. 9B Leptin and Adiponectin were measured in plasma from 4-hour fasted WT mice subjected to a one-time tail vein injection of 10¹¹ viral particles of adenovirus carrying cDNA (under control of the CMV promoter) for FBN1 or GFP (n=6 in each group). Measurements were conducted 10 days after the adenoviral injection. FIG. 9C, FIG. 9D Leptin and Adiponectin were measured in plasma from 4-hour fasted WT mice subjected to daily subcutaneous injection of 2.6 micro molar recombinant Asprosin or GFP for 10 days (n=6 in each group). Measurements were conducted 10 days after the initial injection.

FIGS. 10A-10D: Increased circulating Asprosin results in lower plasma lipids—FIG. 10A, FIG. 10B Triglycerides and Free Fatty Acids were measured in plasma from 4-hour fasted WT mice subjected to a one-time tail vein injection of 10¹¹ viral particles of adenovirus carrying cDNA (under control of the CMV promoter) for FBN1 or GFP (n=6 in each group). Measurements were conducted 10 days after the adenoviral injection. FIG. 10C, FIG. 10D Triglycerides and Free Fatty Acids were measured in plasma from 4-hour fasted WT mice subjected to daily subcutaneous injection of 2.6 micro molar recombinant Asprosin or GFP for 10 days (n=6 in each group). Measurements were conducted 10 days after the initial injection.

FIGS. 11A-11D: Increased circulating Asprosin results in hyperglycemia and hyperinsulinism—FIG. 11A, FIG. 11B Glucose and Insulin were measured in plasma from 4-hour fasted WT mice subjected to a one-time tail vein injection of 10¹¹ viral particles of adenovirus carrying cDNA (under control of the CMV promoter) for FBN1 or GFP (n=6 in each group). Measurements were conducted 10 days after the adenoviral injection. FIG. 11C, FIG. 11D Glucose and Insulin were measured in plasma from 4-hour fasted WT mice subjected to daily subcutaneous injection of 2.6 micro molar recombinant Asprosin or GFP for 10 days (n=6 in each group). Measurements were conducted 10 days after the initial injection.

FIGS. 12A-12B: Higher circulating Asprosin results in increased hepatic lipid accumulation—FIG. 12A, Formalin-fixed liver sections were stained with hematoxylin and eosin, and Oil-Red-O stain for neutral lipid, from 4-hour fasted WT mice subjected to a one-time tail vein injection of 10¹¹ viral particles of adenovirus carrying cDNA (under control of the CMV promoter) for FBN1 or GFP. Sections were taken 10 days after the adenoviral injection. FIG. 12B, Formalin-fixed liver sections were stained with hematoxylin and eosin, and Oil-Red-O stain for neutral lipid, from 4-hour fasted WT mice subjected to daily subcutaneous injection of 2.6 micro molar recombinant Asprosin or GFP for 10 days. Sections were taken 10 days after the adenoviral injection.

FIG. 13: Dominant negative effect of truncated profibrillin on fibrillin-1 secretion—Western blot analysis targeted against fibrillin-1 was performed on cell culture media from human dermal fibroblasts from unaffected control subjects (WT) exposed to adipogenic induction for 7 days, and concurrently exposed to expression constructs driving GFP or mutant, truncated profibrillin (FBN1 NTΔ), along with vehicle or Monensin to block the secretory pathway.

FIG. 14: Dermal fibroblasts from unaffected humans (WT) and patients with NPS (mutant) were differentiated into mature adipocytes using 7-day exposure to an adipogenic medium followed by gene expression analysis. Cells were concurrently exposed to adenovirus carrying no cDNA insert or adenovirus carrying a cDNA insert for Fibrillin-1 C-terminal polypeptide (which may also be referred to herein as asprosin) fused to a signal peptide for 7 days. AP2, CEBPa, Leptin and Adiponectin are adipogenic marker genes. CXCL1, CCL1, CCL3 and TLR2 are inflammogenic marker genes. Only statistical comparison between the ‘Mut+Empty Vector’ group and the ‘Mut+CT Polypeptide’ group is indicated on the figure for clarity. Unpaired student's t-test was used for statistical analysis. One asterisk indicates p<0.05, two asterisks p<0.01, and three asterisks p<0.001.

FIG. 15: Dermal fibroblasts from unaffected humans (WT) and patients with NPS (mutant) were differentiated into mature adipocytes using 7-day exposure to an adipogenic medium followed by gene expression analysis. Cells were concurrently exposed to vehicle or 10 ug of the Fibrillin-1 C-terminal polypeptide for 7 days. AP2, CEBPa, Leptin and Adiponectin are adipogenic marker genes. CXCL1, CCL1, CCL3 and TLR2 are inflammogenic marker genes. Only statistical comparison between the ‘Mut+Vehicle’ group and the ‘Mut+CT Polypeptide’ group is indicated on the figure for clarity. Unpaired student's t-test was used for statistical analysis. One asterisk indicates p<0.05, two asterisks p<0.01, and three asterisks p<0.001.

FIG. 16: Western blot analysis was performed on plasma from C57/Bl6 mice either fed a normal or high fat diet, using a mouse monoclonal antibody that detects the Fibrillin-1 C terminus specifically. The 16 kd band corresponds to the plasma fraction of the Fibrillin-1 C-terminus.

FIG. 17: An increased amount of plasma CT polypeptide (asprosin) results in hyperphagia in mice that have been injected with asprosin.

FIGS. 18A-18E: Neonatal Progeroid syndrome (NPS) mutations reduce plasma insulin levels while maintaining euglycemia in humans. (FIG. 18A) Overnight fasted plasma glucose and insulin levels from 2 NPS patients (NPS) and 4 unaffected control subjects (WT). (FIG. 18B) FBN1 mutations and family pedigrees of the two NPS patients in (FIG. 18A). Standard pedigree symbols are used with affected status noted by filled symbols. (FIG. 18C) 3′ FBN1 mutations in seven NPS patients—two reported herein and five from published case reports. Patient #2 also has a heterozygous missense variant (c.8222T>C) in FBN1 that is predicted to be benign and is not indicated in the figure for clarity. (FIG. 18D) Schematic depicting the clustering of the NPS mutations at the 3′ end of the FBN1 gene. (FIG. 18E) All seven NPS mutations are clustered around the furin cleavage site (RGRKRR [SEQ ID NO: 6] motif highlighted in yellow) and are predicted to result in heterozygous ablation of the 140 amino acid C-terminal polypeptide (asprosin). Non-native amino acids due to frame-shift are shown in red. Patient #2, Case 3 and Case 5 have a mutation in a splice-donor site that has been shown to produce the indicated mutant protein (Jacquinet et al., 2014). Data are represented as the mean±SEM. (SEQ ID NO: 15-22)

FIGS. 19A-19K: Asprosin, the C-terminal cleavage product of profibrillin, is a fasting responsive plasma protein. (FIG. 19A) Asprosin immunoblot on 6 individual human plasma samples (lanes 2-7). Bacterially expressed recombinant asprosin was used as a positive control (lane 8). The molecular weight marker is shown in lane 1. (FIG. 19B) Asprosin sandwich ELISA standard curve. (FIG. 19C) Sandwich ELISA was used to measure plasma asprosin levels in overnight fasted humans, mice and rats (n=7 in each group). (FIG. 19D) Sandwich ELISA was used to measure plasma asprosin levels in unaffected control subjects (WT), two patients with heterozygous FBN1 frame-shift mutations 5′ to the threshold for mRNA nonsense mediated decay (c.6769-6773del5, c.1328-23_c.1339del35insTTATTTTATT) (proximal truncation 1&2) and two NPS patients (distal truncation 1&2). (FIG. 19E) Sandwich ELISA was used to measure plasma asprosin every 4 hours from circadian C57Bl/6 mice entrained to total darkness (n=5). The period of feeding is shaded. (FIG. 19F) Sandwich ELISA was used to measure plasma asprosin levels in ad libitum fed or overnight fasted humans, mice and rats (n=7 in each group). (FIG. 19G) FBN1 expression across all human tissues using the GTEx human RNAseq database. (FIG. 19H) Various WT C57Bl/6 mouse organs were assessed for Fbn1 mRNA expression by qPCR. (FIG. 19I) Plasma asprosin was assessed using sandwich ELISA on plasma from 13-week old, 6-hour fasted, male WT and Bsc12 null mice. (FIG. 19J-19K) PPARγ2 mRNA expression by qPCR, and media asprosin by sandwich ELISA were assessed on cultured 3T3-L1 and C3H10T1/2 cells with or without exposure to an adipogenic cocktail for 7 days. Cells were washed with PBS and then exposed to glucose-free, serum-free media for 24 hours for assessment of secretion. Data are represented as the mean±SEM. See also FIGS. 25, 26, and 27.

FIGS. 20A-20I: Increase in circulating asprosin is associated with elevated blood glucose and insulin in mice (FIG. 20A) Profibrillin (350 kDa) immunoblot on liver lysates 10 days after WT mice were subjected to a one-time tail vein injection of 1011 viral particles of adenovirus carrying cDNA for FBN1 (lanes 3, 4 and 5) or GFP (lanes 1 and 2). Mice were subjected to a 2-hour fast for synchronization prior to sacrifice. (FIG. 20B) Sandwich ELISA was used to measure plasma asprosin levels from mice in (FIG. 20A) (n=5 in each group). (FIG. 20C) Plasma glucose and insulin levels from mice in (FIG. 20A) (n=5 in each group). (FIG. 20D) Plasma glucose and insulin levels were measured 10 days after WT mice were subjected to daily subcutaneous injection of 30 μg recombinant asprosin (validated to result in a 50 nM peak plasma level) or recombinant GFP for 10 days (n=5 in each group). (FIG. 20E) Plasma glucose was measured at the indicated times after a single 30 μg dose of subcutaneous recombinant asprosin or GFP in mice that had been subjected to a 2-hour fast prior to injection (n=6 in each group). Two-way ANOVA with Bonferroni post-test was used to calculate the p value. (FIG. 20F) Plasma insulin was measured 15 minutes after injection from mice in (FIG. 20E) (n=6 in each group). (FIG. 20G) Plasma glucose was measured at the indicated times after a single 30 μg dose of subcutaneous recombinant asprosin or GFP in mice that had been subjected to an overnight (˜16 hours) fast prior to injection (n=6 in each group). Two-way ANOVA with Bonferroni post-test was used to calculate the p value. (FIG. 20H) Plasma insulin was measured 30 minutes after injection from mice in (FIG. 20G) (n=6 in each group). (FIG. 20I) Plasma glucagon, catecholamines and corticosterone were measured 15-20 minutes after a single 30 μg dose of subcutaneous recombinant asprosin or GFP in mice that had been subjected to a 2-hour fast prior to injection (n=6 in each group). Data are represented as the mean±SEM.

FIGS. 21A-21E: In a cell-autonomous effect, asprosin targets the liver to increase plasma glucose (FIG. 21A) Glucose tolerance test was performed 2 hours following subcutaneous injection with 30 μg recombinant asprosin or GFP, in WT mice fasted for 2 hours for synchronization prior to injection (n=6 mice in each group). Two-way ANOVA with Bonferroni post-test was used to calculate the p value. (FIG. 21B) Insulin tolerance test was performed 2 hours following subcutaneous injection with 30 μg recombinant asprosin or GFP, in WT mice fasted for 2 hours for synchronization prior to injection (n=6 mice in each group). Two-way ANOVA with Bonferroni post-test was used to calculate the p value. (FIG. 21C) Basal (18-hour fasted) and clamped hepatic glucose production was measured using the hyperinsulinemic-euglycemic clamp 10 days after WT mice were subjected to a one-time tail vein injection of 1011 viral particles of adenovirus carrying cDNA for FBN1 or GFP (n=7 mice in each group). (FIG. 21D) Glucose disposal rate was measured in mice from (FIG. 21C) (n=7 mice in each group). (FIG. 21E) Media glucose accumulation was measured 2 hours after incubating mouse primary hepatocytes with 0, 4, 8, 16, 32, 64, 138, 275, 550 or 1100 nM recombinant asprosin or GFP, 1 hour following isolation of cells from WT mice, without plating the cells. Data are represented as the mean±SEM.

FIGS. 22A-22E: Asprosin traffics to the liver in vivo and binds the hepatocyte surface with high affinity in a saturable and competitive manner. (FIG. 22A) SPECT scans were performed 15 minutes after intravenous injection with 150 μCi I125-asprosin, boiled I125-asprosin, or free I125 in live, anesthetized mice previously injected with bismuth as a hepatic contrast agent. 3 representative images are shown in axial and coronal planes. (FIG. 22B) Liver asprosin accumulation was measured as liver photon intensity from mice in (FIG. 22A). (FIG. 22C) Tissue radioactivity (normalized to tissue weight) was measured using a γ-counter after sacrificing mice from (FIG. 22A), 45 minutes following injection (n=4 mice). (FIG. 22D) Sandwich ELISA was used to measure plasma His tag (recombinant asprosin contains an N-terminal His tag) in WT mice before injection and 15, 30, 60 and 120 minutes after injection with 30 μg recombinant asprosin. The time taken for peak signal to fall to half-maximal level is indicated by the arrow. (FIG. 22E) The level of biotin at the hepatocyte surface was measured using a colorimetric assay upon incubation of unplated mouse primary hepatocytes with increasing concentration of a recombinant asprosin-biotin conjugate, with (nonspecific binding) or without (total binding) 100-fold excess recombinant asprosin in the media. Specific binding (shown in red) was calculated as the difference between the two curves. Data are represented as the mean±SEM.

FIGS. 23A-23M: Asprosin uses the cAMP second messenger system and activates protein kinase A (PKA) in the liver. (FIG. 23A) Liver cAMP level was measured 15 minutes after a single 30 μg dose of subcutaneous recombinant asprosin or GFP in mice that had been subjected to a 2-hour fast prior to injection (n=6 in each group). (FIG. 23B) Liver PKA activity was measured in mice from (FIG. 23A). (FIG. 23C) Immunoblot analysis for phosphorylated PKA catalytic subunit or for phosphorylated serine/threonine PKA substrate was performed on liver lysates from mice in (FIG. 23A). (FIG. 23D) Hepatocyte cAMP level was measured 10 minutes after incubating mouse primary hepatocytes with 50 nM recombinant asprosin, 1 hour following isolation of cells from WT mice, without plating the cells. (23E) Hepatocyte PKA activity was measured in samples from (FIG. 23D). (FIG. 23F) Hepatocyte PKA activity was measured upon 2 hours of incubation of mouse primary hepatocytes with 0, 4, 8, 16, 32, 64, 138, 275, 550 or 1100 nM recombinant asprosin or GFP, 1 hour following isolation of cells from WT mice, without plating the cells. (FIG. 23G) Media glucose accumulation was measured 2 hours after incubating mouse primary hepatocytes with 50 nM recombinant asprosin or GFP, with or without a G-protein inhibitor (Suramin) (5 μM), 1 hour following isolation of cells from WT mice, without plating the cells. (FIG. 23H) Hepatocyte PKA activity was measured in samples from (FIG. 23G). (FIG. 23I) Media glucose accumulation was measured 2 hours after incubating mouse primary hepatocytes with 50 nM recombinant asprosin or GFP, with or without a competitive antagonist of cAMP induced activation of PKA (cAMPS-Rp) (200 μM), 1 hour following isolation of cells from WT mice, without plating the cells. (FIG. 23J-23K) Media glucose accumulation was measured 2 hours after incubating mouse primary hepatocytes with 50 nM recombinant asprosin or GFP, or 10 μg/ml glucagon, with or without a noncompetitive antagonist of the glucagon receptor (L168,049) (1 μM), or 100 μM epinephrine, with or without an antagonist of the β-adrenergic receptor (Propranolol) (100 μM), 1 hour following isolation of cells from WT mice, without plating the cells. The r GFP and r Asprosin controls are common for J and K. (FIG. 23L) Hepatocyte PKA activity was measured 2 hours after incubating mouse primary hepatocytes with 50 nM recombinant asprosin or GFP, with vehicle or 10 mg/L insulin, 1 hour following isolation of cells from WT mice, without plating the cells. (FIG. 23M) Hepatocyte glucose production was measured in samples from (FIG. 23L). Data are represented as the mean±SEM.

FIGS. 24A-24M: Immunologic or genetic asprosin loss-of-function reduces hepatic glucose production, plasma glucose and plasma insulin. (FIG. 24A) Sandwich ELISA was used to measure plasma asprosin levels in 8 obese, insulin resistant male human subjects and 8 non-obese sex- and age-matched control subjects. Pertinent physiological parameters are also presented. (FIG. 24B) Sandwich ELISA was used to measure plasma asprosin levels in male WT mice that had been subjected to a high fat diet (60% of calories from fat) or normal chow for 12 weeks, and from 5 week old, male Ob/+ or Ob/Ob mice, upon 2 hours of fasting for synchronization (n=5 mice in each group). (FIG. 24C) Sandwich ELISA was used to measure plasma asprosin levels at the indicated times after intra-peritoneal injection of 500 μg of IgG or anti-asprosin monoclonal antibody, with ad libitum feeding following a 2-hour fast for synchronization, in male WT mice that had been subjected to a high fat diet (60% of calories from fat) for 12 weeks (n=6 mice in each group). (FIG. 24D) Plasma glucose was measured in mice from (FIG. 24C). (FIG. 24E) Plasma insulin was measured in mice from (FIG. 24C). (FIG. 24F) Plasma glucose was measured at the indicated times in 5 week old, male WT or Ob/Ob mice after intra-peritoneal injection of 500 μg of IgG or anti-asprosin monoclonal antibody, with ad libitum feeding following a 2-hour fast for synchronization (n=6 mice in each group). (FIG. 24G) Plasma insulin was measured in mice from (FIG. 24F). (FIG. 24H) Sandwich ELISA was used to measure plasma asprosin levels in male WT or homozygous MgR mice following a 2-hour fast for synchronization (n=5 mice in each group). (FIG. 24I) Plasma glucose was measured in male WT or homozygous MgR mice following a 2-hour fast or following a 24-hour fast (n=5-7 mice in each group). (FIG. 24J) Plasma insulin was measured in mice from (FIG. 24I). (FIG. 24K) Basal (18-hour fasted) and clamped hepatic glucose production was measured using the hyperinsulinemic-euglycemic clamp in 10-week old WT or homozygous MgR mice (n=6 mice in each group). (FIG. 24L) Glucose disposal rate was measured in mice from (FIG. 24K) (n=6 mice in each group). (FIG. 24M) Plasma glucose and insulin was measured in WT or homozygous male MgR mice following an overnight fast, 30 minutes after subcutaneous injection of 30 μg recombinant asprosin or GFP (n=5-7 mice in each group). Data are represented as the mean±SEM. See also FIGS. 28, 29, 30, and 31.

FIGS. 25A-25E: Mammalian asprosin is evolutionarily well conserved, has a molecular weight of ˜30 kDa, and is predicted to contain 3 N-linked glycosylation sites, Related to FIG. 19. (FIG. 25A) Human FBN1 gene and its evolutionary conservation across 100 vertebrate species is depicted using the UCSC genome browser. The asprosin coding region is boxed. (FIG. 25B) Base-pair conservation using the PhyloP tool across 100 vertebrate species is depicted for FBN1 exons 1-64, exon 65-66 which encode asprosin and exon 66 alone (which contributes 129 out of the 140 asprosin amino acids). Exon 66 contains the 3′ untranslated region that was excluded from the analysis. (FIG. 25C) Asprosin immunoblot on cell lysates and media from WT and Fbn1 null mouse embryonic fibroblasts. (FIG. 25D) Human asprosin sequence showing the 3 N-linked glycosylation sites (in red) predicted by the NetNGlyc algorithm (SEQ ID NO: 23). (FIG. 25E) Asprosin N-linked glycosylation sites predicted by the NetNGlyc algorithm are shown as a schematic using sequence position and algorithm threshold.

FIGS. 26A-26B: Mammalian asprosin protein can be detected intracellularly in mouse white adipose tissue and cultured 3T3-L1 cells differentiated to mature adipocytes, Related to FIG. 19. (FIG. 26A) Asprosin and profibrillin immunoblots on white adipose tissue lysates from WT C57Bl/6 mice. (FIG. 26B) Asprosin and profibrillin immunoblots on cultured 3T3-L1 cells with and without exposure to an adipogenic cocktail for 7 days. Adipogenesis was confirmed by visualization of lipid droplets (not shown) and expression of the adipogenic master gene—PPARg2 (FIG. 19J).

FIGS. 27A-27E: Development and validation of the asprosin sandwich ELISA, and its use for assessment of the dominant negative effect of mutant profibrillin on asprosin secretion, Related to FIG. 19. (FIG. 27A) Sequence of human recombinant asprosin expressed in E. coli (SEQ ID NO: 24). The N-terminal his tag is shown in yellow and the capture antibody and detection antibody epitopes (for sandwich ELISA) are bolded and underlined. (FIG. 27B) Fbn1 mRNA expression by qPCR on WT and Fbn1 null mouse embryonic fibroblasts. (FIG. 27C) Asprosin sandwich ELISA on serum-free media from WT and Fbn1 null mouse embryonic fibroblasts. Cells were grown in regular media, washed with PBS and then exposed to serum-free media for 24 hours for assessment of secreted proteins. (FIG. 27D) Media glucose accumulation was measured 2 hours after incubating mouse primary hepatocytes with 50 nM recombinant asprosin or GFP, following 1 hour of pretreatment with 50 ug IgG or anti-asprosin monoclonal antibody, 1 hour following isolation of cells from WT mice, without plating the cells. (FIG. 27E) FBN1 mRNA expression by qPCR in WT human dermal fibroblasts transfected with CMV driven vectors encoding GFP or C-terminal truncated human profibrillin (carrying the human NPS mutation c.8206_8207InsA that induces a frame-shift, premature stop codon, and ablation of 136 of the 140 C-terminal profibrillin amino acids). Monensin, a pharmacological secretion blocker, was used as a negative control. (FIG. 27F) Asprosin was assessed by sandwich ELISA on media from (FIG. 27E). Cells were grown in regular media, washed with PBS and then exposed to serum-free media for 24 hours for assessment of secreted proteins. Data are represented as the mean±SEM. For evaluation of statistical significance, unpaired student's t-test was used. *P<0.05, **P<0.01, and ***P<0.001.

FIG. 28: Schematic depicting asprosin action at the hepatocyte surface, leading to use of cAMP as a second messenger, a burst of PKA activity, and glucose release into the circulation, which in turn leads to an insulin response that in time normalizes the plasma glucose. Related to FIG. 24.

FIGS. 29A-29F: White adipose tissue mediated secretion of asprosin is suppressed by glucose in a negative feedback loop, Related to FIG. 24. (FIGS. 29A-29D) PPARg2 mRNA expression by qPCR, and media asprosin by sandwich ELISA were assessed on cultured 3T3-L1 and C3H10T1/2 cells that had been exposed to an adipogenic cocktail for 7 days. Cells were washed with PBS and then exposed to either glucose-free or glucose-containing serum-free media for 24 hours for assessment of secretion. (FIG. 29E) Asprosin immunoblot on cells lysates from cultured 3T3-L1 and C3H10T1/2 cells with or without exposure to an adipogenic cocktail for 7 days. Mature adipocytes were exposed to serum-free media with or without glucose for 24 hours. Preadipocytes were only exposed to serum-free media without glucose for the same duration. (FIG. 29F) Asprosin was assessed by sandwich ELISA on plasma from 12-week old, male, WT C57Bl/6 mice injected with saline or streptozotocin i.p. (injected three times over the course of 2 weeks until blood glucose values by a handheld glucometer were >600 mg/dl). Mice were subjected to a 2-hour fast for synchronization prior to sacrifice. Data are represented as the mean±SEM. For evaluation of statistical significance, unpaired student's t-test was used. *P<0.05, **P<0.01, and ***P<0.001.

FIGS. 30A-30B: Intracellular asprosin is capable of secretion despite absence of an N-terminal signal peptide, Related to FIG. 24. (FIG. 30A) The human asprosin coding sequence (driven by a CMV promoter) or an empty vector were transfected into Fbn1 null mouse embryonic fibroblasts. 48 hours later, asprosin-transfected cells were washed with PBS and then exposed to glucose free or glucose containing serum-free media for 24 hours for assessment of secretion. Empty vector transfected cells were only exposed to glucose free serum-free media for the same duration. Human FBN1 exon 66 (which encodes 129 of the 140 asprosin amino acids) expression was determined by qPCR. (FIG. 30B) Asprosin was assessed by sandwich ELISA on serum-free media from S6A. Cells were grown in regular media, washed with PBS and then exposed to serum-free media with or without glucose for 24 hours for assessment of secreted proteins. Cells transfected with empty vector were only exposed to serum-free media without glucose. Data are represented as the mean±SEM. For evaluation of statistical significance, unpaired student's t-test was used. *P<0.05, **P<0.01, and ***P<0.001.

FIGS. 31A-31D: Insulin resistance results in upregulation of Fbn1 mRNA expression in adipose tissue and skeletal muscle, Related to FIG. 24. (FIG. 31A) Various mouse organs were assessed for Fbn1 mRNA expression using qPCR in 12-week old, male WT and Ob/Ob mice. Mice were subjected to a 2-hour fast for synchronization prior to sacrifice. (FIG. 31B) Various mouse organs were assessed for Fbn1 mRNA expression using qPCR in 12-week old, male ad libitum fed or 24-hour fasted WT C57Bl/6 mice. Mice were subjected to a 2-hour fast for synchronization prior to sacrifice. (FIG. 31C) Various mouse organs were assessed for Fbn1 mRNA expression using qPCR in mice from FIG. 29F. (FIG. 31D) Endotoxin levels in recombinant asprosin and recombinant GFP were determined before and after passing the recombinant proteins through endotoxin depletion columns (for as many attempts as were required to bring the final endotoxin concentration equal to or below 2 EU/ml). Data are represented as the mean±SEM. For evaluation of statistical significance, unpaired student's t-test was used. *P<0.05, **P<0.01, and ***P<0.001.

FIG. 32: Anti-asprosin monoclonal antibody reduces food intake.

FIG. 33: Immunological sequestration of circulating asprosin.

FIGS. 34A-34B: The anti-asprosin monoclonal antibody improves the hyperinsulinism associated with diet-induced obesity. (FIG. 34A) Measurement of plasma glucose levels. (FIG. 34B) Measurement of plasma insulin levels.

FIGS. 35A-35B: The anti-asprosin monoclonal antibody improves the hyperinsulinism associated with the Ob mutation. (FIG. 35A) Measurement of plasma glucose levels. (FIG. 35B) Measurement of plasma insulin levels.

FIGS. 36A-36B: A 10-day course of the anti-asprosin monoclonal antibody improves glucose clearance and body weight in DIO mice. (FIG. 36A) Glucose tolerance test results at day 11 are provided. (FIG. 36B) Body weight at day 11 is shown.

FIGS. 37A-37B: A 10-day course of the anti-asprosin monoclonal antibody improves glucose clearance and body weight in DIO mice. (FIG. 37A) Glucose tolerance test results at day 13 are provided. (FIG. 37B) Body weight at day 13 is shown.

FIGS. 38A-38D: The anti-asprosin monoclonal antibody shows a wide effective-dose range, including at doses of (FIG. 38A) 200 μg; (FIG. 38B) 100 μg; (FIG. 38C) 50 fig; and (FIG. 38D) 25 μg.

FIGS. 39A-39D: The anti-asprosin monoclonal antibody improves hyperglycemia in diabetic mice, using a variety of doses: (FIG. 39A) 200 μg; (FIG. 39B) 100 μg; (FIG. 39C) 50 μg; and (FIG. 39D) 25 μg. 11703178

FIGS. 40A-40B: The anti-asprosin monoclonal antibody is effective against a more severe diabetes model=Db mutation. (FIG. 40A) 100 μg, glucose tolerance test; (FIG. 40B) 100 μg, % body weight.

FIGS. 41A-41C: 24-hour food intake was measured upon 7 days of administration of a daily 100 ug dose of the anti-asprosin monoclonal antibody (SEQ ID NO:4) in leptin receptor knockout mice (db/db). Non-normalized data (FIG. 41A) and data normalized to body weight (FIG. 41B) or lean mass (FIG. 41C) is presented. Sending this data as an attachment.

FIG. 42—Daily body weight measurements were performed upon administration of a single 100 ug dose of the anti-asprosin monoclonal antibody (SEQ ID NO:4) in mice fed a high fat diet for 5 months, Sending this data as an attachment.

FIG. 43—Neonatal Progeroid syndrome (NPS) is associated with hypophagia. Body-mass-indices, reported and measured food intake, and energy expenditure by the doubly labeled water method and indirect calorimetry, in NPS patients compared to reference values for sedentary and active females age 24 and 18 years, respectively (Trumbo et al., 2002).

FIGS. 44A-44G: Asprosin crosses the blood-brain-barrier to stimulate appetite. (FIG. 44A) Endogenous Asprosin was measured in cerebrospinal fluid of ad libitum fed and over-night fasted rats using a sandwich Elisa (n=7 per group). (FIG. 44B) The N-terminal His-tag (on bacterially expressed asprosin) and total asprosin (recombinant+endogenous) were measured in cerebrospinal fluid of fasted rats after intravenous injection of bacterially expressed, His-tagged asprosin using a sandwich Elisa (n=4 per group). (FIG. 44C) Food intake was measured during 24 hours after a single subcutaneous injection of recombinant GFP or bacterially expressed asprosin in mice was determined using the CLAMs system (n=5 per group). Two-way ANOVA with Bonferroni post test was used to calculate the p value. (FIG. 44D) Food intake was measured during 24 hours after a single subcutaneous injection of recombinant GFP or mammalian cell expressed asprosin in mice was determined using the CLAMs system (n=6 per group). Two-way ANOVA with Bonferroni post test was used to calculate the p value. (FIG. 44E) Cumulative food intake during the dark phase (12 hours) of circadian entrained mice after intracerebroventricular (ICV) injection of recombinant GFP or bacterially expressed asprosin (n=8 per group). (FIG. 44F) Cumulative food intake over 24 hours in mice exposed to 10 days of a single daily injection of recombinant GFP or bacterially expressed asprosin was determined using the CLAMs system (n=5 per group). (FIG. 44G) Energy expenditure over 24 hours in mice from (FIG. 44E) was determined using the CLAMs system. Two-way ANOVA with Bonferroni post-test was used to calculate the p value. (FIG. 44H) Fat mass was determined using magnetic resonance imaging in mice before and after 10 days of a single daily injection of recombinant GFP or bacterially expressed asprosin in mice from (FIG. 44F). (FIG. 44I) Cumulative food intake over 24 hours in mice 10 days after adenoviral overexpression of GFP or FBN1 was determined using the CLAMs system (n=5 per group). (FIG. 44J) Energy expenditure was measured over 24 hours in mice from (FIG. 44H) using the CLAMs system. Two-way ANOVA with Bonferroni post-test was used to calculate the p value. (FIG. 44K) Fat mass was determined using magnetic resonance imaging in mice from (FIG. 44H) before and 10 days after injection with the GFP or Fbn1 adenovirus. (FIG. 44L) Fat mass was determined using magnetic resonance imaging in mice 1 week and 3 weeks after injection with the GFP or Fbn1 adenovirus (n=5 per group).

FIGS. 45A-45G—Asprosin activates orexigenic AgRP neurons. (FIG. 45A) Representative action potential (AP) firing traces of AgRP neurons after GFP and bacterially expressed recombinant asprosin treatment. (FIG. 45B) Response ratio of AgRP neurons after GFP, and 1 nM and 34 nM bacterially expressed asprosin treatment (RM≥2 mV defined as depolarized, RM≤−2 mV defined as hyperpolarized, −2 mV<RM<2 mV defined as irresponsive (n numbers as indicated in the figure). (FIG. 45C) Representative traces of miniature excitatory postsynaptic current (mESPC) in AgRP neurons before and after bacterially expressed asprosin treatment (FIG. 45D) mEPSC frequency and amplitude in AgRP neurons before and after bacterially expressed asprosin treatment (n=6 per group). (FIG. 45E) Representative traces of AgRP neuron resting membrane potential in the presence of TTX (1 μM) (top), and inhibitor cocktail (AP-5: 30 μM, CNQX: 30 μM, bicuculline: 50 μM and TTX (bottom). (FIG. 45F) Amplitude changes of resting membrane potential in AgRP neurons after treatment with GFP, 1 nM and 34 nM bacterially expressed asprosin, or 34 nM bacterially expressed asprosin in the presence of TTX and inhibitor cocktail (AP-5: 30 μM, CNQX: 30 μM, bicuculline: 50 μM and TTX 1 μM) (GFP n=8, Asprosin 1 nM n=12, 34 nM n=44, 34 nM+TTX n=11, 34 nM+TTX+CNQX+AP5+bicuculline n=13). (FIG. 45G) Response ratio of AgRP neurons after treatment with bacterially expressed asprosin in the presence of TTX or inhibitor cocktail (AP-5: 30 μM, CNQX: 30 μM, bicuculline: 50 μM and TTX 1 μM) (n numbers as indicated in the figure).

FIGS. 46A-46D—Asprosin employs the Gαₛ-cAMP-PKA pathway to activate AgRP neurons in a dose-responsive manner. (FIG. 46A) AgRP neuron firing frequency and membrane potential changes in response to increasing concentrations of asprosin produced bacterially or in mammalian cells (Firing frequency: bacterial asprosin 0.01 nM n=9, 0.1 nM n=11, 1 nM n=8, 10 nM n=12, 34 nM n=24, 100 nM n=14; mammalian asprosin: 0.01 nM n=12, 0.1 nM n=15, 1 nM n=15, 10 nM n=14, 34 nM n=15, 100 nM n=15. Membrane potential: bacterial asprosin 0.01 nM n=13, 0.1 nM n=11, 1 nM n=13, 10 nM n=10, 34 nM n=33, 100 nM n=15; mammalian asprosin: 0.01 nM n=13, 0.1 nM n=16, 1 nM n=15, 10 nM n=15, 34 nM n=16, 100 nM n=15). (FIG. 46B) Changes of AP firing frequency and membrane potential in AgRP neurons after treatment with GFP, bacterially expressed asprosin, and in the presence of different inhibitors (Firing frequency: GFP n=8, asprosin n=39, asprosin+100 μM NKY80 n=15, asprosin+50 μM suramin n=16, asprosin+20 μM NF449 n=16, asprosin+50 μM PTX n=12, asprosin+1 μM PKI n=23, asprosin+100 μM [D-Lys3]-GHRP-6 n=13. Membrane potential: GFP n=8, asprosin n=44, asprosin+100 μM NKY80 n=15, asprosin+50 μM suramin n=25, asprosin+20 μM NF449 n=16, asprosin+50 μM PTX n=13, asprosin+1 μM PKI n=24, asprosin+100 μM [D-Lys3]-GHRP-6 n=16). Two-way ANOVA with Bonferroni post-test was used to calculate the p value. (FIG. 46C) Response ratio of AgRP neurons after treatment with GFP, bacterially expressed asprosin, and in the presence of different inhibitors (n numbers as indicated in the figure). (FIG. 46D) Cumulative food intake during 24 hours after IP injection of GFP or bacterially expressed asprosin in WT control or AgRP ablated mice (n=6 per group).

FIGS. 47A-47H—Asprosin inhibits anorexigenic POMC neurons. (FIG. 47A) Representative AP firing traces of POMC neurons after GFP and bacterially expressed asprosin treatment. (FIG. 47B) Response ratio of POMC neurons after GFP, 1 nM and 34 nM bacterially expressed asprosin treatment (n numbers as indicated in the figure). (FIG. 47C) Representative miniature inhibitory postsynaptic current (mIPSC) trace of POMC neurons before and after bacterially expressed asprosin treatment in the presence of inhibitors (AP-5: 30 μM, CNQX: 30 μM and TTX: 1 μM) (FIG. 47D) mIPSC frequency and amplitude in POMC neurons before and after bacterially expressed asprosin treatment (n=10 per group). (FIG. 47E) Representative AP firing traces of POMC neurons before and after bacterially expressed asprosin in the presence of various inhibitors. (FIG. 47F) Amplitude changes of POMC membrane potential after GFP or bacterially expressed asprosin treatment only, and in the presence of different inhibitors (AP-5: 30 μM, CNQX: 30 μM and TTX: 1 μM) or (TTX: 1 μM and bicuculline: 50 μM) (GFP n=7, 1 nM asprosin n=15, 34 nM n=21, 34 nM+TTX+CNQX+AP5 n=12, 34 nM+TTX+bicuculline n=10). Two-way ANOVA with Bonferroni post-test was used to calculate the p value. (FIG. 47G) Response ratio of POMC neurons after 34 nM bacterially expressed asprosin treatment in the presence of TTX+bicuculline. (FIG. 47H) Amplitude changes of POMC neuron membrane potential and firing frequency in WT and AgRP neuron ablated mice after treatment with bacterially expressed asprosin (firing frequency: rAsprosin, WT n=8; rAsprosin, AgRP-ablated n=17. Membrane potential: rAsprosin, WT n=6; rAsprosin, AgRP-ablated n=23).

FIGS. 48A-48J—Mouse Neonatal Progeroid Syndrome Phenocopies the human disorder. (FIG. 48A) Schematic depiction of the CRISPR/Cas9 strategy employed to generate NPS mice. A small (10 bp) deletion was introduced at the junction of exon 65 and intron 65, resulting in loss of a splice site, and leading to skipping of exon 65 and truncation of profibrillin, identical to the molecular events in a known NPS patient (Jacquinet et al., 2014). (FIG. 48B) Sandwich Elisa for endogenous asprosin in plasma of WT and NPS mice (WT n=6, NPS n=7). (FIG. 48C) Representative pictures of 5-month-old male WT mice and NPS littermates, on a high fat diet for 3 months. (FIG. 48D) Body composition data using DEXA scans for WT and NPS mice on normal chow (WT n=8, NPS n=7). (FIG. 48E) Body composition data using DEXA scans for WT and NPS mice on a high fat diet for 3 months (n=8 per group). (FIG. 48F) Weight curves of WT and NPS mice 4 to 14 weeks old (n=6 per group). Two-way ANOVA with Bonferroni post-test was used to calculate the p value. (FIG. 48G) Cumulative food intake over 24 hours in mice from (FIG. 48D) in the ad libitum fed and overnight fasted state using the CLAMs system. (FIG. 48H) Energy expenditure was measured over 24 hours mice from (FIG. 48D) using the CLAMs system. Two-way ANOVA with Bonferroni post-test was used to calculate the p value. (FIG. 48I) Firing frequency and membrane potential of AgRP neurons from ad libitum fed and over-night fasted WT and NPS mice (Firing frequency: fed, WT n=24; fed, NPS n=23; fasted, WT n=25; fasted, NPS n=20. Membrane potential: fed, WT n=24; fed, NPS n=23; fasted, WT n=25; fasted, NPS n=28). (FIG. 48J) Cumulative food intake over 24 hours in WT and NPS mice after GFP injection, and NPS mice after bacterially expressed asprosin injection using the CLAMs system (n=5 per group).

FIGS. 49A-49I—Immunologic sequestration of asprosin is protective against obesity. (FIG. 49A) Firing frequency and membrane potential in AgRP neurons from mice 12 hours after isotype matched IgG or anti-asprosin monoclonal antibody injection (firing frequency: IgG n=27, anti-asprosin mAb n=26. Membrane potential: IgG n=33, anti-asprosin mAb n=34). (FIG. 49B) Cumulative food intake over 24 hours in 8-week-old male C57Bl/6 WT mice with one daily injection of isotype matched IgG or anti-asprosin mAb in the ad libitum fed and overnight fasted state using the CLAMs system (n=5 per group). (FIG. 49C) Energy expenditure over 24 hours in ad libitum fed mice from (FIG. 49B) using the CLAMs system. Two-way ANOVA with Bonferroni post-test was used to calculate the p value. (FIG. 49D) Cumulative food intake over 24 hours in 8-week-old male db/db mice with one daily injection of isotype matched IgG or anti-asprosin mAb using the CLAMs system (n=5 per group). (FIG. 49E) Energy expenditure over 24 hours in mice from (FIG. 49D) using the CLAMs system. Two-way ANOVA with Bonferroni post-test was used to calculate the p value. (FIG. 49F) Weight change over time in mice from (FIG. 49D) and littermates (n=6 per group). Two-way ANOVA with Bonferroni post-test was used to calculate the p value. (FIG. 49G) Cumulative food intake during 24 hours in 20-week-old male C57Bl/6 mice fed a high fat diet for 3 months with daily dosing for 5 days of isotype matched IgG or anti-asprosin mAb using the CLAMS system (n=5 per group). (FIG. 49H) Energy expenditure over 24 hours in mice from (FIG. 49G) using the CLAMs system. Two-way ANOVA with Bonferroni post-test was used to calculate the p value. (FIG. 49I) Weight change over time in mice from (FIG. 49G) and littermates with daily dosing for 5 days of isotype matched IgG or anti-asprosin mAb (n=6 per group). Two-way ANOVA with Bonferroni post-test was used to calculate the p value.

FIGS. 50A-50D—Mammalian asprosin is glycosylated and has a plasma half-life of approximately 145 minutes. (FIG. 50A) Immunoblot for asprosin expressed in mammalian cells (lane 2). The same sample was enzymatically deglycosylated and loaded in lane 3. Bacterially expressed asprosin was loaded as control in lane 1. (FIG. 50B) Plasma half-life of mammalian asprosin as determined by ELISA. Half-life was calculated using Graphpad Prism software. (FIG. 50C) Plasma asprosin concentrations in mice with adenoviral overexpression of GFP or FBN1 in the liver was determined using a sandwich ELISA against asprosin. (FIG. 50D) SDS PAGE gel of asprosin expressed in mammalian cells (glycosylated, ˜32 kDa) used for injections and in vitro experiments, asprosin expressed in bacteria (unglycosylated, ˜16 kDa), and GFP used as negative control (˜35 kDa).

FIGS. 51A-51I—NPS mice mimic NPS in humans. (FIG. 51A) Plasma Leptin, Adiponectin, and Ghrelin were measured using ELISA in WT and NPS mice on normal chow (n=6). (FIG. 51B) Plasma Leptin, Adiponectin, and Ghrelin were measured using ELISA in WT and NPS mice after 6 months on high fat diet (n=6). (FIG. 51C) Body weight of WT and NPS mice was measured after 6 months on high fat diet (n=6). (FIG. 51D) Plasma glucose in WT and NPS mice after 6 months on high fat diet (n=6). (FIG. 51E) Glucose tolerance test in WT and NPS mice after 6 months of high fat diet (n=6). (F) Respiratory Exchange Ratio over 24 hours in WT and NPS mice. (FIG. 51G) Heart rate, blood pressure (FIG. 51H) and body temperature (FIG. 51I) of WT and NPS mice (n=5 for NPS, n=7 for WT).

FIGS. 52A-52B—Immunofluorescence staining for c-fos in AgRP neurons. (FIG. 52A) Immunofluorescence for c-fos (top), NPY-GFP (center), and a merged composite (bottom) in the arcuate nucleus of mice receiving either IgG control, or anti-asprosin antibody followed by an overnight fast. (FIG. 52B) Double positive neurons from (FIG. 52A) were counted and quantified.

FIGS. 53A-53E—Characterization of the anti-asprosin neutralizing antibody. (FIG. 53A) SDS Page gel of the anti-asprosin mAb showing heavy chain (top band) and light chain (bottom band). Percentage of contribution to total molecular weight by heavy chain and light chain, respectively, was calculated by densitometry. (FIG. 53B) Epitope mapping for the asprosin epitope detected by the anti-asprosin mAb. The binding epitope is highlighted in red (SEQ ID NOS:25-36). (FIG. 53C) Elisa against asprosin pre-incubated with various concentrations of anti-asprosin mAb. The 50% inhibition dose (IC50) was calculated using Graphpad prism software. (FIG. 53D) Plasma glucose in mice with diet-induced obesity after a single injection of various concentrations of anti-asprosin mAb (n=6). (FIG. 53E) Plasma glucose in mice with chemical ablation of pancreatic β-cells by (Streptozotocin—STZ—treatment) in response to IgG or anti-asprosin mAb.

FIGS. 54A-54K—The anti-asprosin neutralizing antibody reversibly inhibits Asprosin's effect on AgRP and POMC neurons. (FIG. 54A) Representative traces of AgRP neurons in response to a bacterially expressed asprosin puff, followed by asprosin preincubated with 100 fold excess anti-asprosin mAb, followed by asprosin, or the same preincubated with IgG control antibody. (FIG. 54B) Firing frequency response of AgRP neurons to bacterially expressed asprosin, asprosin preincubated with anti-asprosin mAb, and response to asprosin after washout. (FIG. 54C) Membrane potential response of AgRP neurons to bacterially expressed asprosin, asprosin preincubated with anti asprosin mAb, and response to asprosin after washout. (FIG. 54D) Firing frequency response of AgRP neurons to bacterially expressed asprosin and IgG control antibody. (FIG. 54E) Membrane potential response of AgRP neurons to bacterially expressed asprosin and IgG control antibody. (FIG. 54F) Representative traces of POMC neurons in response to a bacterially expressed asprosin puff, followed by asprosin preincubated with 100 fold excess anti-asprosin mAb, followed by asprosin, or the same preincubated with IgG control antibody. (FIG. 54G) Firing frequency response of POMC neurons to bacterially expressed asprosin, asprosin preincubated with anti-asprosin mAb, and response to asprosin after washout. (FIG. 54H) Membrane potential response of POMC neurons to bacterially expressed asprosin, asprosin preincubated with anti-asprosin mAb, and response to asprosin after washout. (FIG. 54I) Firing frequency response of POMC neurons to bacterially expressed asprosin and IgG control antibody. (FIG. 54J) Membrane potential response of POMC neurons to bacterially expressed asprosin and IgG control antibody. (FIG. 54K) Elisa for plasma asprosin in 8-week-old male WT and Db/Db obese mice (WT n=5; Db/Db n=6).

FIGS. 55A-55G—Anthropometric measurements and body composition of two NPS patients. (FIG. 55A) Anthropometric measurements of two NPS patients: Body weight and height were measured and BMI was calculated from these data. * (2016); †Normal values calculated for sedentary and active females age 24 and 18 years, respectively, according to (Trumbo et al., 2002). (FIG. 55B) Body composition was measured using the total body potassium (TBK) method, dual-energy X-ray absorptiometry (DXA), and BioPod air displacement plethysmography. From these data, the body composition was calculated using the Lohman-4C model. (FIG. 55C) Total body water was measured using the doubly-labeled water method and parameters were calculated. (FIG. 55D) Energy expenditure as derived from TBW measurements. (FIG. 55E) Energy expenditure by indirect calorimetry. (FIG. 55F) Energy expenditure during 24 hours and sleep phase by indirect calorimetry. (FIG. 55G) Energy expenditure during walking at various paces and unallocated free time by indirect calorimetry.

FIGS. 56A-56B—Vitals and select hormone levels of two NPS patients. (FIG. 56A) Blood pressure (measured in triplicate), heart rate (measured in triplicate/duplicate), respirations, and body temperature of two NPS patients. * Reference values from (2014). (FIG. 56B) Plasma Leptin, Ghrelin, and Adiponectin in two NPS patients.

FIGS. 57A-57D—Intake of macro- and micronutrients calculated from dietary recall results of two NPS patients. Intake of macro- and micronutrients calculated from dietary recall results: Primary energy sources (FIG. 57A), fat and cholesterol (FIG. 57B), carbohydrates (FIG. 57C), and fiber (FIG. 57D).

FIGS. 58A-58C—Intake of macro- and micronutrients calculated from dietary recall results of two NPS patients. Intake of macro- and micronutrients calculated from dietary recall results: Vitamins (FIG. 58A), carotenoids (FIG. 58B), and minerals (FIG. 58C).

FIGS. 59A-59E—Intake of macro- and micronutrients calculated from dietary recall results of two NPS patients. Intake of macro- and micronutrients calculated from dietary recall results: Fatty acids (FIG. 59A), amino acids (FIG. 59B), isoflavones and similar (FIG. 59C), sugar alcohols/polyols (FIG. 59D), and other food contents (FIG. 59E).

FIGS. 60A-60C—Intake of vitamins, minerals, and energy sources and recommended daily intake of the same of two NPS patients as measured during indirect calorimetry. (FIG. 60A) Percent of energy derived from various energy sources measured in two NPS patients during indirect calorimetry, and % of daily recommended intake. *Recommended Intakes from (Trumbo et al., 2002). (FIG. 60B) Intake of vitamins of two NPS patients measured in two NPS patients during indirect calorimetry, and % of daily recommended intake. *Recommended Intakes from (Trumbo et al., 2002). (FIG. 60C) Intake of minerals of two NPS patients measured in two NPS patients during during indirect calorimetry, and % of daily recommended intake. *Recommended Intakes from (Trumbo et al., 2002).

### DETAILED DESCRIPTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein embodiments which are disclosed and still obtain a like or similar result without departing from the spirit and scope of the invention.

“Effective amount” and “therapeutically effective amount” are used interchangeably herein, and refer to an amount of an antibody or functional fragment thereof, as described herein, effective to achieve a particular biological or therapeutic result such as, but not limited to, the biological or therapeutic results disclosed herein. A therapeutically effective amount of the antibody or antigen-binding fragment thereof may vary according to factors such as the disease state, age, sex, and weight of the individual, and the ability of the antibody or functional fragment thereof to elicit a desired response in the individual. Such results may include, but are not limited to, the treatment of cancer, as determined by any means suitable in the art.

### I. Asprosin

Embodiments of the disclosure include methods and compositions related to asprosin, which is a C-terminal cleavage fragment of fibrillin-1. A sequence of native human asprosin (amino acids 2732-2871 of fibrillin-1) is as follows: STNETDASNIEDQSETEANVSLASWDVEKTAIFAFNISHVSNKVRILELLPALTTLTNHNR YLIESGNEDGFFKINQKEGISYLHFTKKKPVAGTYSLQISSTPLYKKKELNQLEDKYDKD YLSGELGDNLKMKIQVLLH (SEQ ID NO:1). Asprosin may be isolated from human cells, and therefore no longer residing in nature, or it may be recombinant, in certain embodiments. As referred to herein, when the native sequence of SEQ ID NO:1 is generated by recombinant means, the resultant polypeptide may be referred to as a recombinant asprosin. A sequence of another example of a recombinant asprosin includes a label or tag. As an example, a His tag attached at N-terminus along with a methionine to include a start codon for translation in E. coli is as follows: MHHHHHHSTNETDASNIEDQSETEANVSLASWDVEKTAIFAFNISHVSNKVRILELLPAL TTLTNHNRYLIESGNEDGFFKINQKEGISYLHFTKKKPVAGTYSLQISSTPLYKKKELNQL EDKYDKDYLSGELGDNLKMKIQVLLH (SEQ ID NO:2). Embodiments of asprosin include functional derivatives or functional fragments thereof, and the derivative or fragment may be considered functional if it has the ability to increase appetite and/or weight gain in a mammal when provided to the mammal in an effective amount. Such an activity may be measured by any suitable means, including MRI scans to assess increase in adipose mass or measurements of body weight using a weighing scale, for example. In particular embodiments, one can assess functional activity by assaying for promotion of adipocyte differentiation in vitro, for example. In specific embodiments, the asprosin or functional fragment or functional derivative is soluble. The asprosin or functional fragment or functional derivative may be comprised in a fusion protein.

Asprosin proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. An asprosin coding region (such as within fibrillin-1, although it may be separated from fibrillin-1) may be amplified and/or expressed using the techniques disclosed herein or as would be known to those of ordinary skill in the art. Alternatively, various commercial preparations of proteins, polypeptides and peptides are known to those of skill in the art.

In certain embodiments an asprosin (or fragment or derivative thereof) proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide. Biological functional equivalents of asprosin, including such derivatives and fragments, may be employed. As modifications and/or changes may be made in the structure of asprosin polynucleotides and and/or proteins according to the present invention, while obtaining molecules having similar or improved characteristics, such biologically functional equivalents are also encompassed within the present invention.

An asprosin functional derivative or fragment thereof may comprise 1, 2, 3, 4, 5, 6, 7, 8, 9, 10, 11, 12, 13, 14, 15, 16, 17, 18, 19, or 20 or more amino acid alterations compared to SEQ ID NO:1. The asprosin functional derivative or fragment thereof may comprise an N-terminal truncation of SEQ ID NO:1, for example wherein the truncation is no more than 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids or wherein the truncation is at least 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids. The asprosin functional derivative or fragment thereof may comprise a C-terminal truncation of SEQ ID NO:1, such as wherein the truncation is no more than 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids or is at least 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids. The asprosin functional derivative or fragment thereof may comprise an internal deletion in SEQ ID NO:1, such as wherein the internal deletion is no more than 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids or is at least 1, 2, 3, 4, 5, 6, 7, 8, 9, 10, 12, 15, 20, 25, 30, 35, 40, 45, 50, 55, 60, 65, 70, 75, 80, 85, 90, 95, or 100 amino acids. In specific embodiments, an asprosin functional derivative or fragment thereof may comprise sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO:1.

In specific embodiments, an appetite stimulant comprises asprosin or a functional fragment or functional derivative. The stimulant may be specifically formulated with asprosin to stimulate the appetite of a mammalian individual. Such a stimulant may be provided to an individual that is underweight, undernourished, underfed, that is trying to build up mass, to increase mass of agricultural animals (such as cows, pigs, lambs, chickens, etc.), for bodybuilders, and so forth. The stimulant composition may have other stimulants than asprosin.

A. Modified Polynucleotides and Polypeptides

A biological functional equivalent of asprosin may be produced from a polynucleotide that has been engineered to contain distinct sequences while at the same time retaining the capacity to encode the “wild-type” or standard protein. This can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a protein.

In another example, an asprosin polynucleotide made be (and encode) a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or codons) may be substituted.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (0.4); threonine (0.7); serine (0.8); tryptophan (0.9); tyrosine (1.3); proline (1.6); histidine (3.2); glutamate (3.5); glutamine (3.5); aspartate (3.5); asparagine (3.5); lysine (3.9); and/or arginine (4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (0.4); proline (−0.5±1); alanine (0.5); histidine (0.5); cysteine (1.0); methionine (1.3); valine (1.5); leucine (1.8); isoleucine (1.8); tyrosine (2.3); phenylalanine (2.5); tryptophan (3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

B. Altered Amino Acids

The present invention, in many aspects, relies on the synthesis of peptides and polypeptides in cyto, via transcription and translation of appropriate polynucleotides. These peptides and polypeptides will include the twenty “natural” amino acids, and post-translational modifications thereof. However, in vitro peptide synthesis permits the use of modified and/or unusual amino acids. Exemplary, but not limiting, modified and/or unusual amino acids are known in the art.

C. Mimetics

In addition to the biological functional equivalents discussed above, the present inventors also contemplate that structurally or functionally similar compounds may be formulated to mimic the key portions of peptide or polypeptides of the present invention. Such compounds, which may be termed peptidomimetics, may be used in the same manner as the peptides of the invention and, hence, also are functional equivalents. In specific embodiments, the mimetic comprises one or more beta pleats from asprosin.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule. Such peptidomimetics include compounds that do not incorporate any natural amino acids or amino acid side chains, but are designed based on the asprosin peptide sequence and have the ability to functionally replace asprosin.

### II. Inhibitors of Asprosin or of the Asprosin Receptor

Embodiments of the disclosure include one or more inhibitors of asprosin. In specific embodiments, the inhibitor is an antibody or binding fragment thereof, although in some cases the inhibitor is not an antibody. In specific embodiments, the inhibitor may be one or more small molecules, one or more aptamers, one or more non-antibody phage display-derived peptides, a combination thereof, and so forth. In specific embodiment, an inhibitor of asprosin specifically binds and inactivates asprosin. In specific embodiments, the inhibitor is soluble. In some embodiments, there are methods and compositions for soluble receptor-mediated inhibition of asprosin. In particular embodiments, RNAi- and/or microRNA-mediated inhibition may be employed, for example in particular embodiments wherein asprosin has its own transcriptional unit separate from FBN1.

Embodiments of the disclosure include one or more inhibitors of the asprosin receptor(s). In specific embodiments, the inhibitor is an antibody, although in some cases the inhibitor is not an antibody. In specific embodiments, the inhibitor may be one or more small molecules, one or more aptamers, one or more non-antibody phage display-derived peptides, RNAi or microRNA mediated inhibitors, specific inhibitors of its downstream signaling, or a combination thereof, and so forth. In specific embodiment, an inhibitor of the asprosin receptor specifically binds and inactivates asprosin. In one specific embodiment it specifically blocks its expression or otherwise decreases its functional activity. In specific embodiments, the inhibitor is soluble.

In specific embodiments, the inhibitor targets a structural or functional motif, and the asprosin target site of the inhibitor may or may not be known. In specific embodiments, the inhibitor targets one or more beta pleats from asprosin. In specific embodiments, the inhibitor of asprosin is an inhibitor of the receptor for asprosin.

In certain embodiments, there is an appetite suppressant that comprises one or more asprosin inhibitors. The suppressant composition may have other suppressants than asprosin. The suppressant may be specifically formulated with asprosin to suppress the appetite of a mammalian individual. Such a suppressant may be provided to an individual that is overweight, obese, has diabetes, is at risk for becoming overweight, is at risk for becoming obese, and so forth.

In particular embodiments, the inhibitor is an antibody or binding fragment thereof. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference). Antibodies of the disclosure may specifically bind their target. The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

A. Polyclonal Antibodies

Polyclonal antibodies to asprosin generally may be raised in animals by multiple subcutaneous (sc) or intraperitoneal (ip) injections of asprosin or a fragment thereof and an adjuvant. It may be useful to conjugate the asprosin or a fragment containing the target amino acid sequence to a protein that is immunogenic in the species to be immunized, e.g. keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaraldehyde, succinic anhydride, SOCl₂, or R¹N═C═NR, where R and R¹ are different alkyl groups.

Animals may be immunized against the immunogenic conjugates or derivatives by combining 1 mg of 1 μg of conjugate (for rabbits or mice, respectively) with 3 volumes of Freud's complete adjuvant and injecting the solution intradermally at multiple sites. One month later the animals are boosted with ⅕ to 1/10 the original amount of conjugate in Freud's complete adjuvant by subcutaneous injection at multiple sites. 7 to 14 days later the animals are bled and the serum is assayed for anti-asprosin antibody titer. Animals are boosted until the titer plateaus. Preferably, the animal boosted with the conjugate of the same asprosin, but conjugated to a different protein and/or through a different cross-linking reagent. Conjugates also can be made in recombinant cell culture as protein fusions. Also, aggregating agents such as alum are used to enhance the immune response.

B. Monoclonal Antibodies

In specific embodiments, monoclonal antibodies may be generated and employed as inhibitors of asprosin for the use in an individual. In some cases, the antibodies are used in methods of losing weight, treating insulin resistance, type II diabetes, or metabolic syndrome or for use in a person that is obese or overweight. The immunogen for the monoclonal antibodies may be the entire asprosin polypeptide or may be a fragment thereof.

Exemplary sequences of immunogens that may be employed for the generation of monoclonal antibodies are as follows:

(SEQ ID NO: 3)
HuFbn1-2746: 2770 ETEANVSLASWDVEKTAIFAFNISH
(SEQ ID NO: 4)
HuFbn1 2838: 2865 KKKELNQLEDKYDKDYLSGELGDNLKMK

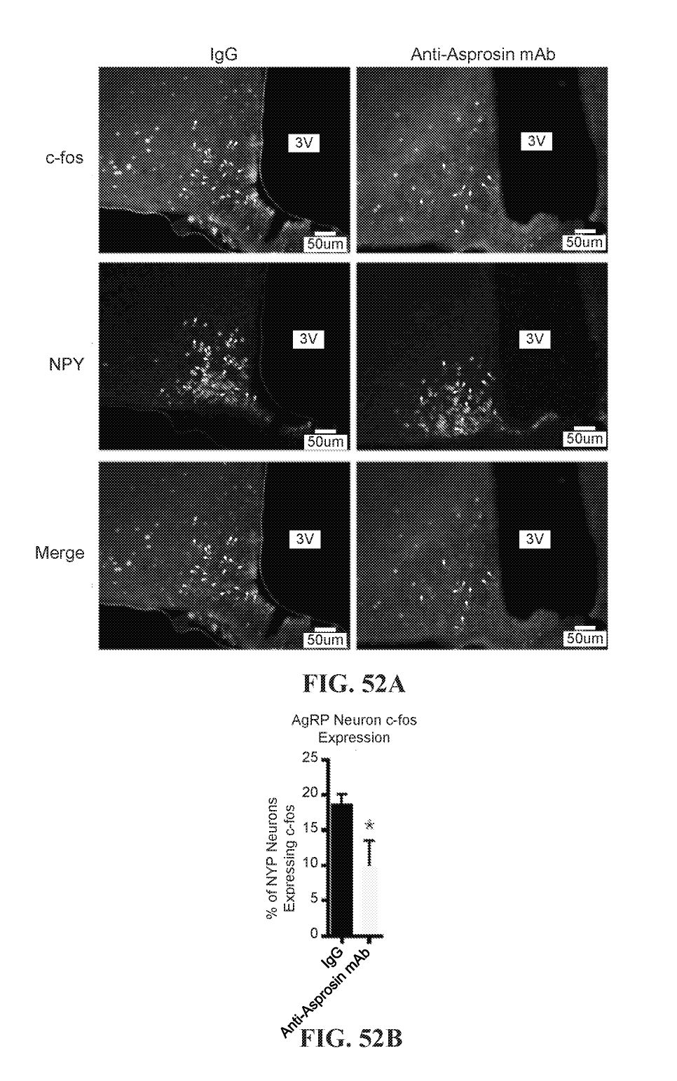

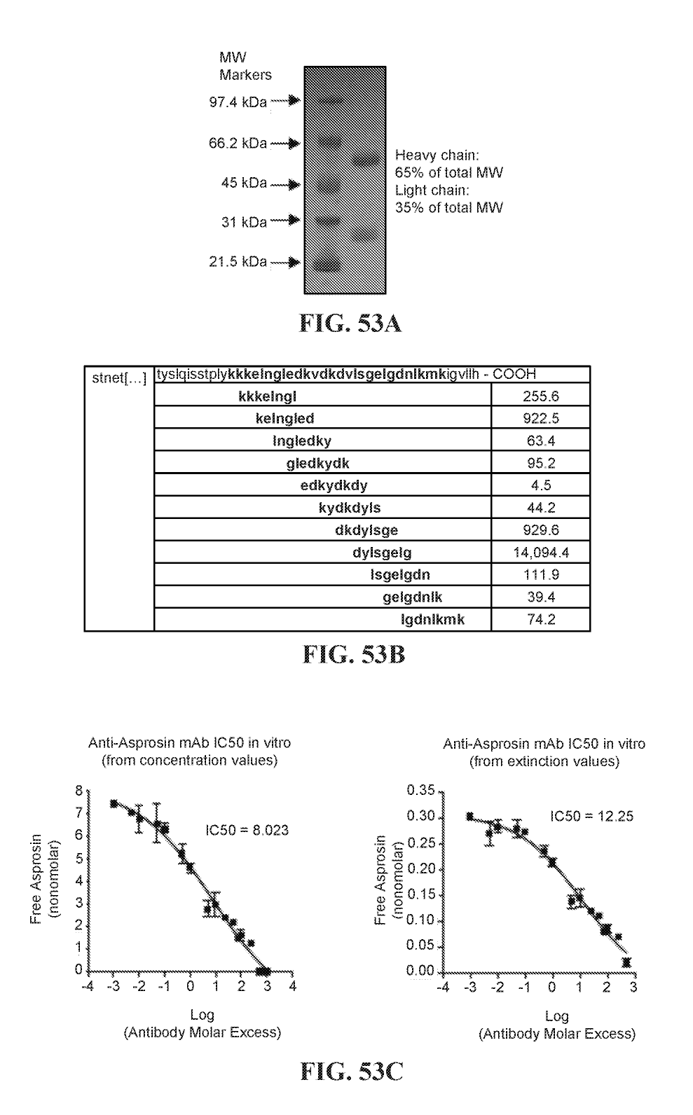

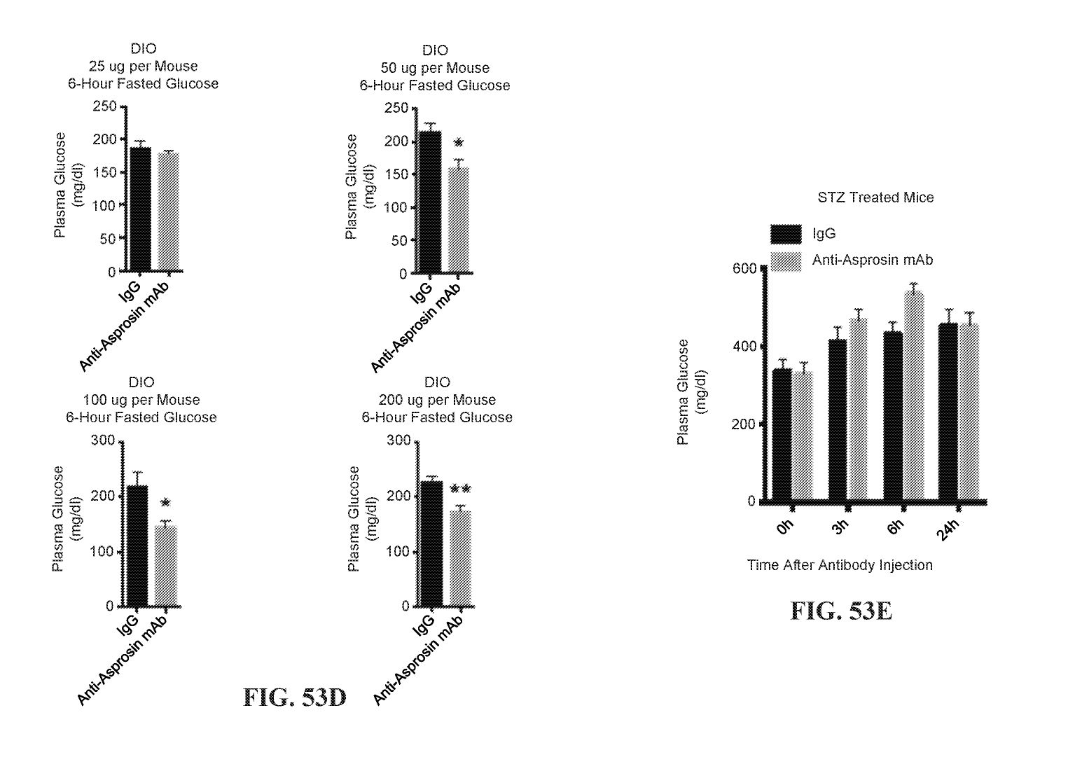

In specific embodiments, an antibody binds an epitope on the amino acid sequence of SEQ ID NO:4. The epitope may be all of the amino acid sequence of SEQ ID NO:4 or it may be a fragment of SEQ ID NO:4. The epitope may be a fragment of SEQ ID NO:4, as noted in FIG. 53B, for example. In specific embodiments, the epitope is a continuous sequence of amino acids, although in some cases the epitope binds a three-dimensional configuration of amino acid sequences that may or may not be continuous in form. In some cases, the epitope is between 5 and 20, 5 and 15, 5 and 10, 8 and 20, 8 and 15, 8 and 10, 10 and 20, or 10 and 15 amino acids in length. The epitope may comprise, consist of, or consist essentially of 5, 6, 7, 8, 9, 10, 11, 12, 13, 14, 15, 16, 17, 18, 19, or 20 or more amino acids of SEQ ID NO:4, and in some embodiments the amino acids are continuous in SEQ ID NO:4 whereas in other cases they are not continuous in SEQ ID NO:4.

In particular embodiments, there is an isolated antibody, including a monoclonal antibody or scFv, for example, that specifically binds a peptide comprising, consisting essentially of, or consisting of SEQ ID NO:4. In some cases, the antibody is an isolated antibody or antigen-binding portion that specifically binds a peptide comprising, consisting essentially of, or consisting of SEQ ID NO:4. Embodiments of the disclosure include antibodies produced by the hybridoma cell line having deposit accession number ATCC PTA-123085. In particular embodiments, the antibody comprises the same heavy and light chain polypeptide sequences as an antibody produced by hybridoma having deposit accession number ATCC PTA-123085. The disclosure also encompasses one or more isolated cells of a hybridoma having deposit accession number ATCC PTA-123085 and also the hybridoma cell line having ATCC deposit number PTA-123085. Antibodies produced by any cell lines of the disclosure (including humanized forms) are encompassed herein. Specific embodiments include isolated and purified monoclonal antibodies produced by the continuous hybridoma cell line having deposit accession number PTA-123085.

Monoclonal antibodies may be obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

For example, the anti-asprosin monoclonal antibodies of the invention may be made using the hybridoma method first described by Kohler & Milstein, Nature 256:495 (1975), or may be made by recombinant DNA methods [Cabilly, et al., U.S. Pat. No. 4,816,567]. In the hybridoma method, a mouse or other appropriate host animal, such as hamster is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell [Goding, Monoclonal Antibodies: Principles and Practice, pp. 59-103 (Academic Press, 1986)].

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies directed against asprosin. Preferably, the binding specificity of monoclonal antibodies produced by hybridoma cells is determined by immunoprecipitation or by an in vitro binding assay, such as radioimmunoassay (MA) or enzyme-linked immunoabsorbent assay (ELISA).

The binding affinity of the monoclonal antibody can, for example, be determined by the Scatchard analysis of Munson & Pollard, Anal. Biochem. 107:220 (1980).

After hybridoma cells are identified that produce antibodies of the desired specificity, affinity, and/or activity, the clones may be subcloned by limiting dilution procedures and grown by standard methods. Goding, Monoclonal Antibodies: Principles and Practice, pp. 59-104 (Academic Press, 1986). Suitable culture media for this purpose include, for example, Dulbecco's Modified Eagle's Medium or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison, et al., Proc. Nat. Acad. Sci. 81, 6851 (1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-asprosin monoclonal antibody herein.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for asprosin and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

For diagnostic applications, the antibodies of the invention typically may be labeled with a detectable moiety. The detectable moiety can be any one which is capable of producing, either directly or indirectly, a detectable signal. For example, the detectable moiety may be a radioisotope such as ³H, ¹⁴C, ³²P, ³⁵S, or ¹²⁵I, a fluorescent or chemiluminescent compound, such as fluorescein isothiocyanate, rhodamine, or luciferin; biotin; radioactive isotopic labels, such as, e.g., ¹²⁵I, ³²P, ¹⁴C, or ³H, or an enzyme, such as alkaline phosphatase, beta-galactosidase or horseradish peroxidase.

Any method known in the art for separately conjugating the antibody to the detectable moiety may be employed, including those methods described by Hunter, et al., Nature 144:945 (1962); David, et al., Biochemistry 13:1014 (1974); Pain, et al., J. Immunol. Meth. 40:219 (1981); and Nygren, J. Histochem. and Cytochem. 30:407 (1982).

The antibodies of the present invention may be employed in any known assay method, such as competitive binding assays, direct and indirect sandwich assays, and immunoprecipitation assays. Zola, Monoclonal Antibodies: A Manual of Techniques, pp. 147-158 (CRC Press, Inc., 1987).

Competitive binding assays rely on the ability of a labeled standard (which may be an asprosin or an immunologically reactive portion thereof) to compete with the test sample analyte (asprosin) for binding with a limited amount of antibody. The amount of asprosin in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex. David & Greene, U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

C. Humanized Antibodies

In particular embodiments, antibodies against asprosin are humanized. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature 321, 522-525 (1986); Riechmann et al., Nature 332, 323-327 (1988); Verhoeyen et al., Science 239, 1534-1536 (1988)], by substituting rodent CDRs or CDR sequences for the corresponding sequences of a human antibody. Accordingly, such “humanized” antibodies are chimeric antibodies (Cabilly, supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding. For further details see U.S. application Ser. No. 07/934,373 filed Aug. 21, 1992, which is a continuation-in-part of application Ser. No. 07/715,272 filed Jun. 14, 1991.

D. Human Antibodies

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984), and Brodeur, et al., Monoclonal Antibody Production Techniques and Applications, pp. 51-63 (Marcel Dekker, Inc., New York, 1987).

It is now possible to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (J.sub.H) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g. Jakobovits et al., Proc. Natl. Acad. Sci. USA 90, 2551-255 (1993); Jakobovits et al., Nature 362, 255-258 (1993).

Alternatively, the phage display technology (McCafferty et al., Nature 348, 552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle.

Because the filamentous particle contains a single-stranded DNA copy of the phage genome, selections based on the functional properties of the antibody also result in selection of the gene encoding the antibody exhibiting those properties. Thus, the phage mimicks some of the properties of the B-cell. Phage display can be performed in a variety of formats; for their review see, e.g. Johnson, Kevin S. and Chiswell, David J., Current Opinion in Structural Biology 3, 564-571 (1993). Several sources of V-gene segments can be used for phage display. Clackson et al., Nature 352, 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222, 581-597 (1991), or Griffith et al., EMBO J. 12, 725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10, 779-783 [1992]). In this method, the affinity of “primary” human antibodies obtained by phage display can be improved by sequentially replacing the heavy and light chain V region genes with repertoires of naturally occurring variants (repertoires) of V domain genes obtained from unimmunized donors. This techniques allows the production of antibodies and antibody fragments with affinities in the nM range. A strategy for making very large phage antibody repertoires (also known as “the mother-of-all libraries”) has been described by Waterhouse et al., Nucl. Acids Res. 21, 2265-2266 (1993), and the isolation of a high affinity human antibody directly from such large phage library is reported by Griffith et al., EMBO J. (1994), in press. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e. the epitope governs (imprints) the choice of partner. When the process is repeated in order to replace the remaining rodent V domain, a human antibody is obtained (see PCT patent application WO 93/06213, published Apr. 1, 1993). Unlike traditional humanization of rodent antibodies by CDR grafting, this technique provides completely human antibodies, which have no framework or CDR residues of rodent origin.

E. Bispecific Antibodies

Bispecific antibodies are monoclonal, preferably human or humanized, antibodies that have binding specificities for at least two different antigens. In the present case, one of the binding specificities is for asprosin, the other one is for any other antigen, and preferably for another receptor or receptor subunit. For example, bispecific antibodies specifically binding asprosin and an asprosin receptor or two different asprosin receptors are within the scope of the present invention.

Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, Nature 305, 537-539 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published May 13, 1993), and in Traunecker et al., EMBO 10, 3655-3659 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in copending application Ser. No. 07/931,811 filed Aug. 17, 1992.

For further details of generating bispecific antibodies see, for example, Suresh et al., Methods in Enzymology 121, 210 (1986).

F. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

### III. Individuals in Need of Weight Gain

Embodiments of the disclosure include methods and compositions for increasing weight in an individual in need of weight gain. The individual may be in need of an increase in adipose mass, for example. The individual may be in need of weight gain for a variety of reasons, including because of a medical condition or state or for another reason. In cases wherein the individual is underweight because of a medical condition, the medical condition may or may not be a genetic condition or may or may not be an inherited condition. The cause of being underweight may be because of genetics, metabolism, and/or illness, in specific embodiments. In specific embodiments, the medical condition has being underweight as a symptom. In some cases, the symptom of being underweight is present in all individuals with the medical condition, although it may be present in less than all individuals with the medical condition. The symptom of being underweight may be because of a defect in pathways related to adipose metabolic regulation, fat storage, and inflammatory processes, although in some cases being underweight is not directly related to adipose metabolic regulation, fat storage, and inflammatory processes. The individual may be underweight because of Neonatal Progeroid Syndrome, Marfan Syndrome, HIV infection, hyperthyroidism, cancer, tuberculosis, gastrointestinal or liver problems, medicine side effect, or mental illness, such as those with anorexia nervosa or bulimia nervose, in some cases. For example, an individual that has cachexia may be subjected to methods and compositions of the disclosure. The cachexia may be the result of any reason, including, for example, from cancer, AIDS, chronic obstructive lung disease, multiple sclerosis, congestive heart failure, tuberculosis, familial amyloid polyneuropathy, mercury poisoning, hormonal deficiency, and so forth.

In specific embodiments, an individual in need of weight gain is an individual with a body mass index (BMI) of under 18.5 or a weight 15% to 20% below that normal for their age and height group. The individual that is subjected to methods and compositions of the disclosure may first be identified by a medical practitioner as being in need of weight gain, and the therapeutic composition may be delivered to the individual for the specific purpose of increasing weight.

### IV. Treatment of Individuals in Need of Weight Gain

In embodiments of the disclosure an individual is determined to be in need of weight gain, such as by measuring their weight and/or by measuring their BMI and/or having an MRI and/or dual-energy x-ray absorptiometry (DEXA) scans for measurement of adipose mass. The individual may be known to be in need of weight gain or suspected of being in need of weight gain or at risk for being in need of weight gain. An individual may determine themselves that they are in need of weight gain and/or it may be determined by a suitable medical practitioner.

Once the individual is known to be in need of weight gain or known to be at risk or susceptible to being in need of weight gain, they may be given a suitable and effective amount of asprosin or a functional derivative or a functional fragment. In specific embodiments, one or more of asprosin or a functional derivative or a functional fragment are provided to the individual, such as in a composition or in multiple compositions. A composition comprising asprosin or a functional derivative or a functional fragment may be specifically formulated for a therapeutic application.

An individual may be provided suitable dose(s) of asprosin on an as needed basis or as part of a routine regimen. The individual may also be taking other measures and/or compositions to gain weight in addition to taking asprosin or a functional derivative or a functional fragment. The individual may take asprosin or a functional derivative or a functional fragment on a daily basis, weekly basis, monthly basis, and so on. The individual may take asprosin or a functional derivative or a functional fragment with consumption of food or on an empty stomach.

The individual may or may not be monitored by a medical practitioner during the course of an asprosin or a functional derivative or a functional fragment regimen. The individual may cease to take asprosin or a functional derivative or a functional fragment once a desirable weight is achieved and may resume taking asprosin or a functional derivative or a functional fragment if the individual becomes in need of gaining weight at a later point in time. In the event that an individual exceeds a suitable amount of asprosin or a functional derivative or a functional fragment such that too much weight is gained, the individual may reduce their weight by any suitable means, including by exercise, reducing caloric intake, and/or taking an inhibitor of asprosin, for example.

V. Individuals in Need of Weight Loss and/or in Need of Improved Glucose Control

Embodiments of the disclosure include methods and compositions for decreasing weight in an individual in need of weight loss. The individual may be in need of a decrease in adipose mass, for example. The individual may be in need of weight loss for a variety of reasons, including because of a medical condition or state or for another reason. In cases wherein the individual is in need of weight loss because of a medical condition, the medical condition may or may not be a genetic condition and may or may not be an inherited condition. The cause of being in need of weight loss may be from genetics, metabolism, and/or illness. In specific embodiments, the medical condition has being overweight or obese as a symptom. In some cases, the symptom of being overweight or obese is present in all individuals with the medical condition, although it may be present in less than all individuals with the medical condition. The symptom of being overweight or obese may be because of a defect in pathways related to adipose metabolic regulation, fat storage, and inflammatory processes, although in some cases being overweight or obese is not directly related to adipose metabolic regulation, fat storage, and inflammatory processes. The individual may be overweight or obese because of diabetes; hypothyroidism; metabolic disorders, including metabolic syndrome; medication side effects; alcoholism; eating disorder; insufficient sleep; limited physical exercise; sedentary lifestyle; poor nutrition; addiction cessation; and/or stress; although in some embodiments such conditions are the result of being overweight or obese.

In some methods, an individual is in need of modulation of hepatic glucose release; such embodiments may modulate (such as activate) pathways that control rapid glucose release into the circulation. In particular embodiments, an individual has a defect in glucose control and is determined to need an improvement in such defect. In specific embodiments, the defect in glucose control is that there is an excessive amount of glucose in the blood of the individual. In particular embodiments, an individual has diabetes or is pre-diabetic and may or may not also be overweight or obese. The individual is provided an effective amount of one or more of any inhibitors of asprosin to improve blood glucose control, in specific embodiments, including to reduce the level of excessive blood glucose. Such treatment is provided to the diabetic or pre-diabetic individual and an improvement in blood glucose control occurs. The decrease in blood glucose level may or may not be too normal blood glucose levels. In particular embodiments, in addition to an improvement in blood glucose control, one or more symptoms of diabetes or pre-diabetes is improved upon administration of one or more inhibitors of asprosin. Some methods of the disclosure treat insulin resistance, such as by reducing levels of asprosin, including plasma levels of asprosin. For pre-diabetic individuals, the onset of diabetes is prevented upon use of one or more inhibitors of asprosin. For insulin-resistant individuals, asprosin inhibition results in restoration or improvement of insulin sensitivity, resulting in better glucose clearance, in specific embodiments.

In specific embodiments, an individual in need of weight loss is overweight (BMI between 25 and 29) or obese (BMI of 30 or more). The individual that is subjected to methods and compositions of the disclosure may first be identified by a medical practitioner as being in need of weight loss, and the therapeutic composition may be delivered to the individual for the specific purpose of decreasing weight.

In embodiments of the disclosure, the administration of asprosin or a functional derivative or a functional fragment to an individual does not result in the onset of diabetes in the individual. In specific embodiments, the individual has diabetes or does not have diabetes.

### VI. Treatment of Individuals in Need of Weight Loss

In embodiments of the disclosure an individual is determined to be in need of weight loss, such as by measuring their weight and/or by measuring their BMI and/or having an MRI and/or DEXA scan for assessment of adipose mass. The individual may be known to be in need of weight loss or suspected of being in need of weight loss or at risk for being in need of weight loss. An individual may determine themselves that they are in need of weight loss and/or it may be determined by a suitable medical practitioner.

Once the individual is known to be in need of weight loss or known to be at risk or susceptible to being in need of weight loss, they may be given a suitable and effective amount of an inhibitor of asprosin. In specific embodiments, one or more asprosin inhibitors are provided to the individual, such as in a composition or in multiple compositions. A composition comprising asprosin inhibitor may be specifically formulated for a therapeutic application.

An individual may be provided suitable dose(s) of asprosin inhibitor on an as needed basis or as part of a routine regimen. The individual may also be taking other measures and/or compositions to lose weight in addition to taking asprosin inhibitor. The individual may take asprosin inhibitor on a daily basis, weekly basis, monthly basis, and so on. The individual may take asprosin inhibitor with consumption of food or on an empty stomach.

The individual may or may not be monitored by a medical practitioner during the course of an asprosin inhibitor regimen. The individual may cease to take asprosin inhibitor once a desirable weight is achieved and may resume taking asprosin inhibitor if the individual becomes in need of losing weight at a later point in time. In the event that an individual exceeds a suitable amount of asprosin inhibitor such that too much weight is lost, the individual may increase their weight by any suitable means, including by increasing caloric intake and/or taking asprosin or a functional fragment or functional derivative, for example.

### VII. Diagnosis of Individuals in Need of Weight Modulation

In certain embodiments, an individual is diagnosed as being in need of an increase in weight or is diagnosed as being susceptible to needing an increase in weight based on the level of asprosin in their body (including in their plasma, for example). A suitable sample may be obtained from the individual and processed either by the party that obtains the sample or by a third party. The sample may be stored and/or transported under suitable conditions prior to analysis. In certain embodiments, when the level of asprosin is determined to be below a certain level, the individual is known to be in need of weight gain or is known to be susceptible to being in need of weight gain, and a suitable amount of asprosin or a functional fragment or functional derivative thereof is provided to the individual. In specific embodiments, a diagnosis is made based on asprosin level not to identify that the individual is in need of weight gain or susceptible to being in need of weight gain but instead for the cause of there being in need of weight gain or susceptibility thereof.

In certain embodiments, an individual is diagnosed as being in need of a decrease in weight or is diagnosed as being susceptible to needing a decrease in weight based on the level of asprosin in their body (including in their plasma, for example). A suitable sample may be obtained from the individual and processed either by the party that obtains the sample or by a third party. The sample may be stored and/or transported under suitable conditions prior to analysis. In certain embodiments, when the level of asprosin is determined to be above a certain level, the individual is known to be in need of weight loss or is known to be susceptible to being in need of weight loss, and a suitable amount of one or more asprosin inhibitors is provided to the individual. In specific embodiments, a diagnosis is made based on asprosin level not to identify that the individual is in need of weight loss or susceptible to being in need of weight loss but instead for the cause of there being in need of weight loss or susceptibility thereof. In specific cases, obese individuals may have duplications of fibrillin-1 (or a region thereof) that causes production of excessive asprosin.

Any suitable means to identify levels of asprosin in the body may be employed. In specific embodiments, sandwich ELISA, western blot, competitive radiolabel binding assay, receptor activity assay, and/or measurement of asprosin-induced intra/extracellular signaling cascades are employed to identify plasma levels of asprosin.

Embodiments of the disclosure utilize antibodies for detection of asprosin in an individual. The antibody may or may not be immobilized on a substrate, such as a plate, well, bead, chip, and so forth. The detection may be qualitative or quantitative, and quantitative methods determine the level of asprosin in an individual or a sample from the individual that is representative of the level of asprosin or representative of a certain medical state or condition. The level may be determined in detecting a complex between an antibody that specifically binds asprosin and asprosin. Examples of methods of measuring the level of asprosin in a sample from an individual include contacting an antibody or antibody fragment that specifically binds asprosin (such as a peptide comprising, consisting of, or consisting essentially of SEQ ID NO:4) with a sample, then forming a complex between the antibody and asprosin from the sample, and then detecting the antibody/asprosin complex and determining the level of asprosin in the sample. The antibody may be that produced by hybridoma cell line deposited with the American Type Culture Collection under accession number ATCC PTA-123085, for example. Once the level of asprosin is determined, one can employ a corresponding therapy for the individual, such as an inhibitor for an individual having elevated levels of asprosin (such as elevated compared to a sample from an individual with normal levels), or one can employ a corresponding therapy for an individual with reduced levels of asprosin (such as reduced compared to a sample from an individual with normal levels, and the level may be compared to a range of normal levels).

### VIII. Pharmaceutical Preparations

Pharmaceutical compositions of the present invention comprise an effective amount of one or more of asprosin (or functional fragment or functional derivative) or of one or more asprosin inhibitors dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one asprosin (or functional fragment or functional derivative) or at least one asprosin inhibitor will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The asprosin (or functional fragment or functional derivative) or asprosin inhibitor may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The asprosin (or functional fragment or functional derivative) or asprosin inhibitor may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include asprosin (or functional fragment or functional derivative) or asprosin inhibitor, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the asprosin (or functional fragment or functional derivative) or asprosin inhibitor may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the asprosin (or functional fragment or functional derivative) or asprosin inhibitor are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, asprosin (or functional fragment or functional derivative) or asprosin inhibitor may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound asprosin (or functional fragment or functional derivative) or asprosin inhibitor may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IX. Kits of the Disclosure

Any of the compositions described herein may be comprised in a kit. In a non-limiting example, asprosin (or functional fragment or functional derivative) and/or asprosin inhibitor may be comprised in a kit. The kits will thus comprise, in suitable container means, an asprosin (or functional fragment or functional derivative) and/or asprosin inhibitor.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the asprosin (or functional fragment or functional derivative) and/or asprosin inhibitor and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The asprosin (or functional fragment or functional derivative) or asprosin inhibitor compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The kits of the present invention will also typically include a means for containing the vials in close confinement for commercial sale, such as, e.g., injection and/or blow-molded plastic containers into which the desired vials are retained.

The kit may comprise asprosin (or functional fragment or functional derivative) or asprosin inhibitor formulated as an appetite stimulant or appetite suppressant, respectively.

In specific embodiments, the kit further comprises one or more compositions for weight loss or weight gain, including appetite suppressants or appetite stimulants, for example. In certain embodiments, the kit comprises one or more apparatuses and/or reagents for obtaining a sample from an individual and/or processing thereof.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### An Adipose-Derived Polypeptide Hormone Critical for Maintaining Optimal Fat Mass

Neonatal Progeroid Syndrome (NPS) Associated Lipodystrophy—

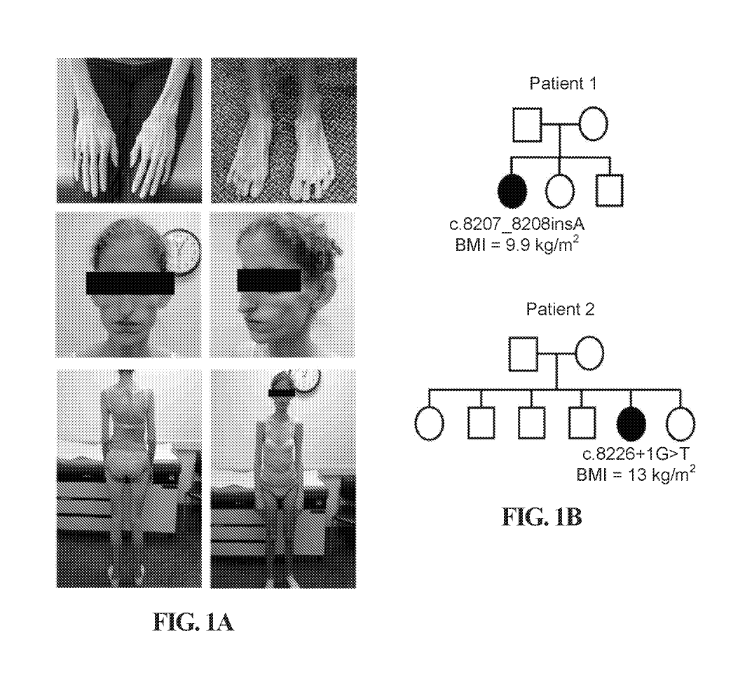

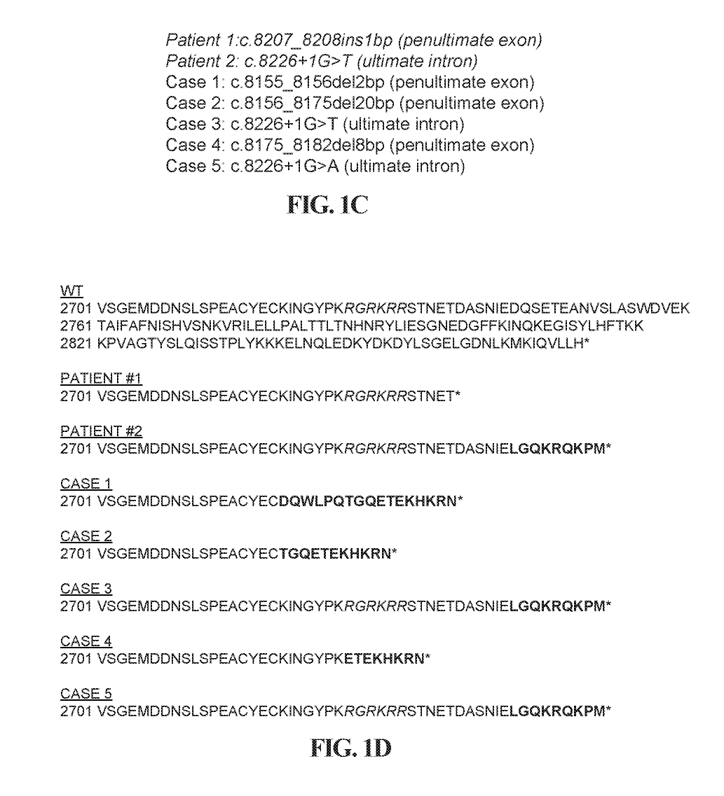

NPS is characterized by congenital, extreme thinness due to a reduction in subcutaneous adipose tissue, predominantly affecting the face and extremities (Hou, et al., 2009; O'Neill, et al., 2007). The phenotype is typically apparent at birth (and even before birth as intrauterine growth retardation) with thin skin and prominent vasculature due to paucity of subcutaneous fat (O'Neill, et al., 2007). Patients display a body mass index (BMI) several standard deviations below normal for age, at all ages (O'Neill, et al., 2007). Although NPS patients appear progeroid, due to facial dysmorphic features and reduced subcutaneous fat, they do not have the usual features of true progeria such as cataracts, premature greying of hair or insulin resistance (O'Neill, et al., 2007). Through clinical examination two individuals were identified with NPS and the mechanism that drives their extreme thinness phenotype is characterized herein. Both patients have extremely low BMIs (FIG. 1B), and grossly display reduced subcutaneous fat predominantly affecting the face and limbs with relative sparing in the gluteal area (FIG. 1A). They are the only affected members of their families, initially suggesting either potential de novo mutation or recessive inheritance (FIG. 1B).

Whole Exome Sequencing Identifies 3′ FBN1 Mutations in NPS—

A combination of whole exome and sanger sequencing identified de novo, heterozygous, 3′ mutations in the FBN1 gene in both patients (FIGS. 1B, 1C). A literature search for similar cases uncovered five case reports describing both an identical phenotype and FBN1 3′ truncating mutations (Graul-Neumann, et al., 2010; Horn & Robinson, et al., 2011; Goldblatt, et al., 2011; Takenouschi, et al., 2013; Jacquinet, et al., 2014). All 7 patients (including those of the disclosure) were diagnosed with NPS and all had truncating mutations within a 71 base pair segment of the approximately 8600 base pair coding region (FIG. 1C). All 7 mutations occur 3′ 50 nucleotides of the penultimate exon (FIG. 1C), are predicted to result in escape from nonsense mediated decay, and lead to C-terminal truncation of the fibrillin-1 protein due to frame-shift (FIG. 1D). FBN1 is the gene associated with Marfan syndrome, a connective tissue disorder that typically affects the eyes, large blood vessels such as the aorta, and the skeleton (Pyeritz, et al., 2009). Patients are typically tall, thin and have a long arm-span relative to their height (Pyeritz, et al., 2009). Although the patients of the disclosure grossly looked very different from classic Marfan syndrome patients, careful physical examination uncovered the majority of the features of Marfan syndrome in the patients of the disclosure, based upon the revised Ghent nosology for the diagnosis of Marfan syndrome (Loeys, et al., 2010). This was corroborated by the five published case reports associating NPS with 3′ mutations in FBN1 (Graul-Neumann, et al., 2010; Horn & Robinson, et al., 2011; Goldblatt, et al., 2011; Takenouschi, et al., 2013; Jacquinet, et al., 2014). Thus, these NPS patients combine the Marfan syndrome phenotype (vascular, ocular and skeletal features) with partial lipodystrophy. Lipodystrophy gives NPS patients a unique appearance and makes the task of diagnosing the associated Marfan syndrome relatively challenging. This may explain why, prior to identification of FBN1 mutations in these patients, for several decades NPS was described as its own unique clinical entity with no connection to Marfan syndrome (OMIM 264090). Fibrillin-1 is a modular protein in that mutations affecting different modules result in different phenotypes (Marfan syndrome, Acromicric dysplasia, Geleophisic dysplasia, Stiff skin syndrome, Weill-Marchesani syndrome) (Pyeritz, et al., 2009; Davis, et al., 2012). Thus, the association of yet another syndrome with fibrillin-1 mutations is not surprising. With a clinical and molecular diagnosis secure, the present example elucidates the mechanism by which fibrillin-1 C-terminal truncating mutations result in lipodystrophy.

FBN1 is Highly and Dynamically Expressed in White Adipose Tissue—

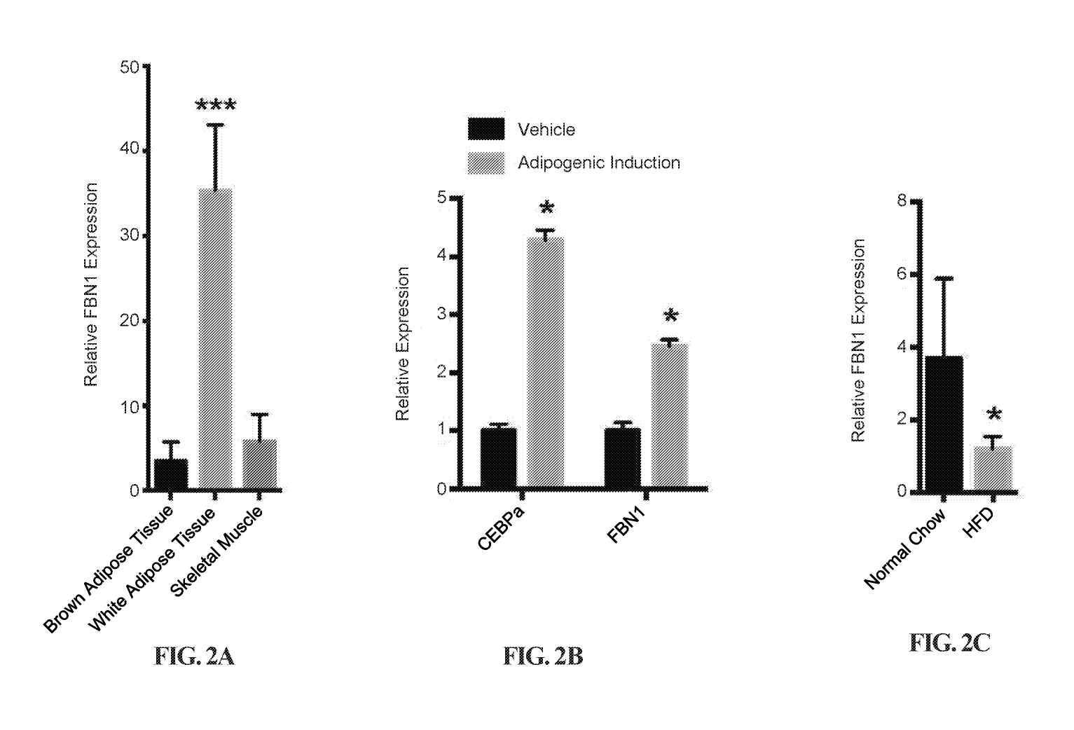

FBN1 is expressed at high levels in human adipose tissue (Biogps.org, Homo sapien probe set: 202765_s_at), in accord with the NPS phenotype of reduced subcutaneous fat. In mice, Fbn1 is specifically expressed in white adipose tissue compared with brown adipose tissue and skeletal muscle (FIG. 2A). Differentiation of human preadipocytes into adipocytes resulted in an increase in FBN1 expression (FIG. 2B), whereas a reduction in Fbn1 expression in inguinal adipose tissue was observed in mice exposed for several weeks to a high fat diet (FIG. 2C).

Asprosin is a Circulating, C-Terminal Cleavage Product of Profibrillin—

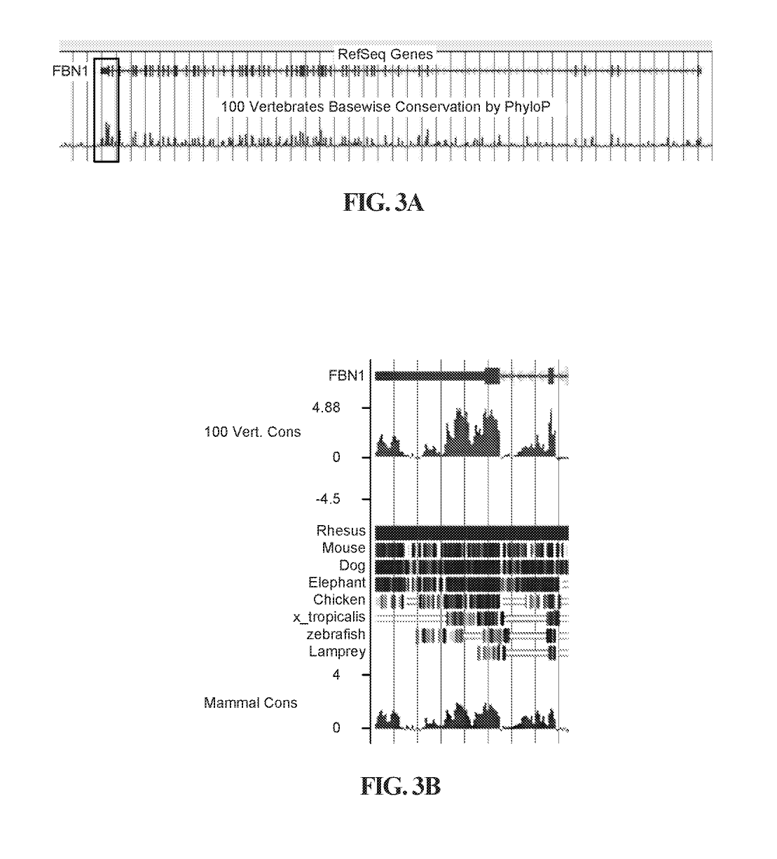

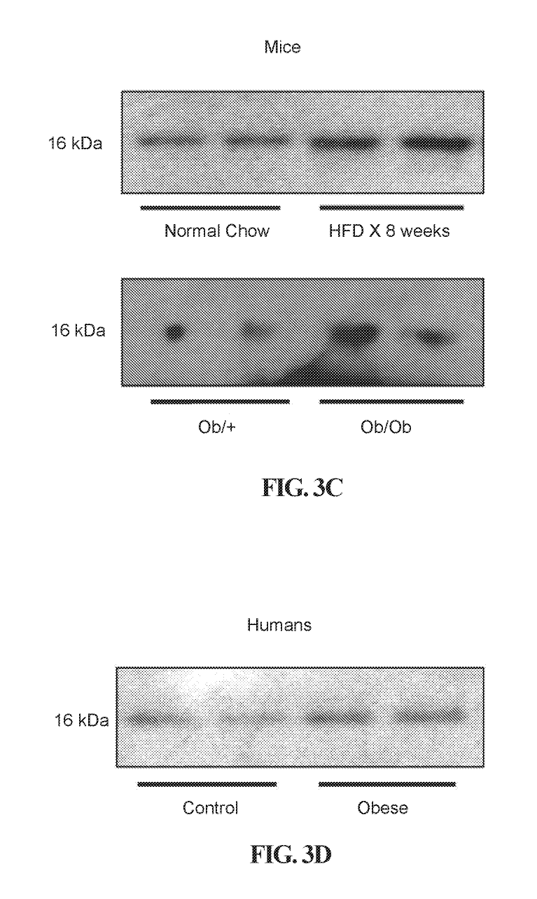

Fibrillin-1 is made as a 2871 amino acid proprotein, which is secreted from cells and cleaved at the C-terminus by an extracellular protease called furin (Milewicz, et al., 1995; Ritty, et al., 1999; Raghunath, et al., 1999; Wallis, et al., 2003). This results in the release of a 140 amino acid C-terminal cleavage product (CT polypeptide), and mature fibrillin-1 that serves as an extracellular matrix component (Milewicz, et al., 1995; Ritty, et al., 1999; Raghunath, et al., 1999; Wallis, et al., 2003). All seven NPS mutations are clustered around the cleavage site, resulting in a heterozygous loss of the CT polypeptide (FIG. 1D). The CT polypeptide shows the highest evolutionary conservation compared with other parts of the protein, and when compared with other species, suggesting an important biological role (FIGS. 3A, 3B). It was considered that under normal physiological conditions the CT polypeptide remains stable and has an independent function related to the NPS phenotype. Western blotting confirmed the presence of a unique, discreet 16-kDa cross-reacting entity in plasma from humans and mice (FIGS. 3C, 3D). Using plasma from obese mice and humans, it was found that the level of the CT polypeptide was proportional to adiposity in both species (FIGS. 3C, 3D). Because FBN1 is highly expressed in white adipose tissue and the NPS phenotype is clinically distinguished by a reduction in white adipose mass, the CT polypeptide was named Asprosin after Aspros, Greek for “white”.

Asprosin Rescues the NPS Associated Adipogenic Differentiation Defect In Vitro—

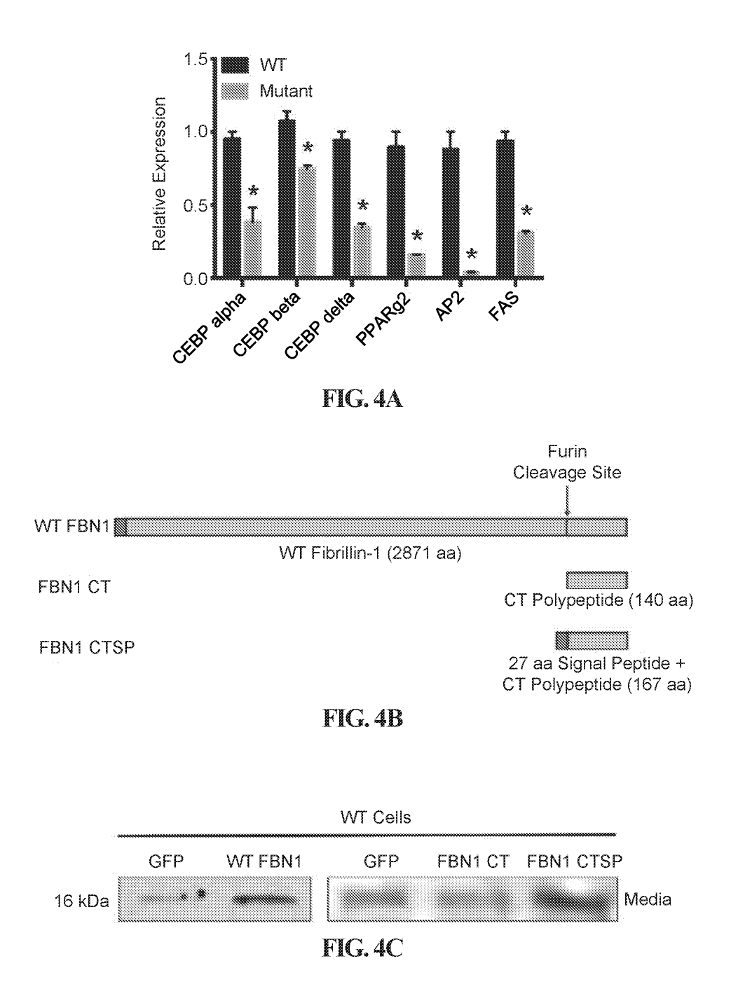

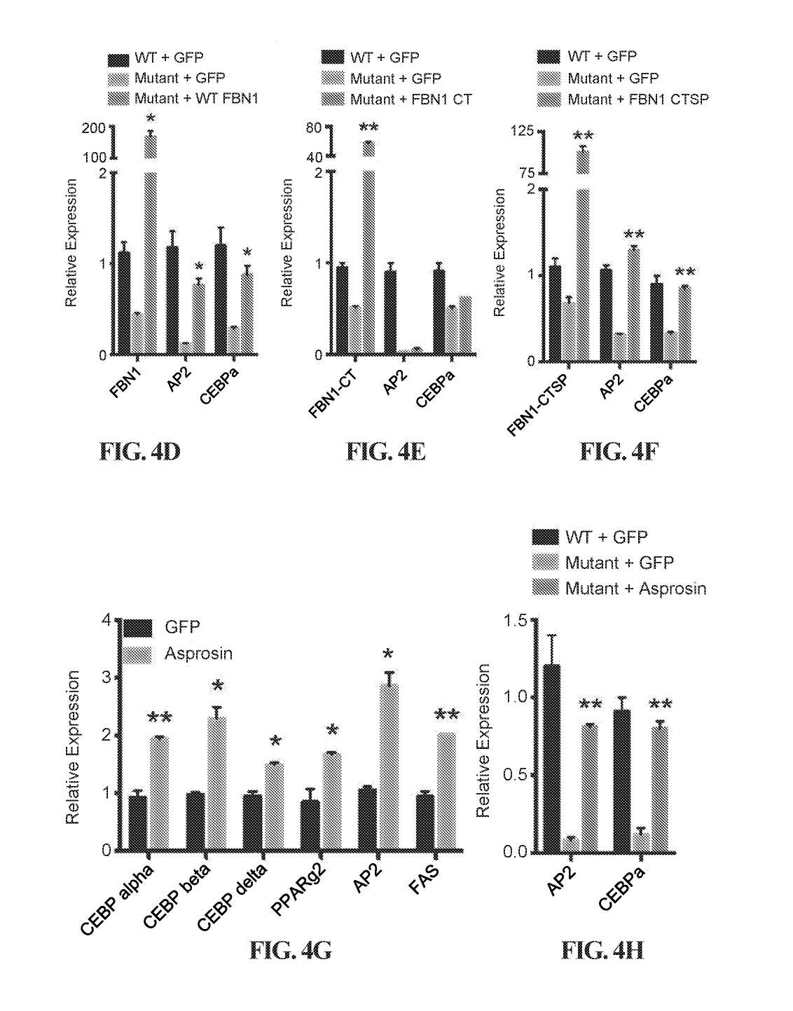

The impact of NPS mutations was tested on adipogenic differentiation of cells in vitro using dermal fibroblasts from patients with NPS and unaffected control subjects. Cells were exposed to an adipogenic induction cocktail for seven days that induces increased expression of a number of transcription factors and fat specific genes (Jaager, et al., 2012). Compared with WT cells, NPS mutant fibroblasts were strikingly defective in adipogenic differentiation (FIG. 4A). This defect could be rescued by overexpressing either WT FBN1 (FIG. 4D) or a secreted form of asprosin, but not by asprosin expressed without a signal peptide resulting in its intracellular entrapment (FIGS. 4C, 4E, 4F). To confirm the extracellular seat of action of asprosin's adipogenic effect, recombinant asprosin was generated in E. coli. Addition of recombinant asprosin to culture media promoted adipogenic differentiation in WT cells (FIG. 4G), and was sufficient to rescue the adipogenic defect in NPS mutant cells (FIG. 4H).

High Circulating Asprosin is Obesogenic and Diabetogenic—

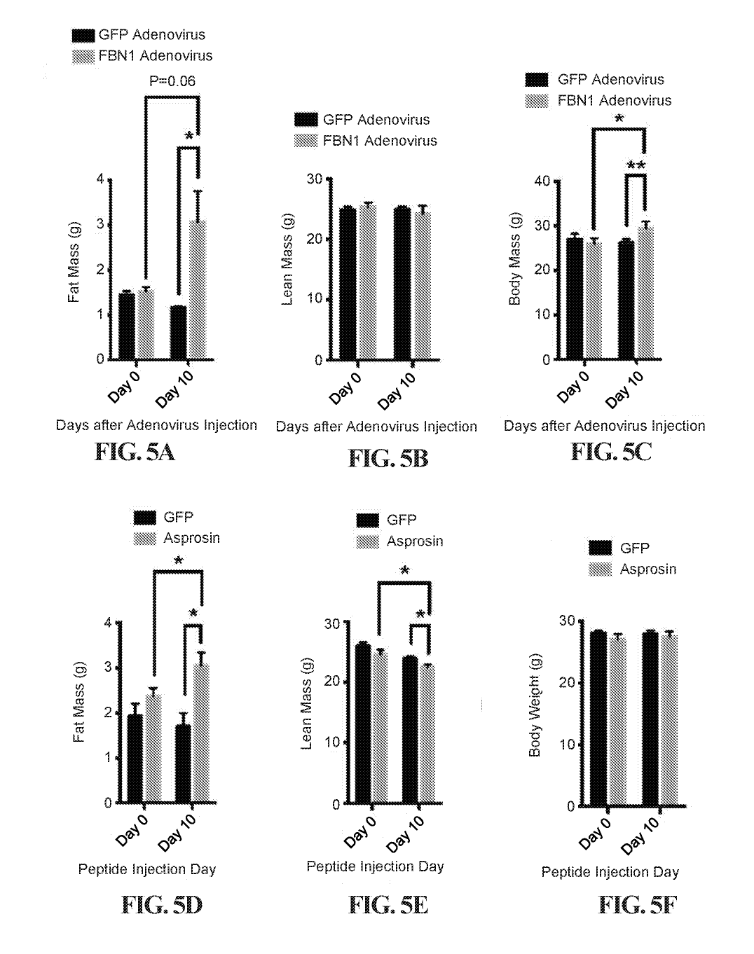

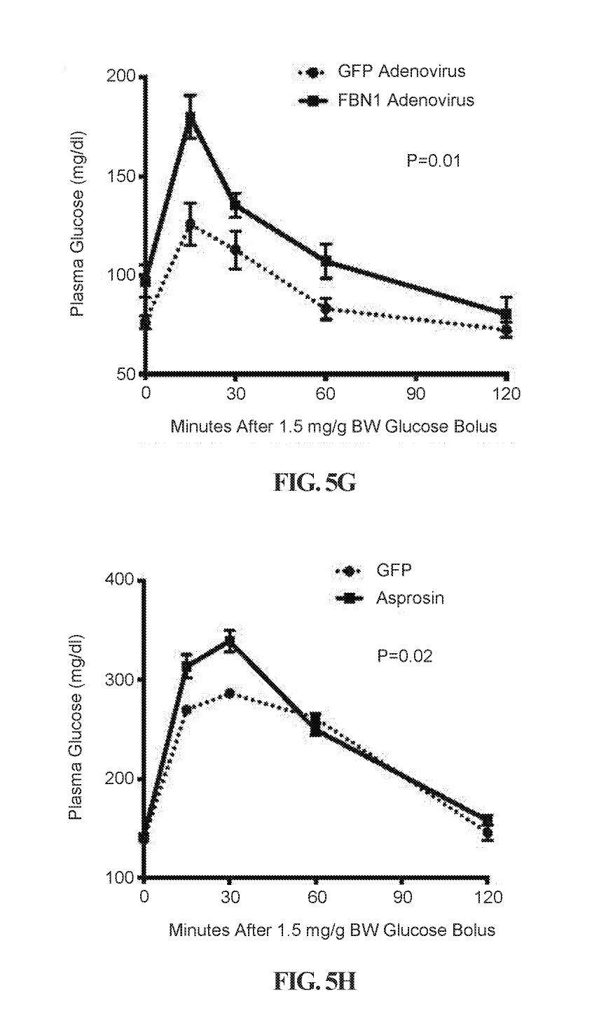

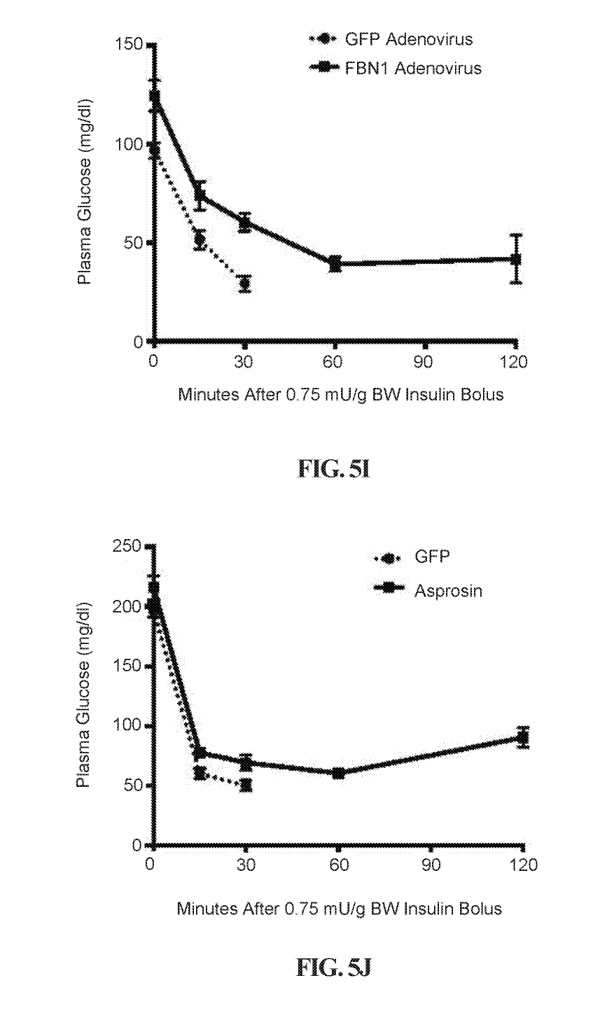

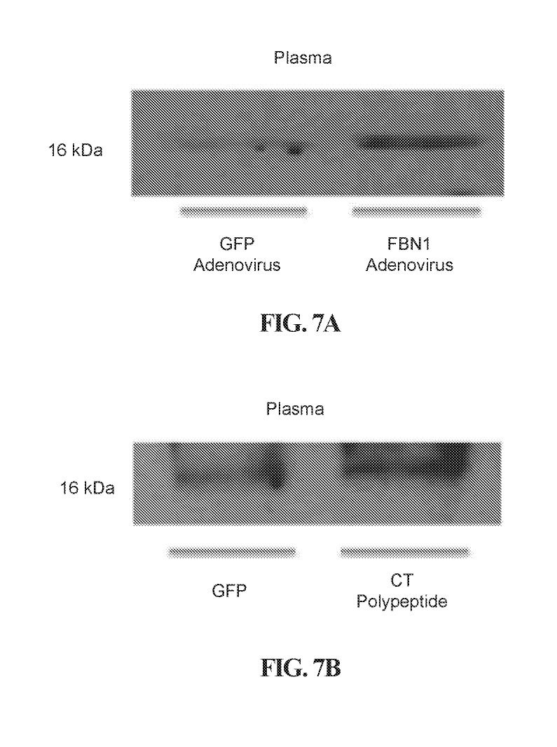

To initially test the effect of asprosin in vivo, it was expressed in the liver using adenoviruses carrying cDNA for WT FBN1 or GFP under control of the CMV promoter in standard-chow fed WT mice. Large amounts of asprosin were present in the circulation in mice exposed to the FBN1 adenovirus (FIGS. 7A-7B), suggesting correct secretion and cleavage of profibrillin by the liver. Ten days following adenoviral injection, MRI scans on the mice showed a 2.5 fold increase in fat mass (FIG. 5A) in mice with greater circulating asprosin, but no change in lean mass (FIG. 5B). The body weight of such mice was proportionally increased over that of control mice (FIG. 5C).

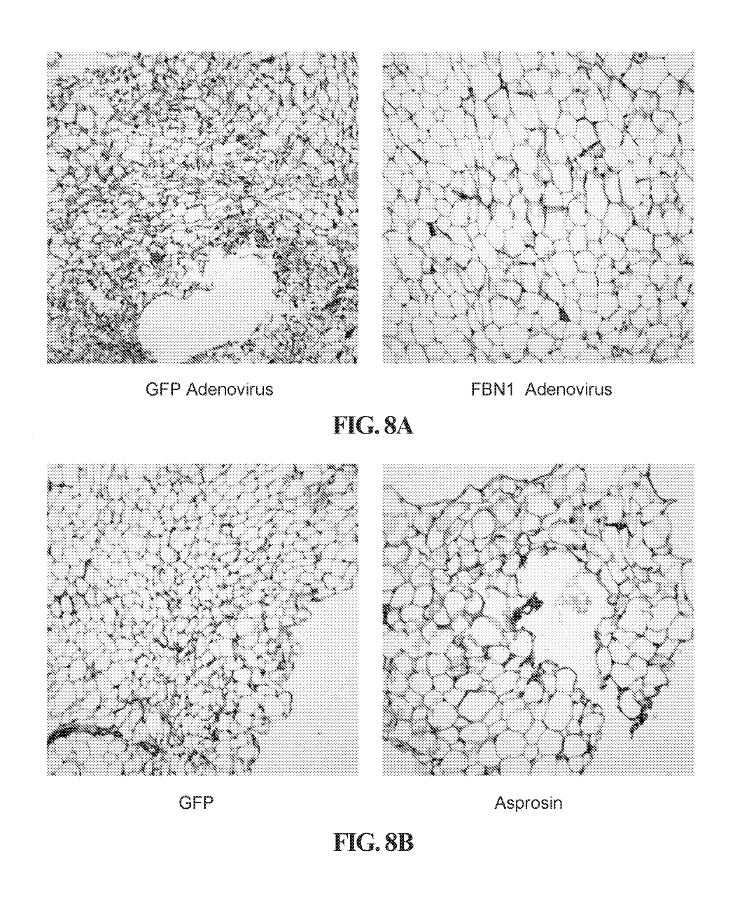

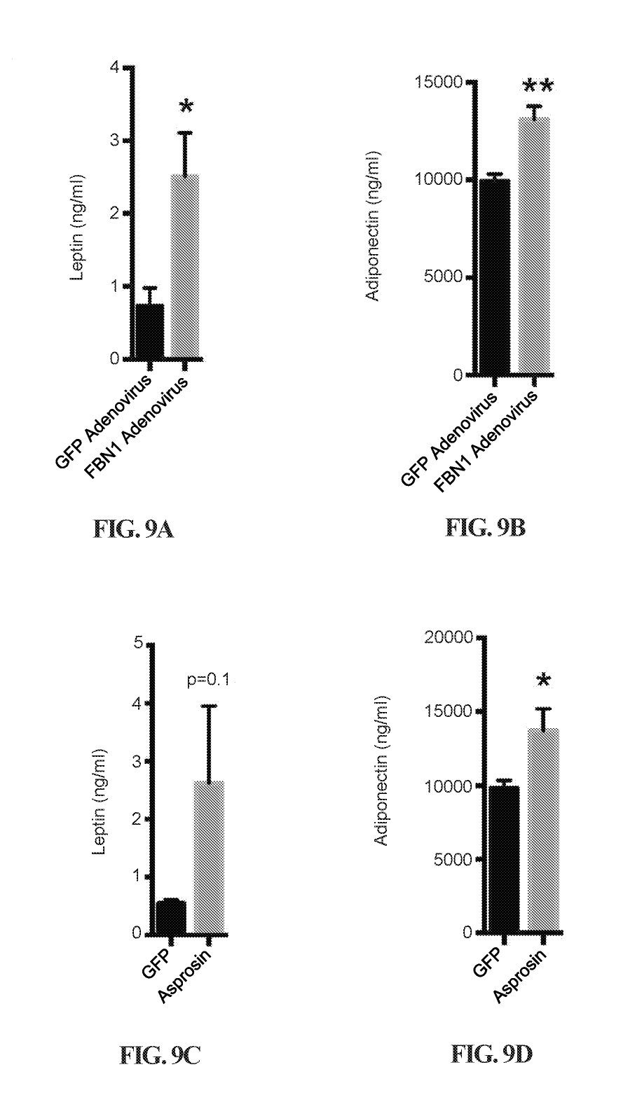

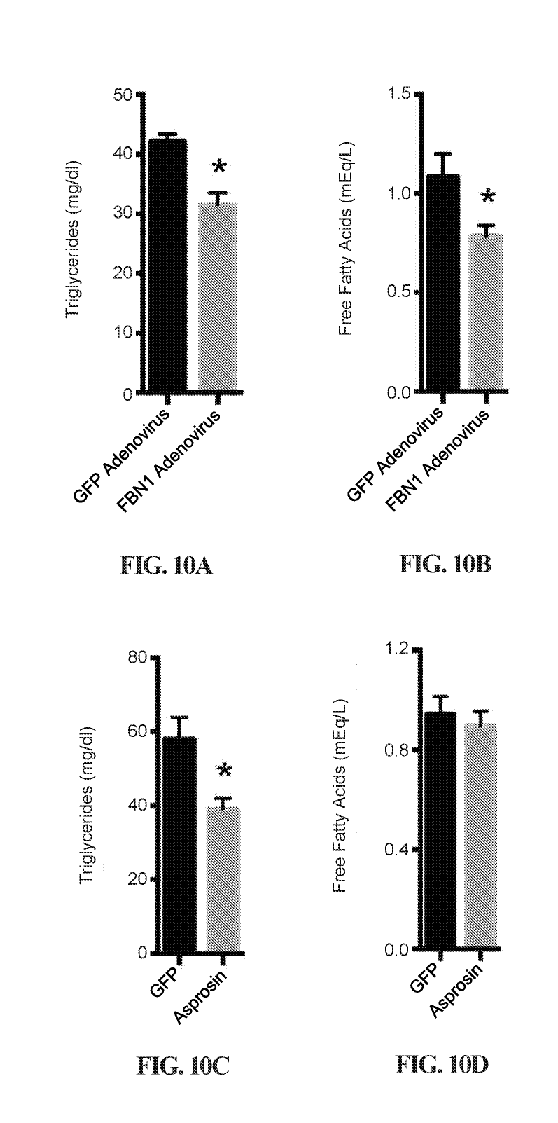

A second approach relied on daily subcutaneous injections of highly purified recombinant asprosin or GFP for ten days in standard-chow fed WT mice. Similar to the adenoviral approach, ten days of daily subcutaneous asprosin injection caused a significant increase in fat mass compared with GFP injection (FIG. 5D). In contrast to the adenovirus experiment, the lean mass of both asprosin and GFP injected mice showed a slight but significant decrease (FIG. 5E) that may reflect the stress imposed upon the mice by daily handling and injection. Regardless, both approaches demonstrated that acutely increasing the amount of circulating asprosin drives fat expansion in vivo. In both experiments, microscopy of inguinal white fat showed a larger fat cell volume in mice exposed to asprosin (FIGS. 8A-8B). Consistent with greater adiposity in these mice, there were higher levels of plasma leptin and adiponectin, adipose-derived hormones whose circulating levels are known to be directly proportional to fat mass (FIGS. 9A-9D). Concurrently, there were lower levels of plasma triglycerides and free fatty acids (FIGS. 10A-10D) that may reflect greater lipid sequestration in the larger adipocytes.

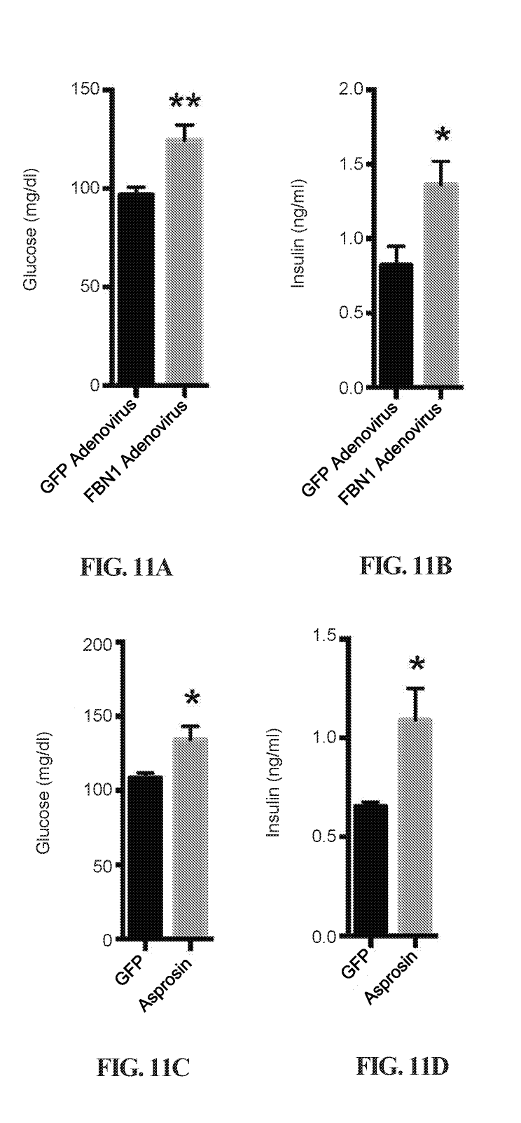

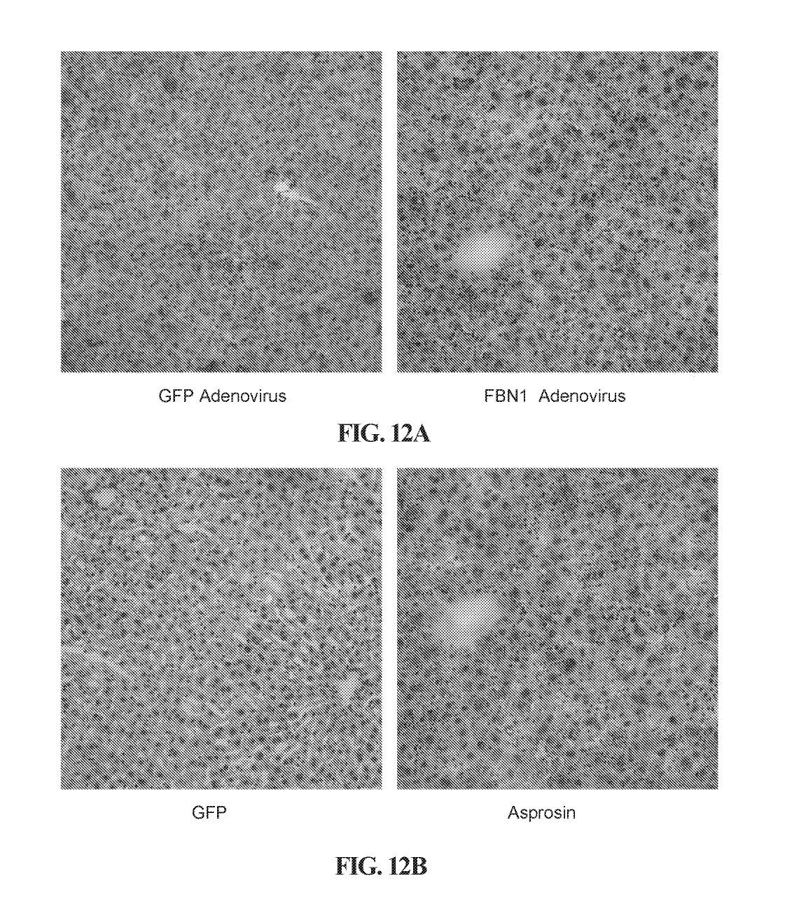

Given that there were the beginnings of obesity in mice exposed to greater circulating asprosin, glucose homeostasis was assayed in these animals. Fasted, asprosin-treated mice showed hyperglycemia and hyperinsulinemia (FIGS. 11A-11D), suggesting insulin resistance. Both, glucose and insulin tolerance tests were consistent with a diabetogenic effect of high circulating asprosin (FIGS. 5G, 5H, 5I, 5J). In accord with a state of obesity and insulin resistance, there was increased lipid accumulation in the livers of animals exposed to greater circulating asprosin (FIGS. 12A-12B). In summary, an acute increase in circulating asprosin was found to have a potent obesogenic and diabetogenic effect in mice.

Dominant Negative Effect of Truncated Profibrillin—

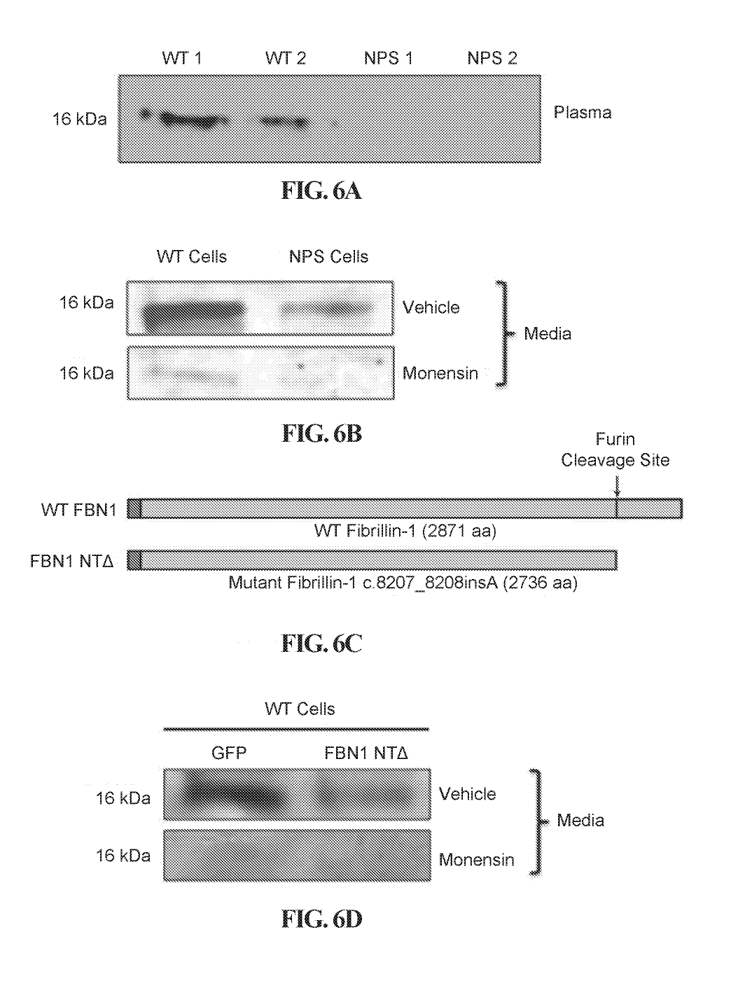

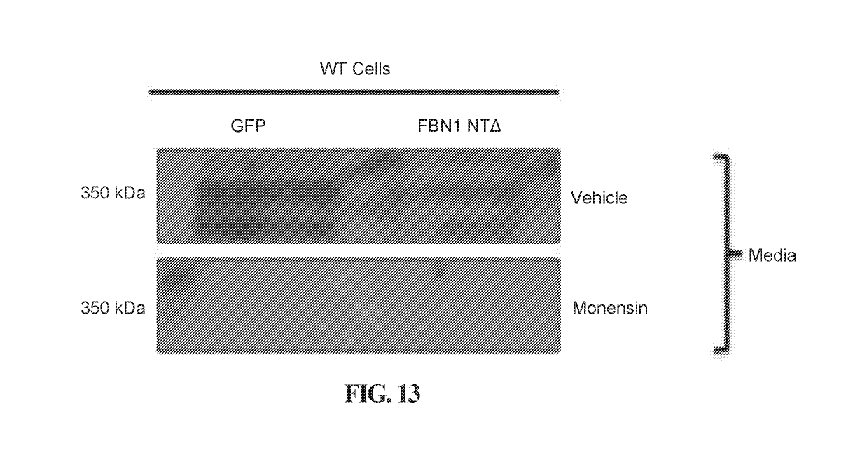

In addition to being extremely lean, NPS patients are also insulin sensitive (O'Neill, et al., 2007). The opposite physiological profile of mice exposed to too much circulating asprosin corroborates that the NPS phenotype is likely due to reduced circulating asprosin levels. Their heterozygous genotype predicts that NPS patients should have half the circulating asprosin compared with unaffected controls, but there was no detectable circulating asprosin at all in these patients (FIG. 6A). It has recently been shown that the CT polypeptide is necessary for profibrillin secretion from cells (Jensen, et al., 2014). In its absence, the truncated profibrillin that escapes nonsense mediated decay remains trapped intracellularly (Jensen, et al., 2014). Thus, it was considered that the mutant, truncated profibrillin in NPS acts in a dominant negative fashion to prevent secretion of profibrillin from the WT allele. This could also explain why the NPS phenotype is different from classic Marfan syndrome, at least in patients with more N-terminal truncations that then undergo nonsense mediated decay, or whole gene deletions—both of which would not express the truncated profibrillin. In order to test this theory, levels of asprosin were assayed in cell culture media from NPS cells, as well as from WT cells with overexpression of mutant, truncated profibrillin. In both instances, there were markedly reduced asprosin levels in the media, as expected (FIGS. 6B, 6D). Additionally, overexpression of mutant profibrillin in WT cells was sufficient to reduce the amount of fibrillin-1 secretion into the media, suggesting a dominant negative mode of pathogenesis for the Marfan syndrome phenotype seen with NPS (FIG. 13).

Methods

Study Subjects and Ethics Statement—

Informed consent was obtained prior to participation from all subjects under one of three Institutional Review Board approved protocols at Baylor College of Medicine.

Clinical Evaluation—

Clinicians assessed study subjects by direct history, physical examination, and family history analysis. Clinical information in the form of chart records and notes was reviewed. Interviews with these subjects were also conducted by telephone. Families were interviewed together with the patients. Whenever available, reports from previous diagnostic studies, operative reports, or radiologic studies were reviewed. After informed consent, skin biopsies for isolation of dermal fibroblasts were performed under appropriate anesthetic and universal precautions.

Whole-Exome Capture and Sequencing—

Genomic DNA from patient #1 and her parents was subjected to whole exome sequencing (trio analysis). Methods utilized for whole-exome sequencing have been previously described in detail (Lupski, et al., 2013). In summary, 1 mg of genomic DNA was fragmented by sonication in a Covaris plate (Covaris, Inc. Woburn, Mass.). Genomic DNA samples were constructed into Illumina paired-end libraries as described (Lupski, et al., 2013). Pre-capture libraries were pooled together and hybridized in solution to the BCM-HGSC CORE exome capture design (Bainbridge, et al., 2011) (52 Mb, Nimble-Gen). Captured DNA fragments were sequenced on an Illumina HiSeq 2000 platform producing 9-10 Gb per sample and achieving an average of 90% of the targeted exome bases covered to a minimal depth of 206 or greater.

Data Analysis—

Produced sequence reads were mapped and aligned to the GRCh37 (hg19) human genome reference assembly using the HGSC Mercury analysis pipeline. Variants were determined and called using the Atlas2 suite to produce a variant call file (VCF). High-quality variants were annotated using an in-house developed suite of annotation tools.

Sanger Sequencing—

Genomic DNA from patient #2 was subjected to sanger sequencing. Primers were designed to encompass exons 65 and 66 including intron-exon boundaries of the FBN1 gene using Primer3. Sanger reads were analyzed using the Lasergene Seqman software.

Animals—

10-week old male WT C57/Bl6 mice were used for all in vivo studies. Mice were housed 2-5 per cage in a 12-hour light/12-hour dark cycle with access to food and water ad libitum. Mice were exposed to adenoviral-mediated transgenesis (10¹¹ virus particles per mouse), via tail-vein injections. Mice were injected with 2.6 micro molar recombinant His tagged Asprosin or recombinant GFP daily for 10 days via subcutaneous injection. Mice were sacrificed and plasma and various organs were isolated 10 days after viral infusion or peptide injection. The Baylor College of Medicine Institutional Animal Care and Utilization Committee approved all experiments.

FBN1 and GFP Adenoviruses—

Adenovirus carrying FBN1 cDNA was created by cloning the FBN1 coding region under control of the CMV promoter using a standard Ad5 vector system. The corresponding GFP adenovirus was purchased from the Vector Development Core at Baylor College of Medicine.

Recombinant Asprosin and GFP—

Human FBN1 (2732-2871 amino acids) cDNA was cloned and subsequently sub-cloned into a pSPE plasmid for expression in E-coli. The fusion protein that was expressed in E. coli is 146 amino acid long comprising of a 6 amino acid His tag on the N-terminus and a 140 amino acid wild type C-terminal FBN1 (2732-2871 amino acids). His-tagged GFP was purchased from Thermo Scientific as the control polypeptide.

Body Composition and Serum Analyses—

Body composition was analyzed with the ECHO-MRI system (Echo medical systems, Texas). Mouse serum was prepared from blood obtained through cardiac puncture and analyzed with the COBAS Integra 400 plus analyzer (Roche). Plasma leptin, FFA, adiponectin and triglyceride levels were measured by using a Mouse Leptin ELISA Kit (Millipore), NEFA C Test Kit (Wako), Mouse Adiponectin ELISA Kit (Millipore) and Serum/plasma triglyceride detection kit (Sigma), respectively.

Histology—

Mouse inguinal adipose tissue samples were fixed in 10% formaldehyde for H&E staining. Frozen livers were used for oil-red-0 staining to evaluate hepatic triglyceride content.

Glucose Tolerance Test (GTT) and Insulin Tolerance Test (ITT)—

For GTT, intraperitoneal injection of 1.5 g of glucose/kg of body weight was performed after a 6-hour fasting period. For ITT, intraperitoneal injection of regular insulin (Humulin R; 0.75 unit/kg of body weight) was administered after a 4-hour fasting period. Blood glucose levels were measured using a glucometer (Life Scan).

Expression Vectors—

WT FBN1 (1-2871 amino acids), 140 amino acid Asprosin (2732-2871 amino acids) and Asprosin with the native 27 amino acid FBN1 signal peptide attached at the N-terminus (amino acid 1-27+amino acid 2732-2871) were sub-cloned under control of the CMV promoter using the pCMV6-Neo vector system. The same vector expressing GFP or empty vector was used as a control.

Cell Culture—

Human dermal fibroblasts isolated from NPS subjects or WT dermal fibroblasts from unaffected control subjects were subjected to adipogenic differentiation using standard protocols. To stimulate adipogenesis, medium was supplemented with 2 uM insulin, 1 uM dexamethasone, 0.25 mM isobutyl methyl xanthine and 10-7 M rosiglitazone for 7 days. Standard transfection methods with expression plasmids were used for in vitro transgenesis.

RNA and Protein Analysis—

Standard RNA extraction procedures (RNeasy Mini Kit from Qiagen) were employed. Reverse transcription was carried out using the Superscript III kit (Invitrogen) using the manufacturer's protocol. For gene expression analysis, QPCR was performed using sequence-specific primers and probes from Roche (Universal Probe Library). TBP was used as an internal control for all gene-expression assays. Western blotting was performed using standard methods on plasma or cell culture media using a mouse monoclonal antibody directed against Asprosin, which was purchased from Abnova (Catalog# H00002200-M01). The mouse monoclonal antibody against fibrillin-1 was purchased from Abcam (Catalog# ab3090). For western blotting on media, cells were subjected to adipogenic differentiation for 7 days followed by replacing the induction media with serum free DMEM supplemented with Cellgro ITS (insulin, transferrin, selenium) from Mediatech for 3 days. At that time, media was concentrated using the Amicon Ultra-2 Centrifugal filter unit before proceeding with western blotting.

Statistical Methods—

All results are presented as mean±SEM. P value was calculated by unpaired Student's t test or ANOVA, as appropriate. *P<0.05, **P<0.01, and ***P<0.001.

### Example 2

### Determine the In Vivo Impact of Gain-of-Function of the Fibrillin-1 C-Terminal Polypeptide

The Fibrillin-1 protein was identified 50 years ago (Guba, et al., 1964). Much is known about its functions in maintenance of the extracellular matrix (particularly in the aortic smooth muscle) and its role in health and disease (Davis & Summers, et al., 2012; Reinhardt, et al., 1995). Its structure is known as “modular”, meaning that mutations in different parts of the protein lead to different clinical outcomes. As such, it has been associated with Marfan Syndrome, Acromicric Dysplasia, Geleophisic Dysplasia, Stiff Skin Syndrome and Weill-Marchesani Syndrome (Davis & Summers, 2012). Using whole exome sequencing, as well as existing literature, it is also associated with a rare, extreme thinness disorder known as Neonatal Progeroid Syndrome (NPS).

NPS is an autosomal-dominant genetic disorder that results in extreme thinness due to a drastic reduction in subcutaneous adipose tissue (FIGS. 1A-1D) (O'Neill, et al., 2007; Hou, et al., 2009). The phenotype of the patients overlaps with, but is distinct from classic Marfan syndrome, especially when it comes to their lipodystrophy (Graul-Neumann, et al., 2010; Takenouchi, et al., 2013; Horn, et al., 2011; Goldblatt, et al., 2011). Thus, the site and type of mutation was characterized to explain the difference. The 2 patients that were identified in the disclosure and the 4 that have been previously described (Graul-Neumann, et al., 2010; Takenouchi, et al., 2013; Horn, et al., 2011; Goldblatt, et al., 2011) all have Cterminal truncating mutations in the penultimate exon of FBN1. These 6 truncating mutations are within 70 bp of each other in an 8600 bp gene. Particularly since lipodystrophy has never been described in association with mutations found in other parts of FBN1, it seems clear that a shared feature of these mutant proteins somehow affects fat biology. The studies have revealed an independently functional Fibrillin-1 C-terminal polypeptide, which is normally cleaved off the parent protein (Ritty, et al., 1999; Raghunath, et al., 1999; Wallis, et al., 2003; Milewicz, et al., 1995) after it is secreted from the cell. Preliminary experiments have shown that haploinsufficiency for the C-terminal polypeptide results in defective fat differentiation. A goal is to characterize whether overexpression of this polypeptide is sufficient to make WT and lipodystrophic mice gain fat mass. This would have direct therapeutic implication for both generalized and localized lipodystrophic conditions that result in a loss of fat mass.

One may test the predicted sufficiency of the Fibrillin-1 C-terminal peptide in fat homeostasis in vivo. These studies can assess the impact of the C-terminal peptide on fat accretion ability in mice treated with recombinant C-terminal polypeptide as well as an adenovirus carrying the cDNA for it. Global gene-expression and metabolomic data sets can be generated and mined to develop testable hypotheses regarding the pathways employed by the Fibrillin-1 C-terminal polypeptide.

Experimental Approach:

A. Inject Recombinant Fibrillin-1 C-Terminal Polypeptide and GFP in Mice:

8-week-old C57/Bl6 WT and PPAR gamma null (lipodystrophic) mice are injected with 20 ug each of recombinant C-terminal polypeptide or recombinant GFP using the subcutaneous approach, every two days for a total of five doses. The recombinant polypeptides have been previously generated using bacterial expression followed by purification and endotoxin removal. The dose of 20 ug each was decided on the basis of preliminary data assessing endogenous plasma levels in mice. 8 mice in each sex-matched group are compared in all assays 10 days after injection.

B. Inject Adenoviral Vectors Carrying the Fibrillin-1 C-Terminal Polypeptide and GFP in Mice:

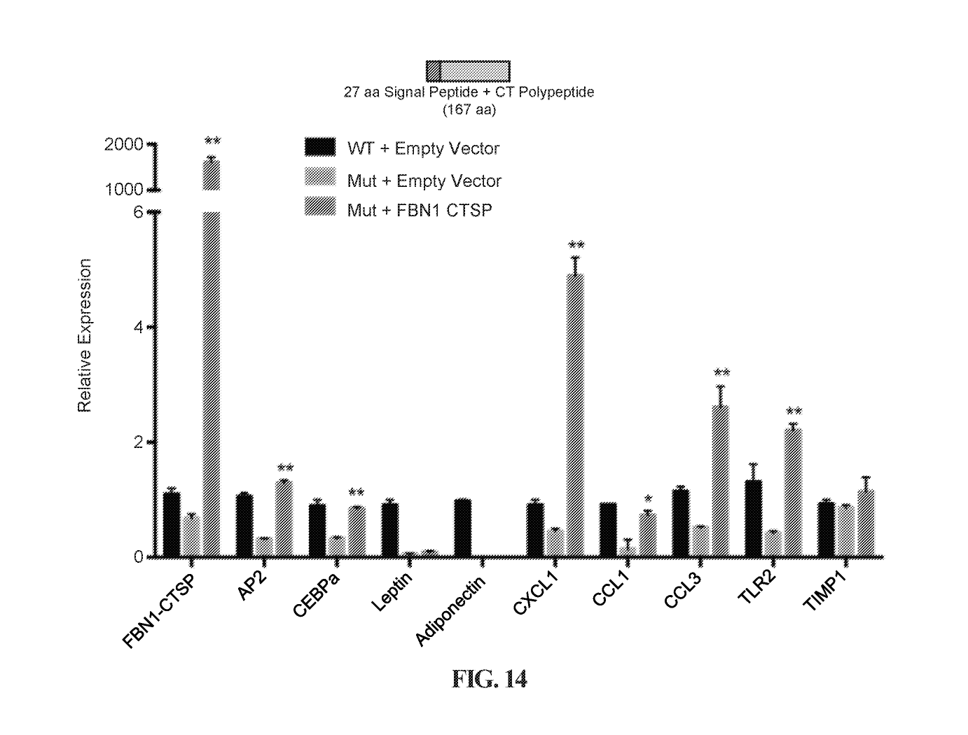

8-week-old C57/Bl6 WT and PPAR gamma null (lipodystrophic) mice are injected with 1011 viral particles each of previously generated adenovirus expressing C-terminal polypeptide fused to a signal-peptide (FIG. 14) or adenovirus expressing GFP. Based on prior experience using this technique, the majority of the adenoviral load will infect the liver (Chopra, et al., 2008; Chopra, et al., 2011). Following overexpression by hepatocytes, the C-terminal polypeptide, which has been fused with the native Fibrillin-1 signal-peptide, should be secreted by the cells. 8 mice in each sex-matched group are compared for plasma levels of the polypeptide two weeks following the injection, followed by other downstream assays.

C. Measure Impact of Overexpression of the C-Terminal Polypeptide on Adiposity:

Mice are anesthetized and weight and length are recorded. They are placed in the DEXA analyzer (Oosting, et al., 2012) and a scout-scan is performed before performing a true measurement-scan. The exposure dose per mouse is set at 300 μSv. For analysis of the data, regions of interest are defined. The analysis may comprise of a whole body measurement excluding head area. The count data are transformed by software into bone and non-bone components. Information is generated about body weight, body length, bone and fat mass, bone mass density and lean mass of each mouse. The DEXA measurements and analysis are performed at the “Mouse Phenotyping Core Facility” at BCM. After euthanasia, inguinal fat pads are extracted, photographed and weighed.

D. Measure Impact of Overexpression of the C-Terminal Polypeptide on Global Metabolic Changes by Performing Unbiased Plasma Metabolite Profiling:

In order to identify organism wide, metabolic changes as a consequence of overexpression of the Fibrillin-1 C-terminal polypeptide, RNAseq is employed. EDTA-Plasma from fasted and fed mice are collected by exsanguination. Frozen, coded samples are sent to Metabolon, Inc. (Durham, N.C.) and accessioned into the Metabolon system by a unique identifier associated with the original source only. Recovery standards are added prior to the first step in the extraction process for quality control purposes. Sample preparation uses a proprietary series of organic and aqueous extractions to remove proteins while allowing maximum recovery of small molecules. Extracted samples are split into equal parts for analysis by gas chromatography/mass spectrometry (GC/MS) and liquid chromatography/mass spectrometry (LC/MS) platforms. Several technical replicate samples are created from a homogeneous pool containing a small amount of each sample. Raw MS data files are loaded into a relational database. Peaks are identified using Metabolon's proprietary peak integration software, and component parts are stored in a separate and specifically designed complex data structure. Compounds are identified by comparison to library entries of purified standards or recurrent unknown entities. Identification of known chemical entities are based on comparison to the over 1,000 commercially available, purified standard compounds registered in LIMS for distribution to both LC and GC platforms. Demographics are presented by frequencies for categorical variables and means±standard deviation (mean±SD) for continuous variables followed by Bonferroni posttest analysis to obtain statistical significance. Approximately 3000 individual plasma metabolites in various classes (acyl-carnitines, organic acids, amino acids, peptides, ions, etc.) can be assayed simultaneously in an unbiased manner using this technique.

E. Assess Impact of Fat-Specific Overexpression of the C-Terminal Polypeptide on Fat Homeostasis at the Level of Global Gene Expression Using RNAseq:

In order to identify genome wide, transcriptomic changes in adipose tissue as a consequence of overexpression of the Fibrillin-1 C-terminal polypeptide, RNAseq is employed. Total RNA is isolated from previously flash frozen inguinal adipose tissue. Sequencing reactions are done on pooled RNA samples from 5 individual mouse inguinal white fat depots. Four lanes of the flowcell are used for the sequencing of the samples on the Genome Analyzer II. The Genome Analyzer (GA) is run for 38 cycles. The images from the GA are analyzed with the GA pipeline software (v1.3, Illumina software) on cycles 1-38 to undertake image analysis, base calling and sequence alignment to the reference genome. Sequences are aligned with the ELAND software. The aligned reads are used as input for the Illumina CASAVA program (v1.0) to count the sequence reads that align to genes, exons and splice junctions of the reference genome. The raw counts of sequences aligning to features (gene, exons and splice junctions) are normalized by CASAVA by dividing the raw count by the length of the relevant feature. The read counts per gene are used as input for DEGseq and DEseq to identify differentially expressed genes. Both tools are available via the statistics package R and Bioconductor. DEGseq and DESeq use different statistical approaches (Poisson distribution, negative binomial distribution) to estimate probabilities for differential gene expression. A P≤0.001 and a 2-fold change (normalized) in expression levels are used as cut-off criteria.

One can expect that studies described herein establish sufficiency of the Fibrillin-1 C-terminal polypeptide for the twin processes of fat accretion and inflammation, in specific embodiments. Two gain-of-function approaches are described herein with the aim of assessing the same endpoints. It is expected that even if one approach fails, the other provides conclusive ends. Because of lack of information on the half-life of the native polypeptide in plasma, the recombinant polypeptide experiment could fail if the majority of the peptide is quickly degraded. In that case, it is expected that the adenovirus-mediated transgenesis approach circumvents this issue through constant production of the polypeptide in sufficient amounts to result in gain-of-function. By their very nature, overexpression experiments have the potential to create physiological states that do not reflect the true functionality of the protein being tested. Thus, they need to be interpreted with caution and, if possible, interpreted in the context of concurrent loss-of-function studies. Collective interpretation of gain-of-function and loss-of-function studies proposed herein can enable one to draw the correct conclusions on the true in vivo functions of the Fibrillin-1 C-terminal polypeptide.

### Example 3

### Determine the In Vivo Impact of Loss-of-Function of the Fibrillin-1 C-Terminal Polypeptide

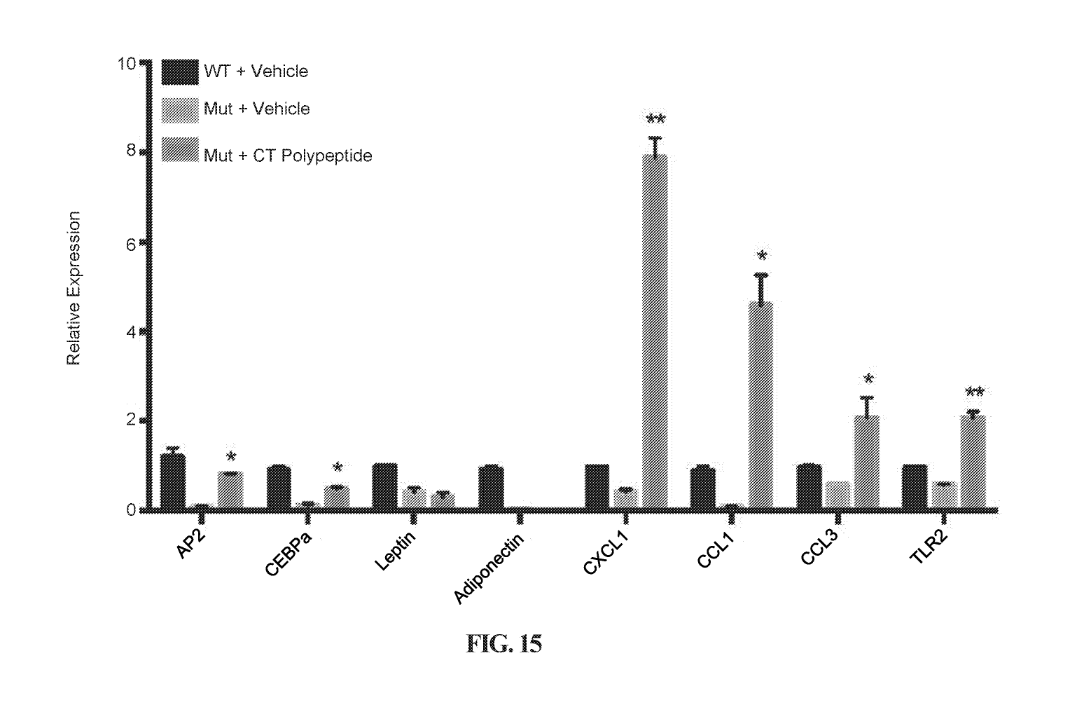

The Fibrillin-1 protein contains a C-terminal cleavage site (RGRKRR [SEQ ID NO:6] motif) that has been shown to undergo proteolytic processing by the Furin/PACE family of enzymes (Ritty, et al., 1999; Raghunath, et al., 1999; Wallis, et al., 2003; Milewicz, et al., 1995). This results in two fragments, functional Fibrillin-1 (2500 amino acids), which is dependent upon the cleavage event for proper insertion into the extracellular matrix (Raghunath, et al., 1999; Milewicz, et al., 1995), and a smaller C-terminal polypeptide (˜140 amino acids) whose independent function is unknown. The common result of all 6 heterozygous mutations in FBN1 that result in an NPS phenotype is a loss of the vast majority of the C-terminal polypeptide. If indeed haploinsufficiency of the C-terminal fragment is responsible for the phenotype, then restoring that fragment to its normal levels should result in rescuing the phenotype. This concept was explored in vitro and it was found that restoring the expression of the C-terminal polypeptide as well as simply exposing the mutant cells to the C-terminal polypeptide by adding it to the media, rescued the NPS associated fat differentiation and inflammogenic defects (FIG. 15).

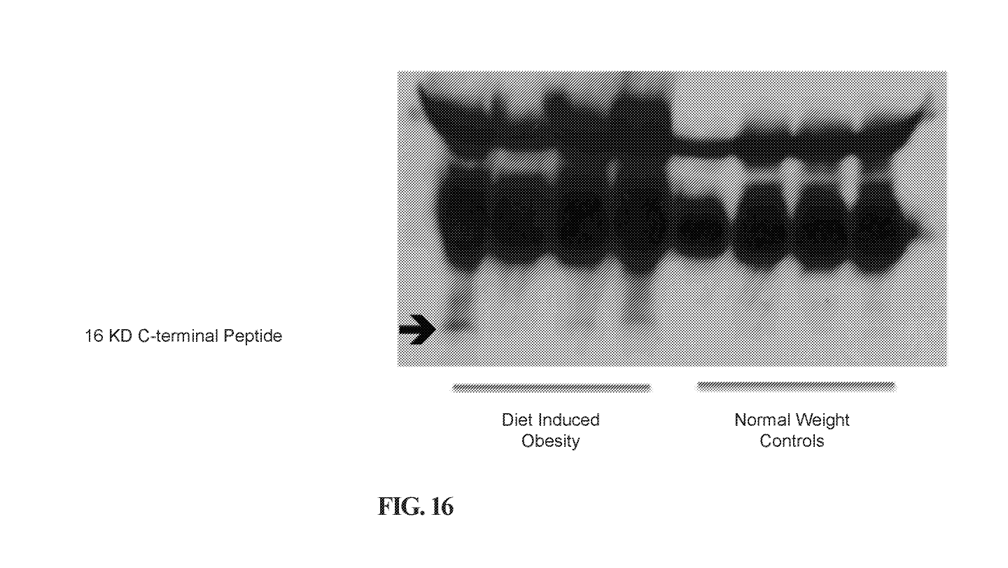

An analogous approach is utilized in vivo. The circulating C-terminal polypeptide (FIG. 16) is immunologically sequestered using a monoclonal antibody to unravel its necessity for fat accretion and potential for protection against obesity and metabolic syndrome. This would have direct therapeutic implication for both obesity and metabolic syndrome, conditions that result from unmitigated fat accretion.

Experimental Approach:

A. Expose WT and Genetically Obese Mice to a Monoclonal Antibody Targeting the Fibrillin-1 Cterminal Polypeptide:

8-week-old C57/Bl6 WT and ob/ob (obese mice with a loss-of-function Leptin mutation) mice are injected with 500 ug of anti-CT-Fibrillin-1 IgG or nonspecific IgG using the intraperitoneal approach, daily for a total of five doses. The monoclonal antibody targeting the Fibrillin-1 C-terminal antibody has been previously obtained from Sigma Inc. and validated in house. 8 mice in each sex-matched group are compared in all assays 10 days after injection.

B. Measure Impact of Loss of the C-Terminal Polypeptide on Adiposity:

The impact of neutralization of the Fibrillin-1 C-terminal polypeptide on adiposity is measured using DEXA scans and inguinal fat-pad weights as described herein. Eight sex-matched, 8-week-old, WT and ob/ob mice exposed to anti-CT-Fibrillin-1 IgG and control IgG are assessed.

C. Measure Impact of Loss of the C-Terminal Polypeptide on Global Metabolic Changes by Performing Unbiased Plasma Metabolite Profiling:

EDTA-Plasma from eight sex-matched, 8-week-old, WT and ob/ob mice exposed to anti-CT-Fibrillin-1 IgG and control IgG are collected by exsanguination. Metabolomics analysis is performed as described herein.

D. Assess Impact of Loss of the Fibrillin-1 C-Terminal Polypeptide in Fat Homeostasis at the Level of Global Gene Expression Using RNAseq:

Total RNA is isolated from previously flash frozen inguinal adipose tissue from fifteen sex-matched, 8-week-old, WT and ob/ob mice exposed to anti-CT-Fibrillin-1 IgG and control IgG. Sequencing reactions are done on pooled RNA samples from 5 individual mouse inguinal white fat depots (N=3). RNAseq analysis is done as described elsewhere herein.

One can expect that studies described herein establish that the Fibrillin-1 C-terminal polypeptide is necessary for fat accretion and protective against obesity, in specific embodiments. The studies considered using the monoclonal antibody targeting the Fibrillin-1 C-terminal polypeptide are not as clean as a genetic ablation study would have been. However, given that a goal is to study the use of such a monoclonal antibody as a therapeutic modality against obesity, it is important to test this compared with a nonspecific antibody, in at least some embodiments. In embodiments wherein this approach establishes a protective role for such an antibody against obesity, those results are confirmed with a genetic knockout study, for example.

### Example 4

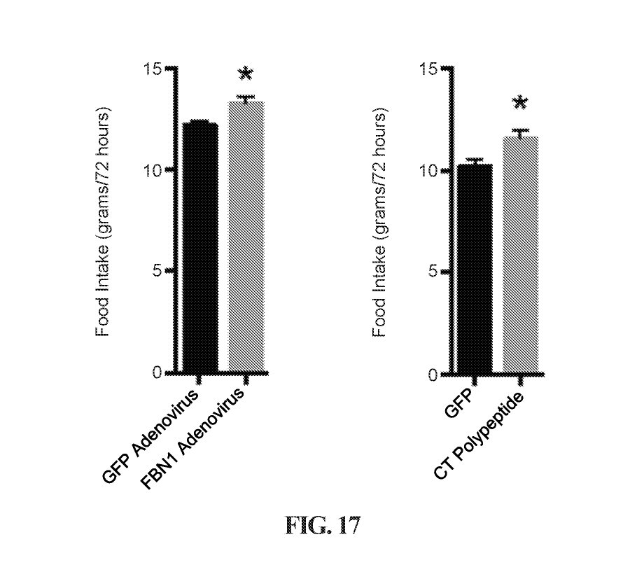

FIG. 17 shows that an increased amount of plasma CT polypeptide (asprosin) results in hyperphagia in mice that have been injected with asprosin. In embodiments of the disclosure, methods involve providing an effective amount of the CT polypeptide to an individual in need of gaining weight or increasing adipose mass.

In individuals with NPS or another medical condition in which the individual has insufficient adipose mass, the individual may consume a reduced daily caloric load compared to individuals that do not have NPS or another such medical condition. In particular embodiments for these individuals, they may be provided an effective amount of asprosin or a functional derivative thereof in order to increase their daily caloric load, such as by increasing their appetite.

### Example 5

### Significance of Embodiments of the Disclosure

The discovery of leptin shows that genetic disorders that result in extremes of body weight have the potential to be very informative in the understanding of obesity, diabetes and metabolic syndrome (Friedman, 2009). Described herein is a new polypeptide hormone, asprosin, that is necessary for maintenance of optimal fat mass, and whose origin is tied to an extracellular matrix protein, fibrillin-1. In that, it resembles endostatin, an angiogenic regulator that is a C-terminal cleavage product of a different extracellular matrix protein, Collagen XVIII (O'Reilly, et al., 1997). Thus, it would be reasonable to consider that some extracellular matrix components may have evolved as carriers of C-terminal cleavage products whose functions are distinct from their parent proteins.

Several previous studies have shown how profibrillin is secreted and likely cleaved extracellularly by the furin protease system (Graul-Neumann, et al., 2010; Horn & Robinson, 2011; Goldblatt, et al., 2011; Takenouchi, et al., 2013; Jacquinet, et al., 2014). This cleavage event is necessary for correct processing of fibrillin-1 and its insertion into the extracellular matrix (Graul-Neumann, et al., 2010; Horn & Robinson, 2011; Goldblatt, et al., 2011; Takenouchi, et al., 2013; Jacquinet, et al., 2014). However, the fate of the other cleavage product—the 140 amino acid C-terminal polypeptide has remained unknown. The genotype of NPS patients suggested the possibility that the C-terminal polypeptide, asprosin, has an important role in adipose biology, in embodiments of the disclosure. The data of this disclosure show that asprosin is present in the circulation and is necessary for maintenance of optimal fat mass. Loss of asprosin in humans results in a lipodystrophy, while in mice too much asprosin results in development of fat expansion and glucose intolerance, features of obesity and poor metabolic health. In fact, there are enhanced levels of circulating asprosin in obese states that are correlated with poor metabolic health in mice and humans. In the opposite direction, the phenotype of NPS patients, who have little to no circulating asprosin, display extreme thinness and insulin sensitivity, indicating that in some embodiments decreasing asprosin favors a positive metabolic profile. This is in contrast to some types of lipodystrophy that result in insulin resistance (Nolis, et al., 2013).

In one embodiment, retention of insulin sensitivity in NPS is the sparing of certain fat depots, especially in the gluteal area, that presumably retain their glucose uptake ability in response to insulin. In another embodiment, Asprosin itself promotes insulin resistance in mice, and thus its absence in NPS may have a direct insulin sensitizing effect.

The data indicate that the extreme reduction in circulating Asprosin in NPS, beyond what would be predicted from the NPS genotype, is at least partly the result of a dominant negative effect of the intracellularly trapped mutant profibrillin that escapes nonsense-mediated decay. In specific embodiments, this is why whole gene deletions, non-truncating mutations, or truncating mutations that are proximal to the furin cleavage site, result in Marfan syndrome but not in the additional feature of lipodystrophy that characterizes NPS (Pyeritz, et al., 2009).

Asprosin is remarkable for two reasons. Mice exposed to exogenous Asprosin displayed expansion of their adipose mass and insulin resistance in just 10 days' time. Of note, this was achieved on standard chow rather than a high fat diet. Second, its coding region displays extremely high evolutionary conservation compared with the rest of profibrillin. This indicates a highly conserved function that is likely to be mediated by a cell-surface receptor. The identity of such a postulated receptor is not yet known. Because, based on its expression profile, adipose tissue is likely to be one of the more prevalent sites of asprosin production and secretion, it might seem paradoxical that asprosin is also necessary for fat cell differentiation. However, there are innumerable examples of molecules that serve to regulate their creating organ. Beyond adipogenic differentiation and expansion of fat mass, in some embodiments asprosin also regulates other functions of adipose, and perhaps other tissues. In fact, whether the asprosin-mediated perturbation of glucose homeostasis is an effect of altered fat mass or altered fat activity remains unknown.

The results provide intriguing therapeutic avenues. The most obvious is simply correcting the deficit in NPS patients. However, recombinant asprosin is useful in patients with cachexia secondary to diverse etiologies such as advanced age, cancer, HIV infection, etc., for example. Such patients have significant frailty from reduced adipose mass (Mueller, et al., 2014; Pureza & Florea, 2013; Gelato, et al., 2007; Agarwal, et al., 2013; Kulstad & Schoeller, 2007) among other causes, and might benefit from the adipose expansion afforded by asprosin. Conversely, decreasing circulating asprosin may bring about a reduction in adipose mass and improved glycemic control in patients with obesity and diabetes. In certain embodiments, NPS associated lipodystrophy and obesity are two ends of the asprosin equation, with too little at one end and too much at the other. In any event, correction of circulating asprosin levels in conditions of pathologically altered fat mass affords significant therapeutic benefit, in particular embodiments of the disclosure.

### Example 6

### Asprosin, a Fasting-Induced Glucogenic Protein Hormone

Introduction

Hormones, their receptors, and associated signaling pathways make compelling drug targets because of their wide-ranging biological significance (Behrens and Bromer, 1958). Protein hormones, as a subclass, have defining characteristics. They usually (but not always) result from cleavage of a larger proprotein, and upon secretion traffic via the circulation to a target organ. There, they bind a target cell using a cell surface receptor, displaying high affinity, saturability and ability to be competed off. They stimulate rapid signal transduction using a second messenger system followed by a measurable physiological consequence. Given the brain's strict dependence on glucose as a fuel, plasma glucose levels are precisely regulated by an array of hormones (Aronoff et al., 2004). Some are secreted in response to nutritional cues, while others respond to glucose itself, producing highly coordinated and precise regulation of circulating glucose levels. Perturbations in this system can cause pathological alteration in glucose levels, often with severe consequences given the brain's dependence on glucose as a fuel.

The present example provides studies related to asprosin, a protein hormone that regulates glucose homeostasis, that is the C-terminal cleavage product of profibrillin (encoded by FBN1). Its absence in humans results in a unique pattern of metabolic dysregulation that includes partial lipodystrophy accompanied by reduced plasma insulin while maintaining euglycemia.

Examples of Results

Neonatal Progeroid Syndrome (NPS) Mutations Reduce Plasma Insulin Levels while Maintaining Euglycemia in Humans

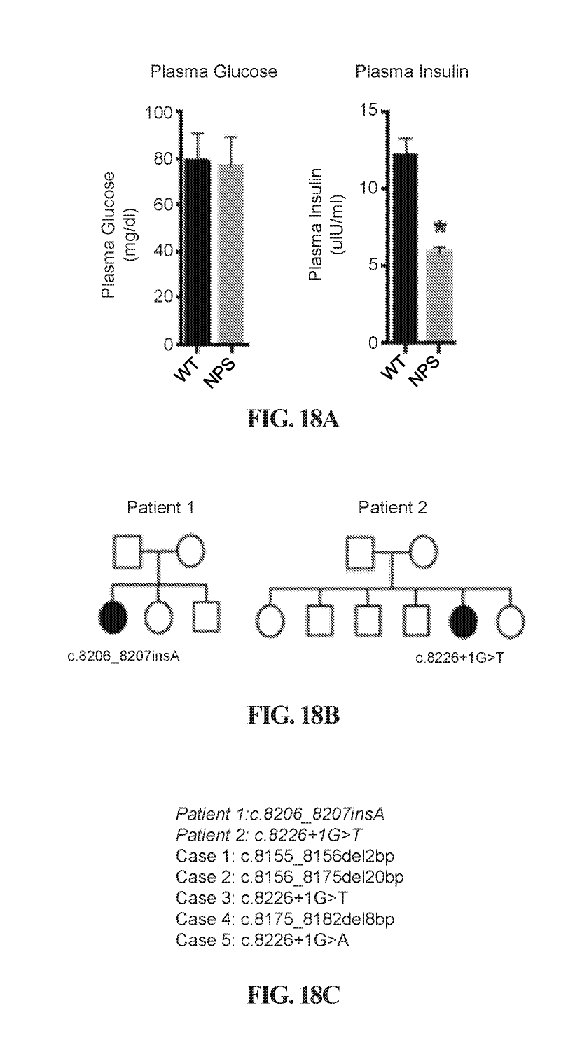

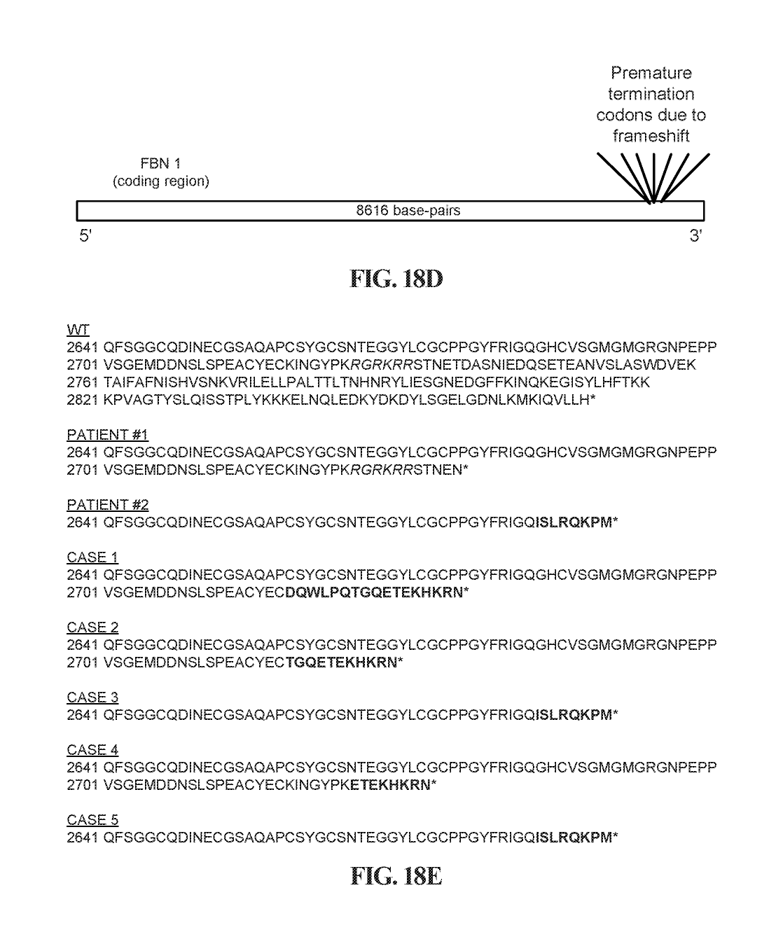

NPS was first described in 1977 (OMIM 264090) and is characterized by congenital, partial lipodystrophy, predominantly affecting the face and extremities (O'Neill et al., 2007). Although NPS patients appear progeroid because of facial dysmorphic features and reduced subcutaneous fat, the term is a misnomer as the patients do not display accelerated aging. Two unrelated individuals with NPS were identified. Their glucose and insulin homeostasis status was examined, since both partial and generalized lipodystrophic disorders are frequently associated with insulin resistance (Bindlish et al., 2015). Contrary to this notion, overnight-fasted plasma insulin levels from the NPS patients were 2-fold lower than unaffected subjects while maintaining euglycemia (FIG. 18A).

Whole-exome and Sanger sequencing identified de novo, heterozygous, 3′, truncating mutations in FBN1 in both patients (FIGS. 18B-18C). Upon reaching the genetic diagnosis, the literature was searched for similar cases and discovered five single-patient case reports of NPS associated with FBN1 3′ truncating mutations (Goldblatt et al., 2011; Graul-Neumann et al., 2010; Horn and Robinson, 2011; Jacquinet et al., 2014; Takenouchi et al., 2013). All 7 subjects, including the two reported herein, were diagnosed with NPS and all have truncating mutations within a 71 base pair segment at the 3′ end of the FBN1 coding region, displaying tight genotype-phenotype correlation (FIG. 18D). All 7 mutations occur 3′ to the last 50 nucleotides of the penultimate exon and are therefore predicted to escape mRNA nonsense mediated decay (NMD), leading to expression of a mutant, truncated profibrillin protein (FIG. 18E).

Profibrillin is translated as a 2871 amino acid proprotein, which is cleaved at the C-terminus by the protease furin (Lonnqvist et al., 1998; Milewicz et al., 1995). This generates a 140 amino acid C-terminal cleavage product, in addition to mature fibrillin-1 (an extracellular matrix component). All seven NPS mutations are clustered around the cleavage site, resulting in heterozygous ablation of the C-terminal cleavage product (asprosin) (FIG. 18E), whose fate and function were unknown.

Asprosin, the C-Terminal Cleavage Product of Profibrillin, is a Fasting Responsive Plasma Protein

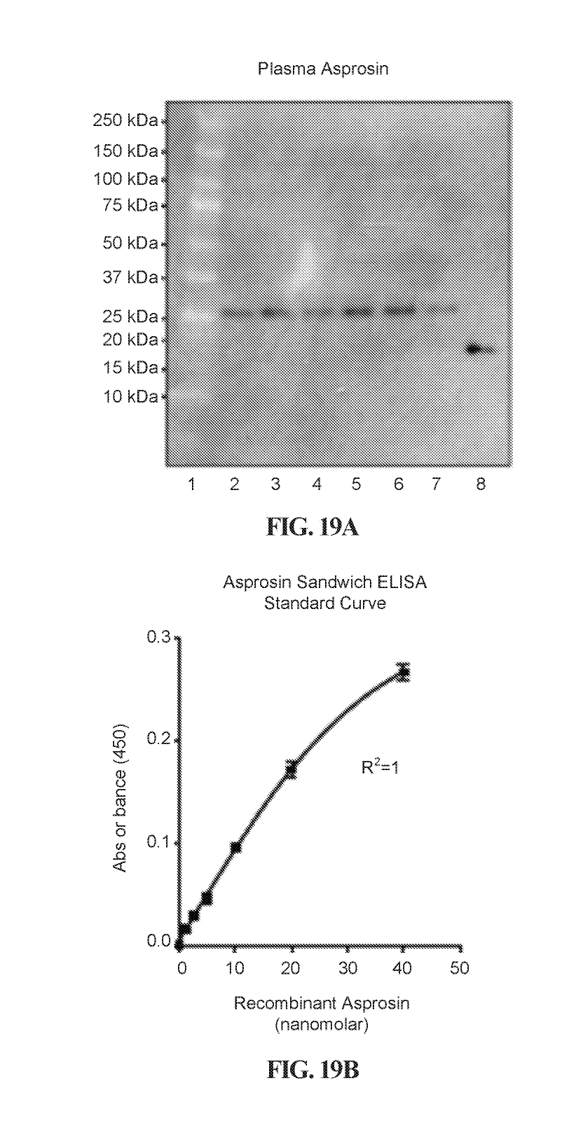

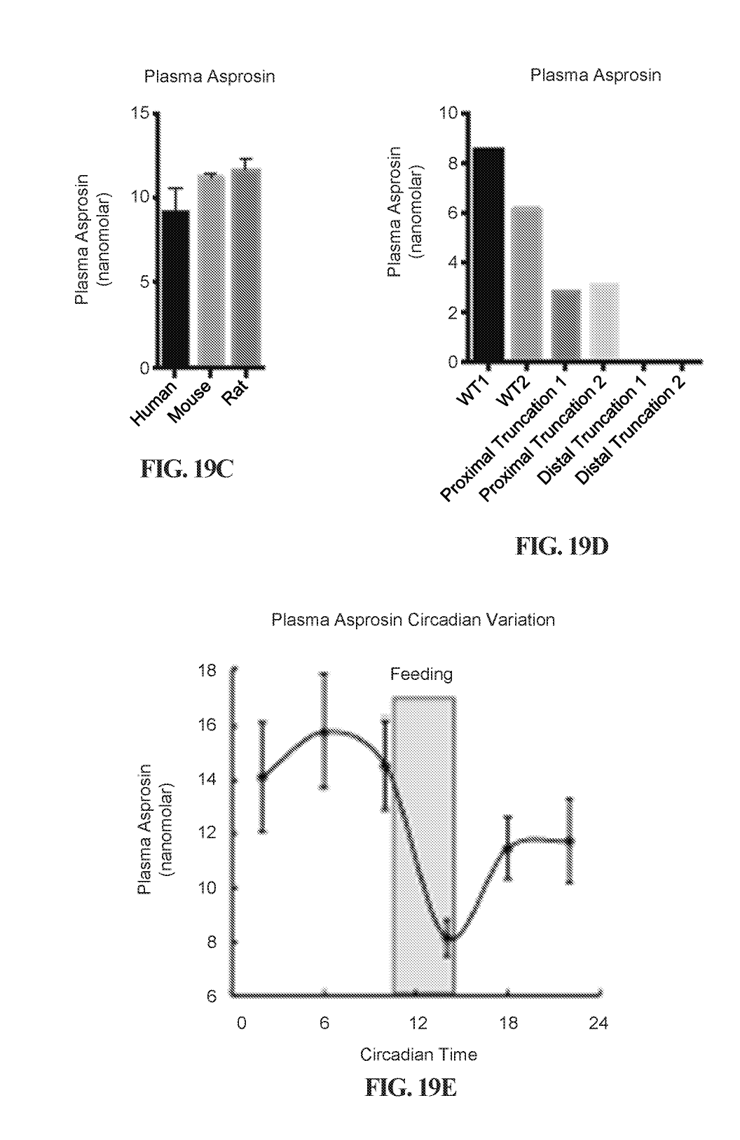

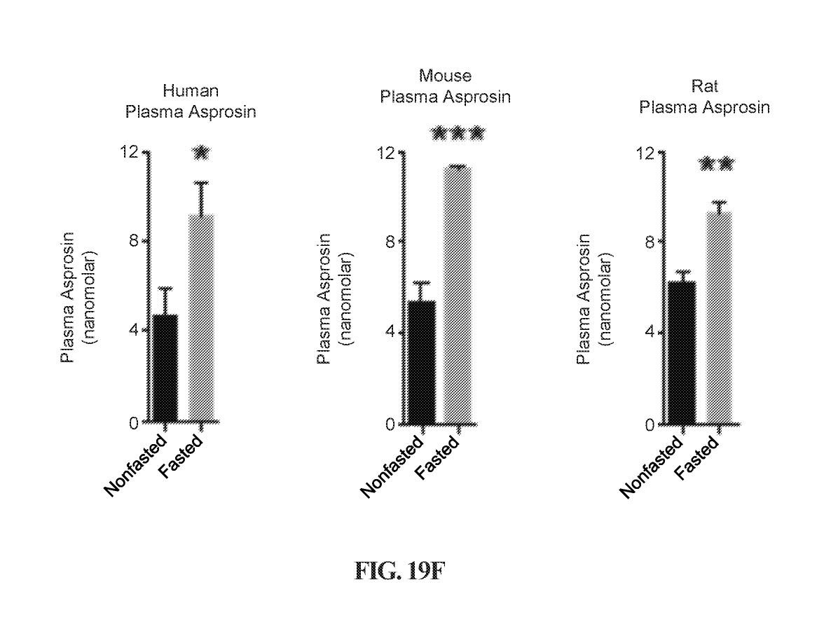

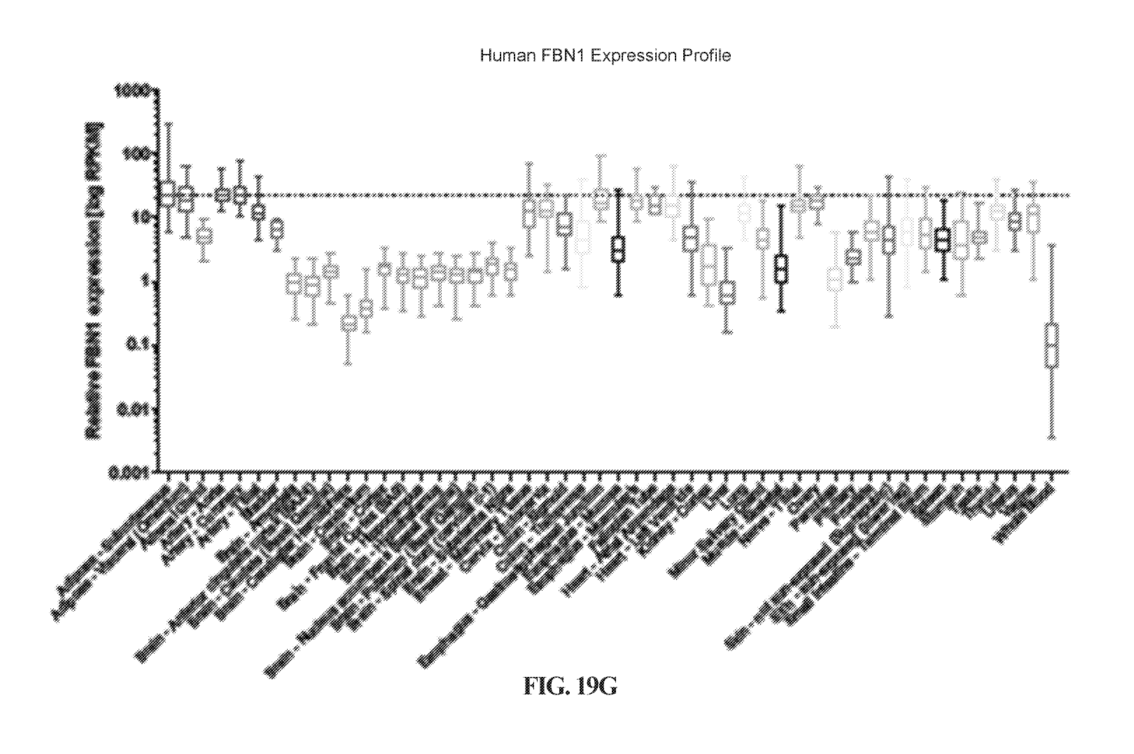

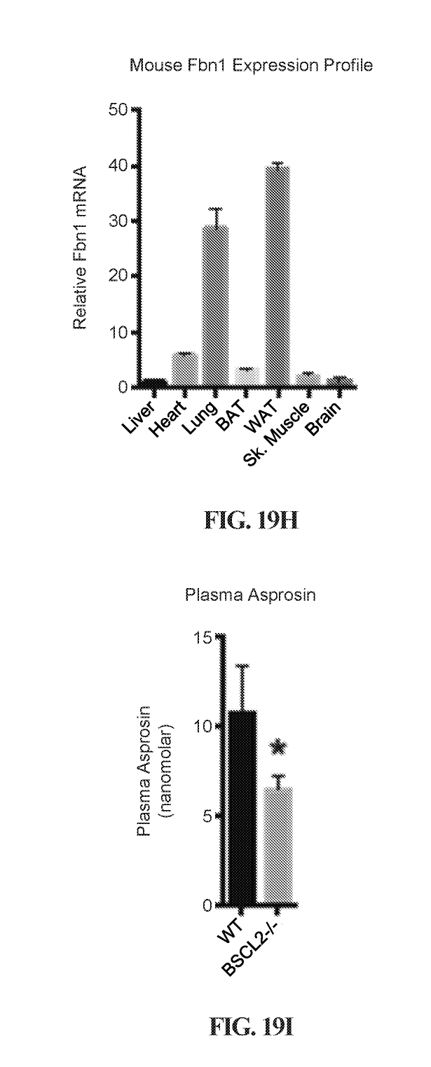

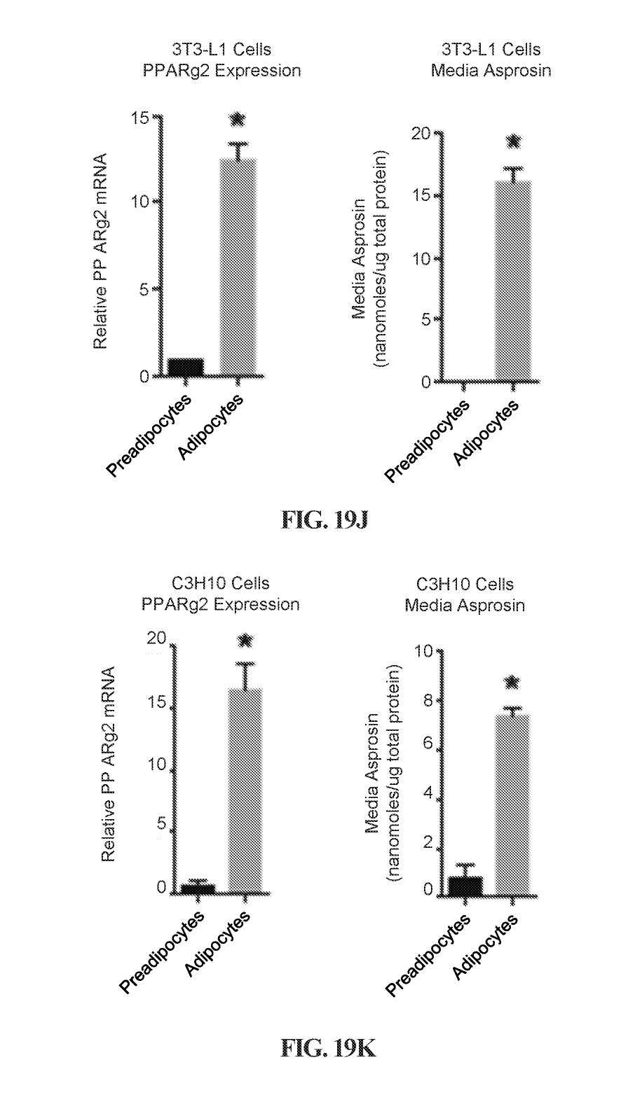

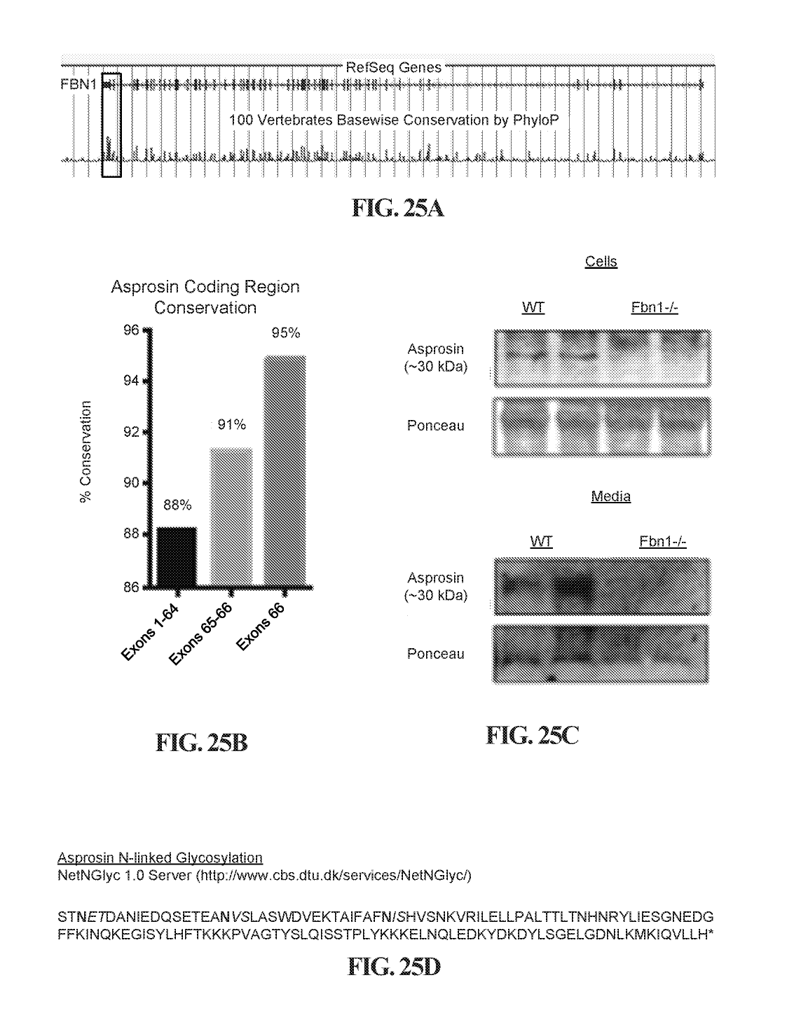

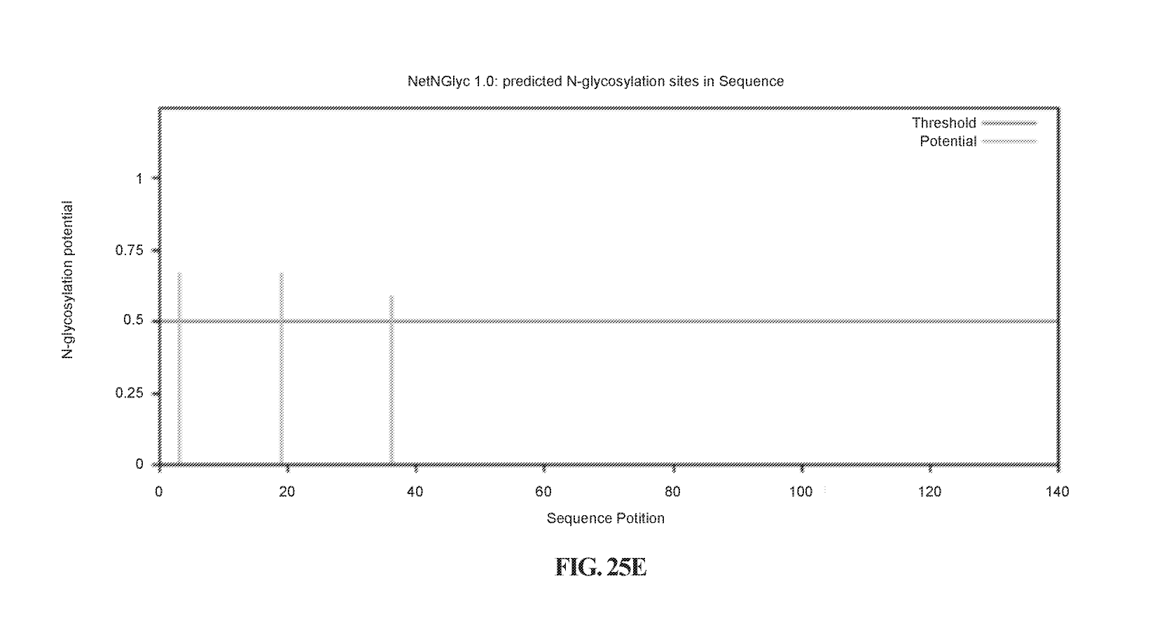

Asprosin is encoded by the ultimate two exons of FBN1. Exon 65 encodes 11 amino acids while exon 66 encodes 129 amino acids. Together those two exons display a somewhat higher vertebrate evolutionary conservation score compared with the rest of the profibrillin coding sequence (FIGS. 25A-25B). An asprosin-specific monoclonal antibody was produced and its specificity for asprosin was validated using Fbn1 WT and null cells (FIG. 25C). Immunoblotting human plasma with the anti-asprosin antibody shows a single protein running on SDS-PAGE at ˜30 kDa, while bacterially expressed recombinant asprosin runs at ˜17 kDa (FIG. 19A). Asprosin is predicted to have three N-linked glycosylation sites, and potentially other post-translational modifications that are lacking in bacteria (FIGS. 25D-25E). This likely explains the difference in molecular weight between mammalian and bacterially expressed asprosin. Indeed, using mammalian cells for expression of asprosin produced a protein that was secreted into the media, and ran on SDS-PAGE at the same molecular weight (˜30 kDa) (Lonnqvist et al., 1998) as was observed in human plasma, cell lysates and media from mouse embryonic fibroblasts, and cell/tissue lysates from cultured adipocytes and mouse white adipose tissue (FIGS. 19A, 25C, 26A-26B).

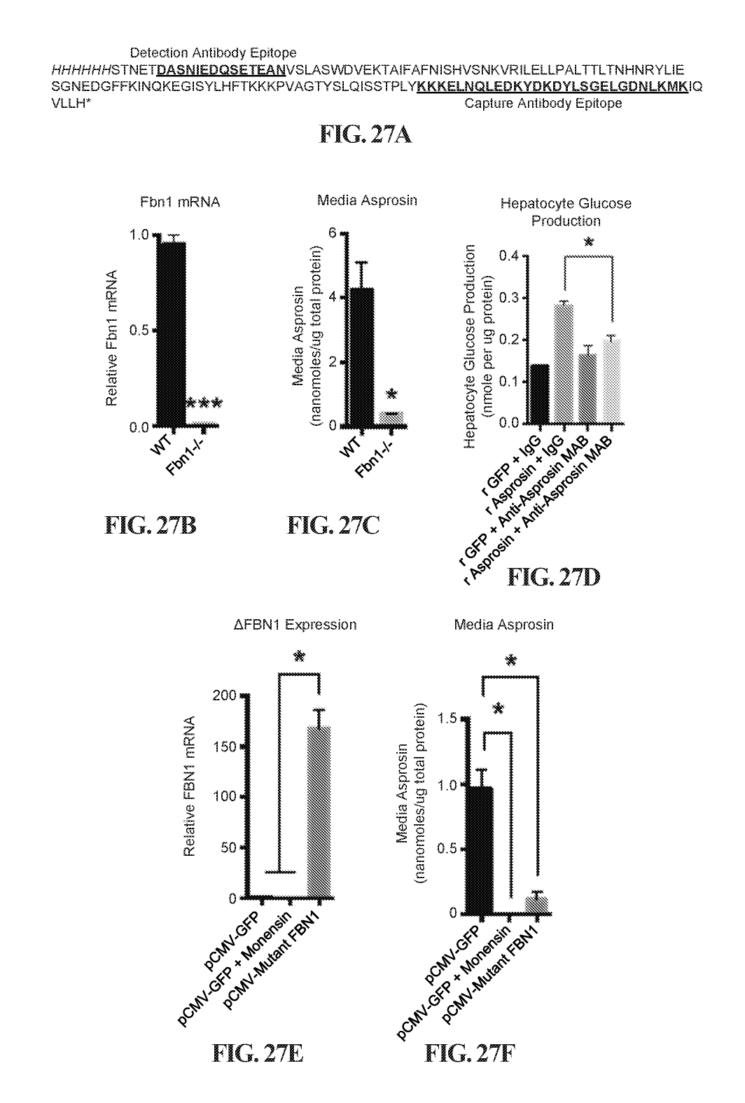

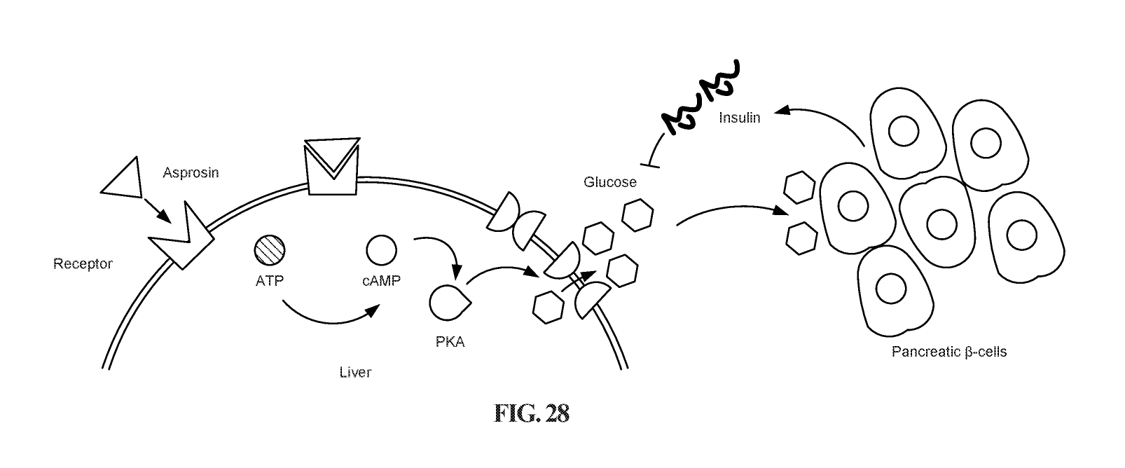

To measure circulating asprosin levels a sandwich ELISA was developed (FIG. 27A). A standard curve was constructed using recombinant asprosin and used it to calculate plasma and media levels (FIG. 19B). As expected, the asprosin sandwich ELISA displayed high specificity using media from WT and Fbn1−/− cells (FIG. 27C). Asprosin was found to be present in plasma at consistent nanomolar levels in humans, mice and rats (FIG. 19C). Interestingly, NPS patients displayed a greater reduction in circulating asprosin level than predicted from their heterozygous genotype, compared not only with WT control subjects, but also when compared with patients that have heterozygous truncations of profibrillin sufficiently N-terminal so as to undergo mRNA nonsense mediated decay (FIG. 19D). This suggests that the mutant profibrillin that is predicted to be expressed in NPS cells (due to escape from mRNA NMD) exerts a dominant negative effect on secretion of asprosin from the WT allele. This concept was characterized by overexpressing the truncated, mutant version of profibrillin in WT cells and it was found that this interfered with the ability of those cells to secrete asprosin into the media, compared with overexpression of an irrelevant protein such as GFP (FIGS. 27E-27F).

To assess daily fluctuations in circulating asprosin concentrations, mice were kept in a 12-h light/12-h dark cycle for seven days to establish entrainment, and were subsequently kept in constant darkness. Plasma was then isolated from these mice at 4-hour intervals and subjected to asprosin ELISA analysis. Plasma asprosin displays circadian oscillation with an acute drop in levels coinciding with the onset of feeding (FIG. 19E). In the opposite direction, overnight fasting in humans, mice and rats resulted in increased circulating asprosin (FIG. 19F).

Adipose Tissue Generates and Secretes Asprosin

The FBN1 mRNA profile was examined across all human tissues using the Genotype-Tissue Expression Project (GTex) RNAseq dataset, and it was found that adipose tissue demonstrated the highest FBN1 mRNA expression across all tissues (FIG. 19G). To confirm this in mice, the Fbn1 expression profile was assessed across various metabolically important organs. Consistent with the human profile, white adipose tissue displayed the highest Fbn1 mRNA expression (FIG. 19H). Given that white adipose tissue is a well-known endocrine organ (Trayhurn et al., 2006), it was examined whether it could serve as a source of circulating asprosin. Plasma levels of asprosin were assessed in mice that had been subjected to genetic ablation of adipose tissue. Bscl2−/− mice were used for this purpose. BSCL2 deficiency results in Berardinelli-Seip congenital lipodystrophy in humans (knockout mice mimic this phenotype) with a 60-70% reduction in adipose tissue (Cui et al., 2011). In such mice there was a ˜2-fold reduction in plasma asprosin (FIG. 19I). The next experimental strategy employed was to assess whether adipocytes in culture were capable of generating and secreting asprosin. For this, two distinct adipogenic cell lines, 3T3-L1 and a mesenchymal stem cell line—C3H10T1/2, were differentiated into mature adipocytes (FIGS. 19J-19K) and the cell culture media was subjected to asprosin protein analysis. There was robust accumulation of asprosin in serum-free culture media from mature adipocytes but not from preadipocytes (FIGS. 19J-19K), suggesting that adipocytes are capable of generating and secreting asprosin.

A Single Dose of Recombinant Asprosin Elevates Blood Glucose and Insulin in Mice

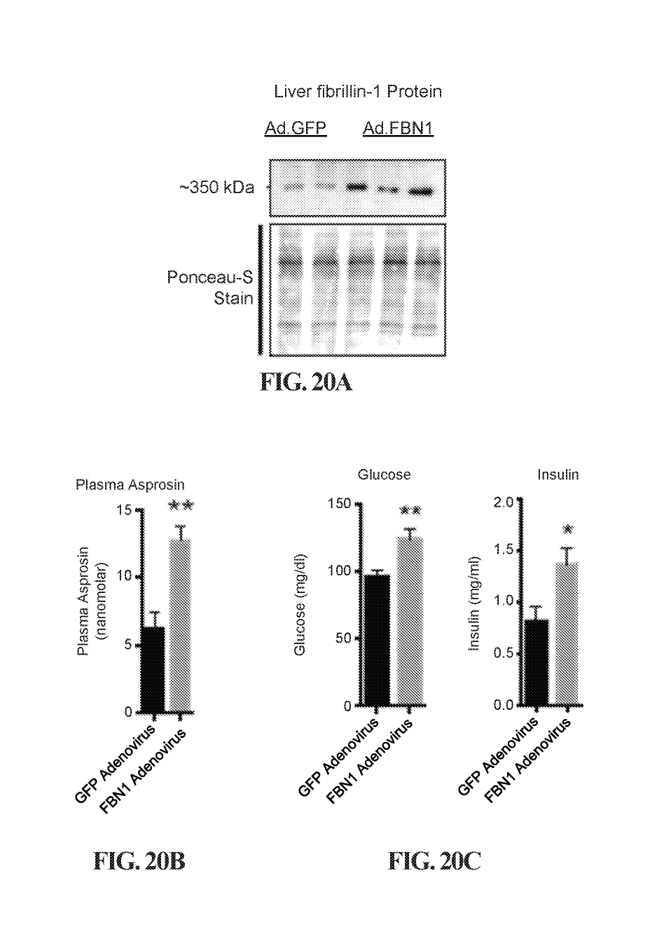

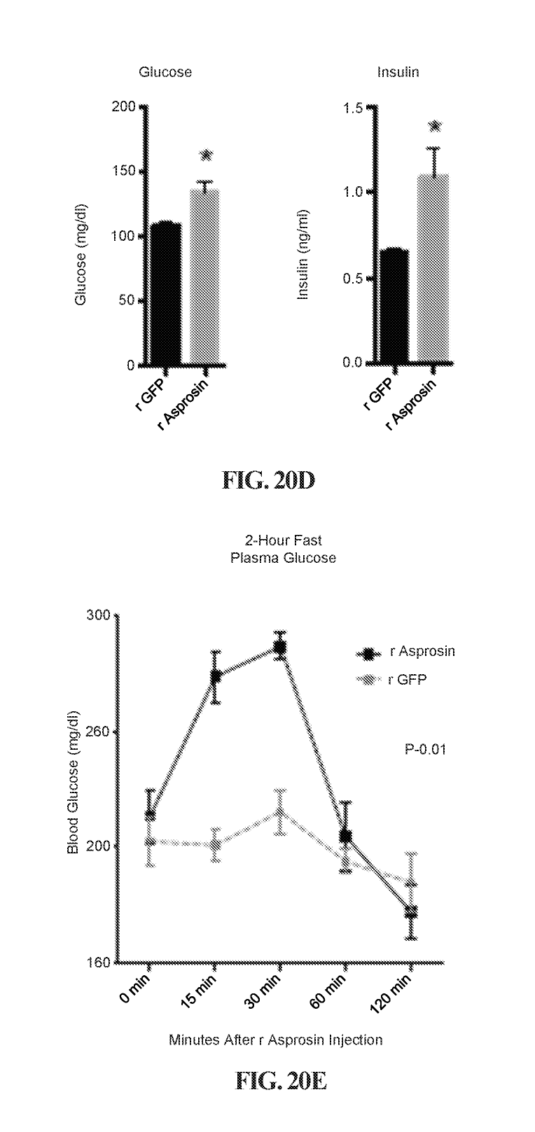

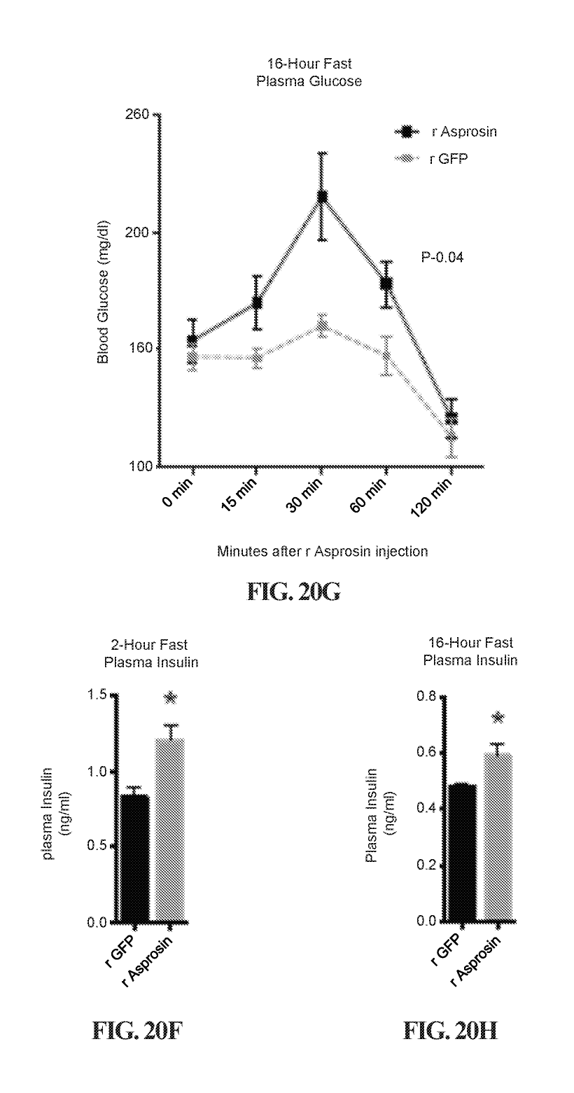

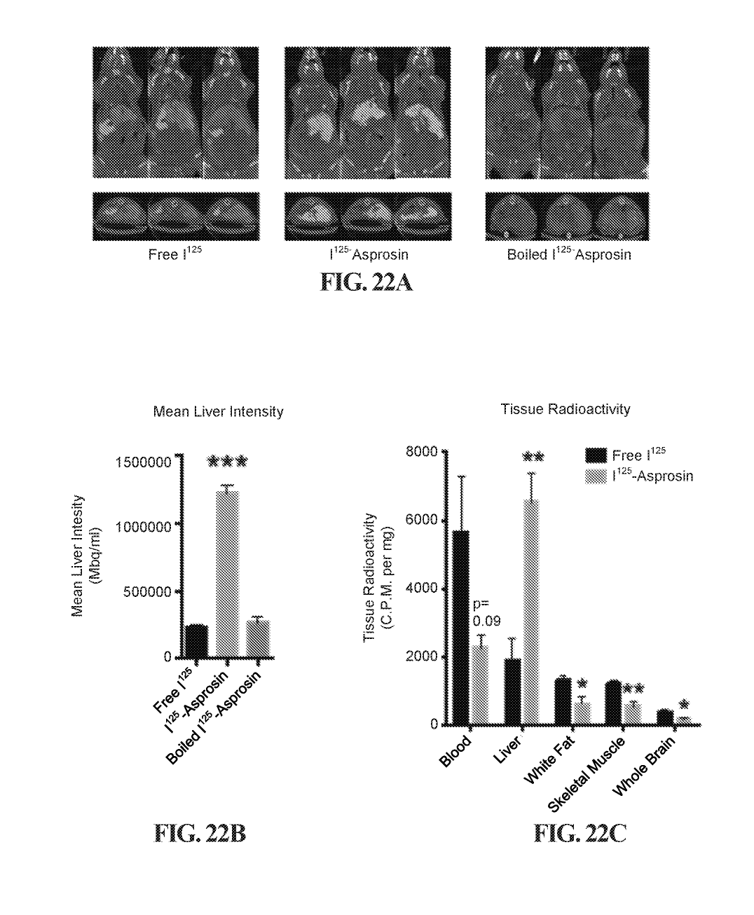

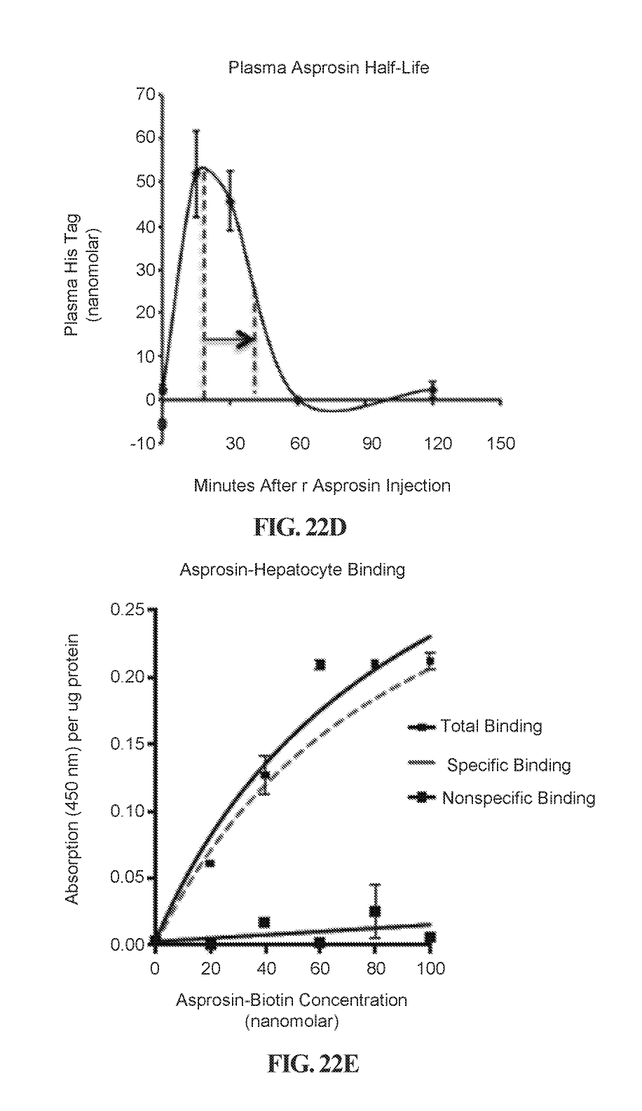

Ectopic overexpression of full length FBN1 was employed using an adenovirus in the hope that the transduced organ (the liver in this case, which normally shows low endogenous FBN1 expression (FIGS. 19G-19H), and is the primary target of adenoviral infection) would process the resulting profibrillin and secrete asprosin into the circulation. This strategy showed robust overexpression of profibrillin protein in the liver and a 2-fold elevation in plasma asprosin (FIGS. 20A-20B). The second strategy involved daily, subcutaneous injection of bacterially expressed asprosin (validated to result in a 50 nM peak level 20 minutes after injection—FIG. 22D) or recombinant GFP as a control. 10 days of exposure to increased plasma asprosin in either a continuous (adenoviral overexpression) or pulsatile fashion (daily recombinant asprosin injection) resulted in elevated glucose and insulin levels in 2-hour fasted mice using both experimental strategies (FIGS. 20C-20D). This result demonstrated that bacterially expressed recombinant asprosin retains the biological activity displayed by its endogenously expressed counterpart, and that elevation of circulating asprosin is sufficient to increase blood glucose and insulin levels.

In order to understand acute responses, a single dose of recombinant asprosin was injected subcutaneously in mice that had been subjected to a 2-hour preceding fast, and measured plasma glucose at 15, 30, 60 and 120 minutes post-injection. Mice were denied access to food through the length of the experiment. A single asprosin dose resulted in an immediate spike in blood glucose levels (FIG. 20E). This resulted in compensatory hyperinsulinemia (measured at the 15 minute time-point) (FIG. 20F) which normalized blood glucose levels by 60 minutes post injection (FIG. 20E). Similar results were obtained in mice that were subjected to an overnight preceding fast although the rate of the resulting blood glucose spike was somewhat slower, likely due to fasting-induced depletion of glucogenic substrates (FIGS. 20G-20H). These results implicated the liver as the target organ for asprosin due to its role as the primary site for stored glucose (as glycogen) that is rapidly released into the circulation during fasting. Interestingly, asprosin treatment had no effect on plasma levels of catabolic hormones (glucagon, catecholamines, glucocorticoids) known to induce hepatic glucose release (FIG. 20I).

Asprosin Targets the Liver to Increase Plasma Glucose in a Cell-Autonomous Manner

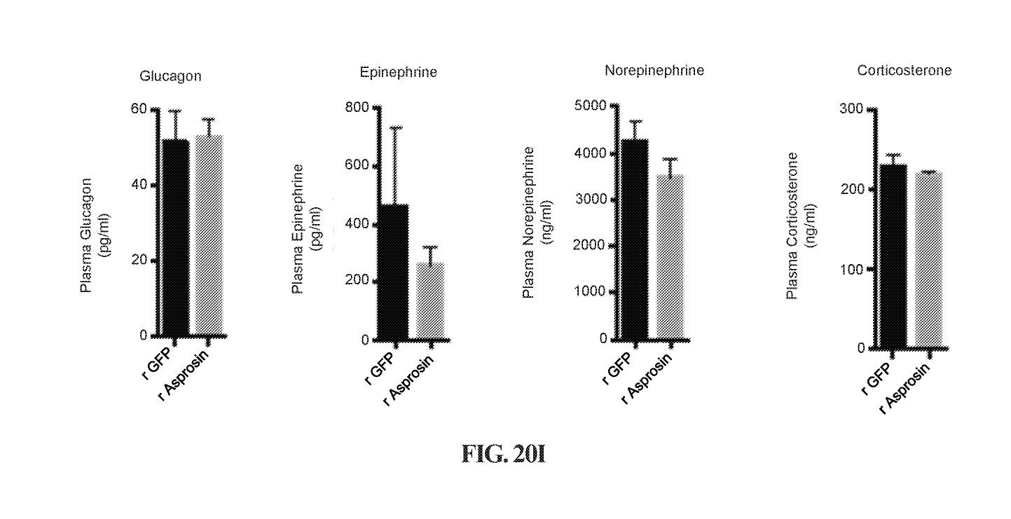

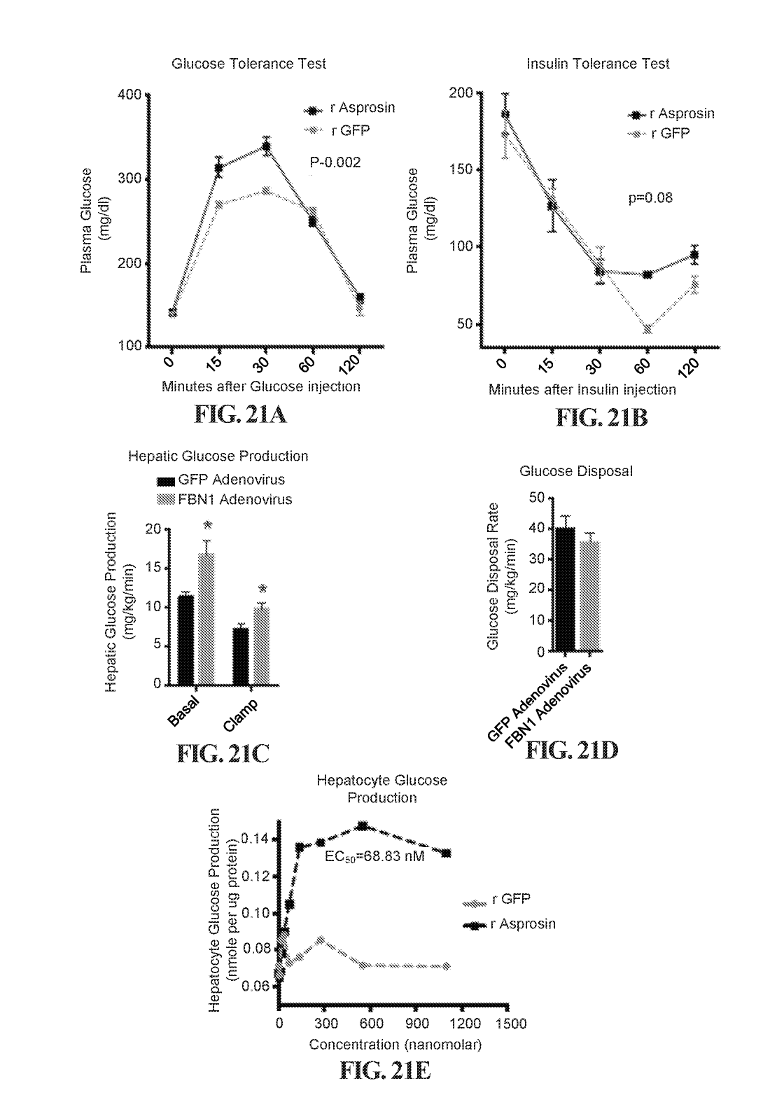

Glucose- and insulin-tolerance tests in mice exposed to a single dose of recombinant asprosin showed little evidence of altered glucose uptake (in response to insulin) in peripheral organs such as muscle or fat (unchanged slope of glucose disposal), but showed altered peak glucose levels, again implicating the liver (FIGS. 21A-21B). To confirm the liver as the site of asprosin action, the hyperinsulinemic-euglycemic clamp was performed. This test showed unequivocally that elevated plasma asprosin results in increased hepatic glucose production (FIG. 21C), but has no impact on the ability of peripheral organs to take up glucose in response to insulin (FIG. 21D). To test whether the effect of asprosin on the liver is cell-autonomous, isolated primary mouse hepatocytes were exposed to increasing concentration of recombinant asprosin or GFP for 2 hours. Media from cells exposed to asprosin showed an increase in glucose concentration in a dose-dependent manner, demonstrating a direct effect of asprosin on hepatocytes (FIG. 21E).

Asprosin Traffics to the Liver In Vivo and Binds the Hepatocyte Surface with High Affinity in a Saturable and Competitive Manner

Recombinant asprosin was labeled with iodine-125 (I¹²⁵) and injected intravenously in mice, followed by single-photon emission computerized tomography (SPECT) scans to identify sites of accumulation. An equivalent amount of free I¹²⁵, or I¹²⁵-Asprosin that was boiled for 5 minutes (to induce loss of the asprosin tertiary structure), were used as controls. In contrast to the accumulation patterns for free I¹²⁵ and boiled I¹²⁵-Asprosin, SPECT scans in coronal and axial planes (FIG. 22A), and mean liver photon intensity (FIG. 22B), both showed that I¹²⁵-Asprosin trafficked primarily to the liver, and that asprosin's tertiary structure was essential for its liver recruitment. In accord with liver trafficking, gamma counting of blood and viscera showed that recombinant blood asprosin levels decrease in concert with the increased liver levels (FIG. 22C). To measure plasma half-life, a sandwich ELISA system was used targeting the N-terminal His-tag on the recombinant asprosin protein at 15, 30, 60 and 120 minutes following subcutaneous injection. Consistent with results using IV infusion of I¹²⁵-Asprosin, plasma His-tagged asprosin showed a half-life of approximately 20 minutes and a peak level of 50 nM that was achieved 20 minutes post-injection (FIG. 22D).

To examine specific binding of asprosin by hepatocytes, mouse primary hepatocytes were incubated with an increasing amount of an asprosin-biotin conjugate, washed with PBS, and the relative level of biotin at the hepatocyte surface was measured. Asprosin bound the hepatocyte surface in a dose responsive and saturable manner (FIG. 22E). Repeating the same procedure in the presence of 100-fold excess unconjugated asprosin abolished the effect, suggesting competition for potential receptor binding sites (FIG. 22E).

Asprosin Uses the cAMP Second Messenger System and Activates Protein Kinase A (PKA) in the Liver

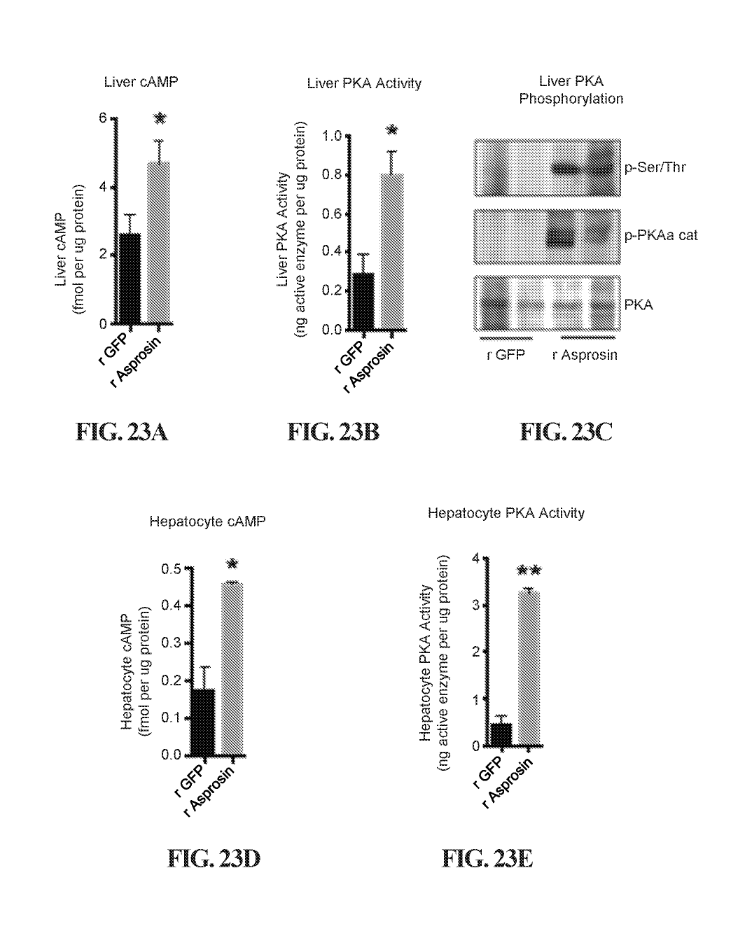

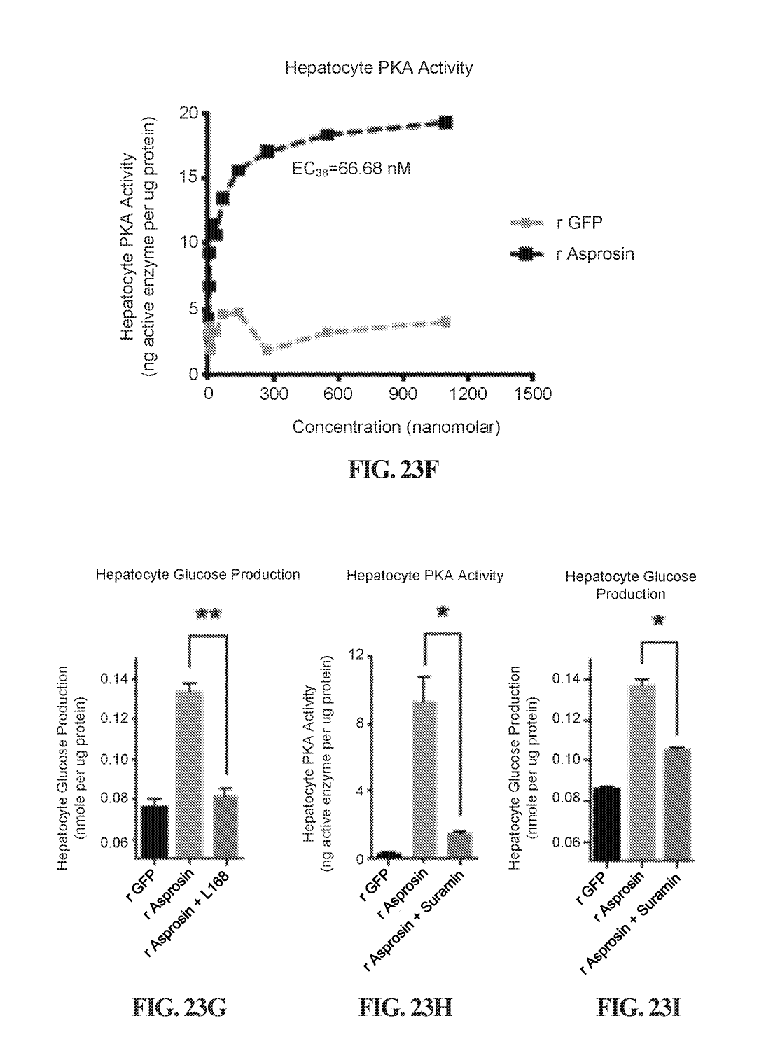

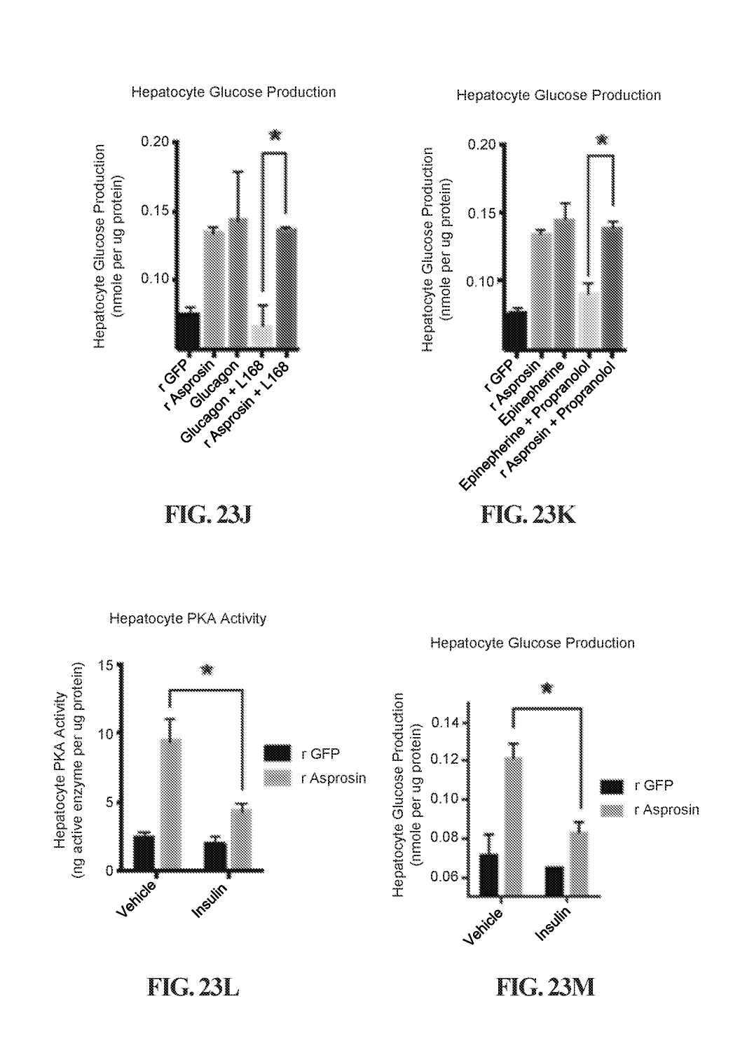

Exposing mice to a single 30 μg dose of recombinant asprosin for 20 minutes (validated to result in a 50 nM peak level) was sufficient to increase liver cAMP and protein kinase A activity (FIGS. 23A-23C). Identical results were obtained upon incubating mouse primary hepatocytes with recombinant asprosin for 10 minutes (FIGS. 23D-23E). Hepatocyte PKA activity increased in a dose responsive manner upon addition of recombinant asprosin (FIG. 23F), similar to what was observed with hepatocyte glucose release (FIG. 21E). The effects of asprosin on both hepatocyte glucose release and PKA activation were blocked by suramin, a general heterotrimeric G-protein inhibitor (FIGS. 23G-23H). In addition, asprosin mediated hepatocyte glucose release could be blocked by using cAMPS-Rp, a competitive antagonist of cAMP binding to PKA (FIG. 23I). These results demonstrate that asprosin increases hepatocyte glucose release by employing the G-protein-cAMP-PKA axis, in vivo and in vitro. Because glucagon and catecholamines also employ the same intracellular signaling axis, the impact was tested of inhibiting the glucagon receptor or the β-adrenergic receptor on the ability of asprosin to enhance hepatocyte glucose release. While the respective inhibitors completely blocked the effects of glucagon or epinephrine, they had no impact on the ability of asprosin to influence hepatocyte glucose release (FIGS. 23J-23K). This suggests that asprosin uses a cell-surface receptor system that is distinct from those used by glucagon and catecholamines. Since insulin is known to induce a reduction in intracellular cAMP (via activation of the G_{αi} pathway), it was tested whether insulin would oppose asprosin's effect on hepatocyte PKA activation and glucose release, which is demonstrated to be due to an increase in intracellular cAMP. Indeed, insulin suppressed asprosin-mediated hepatocyte PKA activation (FIG. 23L) and glucose release (FIG. 23M).

Asprosin Immunologic Sequestration is Protective Against Metabolic Syndrome Associated Hyperinsulinism

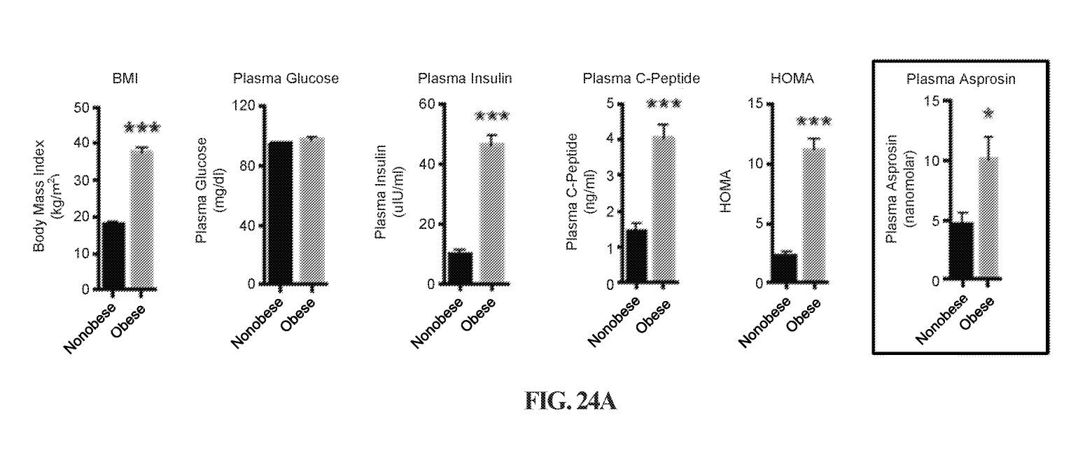

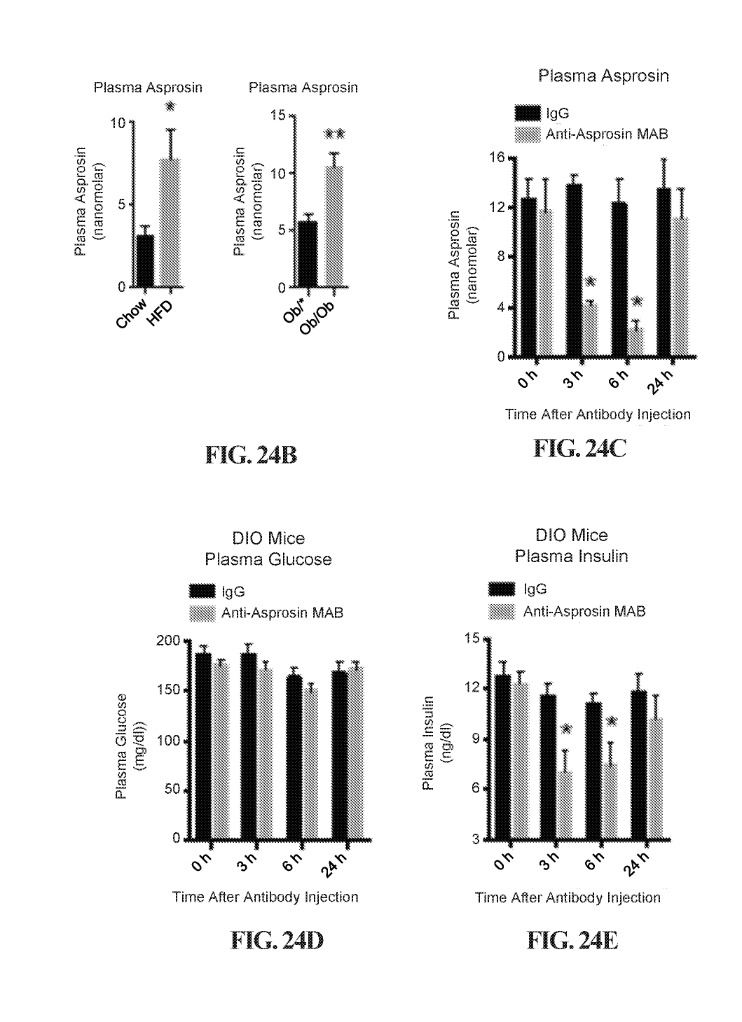

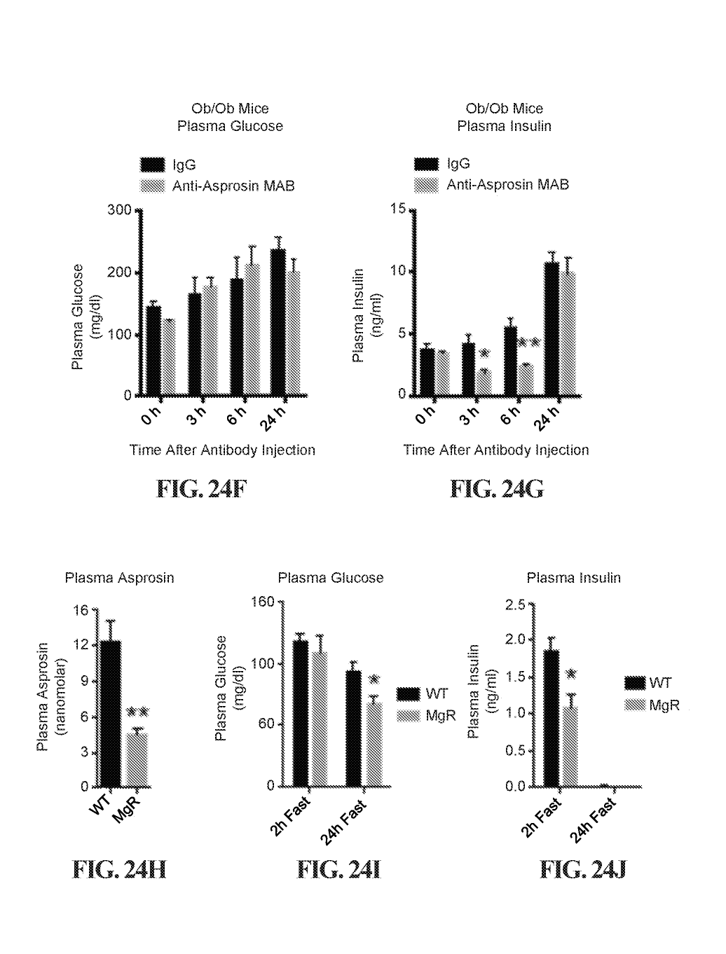

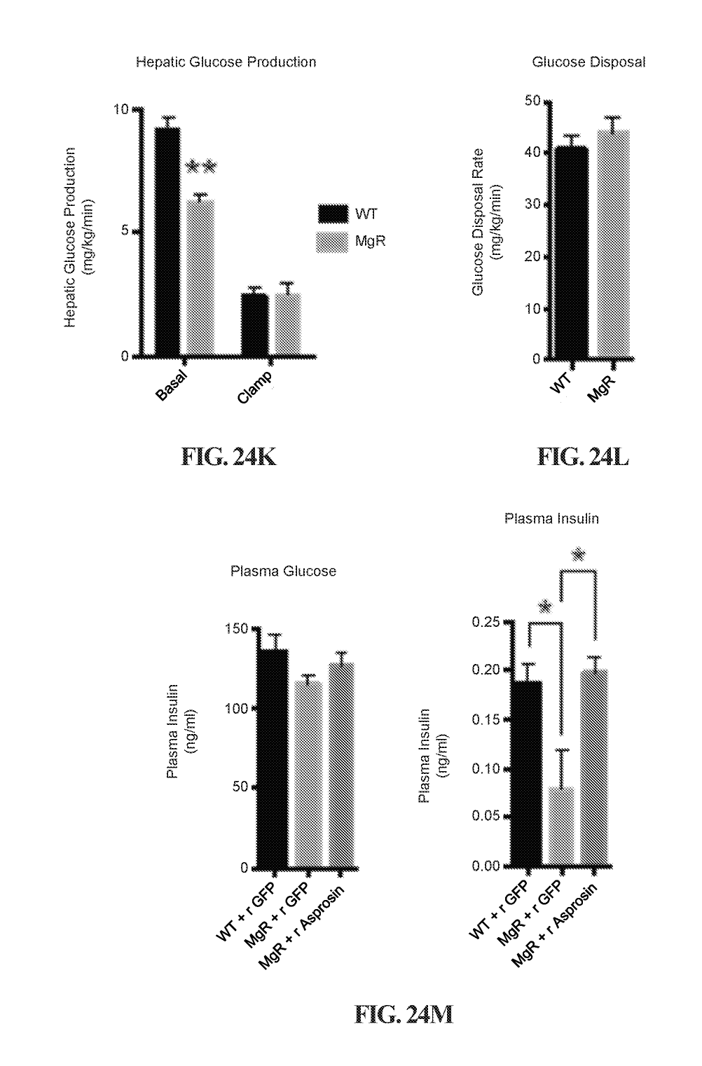

Plasma asprosin levels are pathologically elevated in human subjects with insulin resistance (FIG. 24A). Similar elevations were seen in two independent mouse models of insulin-resistance (diet induced obesity and Ob mutation) (FIG. 24B). Intra-peritoneal injection of a single dose of an asprosin specific monoclonal antibody was sufficient to acutely drop plasma asprosin levels at 3 and 6 hours post-injection, with recovery to normal levels at 24 hours (FIG. 24C). Both ad libitum fed (following a 2-hour fast for synchronization) models of mouse insulin-resistance showed an acute reduction in plasma insulin levels (while maintaining euglycemia), concurrent with plasma asprosin depletion (FIGS. 24D-24G). To directly test the effect of loss of asprosin on hepatocyte glucose production without the potential insulin compensatory effect, mouse primary hepatocytes were treated with the asprosin specific antibody prior to incubating them with asprosin. As expected, the asprosin specific antibody blocked asprosin mediated hepatocyte glucose release, while a nonspecific control antibody had no effect (FIG. 27D).

To validate immunologic sequestration as a legitimate loss-of-function strategy, FBN1 hypomorphic mice (homozygous MgR mice) were tested, which express only ˜20% of the WT FBN1 transcript (Pereira et al., 1999). MgR mice displayed a 70% decrease in circulating asprosin (FIG. 24H). Upon 2 hours of fasting, MgR mice displayed a 2-fold deficit in plasma insulin while maintaining euglycemia (similar to what was observed with immunologic sequestration of asprosin in ad libitum fed mice) (FIG. 24I-24J). However, upon 24 hours of fasting, a physiologic situation that eliminates insulin from the circulation of mice (FIG. 24J), MgR mice displayed fasting hypoglycemia (FIG. 24I), suggesting that insulin's buffering effect needs to be eliminated (via a long fast) to unmask the reduction in plasma glucose induced by asprosin loss-of-function. To confirm this, a hyperinsulinemic-euglycemic clamp study was performed on MgR mice that had been fasted for ˜18 hours (basal). Under such conditions, there was an acute deficit in hepatic glucose production (HGP) in MgR mice compared with WT mice (FIG. 24K). This result is consistent with clamp results showing an increase in HGP upon asprosin gain-of-function (FIG. 21C-21D). Expectedly, neither clamp study demonstrated a change in whole-body glucose disposal (insulin sensitivity) (FIGS. 21D, 24L), suggesting that asprosin's effect on glucose homeostasis is limited to serving as a stimulator of HGP, and any change in plasma insulin levels are indirect and downstream of the change in HGP.

Finally, a single subcutaneous administration of asprosin in overnight fasted MgR mice was sufficient to completely rescue the insulin deficit displayed by these mice (FIG. 24M). This result demonstrates that the insulin deficit displayed by MgR mice is entirely due to a deficiency in circulating asprosin, and not to some indirect effect of their decreased expression of functional fibrillin protein.

Significance of Certain Embodiments

Whether circulating asprosin concentration is experimentally decreased (genetic depletion in NPS patients, genetic depletion in MgR mice, acute removal via immunologic sequestration in mice), or increased (adenovirus mediated overexpression, direct recombinant protein injection) the result is a corresponding change in plasma glucose and insulin. With asprosin loss-of-function, hypoglycemia is only unmasked upon elimination of β-cell mediated corrective action, by fasting mice long enough to drive insulin levels close to zero, leaving little room for β-cells to further decrease insulin secretion and normalize plasma glucose.

One might consider it unexpected that a nutritionally responsive hormone that displays circadian oscillation (FIG. 19E) would be derived from what would seem a relatively “static” structural/ECM protein. This led the inventors to examine the profile of the FBN1 transcript using a publicly available circadiomics database (http://circadiomics.igb.uci.edu). Interestingly, the Fbn1 transcript displays robust daily circadian oscillation in several tissues such as the heart, adrenal, lung, white fat and kidney. The notion that fibrillin-1 is a static, structural molecule can be further examined. One can also further examine the primary tissue of origin of asprosin. It is demonstrated that adipose is at least one of the sources of plasma asprosin. This observation is consistent with the known function of adipose as an endocrine organ and a sensor/modulator of energy homeostasis. However, other organs besides adipose could also serve as sources of plasma asprosin given the fairly high expression of FBN1 in several organs. In specific embodiments, sources of asprosin include pancreatic islet cells, lungs, heart, vascular smooth muscle, adrenal gland, visceral smooth muscle, ovaries, uterus, fallopian tubes, placenta, cervix, esophagus, breast, brain, white adipose, brown adipose, skeletal muscle, etc.

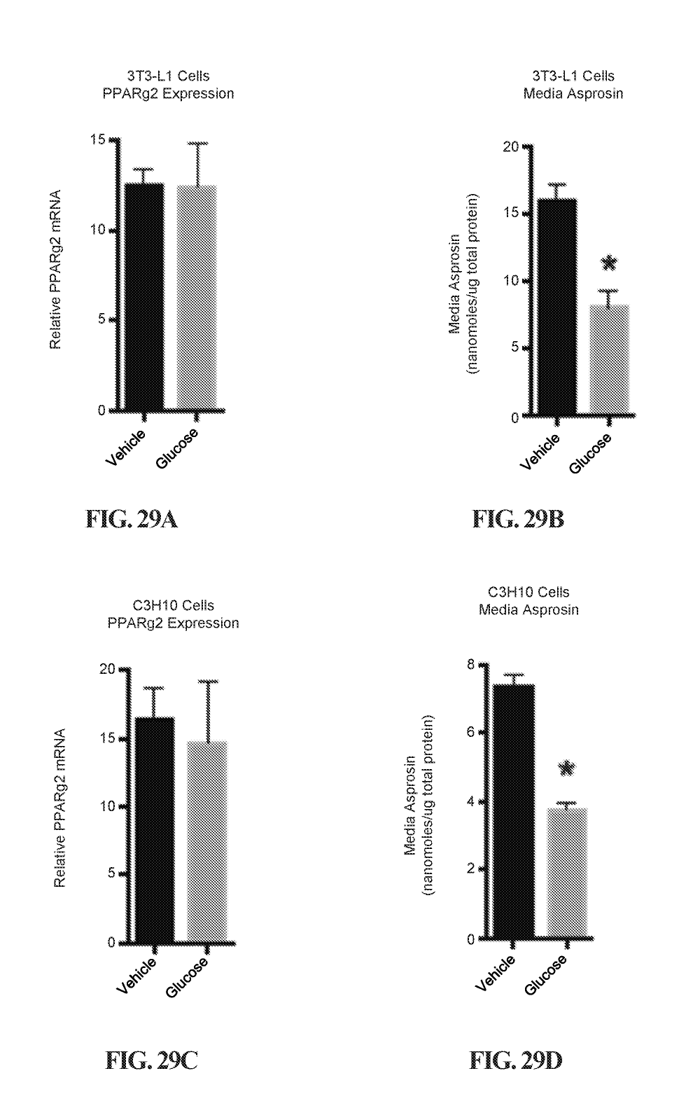

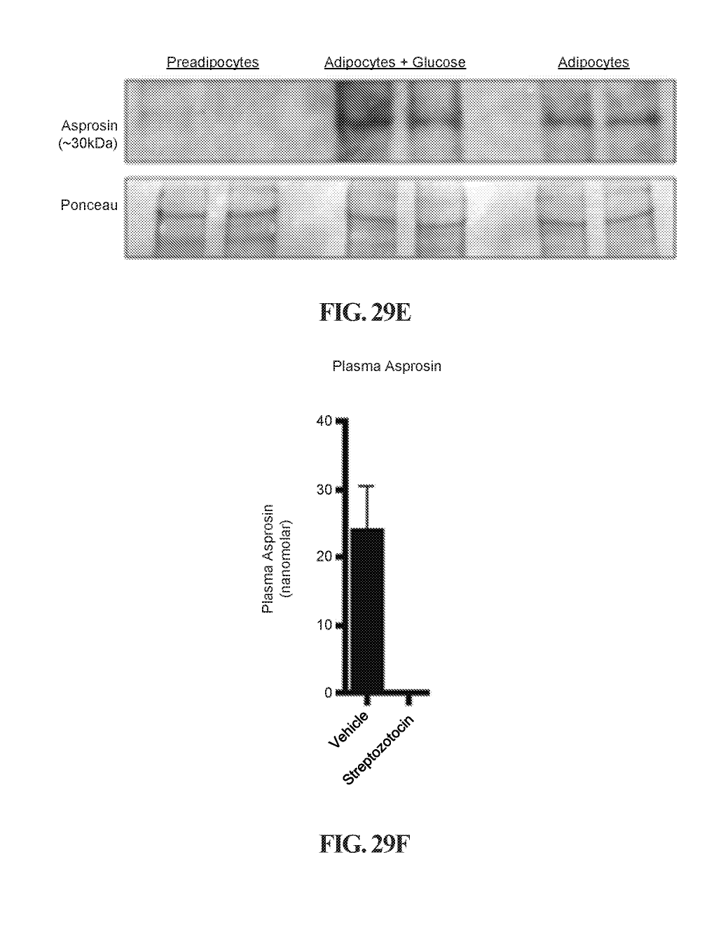

Because asprosin functions to increase plasma glucose levels, and circulating asprosin levels are increased by fasting (a baseline glucose condition) (FIG. 19F) and decreased by feeding (a high glucose condition) (FIG. 19E), it was considered that glucose could serve as a suppressor of plasma asprosin levels in a negative feedback loop, in some embodiments. To determine this, mature adipocytes were subjected in culture to high glucose levels and this treatment was sufficient to strongly inhibit the accumulation of asprosin in media, compared with adipocytes subjected to glucose free conditions (FIGS. 29B, 29D). There was no decrease in intracellular asprosin protein with glucose addition (FIG. 29E), suggesting that glucose-mediated down regulation of extracellular asprosin level does not occur at the level of transcription, biosynthesis or processing. To confirm this result in vivo, WT mice were subjected to Streptozotocin (STZ) treatment, which is known to ablate pancreatic β-cells resulting in high blood glucose. In such mice plasma asprosin was found to be far lower than in mice with normal blood glucose (FIG. 29F). Together, these in vitro and in vivo results are consistent with the notion that glucose serves as a negative influencer of plasma asprosin levels in a negative-feedback loop, and is consistent with the regulation of other major hormones (for example, calcium suppresses parathyroid hormone secretion and glucose suppresses glucagon secretion) (Campbell and Drucker, 2015; Dumoulin et al., 1995).

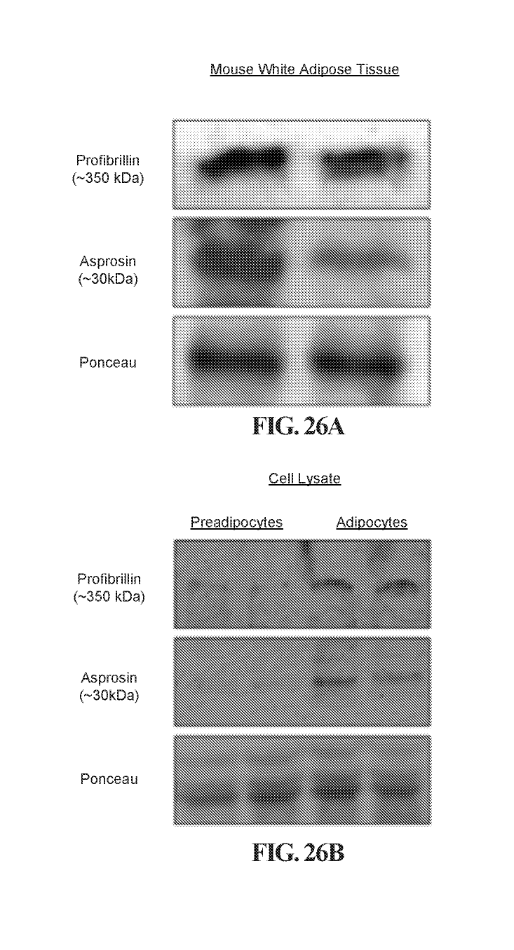

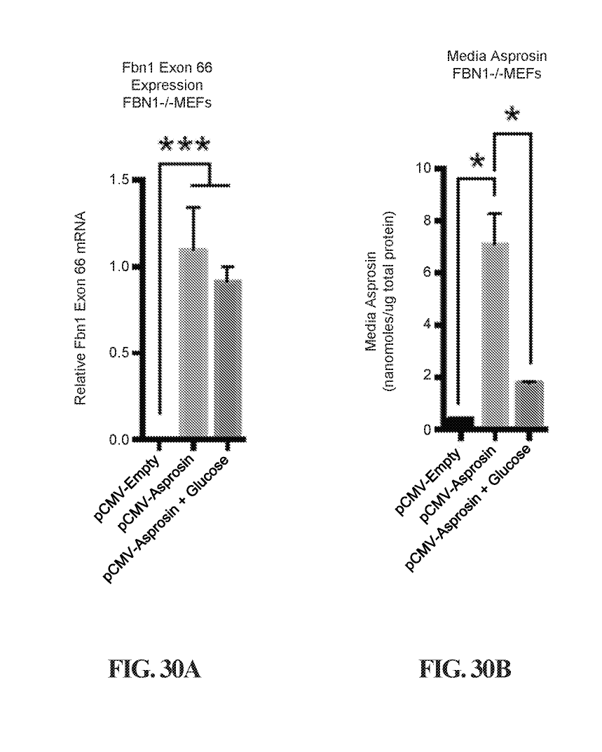

Generally, protein hormones are processed via endoplasmic reticulum and Golgi pathways and stored in intracellular granules, followed by secretion in response to appropriate cues. Consistent with this, there was detected processed asprosin intracellularly in cultured fibroblasts, mouse white adipose tissue and cultured adipocytes (FIGS. 25C, 26A-26B). Asprosin has been shown to retain the ability to be secreted from the cell despite the lack of a signal peptide. This was demonstrated by overexpressing just the asprosin coding exons in mammalian cells followed by detection of asprosin in the media (Lonnqvist et al., 1998). This assay was repeated by overexpressing asprosin-encoding cDNA in Fbn1−/− cells (to prevent contamination from endogenous asprosin) and detected asprosin secretion into the media (FIGS. 30A-30B). Furthermore, asprosin secretion could be suppressed by glucose (FIG. 30B), consistent with the phenomenon observed in cultured adipocytes (FIGS. 29B, 29D). Several extracellular proteins such as FGF-1, FGF-2 and IL-1β lack an N-terminal signal peptide and are secreted using non-classical or leaderless secretion (Nickel, 2003), as demonstrated by asprosin.

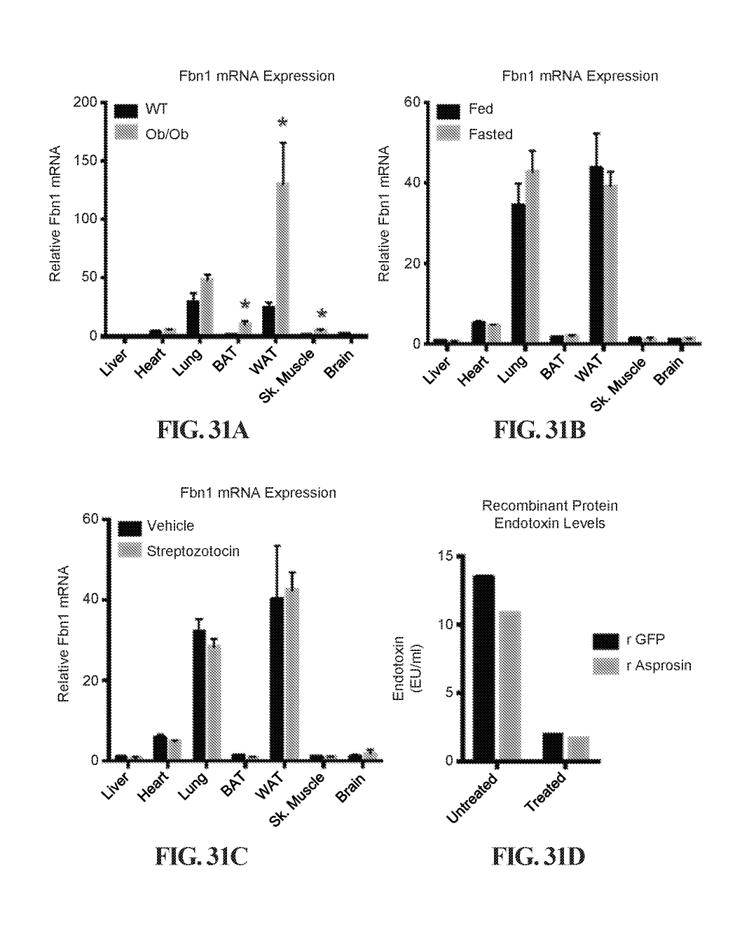

To assess the tissue sources of elevated asprosin with insulin resistance, the Fbn1 mRNA profile was assessed across various mouse tissues from WT and Ob/Ob mice. There was strong upregulation of the Fbn1 mRNA in white adipose tissue, brown adipose tissue and skeletal muscle (FIG. 31A), three organs that are frequently implicated in the pathogenesis of insulin resistance. The upregulation in white adipose tissue was especially potent, again implicating it as a major tissue source of plasma asprosin. The mechanism of upregulation of plasma asprosin via increased Fbn1 mRNA in adipose and skeletal muscle seems unique to the pathogenesis of insulin resistance because the inventors did not detect any changes in Fbn1 mRNA in any of the organs with fasting and streptozotocin treatment (FIGS. 31B-31C), two other manipulations associated with major changes in plasma asprosin. Type II diabetes remains a major cause of morbidity, in isolation, and as a part of metabolic syndrome. Inappropriately elevated glucose production by the insulin-resistant liver is a major factor underlying its pathogenesis (Magnusson et al., 1992). It is possible that the elevated asprosin levels observed in insulin-resistant humans and mice contribute to this. The insulin lowering effects of immunologic sequestration of asprosin in obese, insulin resistant mice suggest decreased asprosin activity as a unique approach to acutely counteract this pathologic effect. Accordingly, asprosin depletion may represent an important therapeutic strategy against type II diabetes.

Examples of Experimental Procedures

Study Subjects and Ethics Statement.

Informed consent and permission to use biological materials for research was obtained prior to participation from all subjects under one of four Institutional Review Board approved protocols at Baylor College of Medicine.

Clinical Evaluation.

Study subjects were assessed by clinical history, physical examination, and family history. After informed consent plasma isolation, body mass index measurements and body fat percentage measurements (DEXA) were carried out with standard universal precautions.

Whole-Exome Capture and Sequencing.

Genomic DNA from patient #1 and her parents was subjected to whole exome sequencing (trio analysis). Variants were annotated and analysis was performed in the trio to look for potential recessive (homozygous and compound heterozygous) and de novo variants.

Sanger Sequencing.

Genomic DNA from both patients was subjected to Sanger sequencing. Primers were designed to encompass exons 65 and 66, including intron-exon boundaries, of the FBNJ gene using Primer3. Sanger reads were analyzed using the Lasergene Seqman software. For patient #1, Sanger sequencing was performed on genomic DNA from both parents and unaffected siblings to confirm de novo occurrence and segregation with the phenotype.

Animals.

The inventors used 12-week-old male WT C57Bl/6 mice for in vivo studies. MgR heterozygous mice were obtained from Jackson labs, and bred to obtain male MgR homozygous mice and WT littermates. 5-week-old male Ob/Ob mice were obtained from Jackson labs. Mice were housed 2-5 per cage in a 12-hour light/12-hour dark cycle with ad libitum access to food and water. For diet induced obesity studies, mice were placed on the adjusted calories diet providing 60% of calories from fat by Harlan-Teklad for 12 weeks. Mice were exposed to adenoviral-mediated transgenesis (10¹¹ virus particles per mouse), via tail-vein injections. Mice were exposed to 30 μg recombinant His-tagged asprosin or recombinant Green Fluorescent Protein (GFP) daily for 10 days via subcutaneous injection. Mice were sacrificed and plasma and various organs were isolated 10 days after virus or peptide injection. For single dose injections, the same protocol was followed as that for daily injections, followed by collection of plasma at the indicated times via tail bleeds for insulin and glucose measurement. Insulin and glucose tolerance tests (ITT and GTT) were performed using standard procedures. A 0.5 U/kg insulin bolus was used for the ITT and a 1.5 mg/g glucose bolus was used for the GTT. For immunologic sequestration experiments, mice were injected intra-peritoneally with a 500 dose in saline of a custom-built (Thermo Scientific Inc.) anti-asprosin mouse monoclonal antibody directed against amino acids 106-134 (human profibrillin amino acids 2837-2865) or an equivalent dose of isotype matched IgG (Southern Biotech, Inc.). Hyperinsulinemic-euglycemic clamp studies were performed in unrestrained mice using regular human insulin (Humulin R, doses: 2.5 mu/kg body weight) in combination with HPLC purified [3-3H] glucose as described previously (Saha et al., 2010). The Baylor College of Medicine Institutional Animal Care and Utilization Committee approved all experiments.

Plasma Metabolic Parameters.

Human plasma insulin was measured using a human insulin ELISA kit by Abcam. Mouse plasma insulin was measured using a mouse insulin ELISA kit by Millipore. Mouse plasma glucagon, epinephrine, norepinephrine and corticosterone were measured by the Vanderbilt University Hormone Assay & Analytical Services Core.

Recombinant Asprosin and GFP.

Human FBN1 (2732-2871 amino acids) cDNA was cloned and subsequently sub-cloned into a pET-22B vector for expression in E. coli. The fusion protein that was expressed in E. coli is 146 amino acid long comprising of a 6 amino acid His tag on the N-terminus and a 140 amino acid wild type asprosin. His-tagged GFP expressed in E. coli was obtained from Thermo Scientific as the control protein. The His-Asprosin and His-GFP were isolated from E. coli and allowed to bind to Ni-NTA His-Bind column. After extensive washing of the column in order to remove contaminating proteins, His-Asprosin and His-GFP were eluted from the column using a 150 mM imidazole buffer. The recombinant proteins were further purified using size exclusion columns and polymyxin B based endotoxin depletion columns (Detoxi-Gel™ Endotoxin Removing Gel by Thermo Scientific Inc.) with as many passages as required to bring the final endotoxin concentration equal to or below 2 EU/ml, and buffer exchanged into a PBS-Glycerol buffer or a 20 mM MOPS, pH 7.0, 300 mM NaCl, 150 mM Imidazole buffer. The purified proteins were subjected to SDS-PAGE analysis in order to determine the purity level. The His-GFP and His-asprosin proteins used in all recombinant protein experiments were >90% pure with endotoxin levels (determined using the Pierce™ LAL Chromogenic Endotoxin Quantitation Kit) as indicated (FIG. 31D) before and after passage through endotoxin depletion columns.

Cell Culture.

Primary mouse hepatocytes were isolated from 8- to 12-week-old WT mice using standard methodology. Within a few minutes of isolation, cells were placed in glucose free media in Eppendorf tubes and subjected to treatment with recombinant 50 nM asprosin, 50 nM recombinant GFP, 5 μM Suramin (Tocris), 200 μM cAMPS-Rp (Tocris), 1 μM L168,049 (Tocris), 100 μM Epinephrine (Sigma), 10 μg/ml Glucagon (Sigma), 10 mg/L Insulin (Sigma) or 100 μM Propranolol (Sigma). The cells were treated with 50 nM recombinant asprosin or GFP for 10 minutes for cAMP and PKA assays and for 2 hours for the in vitro glucose production assay. Cells were pretreated with various inhibitors for 1 hour prior to treatment with asprosin, GFP, Glucagon or Epinephrine. cAMP was measured from cell lysates using the cAMP direct immunoassay kit from Cell Biolabs. PKA activity was measured from cell lysates using the PKA kinase activity kit from Enzo Lifesciences, Inc. Media glucose content was measured using the Glucose Colorimetric Assay Kit from Biovision. Results were normalized to protein content.

Recombinant asprosin was conjugated with biotin using the Basic Biotinylation Sulfo-NHS Kit from Pierce. Primary hepatocytes were incubated with increasing concentration of the asprosin-biotin conjugate at 4° C., alone, or in the presence of 100-fold excess unconjugated asprosin for 30 minutes. The cells were washed 3 times with PBS without lysis, followed by addition of streptavidin-HRP. The resulting absorbance was measured colorimetrically and the results were normalized to protein content.

3T3-L1 and C3H10T1/2 preadipocyte cells were exposed to an adipogenic cocktail (1 μM insulin, 1 μM dexamethasone, 0.5 mM isobutyl methyl xanthine and 3 μM rosiglitazone) for 7 days. Adipogenesis was confirmed by visualization of lipid droplets and PPARg2 mRNA expression.

For measurement of glucose mediated influence on secretion of asprosin, serum free DMEM with or without 4.5 g/L glucose was used.

WT human 140 amino acid asprosin (profibrillin amino acids 2732-2871), and mutant profibrillin carrying the c.8206_8207InsA mutation that induces a frame-shift and C-terminal truncation were sub-cloned under control of the CMV promoter using the pCMV6-Neo vector system.

Sandwich ELISA and Western Blot.

For the endogenous asprosin sandwich ELISA, a custom-built (Thermo Scientific Inc.) mouse monoclonal anti-asprosin antibody against asprosin amino acids 106-134 (human profibrillin amino acids 2838-2865) was used as the capture antibody and a goat anti-asprosin polyclonal antibody against asprosin amino acids 6-19 (human profibrillin amino acids 2737-2750) by Abnova was used as the detection antibody. An anti-goat secondary antibody linked to HRP was used to generate a signal. For the His-tag sandwich ELISA, the same procedure was used except for the use of a goat anti-His polyclonal antibody (Abcam) as the detection antibody. Increasing amounts of recombinant asprosin (which contains an N-terminal His tag) were used to generate a standard curve for both assays. EDTA-plasma was used for plasma sandwich ELISAs and serum-free DMEM concentrated using Vivaspin protein concentrator spin columns by GE Life Sciences Inc. was used for media sandwich ELISAs.

Plasma western blotting for asprosin was done using a custom-built (Thermo Scientific Inc.) mouse monoclonal anti-asprosin antibody against asprosin amino acids 106-134 (human profibrillin amino acids 2837-2865). Plasma was depleted of immunoglobulins and albumin using an Albumin/IgG removal kit by Pierce.

Western blotting for profibrillin was done using the mouse monoclonal anti-fibrillin-1 antibody against profibrillin amino acids 451-909 by Abcam.

Western blotting for phospho-PKA and total PKA was carried out using antibodies from Santa Cruz Biotechnology, Inc.

Statistical Methods.

All results are presented as mean±SEM. P values were calculated by unpaired Student's t-test for all results except where indicated by two-way ANOVA. *P<0.05, **P<0.01, and ***P<0.001.

### Example 7

### Anti-Asprosin Monoclonal Antibody Studies

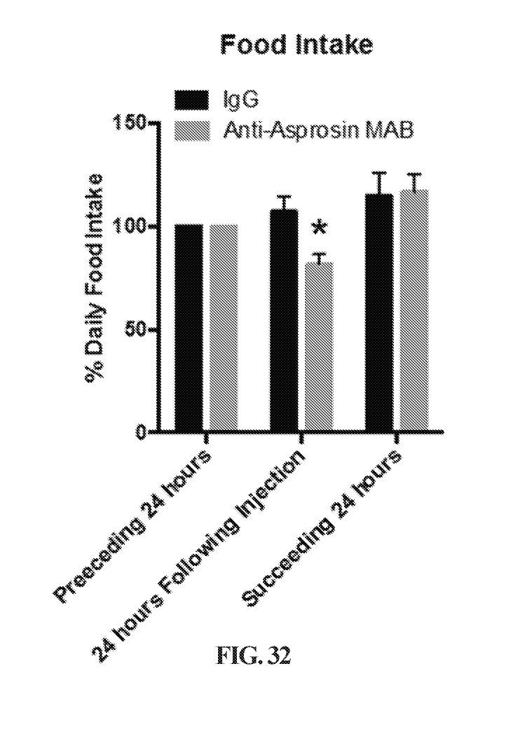

FIG. 32—Food intake was measured at the indicated times, for 24 hours each, before, during and after administration of a single dose of anti-asprosin monoclonal antibody (SEQ ID NO:4).

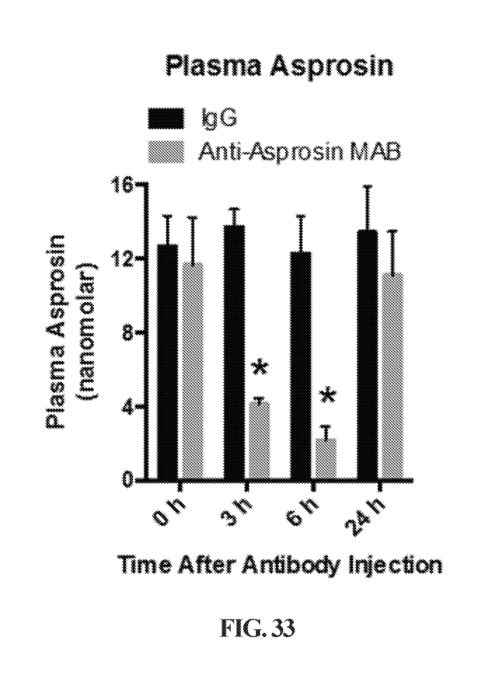

FIG. 33—Plasma asprosin was measured at the indicated times after administration of a single dose of anti-asprosin monoclonal antibody (SEQ ID NO:4) in mice fed a high fat diet for 3 months.

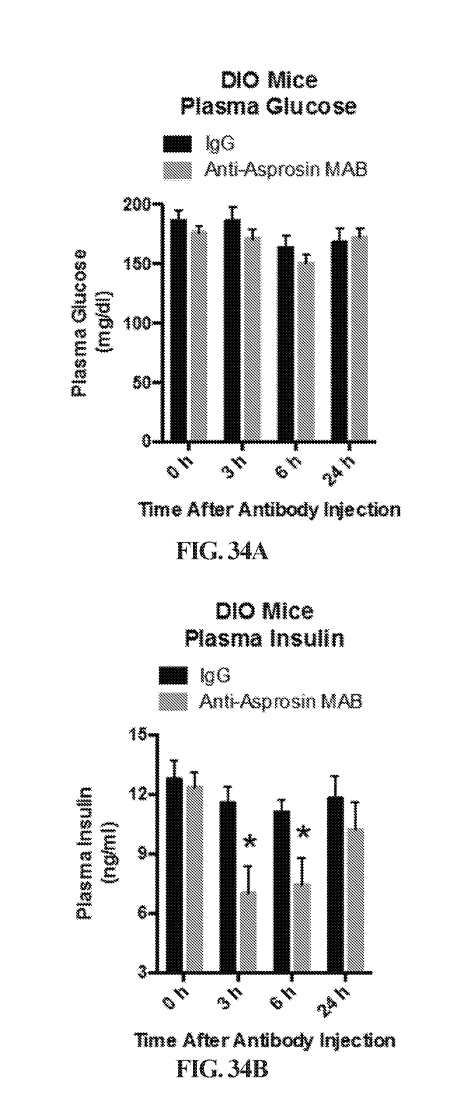

FIG. 34A—Plasma glucose was measured at the indicated times after administration of a single dose (500 ug/mouse) of anti-asprosin monoclonal antibody (SEQ ID NO:4) in mice fed a high fat diet for 3 months.

FIG. 34B—Plasma insulin was measured at the indicated times after administration of a single dose (500 ug/mouse) of anti-asprosin monoclonal antibody (SEQ ID NO:4) in mice fed a high fat diet for 3 months.

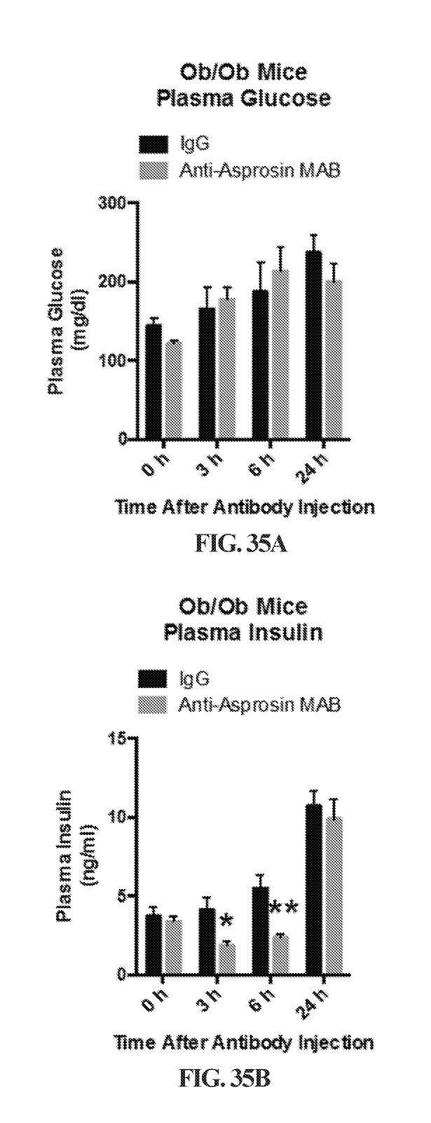

FIG. 35A—Plasma glucose was measured at the indicated times after administration of a single dose (500 ug/mouse) of anti-asprosin monoclonal antibody (SEQ ID NO:4) in ob/ob and WT mice.

FIG. 35B—Plasma insulin was measured at the indicated times after administration of a single dose (500 ug/mouse) of anti-asprosin monoclonal antibody (SEQ ID NO:4) in ob/ob mice.

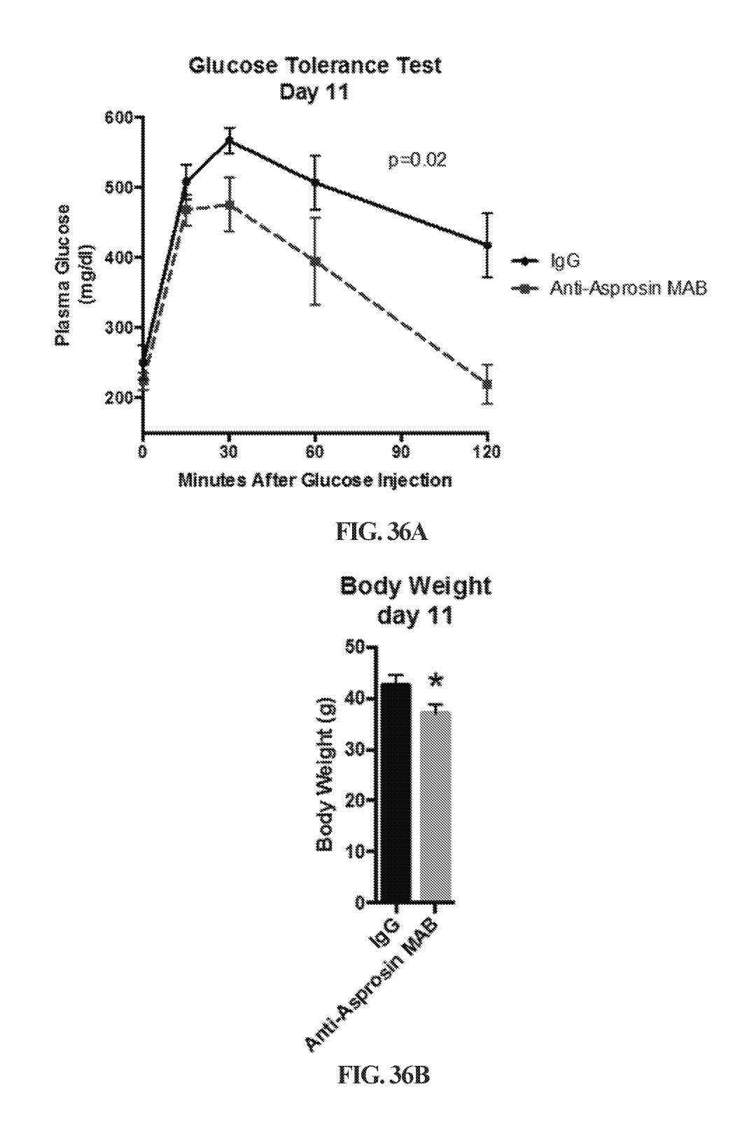

FIG. 36A—Glucose tolerance test was performed on day 11 after administration of a 10 single daily doses (500 ug/mouse) of the anti-asprosin monoclonal antibody (Abnova) in mice fed a high fat diet for 5 months.

FIG. 36B—Body weight was measured on day 11 after administration of a 10 single daily doses (500 ug/mouse) of the anti-asprosin monoclonal antibody (Abnova) in mice fed a high fat diet for 5 months.

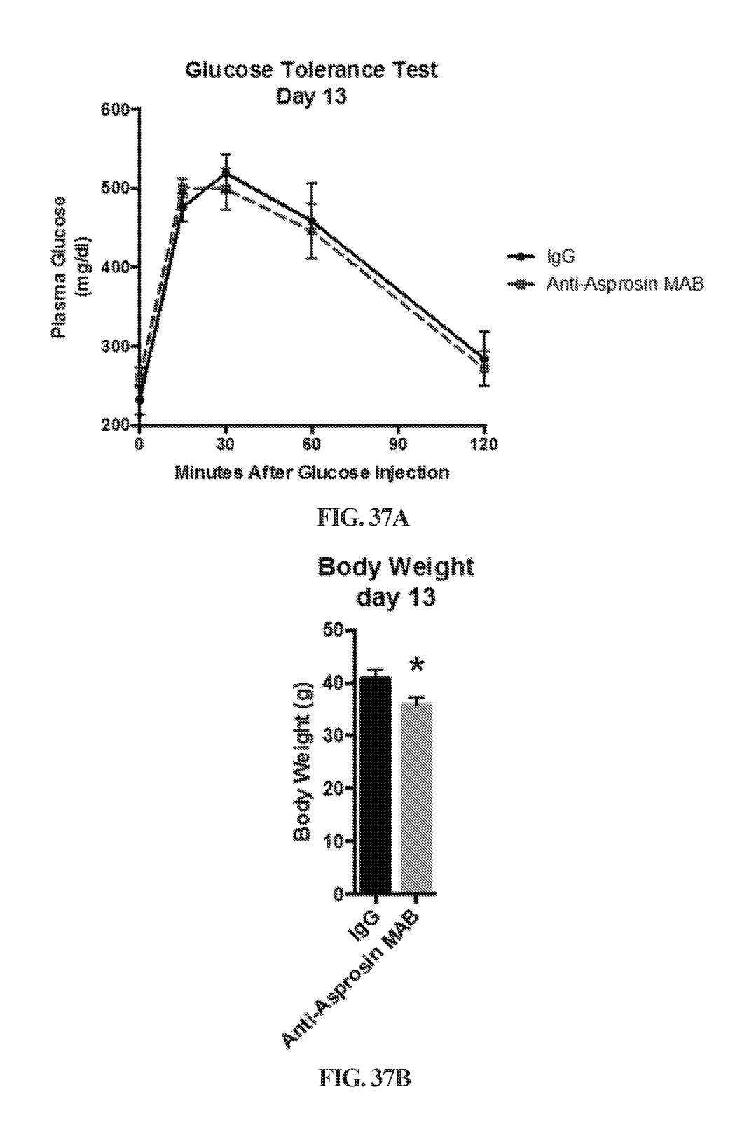

FIG. 37A—Glucose tolerance test was performed on day 13 after administration of a 10 single daily doses (500 ug/mouse) of the anti-asprosin monoclonal antibody (Abnova) in mice fed a high fat diet for 5 months.

FIG. 37B—Body weight was measured on day 13 after administration of a 10 single daily doses (500 ug/mouse) of the anti-asprosin monoclonal antibody (Abnova) in mice fed a high fat diet for 5 months.

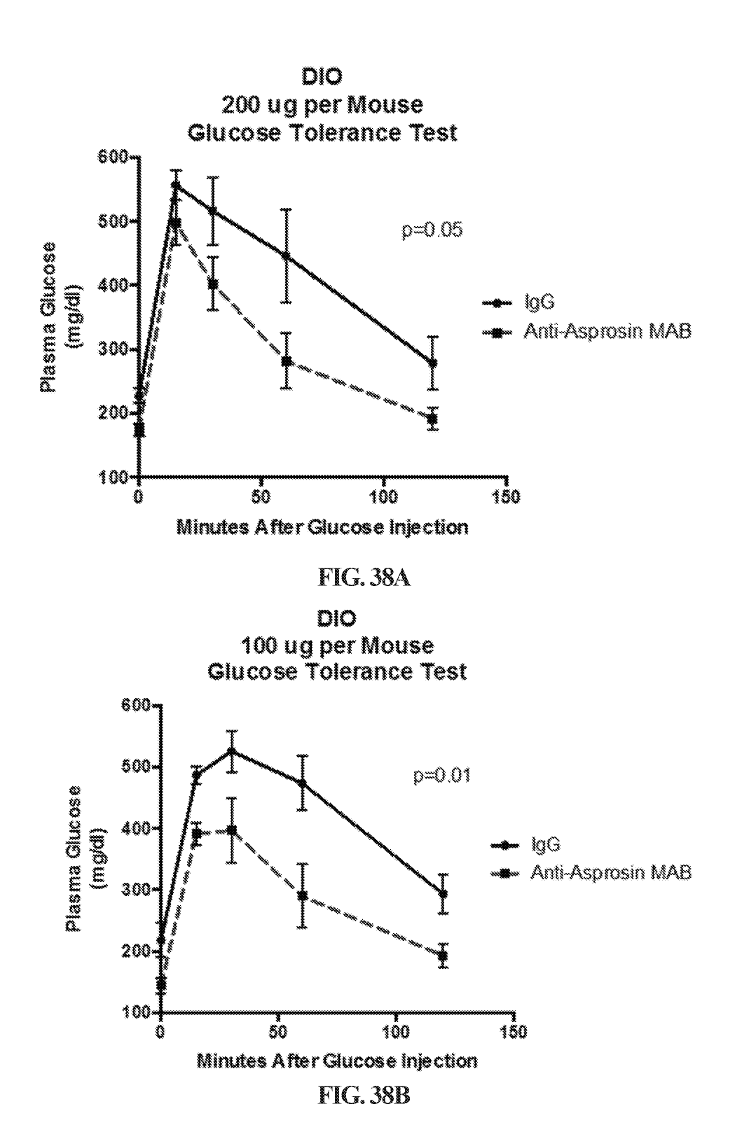

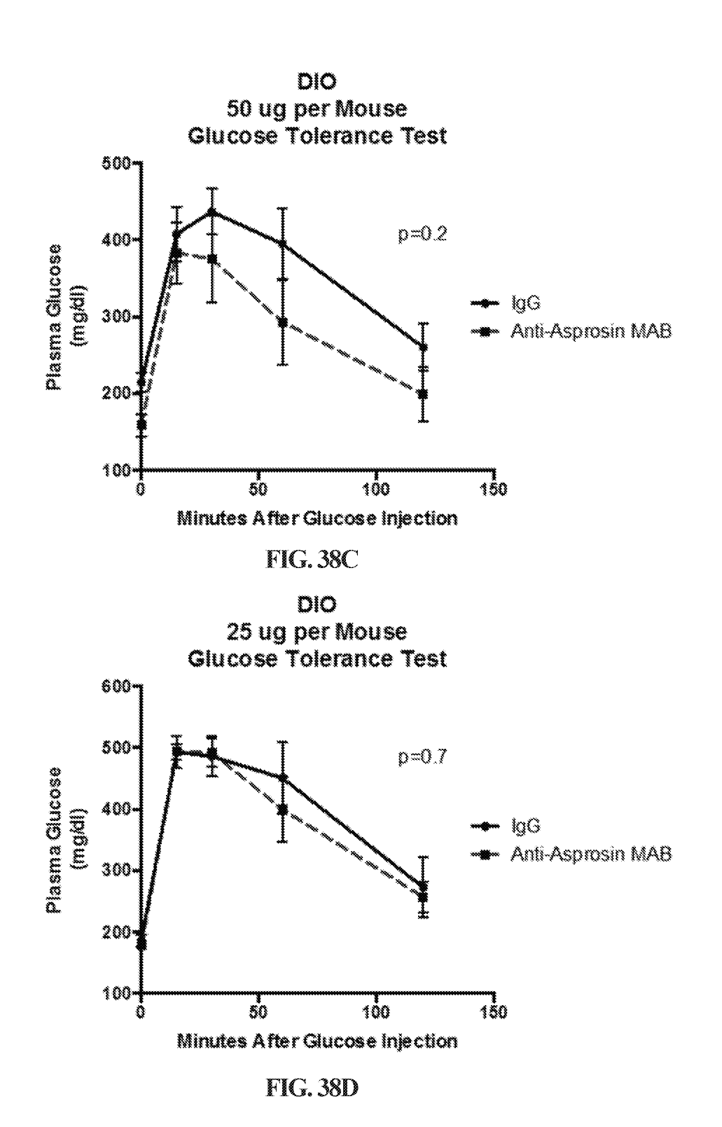

FIG. 38A—Glucose tolerance test was performed after administration of a single 200 ug dose of the anti-asprosin monoclonal antibody (SEQ ID NO:4) in mice fed a high fat diet for 5 months.

FIG. 38B—Glucose tolerance test was performed after administration of a single 100 ug dose of the anti-asprosin monoclonal antibody (SEQ ID NO:4) in mice fed a high fat diet for 5 months.

FIG. 38C—Glucose tolerance test was performed after administration of a single 50 ug dose of the anti-asprosin monoclonal antibody (SEQ ID NO:4) in mice fed a high fat diet for 5 months.

FIG. 38D—Glucose tolerance test was performed after administration of a single 25 ug dose of the anti-asprosin monoclonal antibody (SEQ ID NO:4) in mice fed a high fat diet for 5 months.

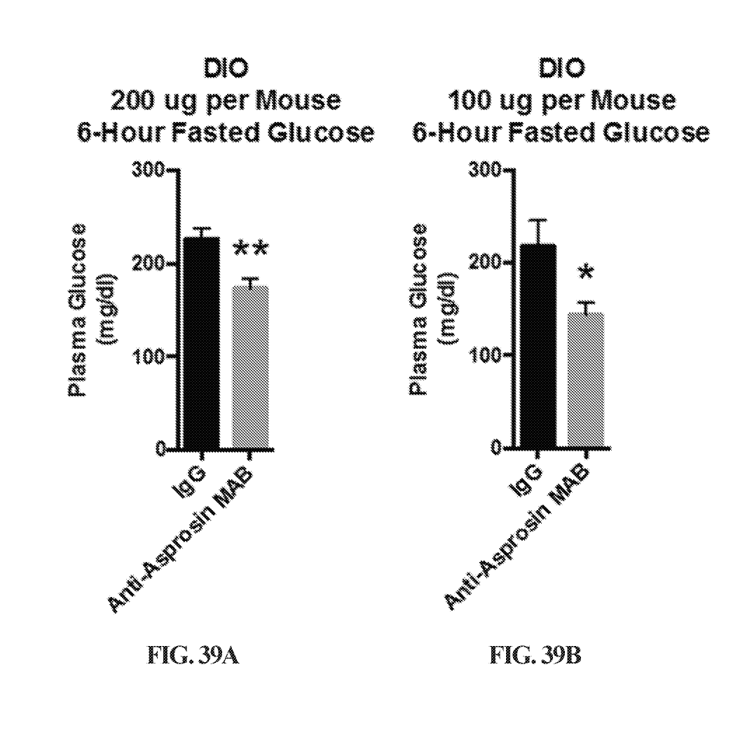

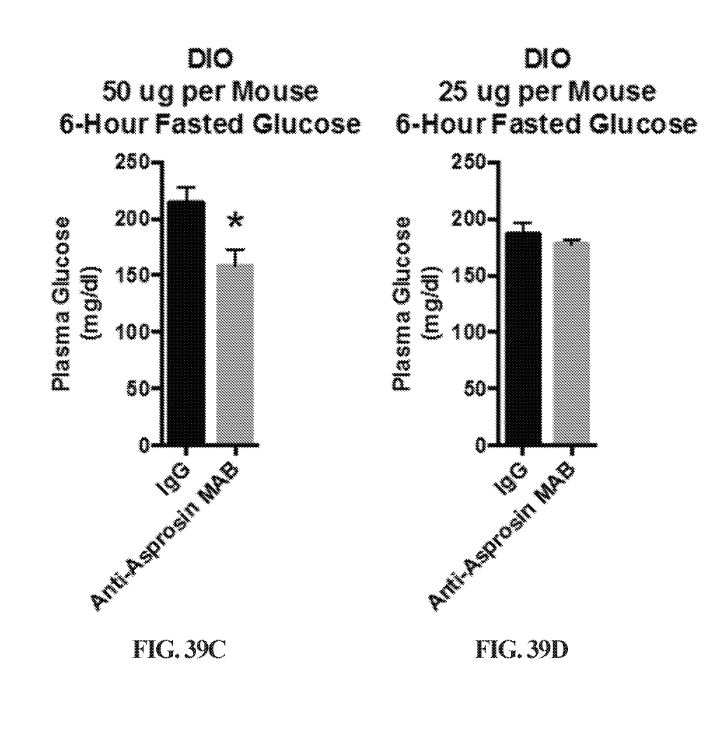

FIG. 39A—Plasma glucose was measured 6 hours after administration of a single 200 ug dose of the anti-asprosin monoclonal antibody (SEQ ID NO:4) in mice fed a high fat diet for 5 months.

FIG. 39B—Plasma glucose was measured 6 hours after administration of a single 100 ug dose of the anti-asprosin monoclonal antibody (SEQ ID NO:4) in mice fed a high fat diet for 5 months.

FIG. 39C—Plasma glucose was measured 6 hours after administration of a single 50 ug dose of the anti-asprosin monoclonal antibody (SEQ ID NO:4) in mice fed a high fat diet for 5 months.

FIG. 39D—Plasma glucose was measured 6 hours after administration of a single 25 ug dose of the anti-asprosin monoclonal antibody (SEQ ID NO:4) in mice fed a high fat diet for 5 months.

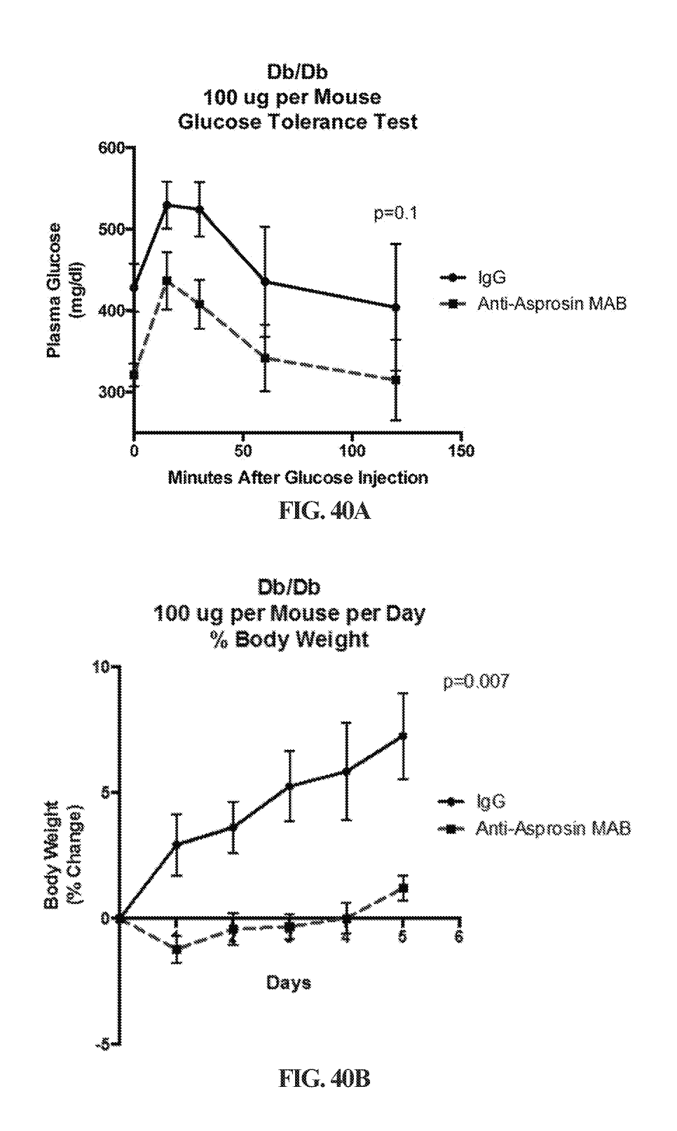

FIG. 40A—Glucose tolerance test was performed after administration of a single 100 ug dose of the anti-asprosin monoclonal antibody (SEQ ID NO:4) in leptin receptor knockout mice (db/db).

FIG. 40B—Daily body weight measurements were performed upon administration of a single 100 ug dose of the anti-asprosin monoclonal antibody (SEQ ID NO:4) in leptin receptor knockout mice (db/db).

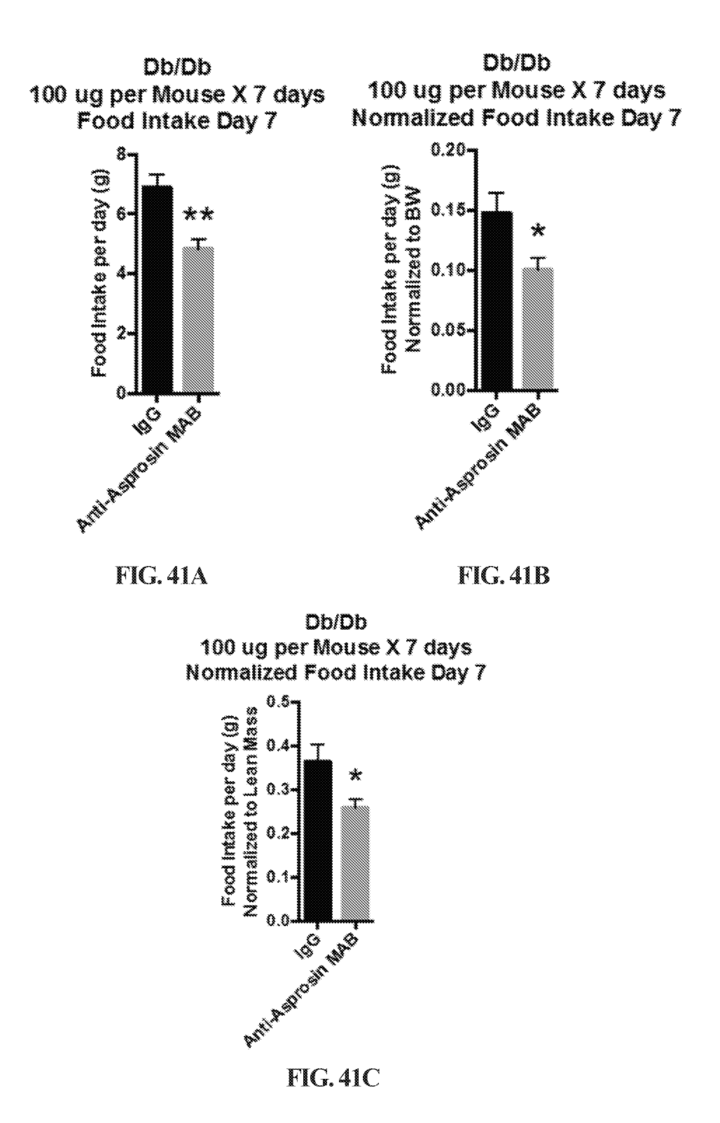

FIGS. 41A-41C—24-hour food intake was measured upon 7 days of administration of a daily 100 ug dose of the anti-asprosin monoclonal antibody (SEQ ID NO:4) in leptin receptor knockout mice (db/db). Non-normalized data (41A) and data normalized to body weight (41B) or lean mass (41C) is presented. Sending this data as an attachment.

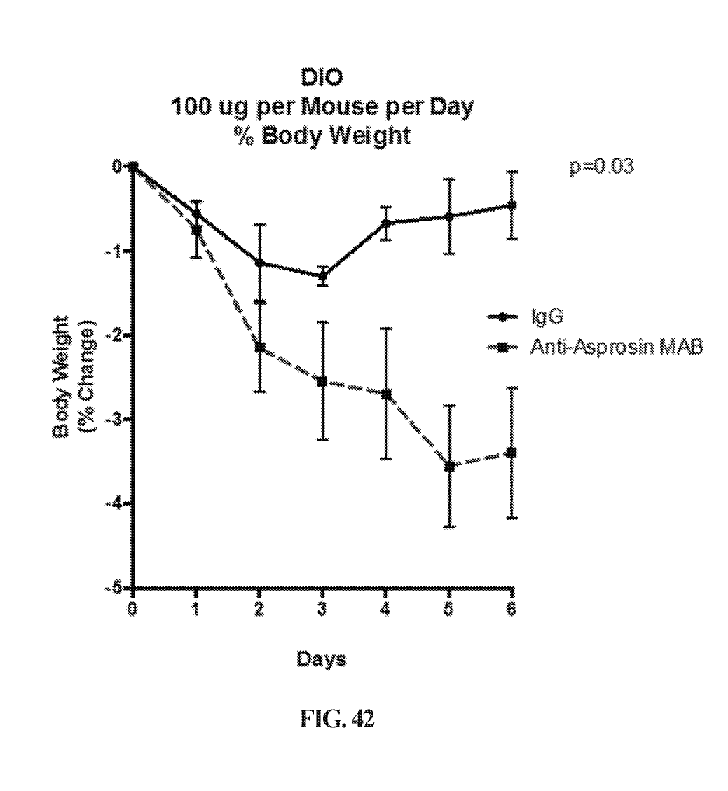

FIG. 42—Daily body weight measurements were performed upon administration of a single 100 ug dose of the anti-asprosin monoclonal antibody (SEQ ID NO:4) in mice fed a high fat diet for 5 months.

### Example 8

### Title: Asprosin Activates the Hypothalamic Hunger-Circuitry

Introduction—

The ability to survive limited periods without food is the cornerstone of evolution and life on earth. Mammals respond to fasting by activating an enormous cascade of interconnected processes that are precisely coordinated by an array of hormones. Two such coordinated processes are appetite stimulation and hepatic glucose release into the circulation, which together, ensure the drive to obtain food, and keep the brain nourished and alert while that is accomplished. Through study of a rare genetic condition in humans—Neonatal Progeroid syndrome (Marfan Lipodystrophy syndrome, OMIM: 616914), a ˜30-kDa fasting-induced hormone, asprosin, was recently discovered that is highly expressed in adipose tissue, and upon secretion, stimulates hepatic glucose release (Romere et al., 2016). Consistent with the necessity for hepatic glucose release during fasting, circulating asprosin rises with fasting and drops with feeding in an acute manner, displaying circadian rhythmicity in coordination with the nutritional state. Now, it is demonstrated herein that peripheral asprosin crosses the blood-brain-barrier to activate the hypothalamic feeding circuitry, leading to appetite stimulation, and over the long term to maintenance of adiposity. The results demonstrate coordination between two critical pillars of the mammalian fasted state—appetite stimulation and hepatic glucose release—via the same fasting-induced hormone, asprosin, through spatiotemporally distinct mechanisms occurring at the liver and the hypothalamus.

Results

Neonatal Progeroid Syndrome (NPS) Patients Display Hypophagia—

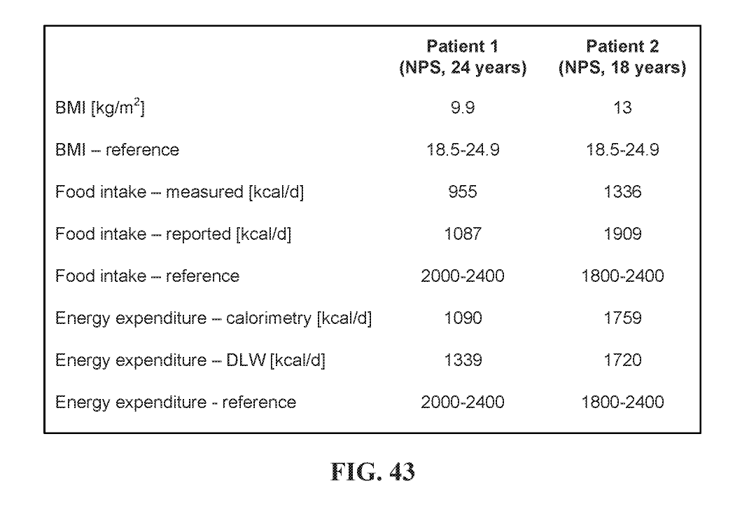

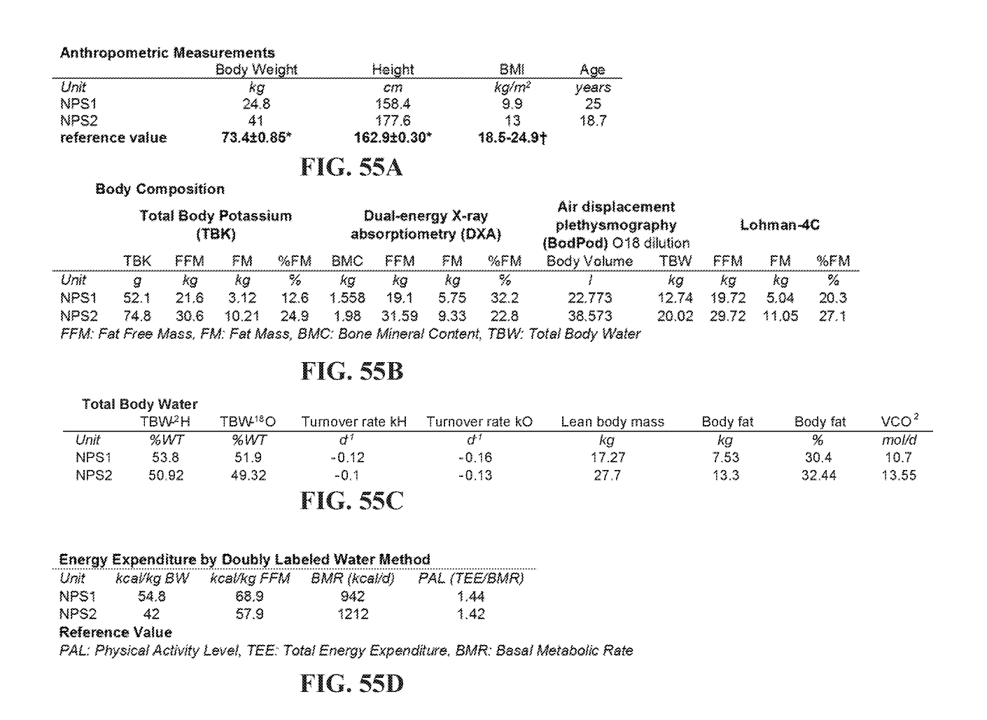

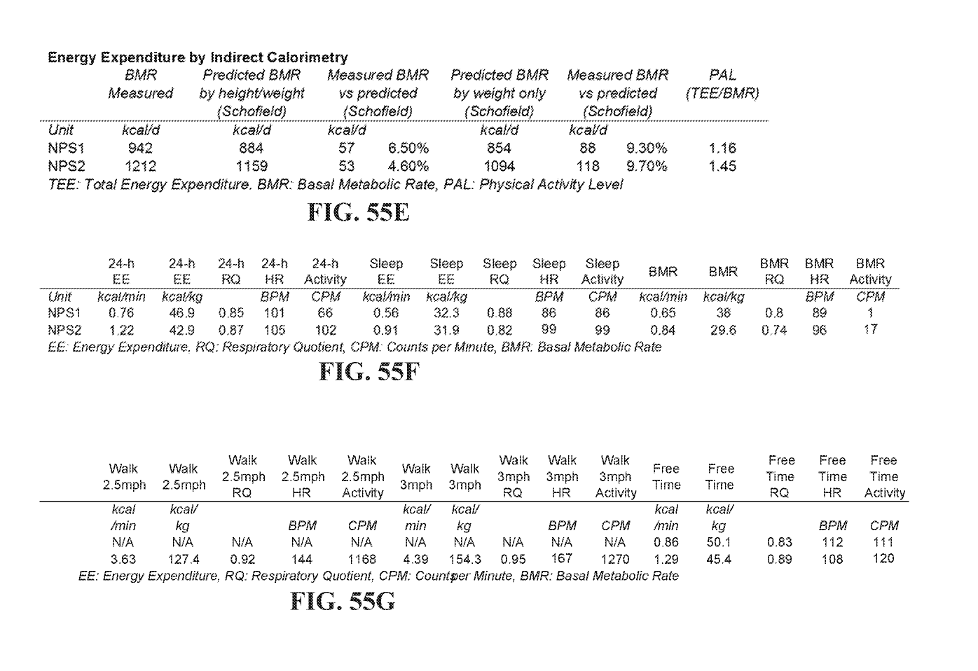

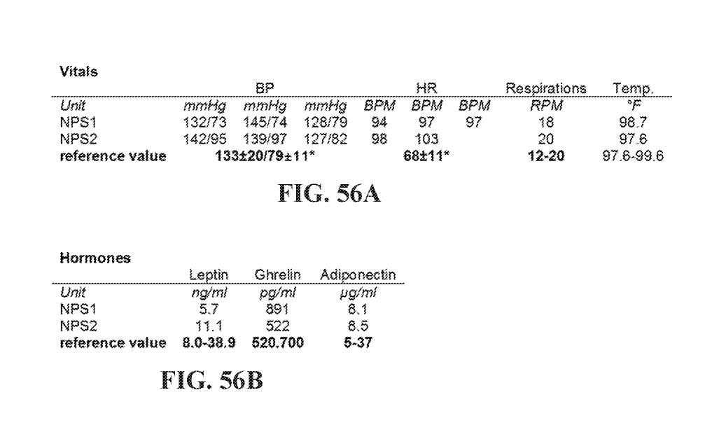

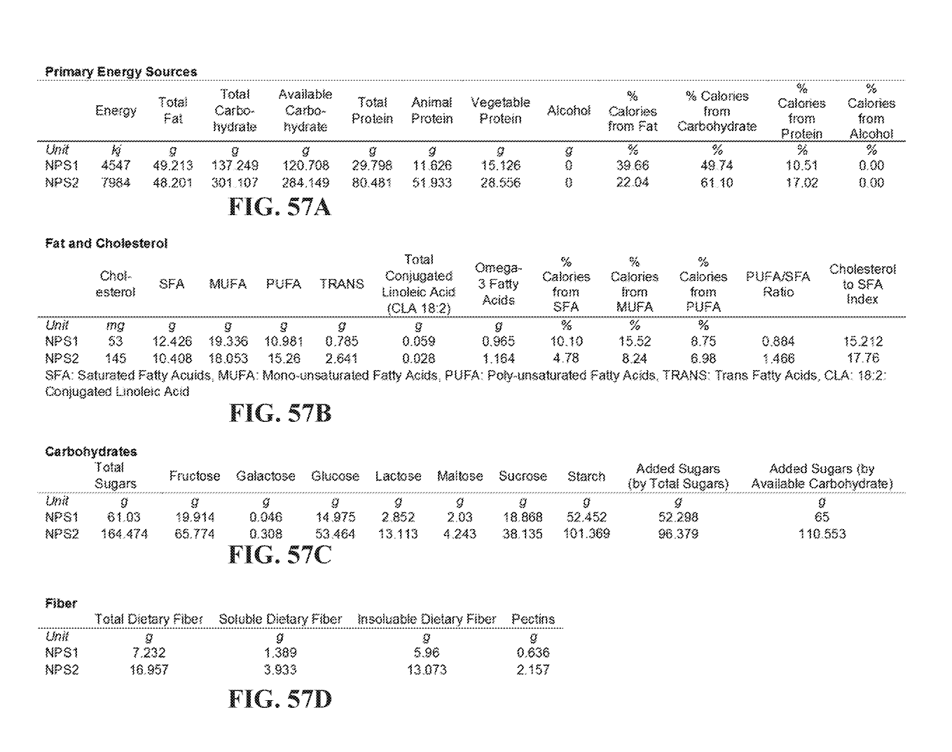

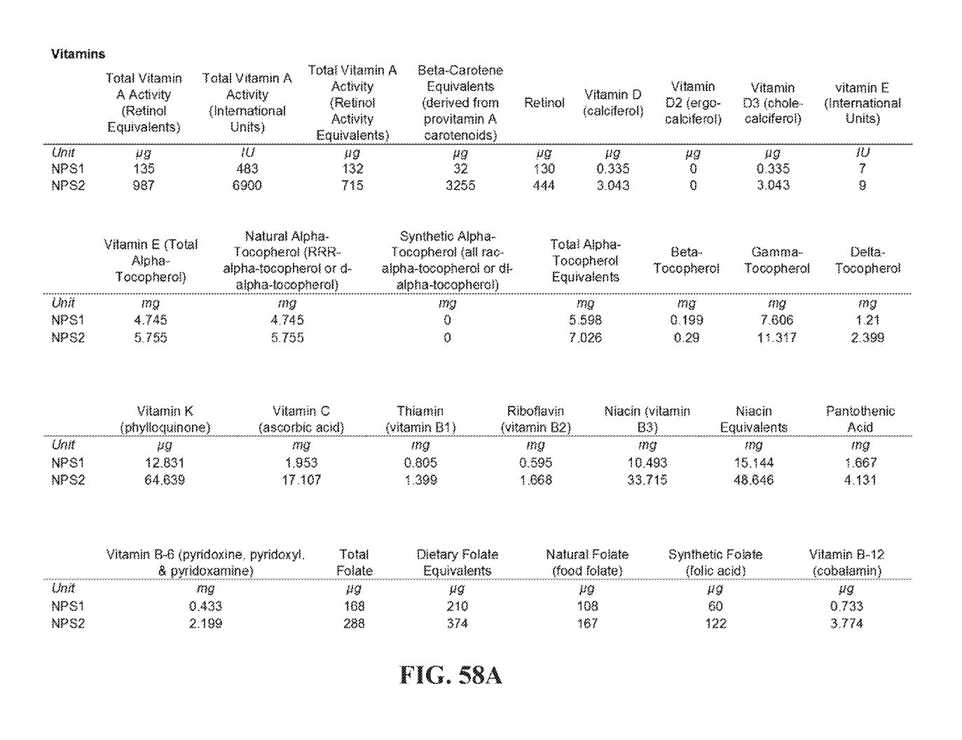

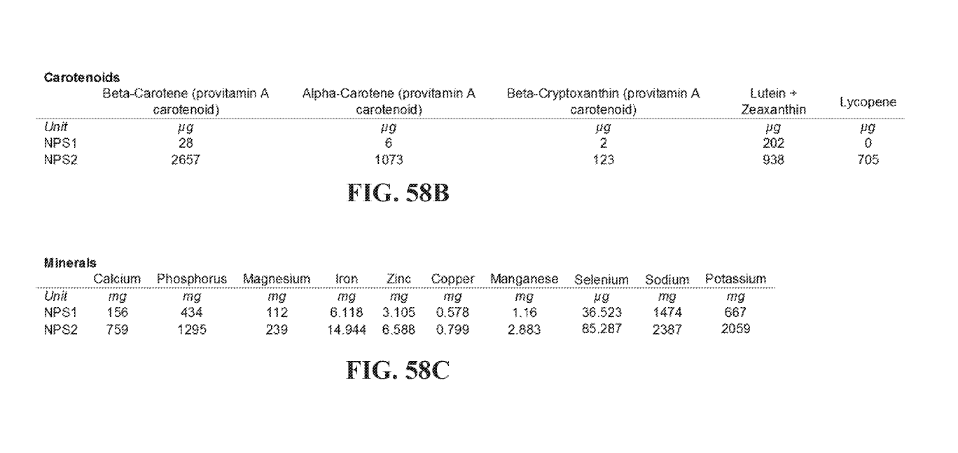

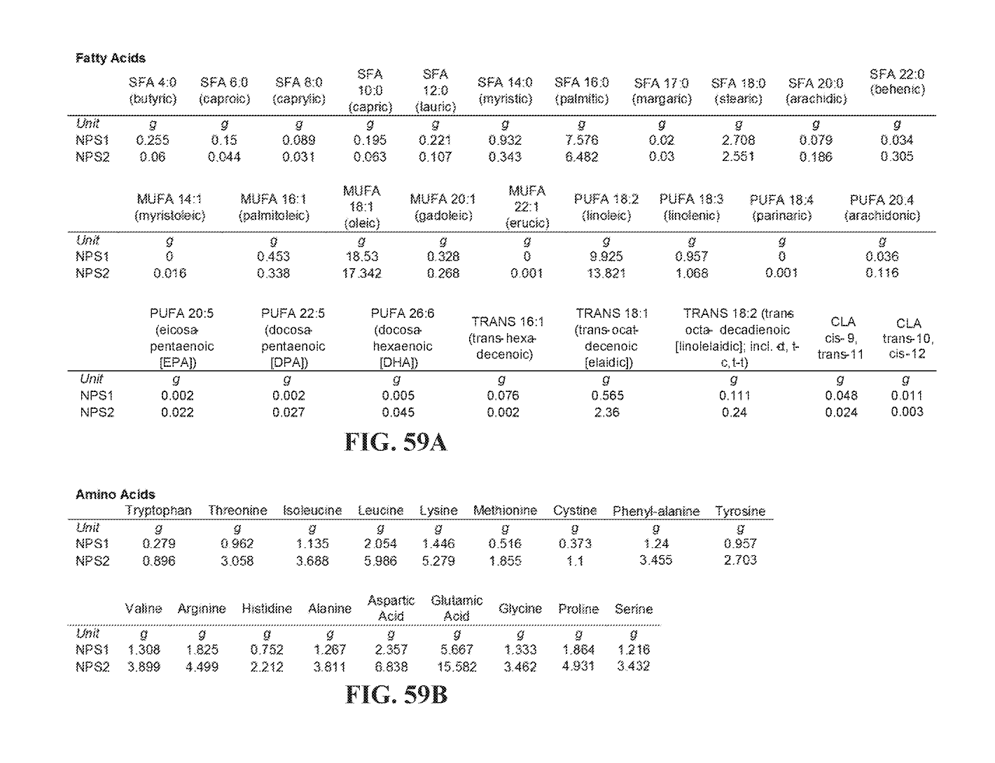

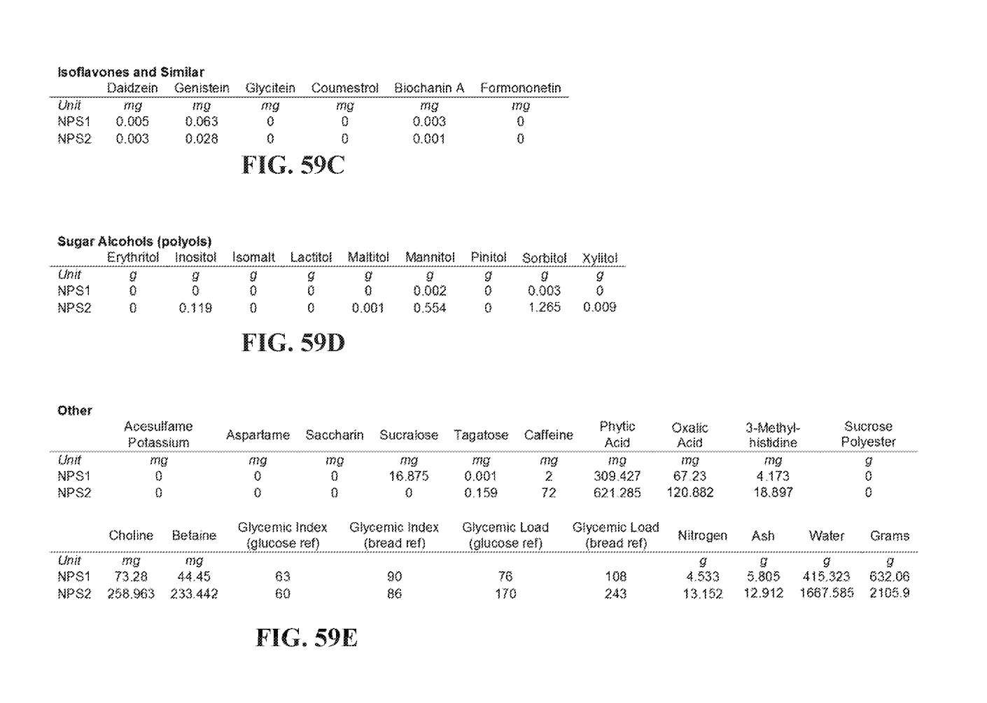

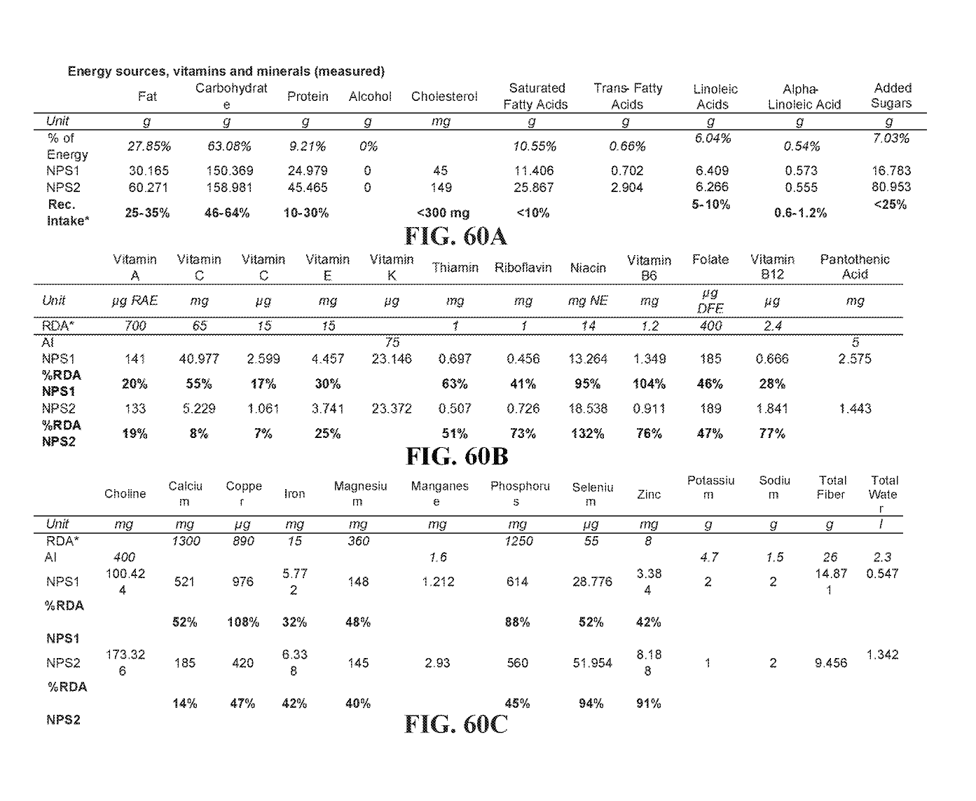

It was previously elucidated the complex molecular genetics of NPS that led the inventors to the protein hormone—Asprosin (Romere et al., 2016). Individuals with NPS display a deficiency in plasma asprosin (Romere et al., 2016) associated with extreme leanness (FIG. 1A) (O'Neill et al., 2007; Romere et al., 2016), reduced subcutaneous adipose mass (FIG. 1A) (O'Neill et al., 2007) and maintenance of insulin sensitivity despite partial lipodystrophy (Romere et al., 2016). In order to better understand the energy balance equation in NPS within the context of the observed leanness, food intake and energy expenditure was measured using two independent methods each. Both individuals consumed fewer calories on a daily basis than their age/sex matched peers in the home as well as the lab setting (FIG. 43). Their hypophagia was matched by a subnormal daily energy expenditure when measured using indirect calorimetry or the doubly-labeled water method (FIG. 43), resulting in a balanced energy equation and a stable but low body weight. In addition to 24-hr caloric intake and 24-hr energy expenditure, ˜270 other parameters were measured related to energy balance so as to obtain a comprehensive physiological view of NPS (FIGS. 55A-55G; FIGS. 56A-56B; FIGS. 57A-57D; FIGS. 58A-58C; FIGS. 59A-59E; and FIGS. 60A-60C). Based on these results it was considered that NPS-associated leanness could at least partially be explained by asprosin deficiency, and that asprosin is necessary for normal levels of appetite in humans.

Asprosin is Present in the Cerebrospinal Fluid (CSF) and Stimulates Appetite in Rodents—

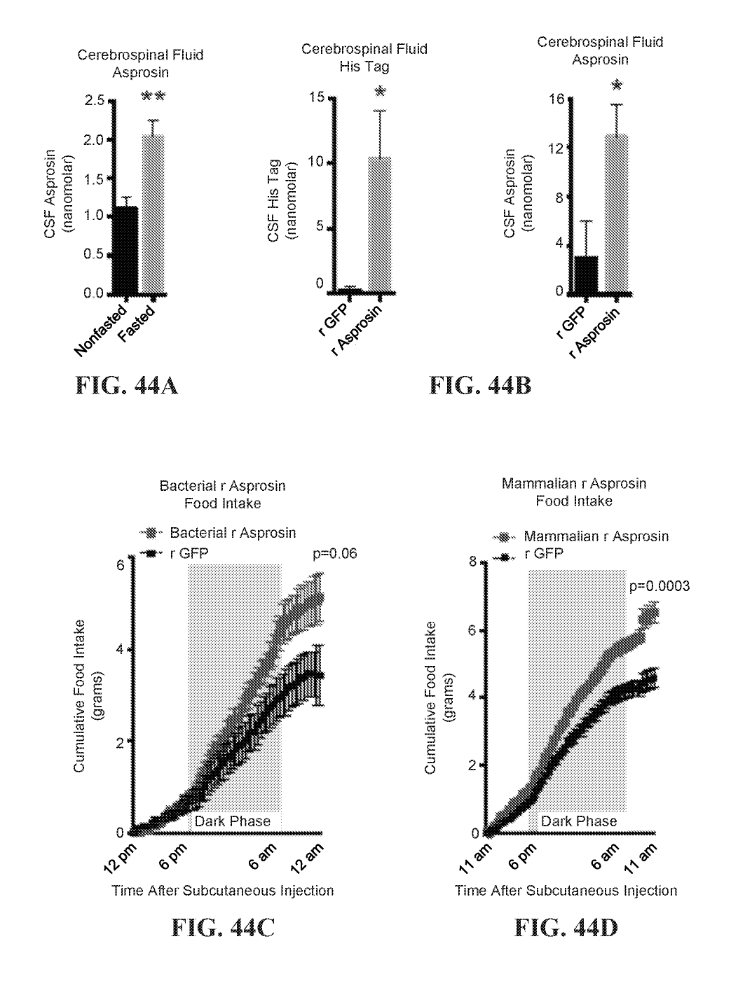

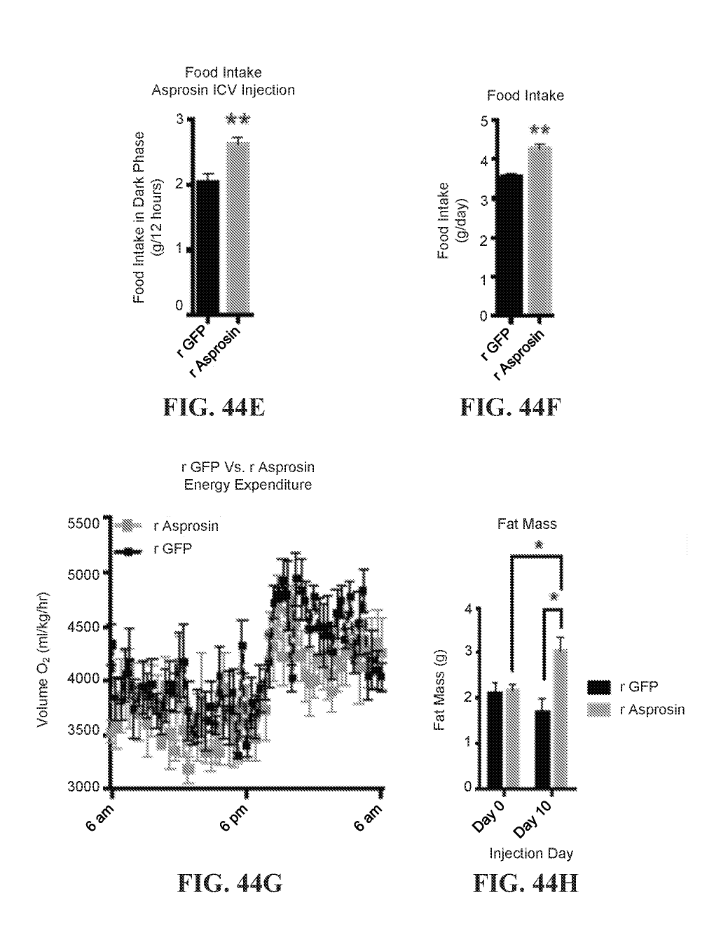

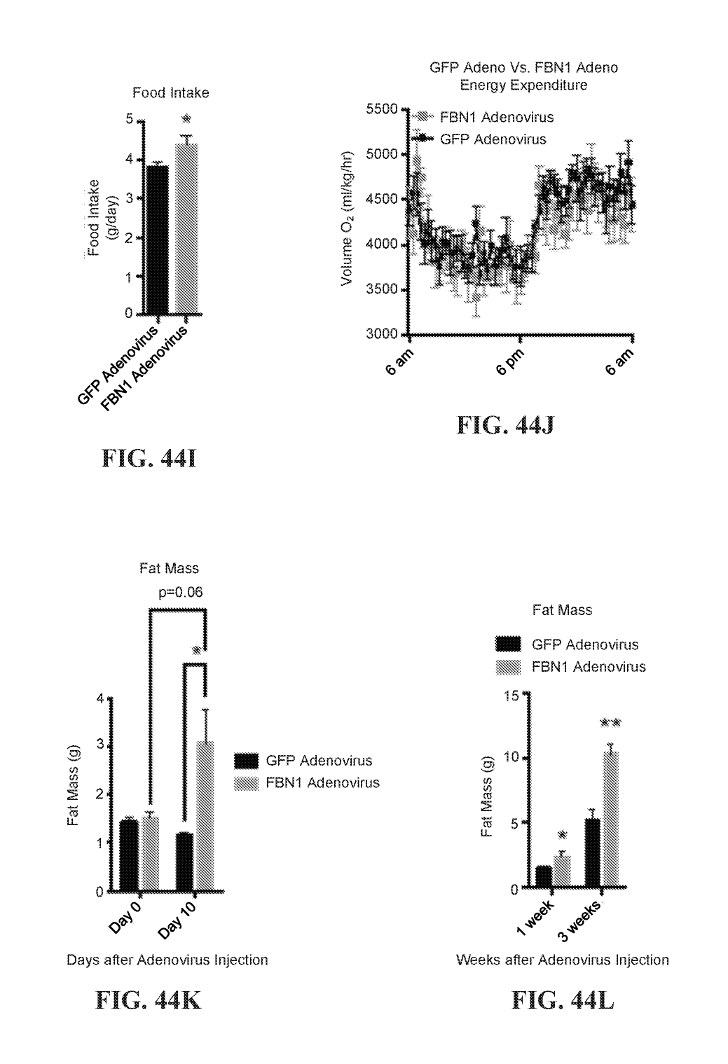

Asprosin levels were assessed in rat CSF using an asprosin-specific (Romere et al., 2016) sandwich ELISA, and it was present in CSF at levels 4-5 fold below those in the plasma (FIG. 44A) (Romere et al., 2016). Similar to plasma asprosin, CSF asprosin was induced by overnight fasting (FIG. 44A). In order to assess whether plasma asprosin could cross the blood-brain-barrier and enter the CSF, rats were injected intravenously with recombinant asprosin and looked for presence of the N-terminal his-tag in the CSF. 1-hr after IV injection a strong his-tag signal in the CSF was detected matched by a 6-fold elevation in CSF asprosin, suggesting robust entry of plasma asprosin into the CSF (FIG. 44B).

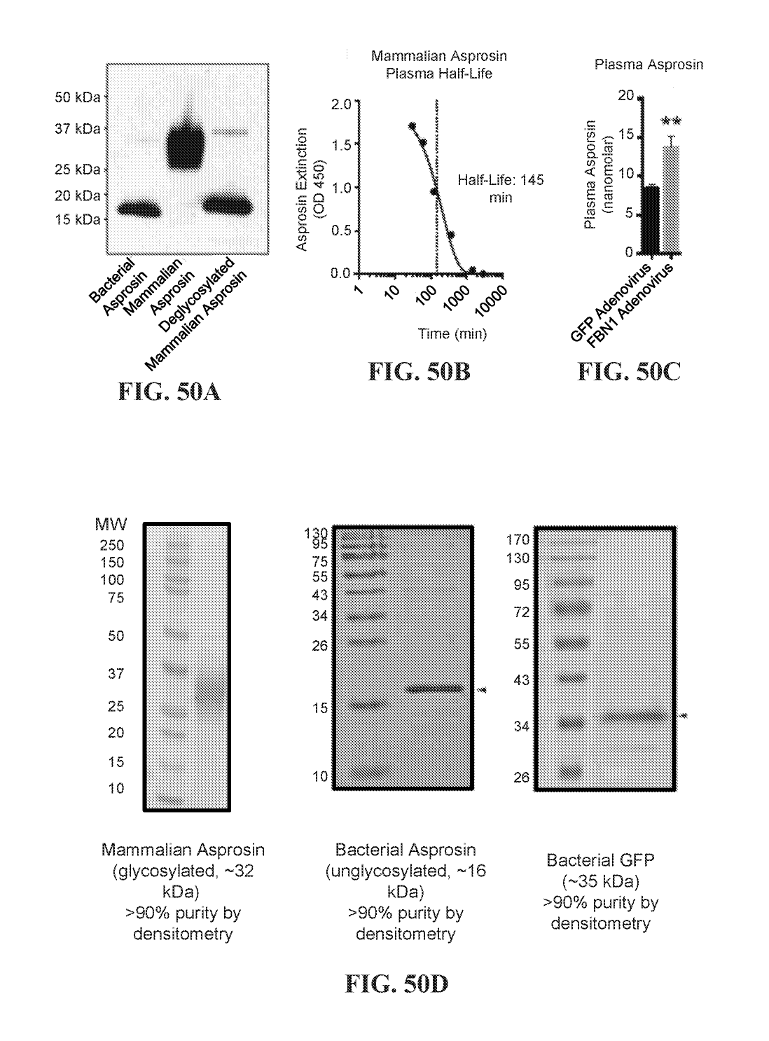

To ascertain whether asprosin stimulates appetite, a single dose was administered FIG of bacterially-expressed or mammalian-expressed recombinant asprosin or GFP subcutaneously to C57Bl/6 mice. Asprosin injected mice displayed greater food intake over the next 24-hr irrespective of which recombinant asprosin preparation was used, compared with GFP injected mice (FIGS. 44C-44D). Of note, mammalian-generated asprosin is about twice the molecular weight of the bacterial-generated asprosin, and, as predicted previously (Romere et al., 2016), this difference is largely due to glycosylation of the mammalian variety (FIG. 50A). The plasma half-life of the mammalian-generated asprosin is ˜145 minutes (FIG. 50B) compared with ˜20 minutes for the bacterially-generated version (Romere et al., 2016). Interestingly, asprosin consistently displayed a latent phase of several hours prior to exerting its orexigenic effect (FIGS. 44C-44D), which differentiates asprosin from more acute acting agents such as ghrelin (Nakazato et al., 2001). Consistent with subcutaneous injection, there was an orexigenic effect of asprosin upon direct introduction into the CSF via intracerebroventricular (ICV) injection (FIG. 44E). In order to understand chronic responses, C57Bl/6 mice were treated with daily subcutaneous doses of recombinant asprosin for 10 days. In addition to the expected hyperphagia (FIG. 44F), there was no change in energy expenditure (FIG. 44G) and an increase in adiposity (FIG. 44H), demonstrating a tilting of the energy balance equation in favor of increased energy intake. A second 10-day gain-of-function strategy using adenovirus mediated FBN1 transgenesis that results in a ˜2-fold elevation in plasma asprosin (Romere et al., 2016) (FIG. 50C), showed a similar hyperphagic response (FIG. 44I) and no change in energy expenditure (FIG. 44J), accompanied by an increase in adiposity on normal chow (FIG. 44K) that was potentiated by putting the animals on a high fat diet (FIG. 44L).

Asprosin Activates Orexigenic AgRP Neurons—

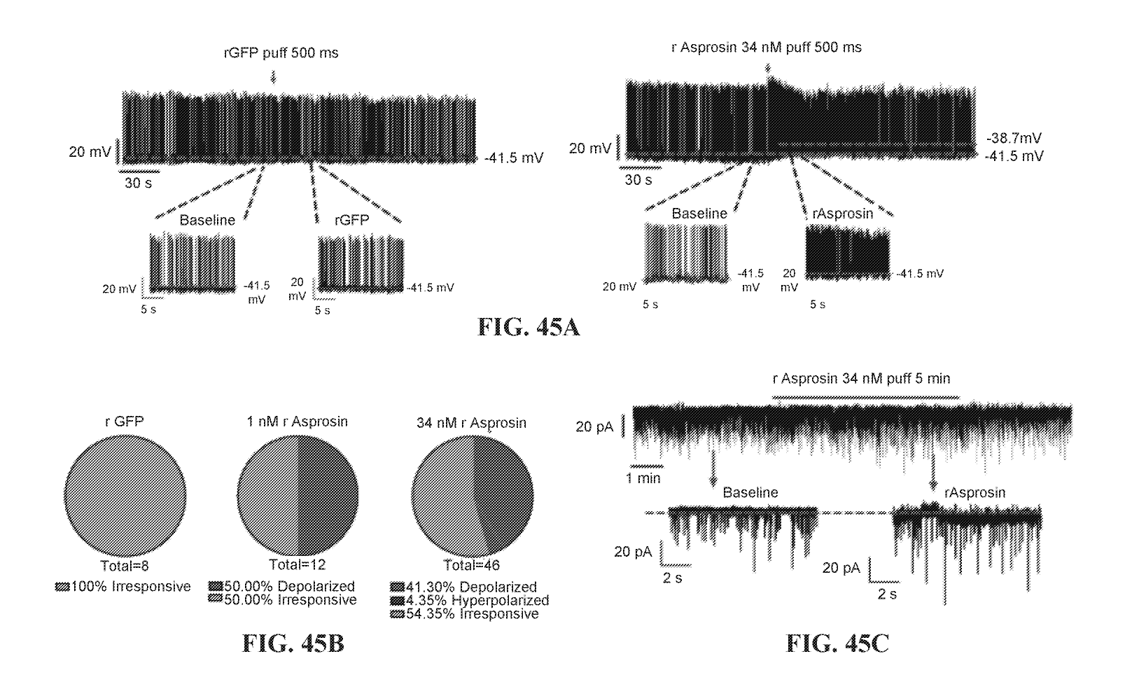

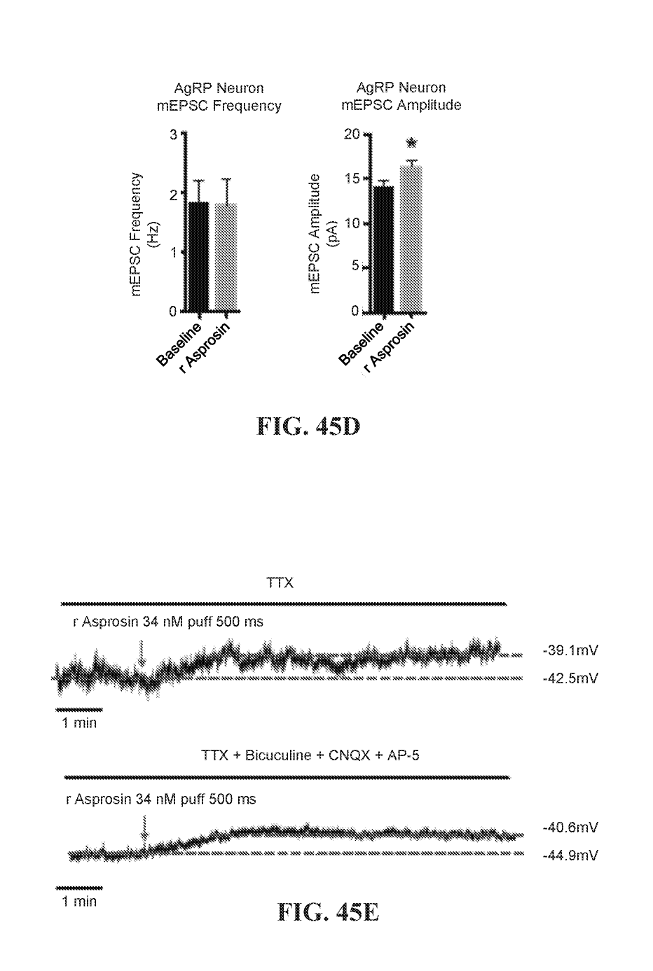

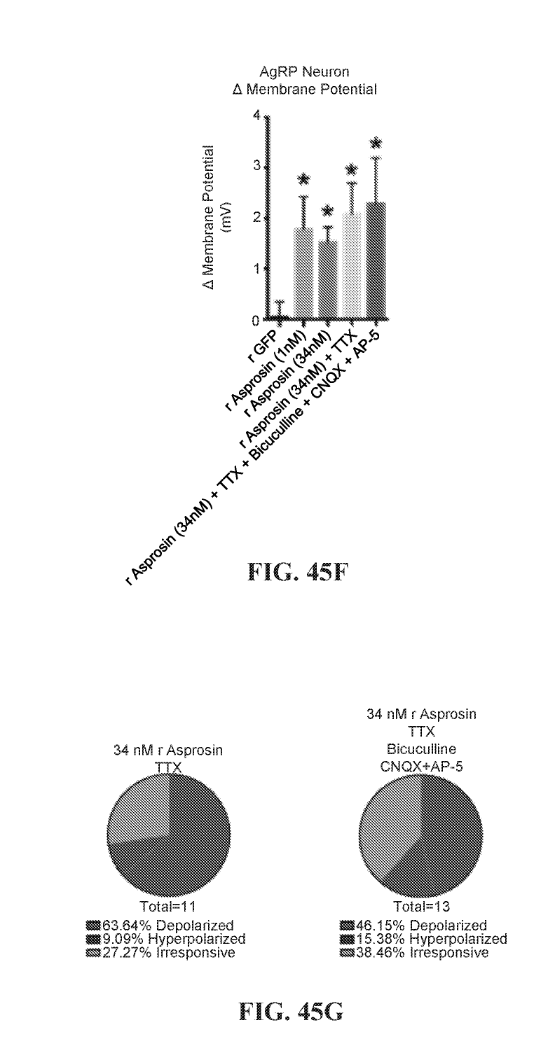

In order to probe the mechanisms underlying asprosin's orexigenic function, the hypothalamic feeding center in the arcuate nucleus was focused on. AgRP neurons are a well-studied population of orexigenic neurons (Aponte et al., 2011; Krashes et al., 2011; Luquet et al., 2005), and recombinant asprosin acutely induced their activation via an increase in firing frequency and an increase in membrane potential, while expectedly, recombinant GFP displayed no activity at all (FIG. 45A). It was consistently observed that only about 50% of AgRP neurons were asprosin-responsive, suggesting that only a subset contain the components necessary for transducing the asprosin signal (FIG. 45B). In addition, asprosin increased the amplitude but not frequency of the miniature excitatory post-synaptic current (mEPSC) of AgRP neurons, suggesting that asprosin activates AgRP neurons via post-synaptic rather than presynaptic mechanisms (FIGS. 45C-45D). This was confirmed by pharmacologically inhibiting synaptic inputs to the AgRP neurons and there was no decrease in asprosin-mediated activation (FIGS. 45E-45G).

The Gαₛ-cAMP-PKA Axis is Necessary for Asprosin-Mediated AgRP Neuron Activation—

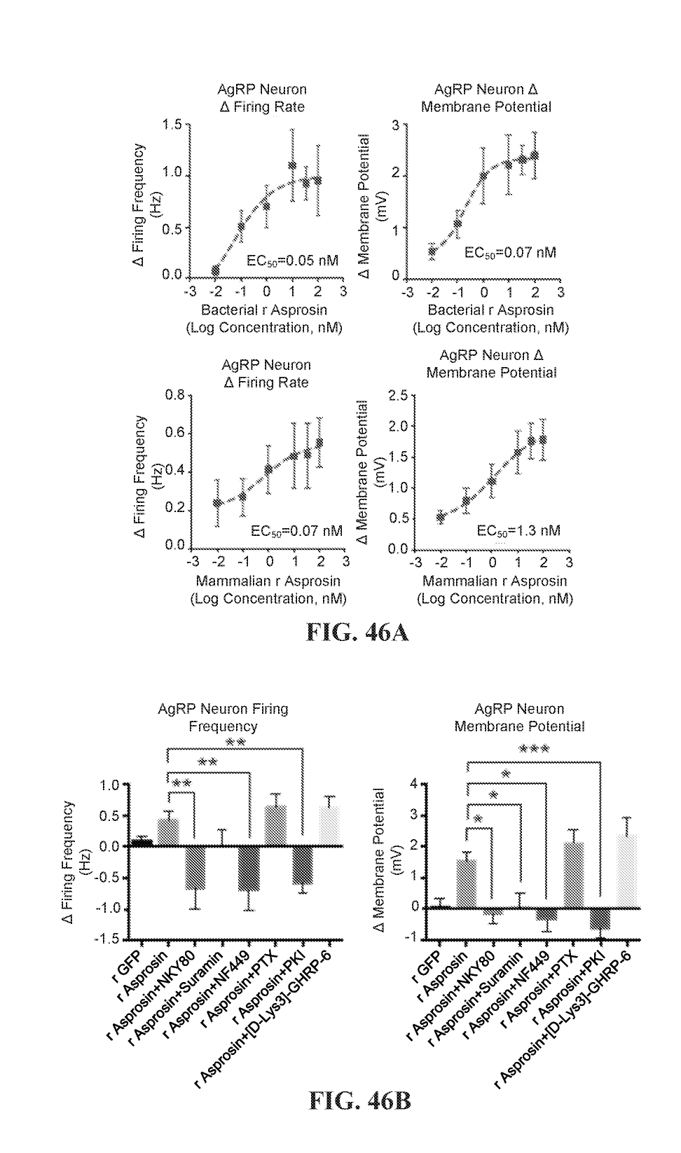

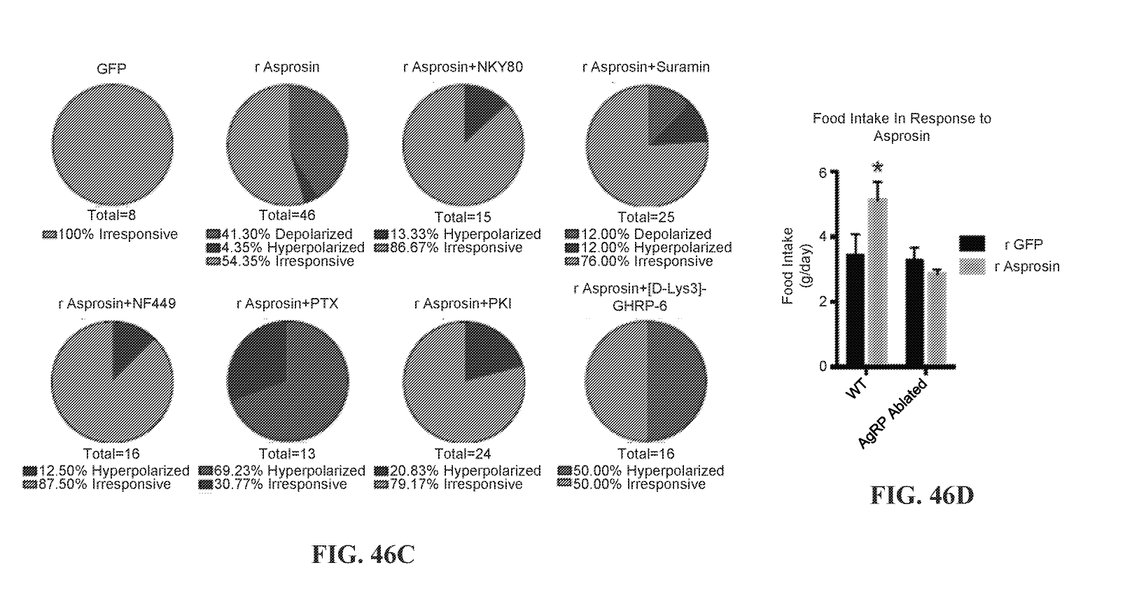

Dose-dependency was assessed of asprosin action in AgRP neurons by exposing intact slices to either bacterial- or mammalian-generated recombinant asprosin. There was a dose-dependent activation of AgRP neurons at the levels of firing frequency and membrane potential for both varieties of asprosin (FIG. 46A). The EC₅₀ values for both varieties of asprosin were found to be in the nanomolar to subnanomolar range, showing great sensitivity of action, and are well within the range in which endogenous asprosin exists in the CSF (FIG. 46A).

Asprosin-mediated AgRP neuron activation, at the level of both firing frequency and resting membrane potential, could be completely prevented by pre-treating with Suramin (heterotrimeric G-protein inhibitor), NF449 (Gαₛ inhibitor), NKY80 (adenylate cyclase inhibitor), and PKI (protein kinase A inhibitor) (FIG. 46B). Conversely, pre-treating with PTX (Gαᵢ inhibitor) or with [D-Lys3]-GHRP-6 (ghrelin receptor inhibitor) had no impact on asprosin's ability to activate AgRP neurons (FIG. 46B). These results suggest that signaling via G-proteins (specifically Gαₛ), adenylate cyclase, cAMP and PKA is necessary for asprosin-mediated AgRP neuron activation, while Gαᵢ and ghrelin receptor are dispensable. The same pattern was observed at the level of the proportion of AgRP neurons activated by asprosin (FIG. 46C).

To ascertain the necessity for AgRP neurons for asprosin-mediated appetite stimulation, asprosin was injected in WT and mutant mice with genetic ablation of AgRP neurons (Luquet et al., 2005). As expected, AgRP neuron ablation completely eliminated asprosin's orexigenic effect (FIG. 46D).

Asprosin Inhibits Anorexigenic POMC Neurons—

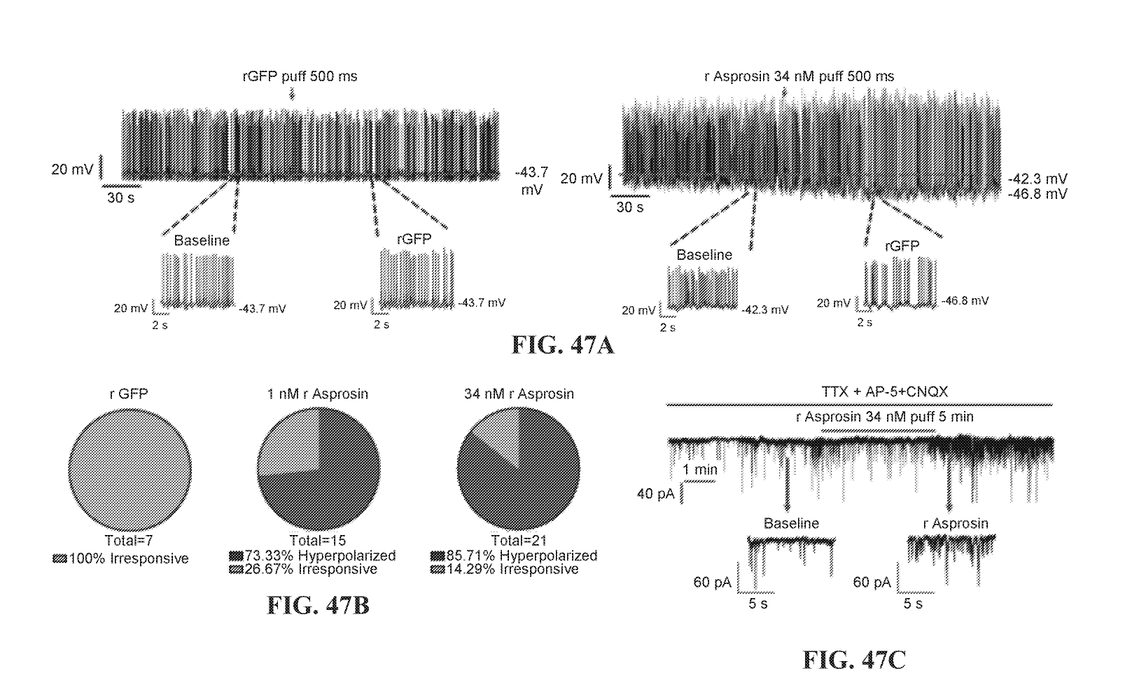

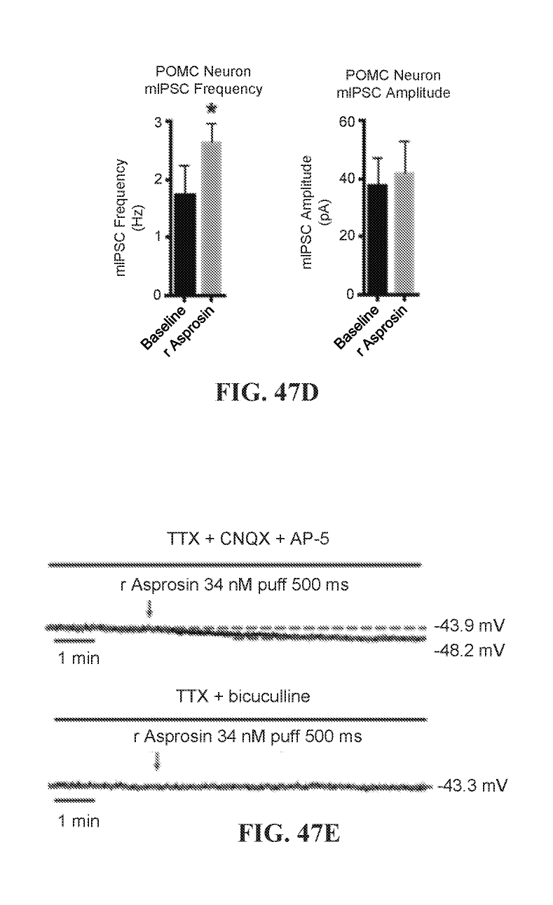

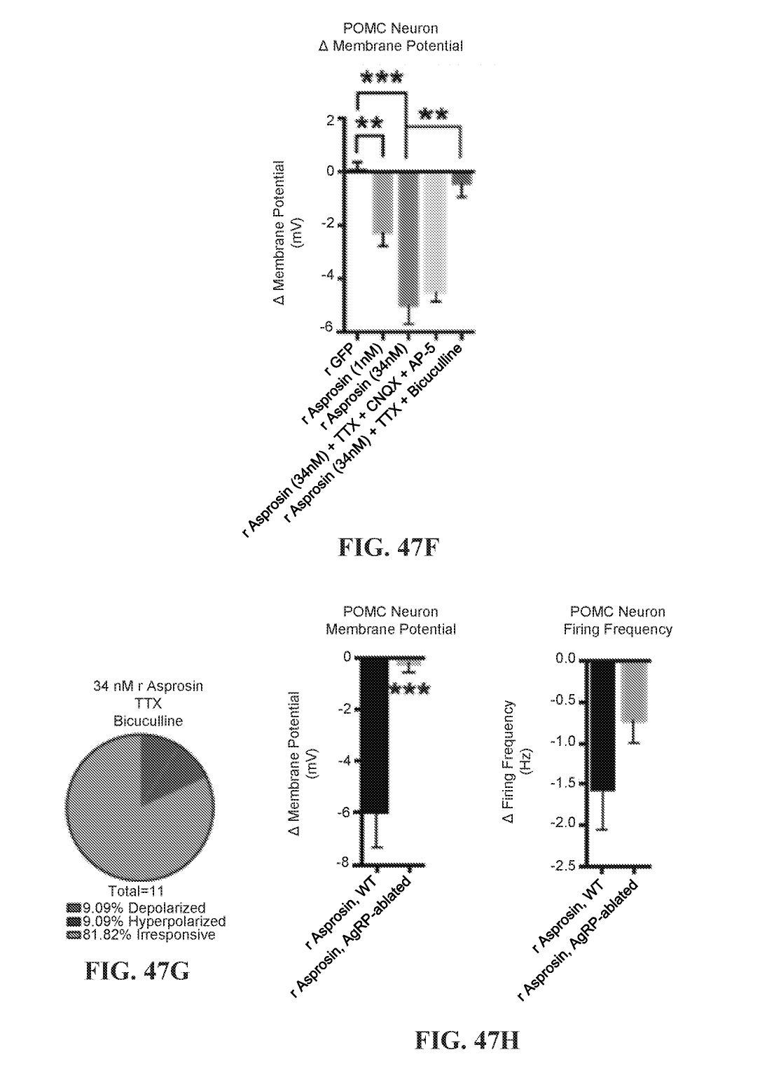

POMC neurons are an anorexigenic population of neurons within the arcuate nucleus that function coordinately with AgRP neurons. Asprosin acutely inhibited ˜85% of the POMC neurons by reducing their resting membrane potential and firing frequency (FIGS. 47A-47B). In addition, asprosin increased the frequency but not amplitude of the miniature inhibitory post-synaptic current (mIPSC) in POMC neurons (FIGS. 47C-47D). In contrast to the demonstrated direct action on AgRP neurons, asprosin's ability to hyperpolarize POMC neurons was dependent on intact GABAergic input, suggesting indirect action via surrounding GABAergic neurons (FIGS. 47E-47G). Blockade of glutamatergic inputs on the other hand, did not affect asprosin-mediated POMC neuron hyperpolarization (FIG. 47F). Since AgRP neurons are one such GABAergic population that projects to POMC neurons (Atasoy et al., 2012; Tong et al., 2008), the effects were tested of AgRP neuron ablation on asprosin-mediated POMC hyperpolarization. AgRP neuron ablation completely prevented asprosin-mediated hyperpolarization in POMC neurons, with little effect observed at the level of firing frequency (FIG. 47H). This suggests that AgRP neurons are at least one population of upstream GABAergic neurons that transduce asprosin's signal to POMC neurons.

Mouse Neonatal Progeroid Syndrome Phenocopies the Human Disorder—

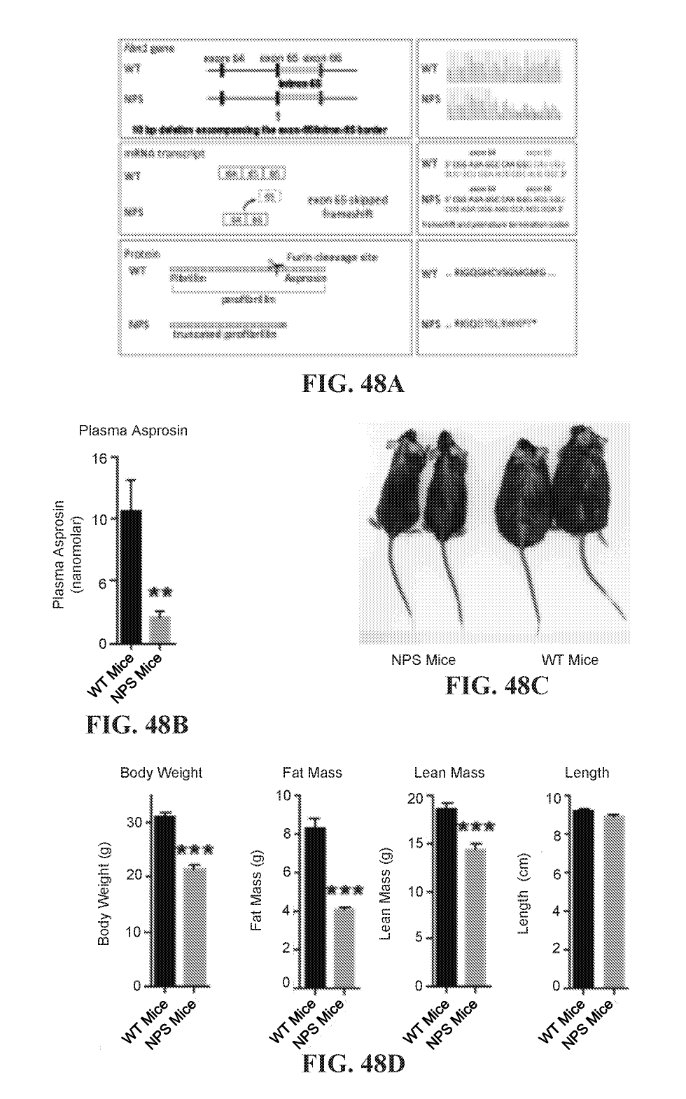

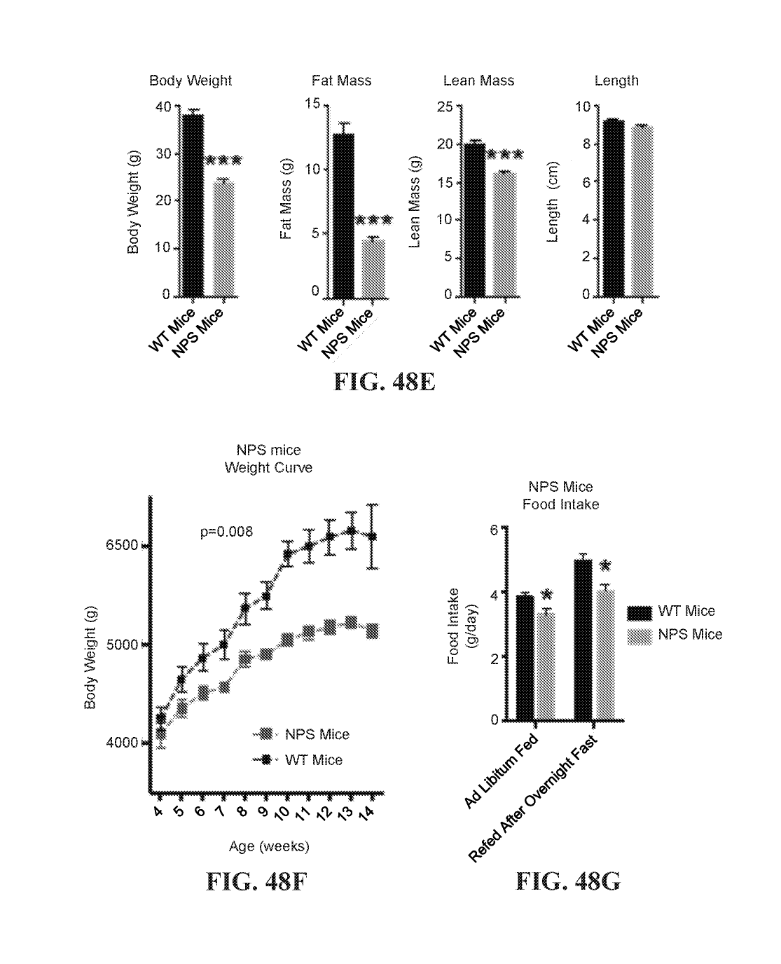

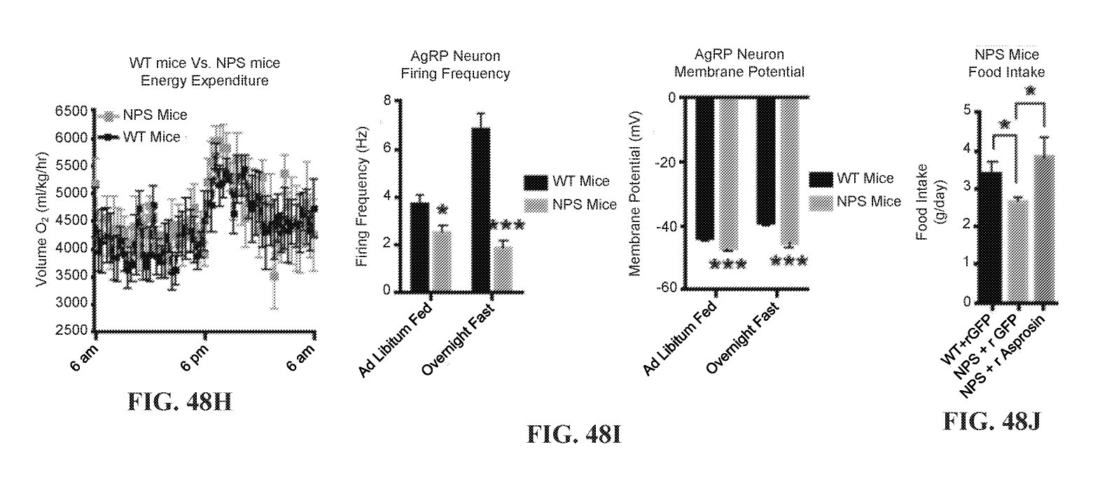

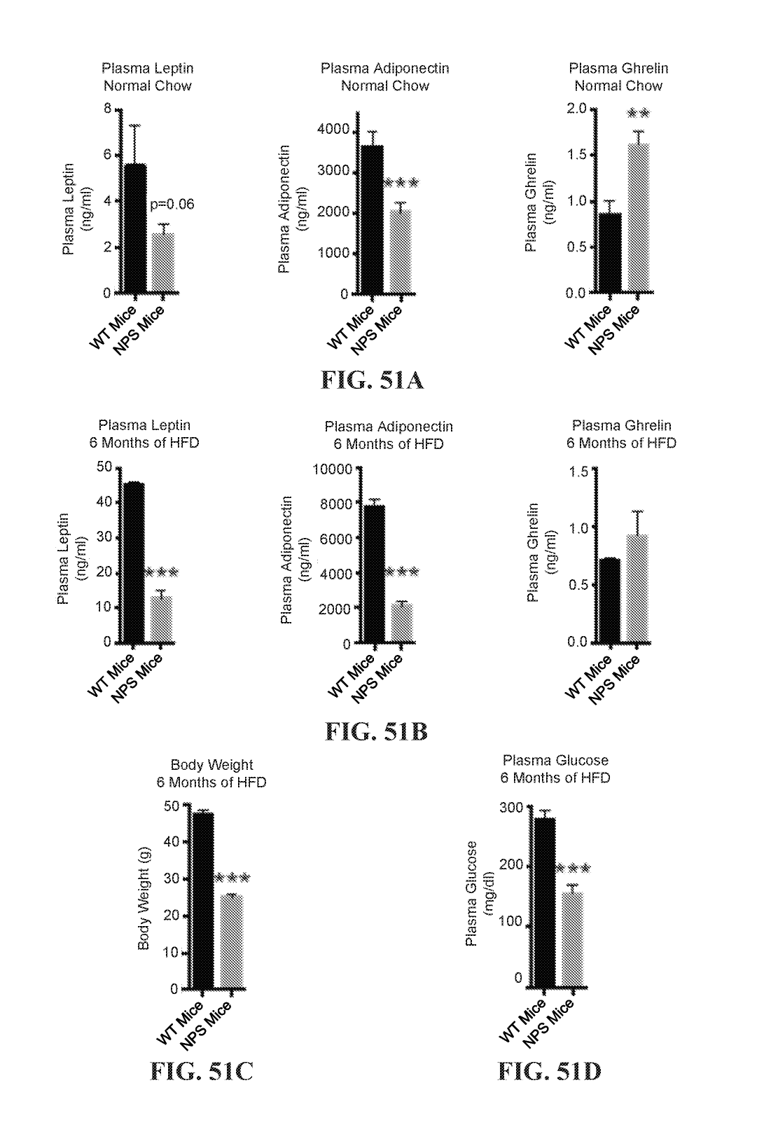

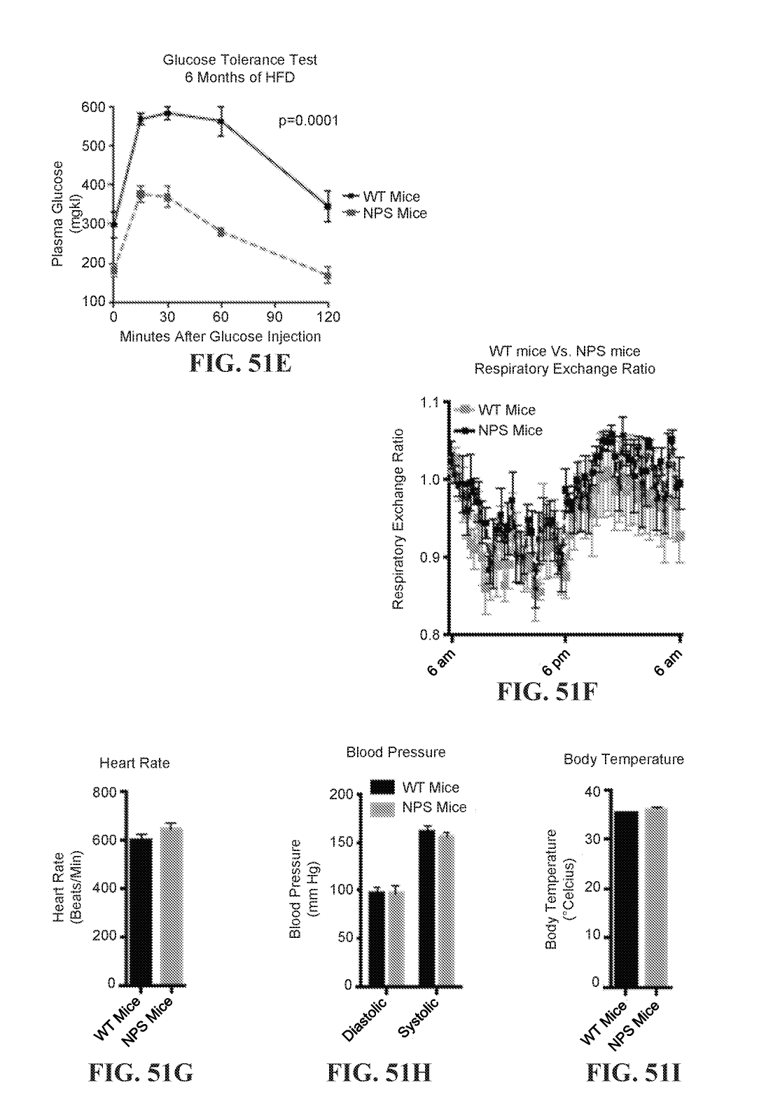

The genetic architecture of human NPS in mice was recapitulated FIG using the CRISPR/Cas9 system. The inventors introduced a small deletion (10 bp) encompassing the exon-65/intron-65 border (FIG. 48A). This resulted in skipping of exon 65 and a frame-shift leading to heterozygous ablation of the asprosin coding region (FIG. 48A), identical to documented molecular events in a known NPS patient (Jacquinet et al., 2014). Similar to human NPS, asprosin levels were far lower than 50% despite heterozygosity (FIG. 48B), presumably due to previously postulated dominant negative mechanism of action (Romere et al., 2016). Like the patients, NPS mice display extreme leanness (FIG. 48C), which was confirmed using DEXA scans to be due to reduction in both fat mass and lean mass with no observed change in length (FIG. 48D) (O'Neill et al., 2007). The reduction in lean mass is not surprising given the underlying Marfan syndrome, which is associated with a reduction in muscle mass and is a part of the NPS phenotype due to mutated fibrillin-1 protein (Judge and Dietz, 2005). Plasma leptin and adiponectin levels were significantly lower in NPS mice, consistent with their lipodystrophy (FIGS. 51A-51B).

Putting the mice on a high fat diet for 3 months showed a widening difference in body weight and fat mass between WT and NPS mice (FIG. 48E). On standard chow, the body weight curves of NPS mice separated from 3 weeks of age, culminating in a 10 g weight difference at 10 weeks (FIG. 48F). Putting the mice under severe diabetogenic and obesogenic stress (high fat diet for 6 months, 60% calories from fat) showed that NPS mice were completely protected from both obesity and diabetes, compared with WT mice (FIGS. 51C-51E). Similar to the human disorder, NPS mice displayed daily hypophagia without a change in energy expenditure, tilting the energy balance equation towards reduced energy intake to go with the observed reduction in adiposity and body weight (FIGS. 48G-48H). AgRP neuron activity was found to be significantly lower in NPS mice compared with WT littermates, demonstrated by decreased firing frequency and resting membrane potential (FIG. 48I). Finally, a single subcutaneous dose of recombinant asprosin was sufficient to completely rescue the hypophagia, demonstrating that NPS associated hypophagia is due to a deficiency of plasma asprosin and not due to some indirect effect of mutated profibrillin (FIG. 48J). Assessing the respiratory exchange ratio of NPS mice did not display a significant difference in substrate preference compared with WT mice (FIG. 51F). Basic vital signs such as heart rate, blood pressure and body temperature also remained unaltered (FIGS. 51G-51I).

Immunologic Sequestration of Asprosin Reduces Food Intake and Body Weight in Obese Mice—

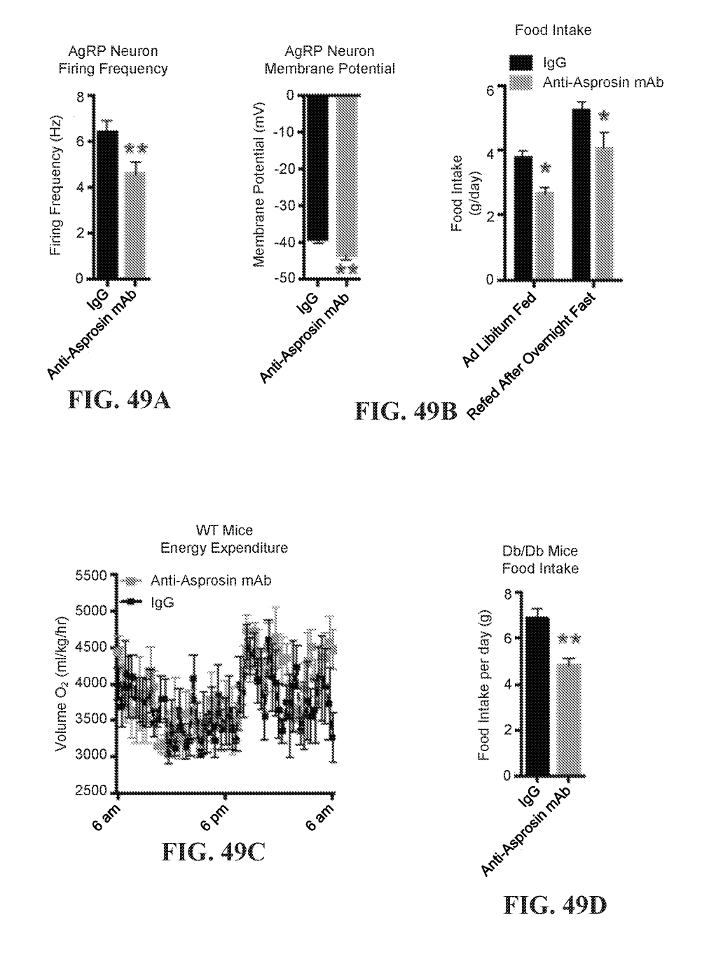

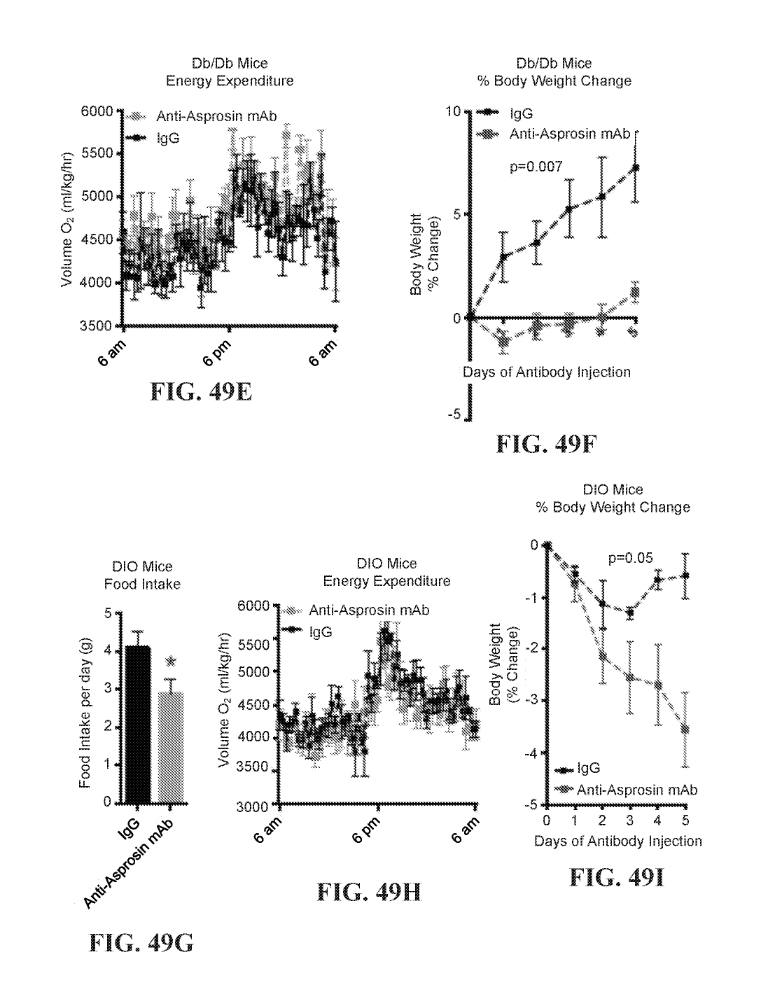

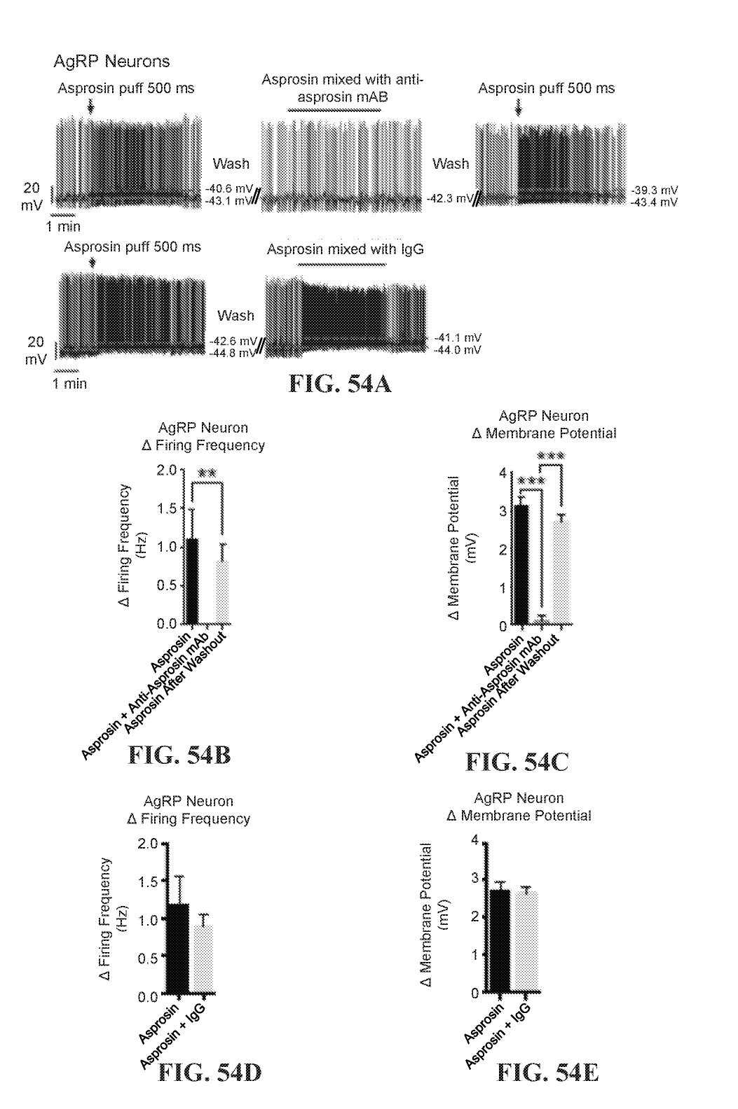

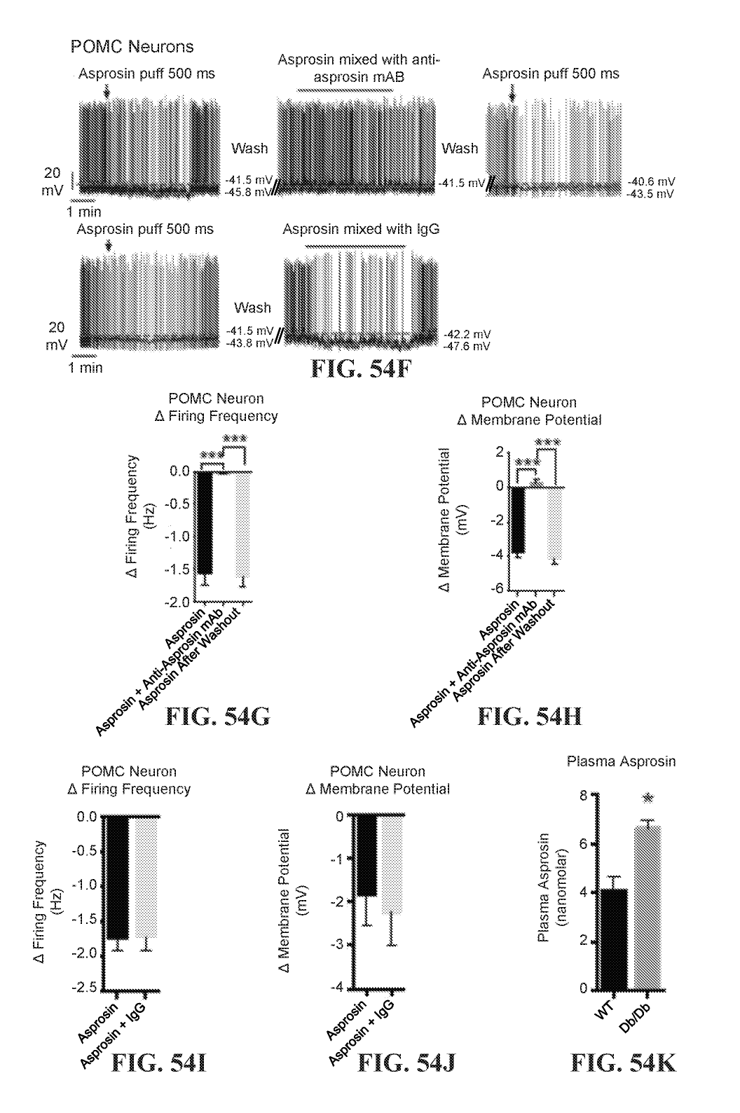

Immunologic sequestration of asprosin using a monoclonal antibody resulted in a reduction in AgRP neuron firing frequency and membrane potential in normal chow fed mice (FIG. 49A). This was also observed in vivo using c-fos protein expression as a marker of AgRP neuron activity (FIGS. 52A-52B). This was accompanied by a reduction in daily food intake without an associated change in energy expenditure (FIGS. 49B-49C). This antibody (FIG. 53A) has been previously validated by us for asprosin specificity (Romere et al., 2016), and detailed epitope mapping was performed for it (FIG. 53B). The neutralization activity of this antibody was tested in vitro that allowed calculation of its IC₅₀ (FIG. 53C), and that observation was extended to a calculation of the likely minimum efficacious dose in diabetic mice (FIG. 53D). For proof-of-concept in vivo, there was a complete lack of neutralization activity in mice treated with streptozotocin (FIG. 53E) given that such treatment results in a near absence of circulating asprosin (Romere et al., 2016). Additionally, the anti-asprosin antibody completely neutralized asprosin's ability to activate AgRP neurons and inhibit POMC neurons, while an irrelevant antibody (isotype matched IgG) had no effect (FIGS. 54A-54J).

It was previously demonstrated that human and mouse (high fat diet, homozygous ob mutation) obesity is associated with a pathological increase in plasma asprosin (Romere et al., 2016). Those results were extended to another model of mouse obesity—homozygous db mutation (FIG. 54K). Similar to previously demonstrated improvements in the glycemic profile of insulin resistant mice (Romere et al., 2016), asprosin immunologic sequestration reduced daily food intake without affecting energy expenditure in db/db mice (FIGS. 49D-49E). Daily intraperitoneal dosing over 5 days showed improvement in body weight to go along with the reduced food intake (FIG. 49F). Virtually identical results were obtained using mice on HFD (FIGS. 49G-49I). These results suggest that chronic asprosin depletion using a pharmacological entity such as a monoclonal antibody produces food intake and body weight reductions, as would be predicted from human and mouse genetic studies of asprosin depletion.

Significance of Certain Embodiments

Whether circulating asprosin concentration is experimentally decreased (genetic depletion in NPS patients, genetic depletion in NPS mice, or acute removal via immunologic sequestration in mice) or increased (adenovirus-mediated overexpression or recombinant protein injection), the result is a corresponding consistent change in food intake and adiposity. Asprosin mediated AgRP neuron activation appears to be central to these effects, since AgRP neuron ablation renders asprosin's orexigenic drive ineffective. However, it remains possible that asprosin could also exert its appetite stimulating action via other populations of orexigenic and anorexigenic neurons in addition to AgRP neurons.

Similar to what was observed in the liver (Romere et al., 2016), the Gαₛ-cAMP-PKA axis is necessary for asprosin-mediated AgRP neuron activation. This is consistent with the known orexigenic effect of Gαₛ and cAMP signaling in AgRP neurons (Nakajima et al., 2016), demonstrating a previously unknown circulating factor whose orexigenic activity is centered on this pathway. Furthermore, asprosin inhibits anorexigenic POMC neurons, which depends on intact GABAergic synaptic input. AgRP neurons are GABAergic neurons that project to POMC neurons, and their functional relevance is confirmed by the observation that their ablation largely prevents asprosin-mediated POMC neuron inhibition.

Individuals with NPS display a significant plasma asprosin deficit (Romere et al., 2016) along with phenotypic parameters of hypophagia (FIG. 43B), reduced subcutaneous adipose mass (FIG. 1A) (O'Neill et al., 2007) and very low body-mass-indices (Jacquinet et al., 2014; Passarge et al., 2016). Introducing a heterozygous NPS mutant allele in mice results in a virtual phenocopy of human NPS, with a 75% reduction in plasma asprosin, hypophagia, low adipose mass and body weight. The mice also display reduced levels of AgRP neuron activity. Replenishment of plasma asprosin, via a single subcutaneous injection, completely rescues hypophagia in NPS mice, demonstrating the dependence of that phenotype on plasma asprosin deficiency.

Given that asprosin is a circulating hormone, immunologic sequestration using monoclonal antibodies is an attractive depletion strategy for potential therapeutic applications. The efficacy of this strategy was demonstrated against the glucogenic effects of asprosin in insulin resistant mice, with monoclonal antibody treatment acutely lowering insulin levels in high fat fed and ob/ob mice (Romere et al., 2016). Here, it was found that immunologic asprosin sequestration reduces baseline AgRP neuron activity and results in a 20% reduction in daily food intake. Those findings were extending to obesity by demonstrating that chronic immunologic sequestration of asprosin in two independent mouse models of obesity (diet induced obesity and homozygous db mutation) results in a 20-30% reduction in daily food intake along with a reduction in body weight. Interestingly, the absence of leptin signaling in the setting of the db mutation did not affect the result, suggesting distinct mechanisms of action for the two hormones at AgRP and POMC neurons. No effects on energy expenditure were observed in any of the experiments suggesting that the effect of asprosin on the energy balance equation is limited to stimulation of appetite. Notably, AgRP neurons are known to suppress energy expenditure, in addition to their appetite-stimulatory function (Krashes et al., 2011). However, it is possible that the subset of AgRP neurons activated by asprosin has a greater impact on appetite than on energy expenditure, compared with the asprosin-unresponsive subset.

FBN1 mRNA is present at much lower levels in the brain relative to other tissues (Romere et al., 2016), and since robust crossing of the blood-brain-barrier by plasma asprosin was observed, in specific embodiments peripherally generated asprosin serves as a central appetite-modulating signal, similar to leptin. Organ-specific ablation of asprosin, particularly in adipose, should help address this and other important questions. One consideration is how asprosin signaling fits in the balance exerted by existing orexigenic and anorexigenic hormones such as ghrelin, leptin and insulin. It was demonstrated that the ghrelin receptor is dispensable for asprosin-mediated AgRP neuron activation (FIG. 46B), leptin receptor (db) is dispensable for asprosin loss-of-function induced reduction in food intake and body weight (FIGS. 49D-49F), and cross-talk with insulin's gluco-modulatory actions at the liver was previously demonstrated (Romere et al., 2016).

Thus, in one embodiment asprosin is as a glucogenic and orexigenic hormone that originates in adipose and is exquisitely sensitive to the whole-body energetic status, rising with fasting and abating with feeding. It performs both fasting-related functions using the same cAMP second messenger system, albeit in a distinct spatiotemporal manner. In specific embodiments, asprosin action at the AgRP neurons modulates its hepatic actions or vice versa. In particular embodiments, pathologic elevation of asprosin in human insulin resistance and obesity, and the observed efficacy of asprosin immunologic sequestration against insulin resistance (Romere et al., 2016) and obesity in mice, indicates that asprosin depletion serves as a unique therapeutic avenue against such diseases.

Methods

Study Subjects and Ethics Statement—

Informed consent and permission to use photographs and biological materials for research was obtained prior to participation from all subjects under Institutional Review Board-approved protocols at Baylor College of Medicine. Study subjects were assessed and genomic DNA was analyzed by either whole-exome or Sanger sequencing as reported previously (Romere et al., 2016).

Doubly Labeled Water Method (DLW)—

Total energy expenditure (TEE) was measured over a 10 day period using the DLW method (Butte et al., 2001; Roberts, 1989; Schoeller, 1988). After collection of the baseline urine samples, each participant received by mouth, 0.086 g/kg body weight of ²H₂O at 99.9 atom % ²H and 1.38 g/kg body weight of H₂¹⁸O at 10 atom % ¹⁸O (Isotec, Miamisburg, Ohio). Seven postdose urine samples (1 mL) were collected at home on days 1-10. The urine samples were stored frozen prior to analysis by Gas-Isotope-Ratio Mass Spectrometry. For stable hydrogen isotope ratio measurements, 10 μL of urine without further treatment were reduced to hydrogen gas with 200 mg zinc reagent at 500° C. for 30 min. The ²H/¹H isotope ratios of the hydrogen gas were measured with a Finnigan Delta-E gas-isotope-ratio mass spectrometer (Finnigan MAT, San Jose, Calif.). For stable oxygen isotope ratio measurements, 100 μL of urine was allowed to equilibrate with 300 mbar of CO₂ of known ¹⁸O content at 25° C. for 10 h using a VG ISOPREP-18 water-CO₂ equilibration system (VG Isogas, Limited, Cheshire, UK). At the end of the equilibration, the ¹⁸O/¹⁶O isotope ratios of the CO₂ were measured with a VG SIRA-12 gas-isotope-ratio mass spectrometer (VG Isogas, Limited, Cheshire, UK).

Room Respiration Calorimetry (Indirect Calorimetry)—

Energy expenditure was measured for 24 hours in a large (34 m³) calorimeter. During the 24 h calorimetry, subjects adhered to a schedule of physical activity (treadmill walking), feeding and sleeping. Heart rate and physical activity were recorded using Actiheart (CamNtech, Cambridge, UK). TEE, nonprotein energy expenditure (NPEE), respiratory quotient (RQ), and net substrate utilization were calculated from VO₂, VCO₂, and urinary nitrogen excretion. BMR was measured after a 12 h fast upon awakening for 30 minutes. Sleeping EE was measured for the entire night sleep period, confirmed by heart rate and motion sensors. Activity energy expenditure (AEE) was computed as TEE-BMR-0.1TEE assuming diet-induced thermogenesis to be 10% of TEE. Physical activity level (PAL) was defined as TEE/BMR. Energy cost of walking for one patient was measured while walking at 2.5 and 3.5 mph for 15 minutes on a treadmill (Vision Fitness T9600).

Dietary Recall—

A multiple-pass 24-h dietary recall was recorded in person by a registered dietitian using Nutrition Data Systems for Research (NDSR) (Database version 2005, Nutrition Coordinating Center, University of Minnesota, Minneapolis) (Johnson et al., 1996) and food models and household measures/dishware. The 24-h recall was obtained without prior notice. The multiple-pass 24-h recall method uses 3 distinct passes to garner information about a subject's food intake during the preceding 24 hours. Water consumption and vitamin mineral supplements were not included in the dietary assessment. The dietary recall was analyzed by NDSR, nutrient intakes were computed and these measures were used to evaluate diet quantity/quality against the standards set by the Dietary Reference Intakes (DRI).

Animals—

The inventors used 6 to 12-week-old male WT C57Bl/6 mice for in vivo studies. db/db obese mice were purchased from Jackson Laboratories. AgRP-ablated mice were generated by injecting AgRP-DTR mice with diptheria toxin [50 ng/g, subcutaneous (s.c), Sigma Aldrich D0564] (DT) during the first week after birth (Denis et al., 2015; Luquet et al., 2005), and AgRP-DTR mice received saline injection were served as control mice. NPS mice were generated at the Baylor College of Medicine Mouse Embryonic Stem Cell Core using a Crispr/Cas9 approach and a colony was maintained in house. Crispr/Cas9 mutagenesis was confirmed by running PCR using primers flanking the mutation site, followed by sequencing of the PCR product. Male, 6-12 weeks old, heterozygous, WT and NPS littermates were used for all experiments. Mice were housed 2-5 per cage in a 12-hr light/12-hr dark cycle with ad libitum access to food and water. For diet-induced obesity studies, mice were placed on an adjusted-calories diet providing 60% of calories from fat (Harlan-Teklad) for 12-16 weeks. Mice were exposed to adenoviral-mediated transgenesis (10¹¹ virus particles per mouse) via tail vein injections as described previously (Romere et al., 2016). In order to obtain CSF for asprosin analysis, the inventors had 8-12 week old male rats injected intravenously with His-tagged recombinant asprosin followed by sacrificing them and obtaining CSF 1 hour after injection. Flash frozen CSF was provided to the inventors for further analysis. Glucose tolerance tests (GTT) were performed using standard procedures. A 1.5 mg/g glucose bolus was used. Streptozoticin-induced diabetic mice were generated as reported previously (Romere et al., 2016).

Electrophysiology—

Whole-cell patch clamp recordings were performed on identified AgRP neurons or POMC neurons in the brain slices containing the arcuate nucleus of the hypothalamus (ARH). In particular, to identify AgRP neurons, the inventors crossed Rosa26-tdTOMATO allele onto the regular AgRP-Cre mice (Tong et al., 2008) to generate AgRP-Cre/Rosa26-tdTOMATO mice, which express TOMATO selectively in AgRP/NPY neurons. In some studies, the inventors crossed NPY-GFP mice (Pinto et al., 2004) with NPS mice to generate NPY-GFP mice with or without the NPS mutation, and GFP-labelled neurons in the arcuate nucleus were recorded. In order to record POMC neurons, the inventors crossed Rosa26-tdTOMATO allele onto the POMC-CreERT2 mice (Berglund et al., 2013) to generate POMC-CreERT2/Rosa26-tdTOMATO mice, which express TOMATO selectively in mature POMC neurons upon tamoxifen induction (0.2 mg/g, i.p. 6 weeks of age). In some studies, the inventors also crossed the AgRP-DTR allele onto POMC-GFP mice, and these mice received DT or saline injections (described above) to generate mice with or without AgRP neurons ablated.

Six to twelve-week old mice were deeply anesthetized with isoflurane and transcardially perfused with a modified ice-cold sucrose-based cutting solution (pH 7.3) containing 10 mM NaCl, 25 mM NaHCO₃, 195 mM Sucrose, 5 mM Glucose, 2.5 mM KCl, 1.25 mM NaH₂PO₄, 2 mM Na-Pyruvate, 0.5 mM CaCl₂, and 7 mM MgCl₂, bubbled continuously with 95% O₂ and 5% CO₂ (Ren et al., 2012). The mice were then decapitated, and the entire brain was removed and immediately submerged in the cutting solution. Slices (250 μm) were cut with a Microm HM 650V vibratome (Thermo Scientific). Three brain slices containing the arcuate nucleus were obtained for each animal (Bregma −2.06 mm to −1.46 mm; Interaural 1.74 mm to 2.34 mm), and recordings were made at levels throughout this brain region. The slices were recovered for 1 h at 34° C. and then maintained at room temperature in artificial cerebrospinal fluid (aCSF, pH 7.3) containing 126 mM NaCl, 2.5 mM KCl, 2.4 mM CaCl₂, 1.2 mM NaH₂PO₄, 1.2 mM MgCl₂, 11.1 mM glucose, and 21.4 mM NaHCO₃) saturated with 95% O₂ and 5% CO₂ before recording.

Slices were transferred to a recording chamber and allowed to equilibrate for at least 10 min before recording. The slices were superfused at 34° C. in oxygenated aCSF at a flow rate of 1.8-2 ml/min. GFP or TOMATO-labeled neurons in the ARH were visualized using epifluorescence and IR-DIC imaging on an upright microscope (Eclipse FN-1, Nikon) equipped with a moveable stage (MP-285, Sutter Instrument). Patch pipettes with resistances of 3-5 MΩ were filled with intracellular solution (pH 7.3) containing 128 mM K-Gluconate, 10 mM KCl, 10 mM HEPES, 0.1 mM EGTA, 2 mM MgCl₂, 0.05 mM Na-GTP and 0.05 mM Mg-ATP. Recordings were made using a MultiClamp 700B amplifier (Axon Instrument), sampled using Digidata 1440A and analyzed offline with pClamp 10.3 software (Axon Instruments). Series resistance was monitored during the recording, and the values were generally <10 MΩ and were not compensated. The liquid junction potential was +12.5 mV, and was corrected after the experiment. Data were excluded if the series resistance increased dramatically during the experiment or without overshoot for action potential. Currents were amplified, filtered at 1 kHz, and digitized at 20 kHz. Current clamp was engaged to test neural firing frequency and resting membrane potential (RM) at the baseline and puff delivered recombinant Asprosin (500 ms at various concentrations indicated in the figures). For the asprosin antibody pre-incubation experiment, asprosin and anti-asprosin mAB or IgG were gently mixed at a 1:100 ratio, and then kept on ice for one hour before recording. After AgRP or POMC neuron response to asprosin alone was confirmed, the mixture of recombinant asprosin and anti-asprosin mAB or IgG was perfused to treat the AgRP or POMC neurons for 4 minutes. In some experiments, the aCSF solution also contained 1 μM tetrodotoxin (TTX) (Sohn et al., 2013) and a cocktail of fast synaptic inhibitors, namely bicuculline (50 μM; a GABA receptor antagonist) (Liu et al., 2013) DAP-5 (30 μM; an NMDA receptor antagonist) (Liu et al., 2012) and CNQX (30 μM; an NMDA receptor antagonist) to block the majority of presynaptic inputs; in some experiments, DAP-5 (30 μM) and CNQX (30 μM) were included in the aCSF solution to block glutamatergic inputs; in some experiments, bicuculline (50 μM) was included in the aCSF solution to block GABAergic inputs. For the miniature excitatory postsynaptic current (mEPSC) recordings, the internal recording solution contained: 125 mM CsCH3SO3; 10 mM CsCl; 5 mM NaCl; 2 mM MgCl2; 1 mM EGTA; 10 mM HEPES; 5 mM (Mg)ATP; 0.3 mM (Na)GTP (pH 7.3 with NaOH). mEPSC in AgRP neurons was measured in the voltage clamp mode with a holding potential of −60 mV in the presence of 1 μM TTX and 50 μM bicuculline. mIPSC in POMC neurons was measured in the voltage clamp mode with a holding potential of −60 mV in the presence of 1 μM TTX and DAP-5 (30 μM; an NMDA receptor antagonist) and CNQX (30 μM; an NMDA receptor antagonist). Frequency and peak amplitude were measured using the Mini Analysis program (Synaptosoft, Inc.). The values for RM, firing frequency, mEPSC and mIPSC were averaged within 2-min bin at the baseline or after Asprosin treatment. A neuron was considered depolarized or hyperpolarized if a change in membrane potential was at least 2 mV in amplitude and this response was associated temporally with Asprosin. After recording, slices were fixed with 4% formalin in PBS at 4° C. overnight and then subjected to post hoc identification of the anatomical location of the recorded neuron within the ARH.

C-Fos Immunoreactivity in AgRP Neurons—

NPY-EGFP transgenic male adult mice were injected s.c. with either 100 ug IgG or 100 ug Anti-asprosin antibody at 6 pm (the onset of dark cycle), and food was removed. Sixteen hours later, mice were perfused with 10% formalin. Brain sections were cut at 25 μm (1:5 series). The fixed brains were collected and cut into 25 μm coronal sections. These brain sections were subjected to immunofluorescence staining for c-fos, as previously (Yan et al., 2016). Briefly, the sections were incubated in primary rabbit anti-c-fos antibody (1:1000; catalog #2250, Cell Signaling) overnight, followed by the donkey anti-rabbit AlexaFluor 594 (1:1000; catalog A-21207, Invitrogen) for 1.5 hours. Slides were cover-slipped and analyzed using a Leica DM5500 fluorescence microscope with OptiGrid structured illumination configuration. AgRP/NPY neurons were visualized as GFP-labelled neurons in the ARH, and the numbers of these AgRP/NPY neurons co-labelled c-fos immunoreactivity (red fluorescence) were counted. For each mouse, neurons were counted in 10-12 consecutive brain sections containing the ARH, and the average was treated as the data value for that mouse.

Mouse Food Intake and Energy Expenditure—

Food intake and energy expenditure were measured using the CLAMS System (Comprehensive Lab Animal Monitoring System, Columbus Instruments). Animals were acclimatized in the recording chambers for 48-72 hours, and measurements were taken subsequently for 24 hours during the light cycle and dark cycle. Mice received food and water ad libitum, or after an overnight fast where indicated. Oxygen consumption and food intake were recorded.

As previously described (Yan et al., 2016), male mice were anesthetized with inhaled isoflurane and stainless steel cannulas (Plastics One) were inserted into the lateral ventricles (0.34 mm caudal and 1 mm lateral from bregma; depth, 2.3 mm). Intracerebroventricular (ICV) cannulation was confirmed by demonstration of increased drinking and grooming behavior within 5 min after administration of angiotensin II (10 ng). These mice received 10 ng GFP (1 μg saline) twice a day for 3 continuous days, and 10 ng asprosin (in 1 μg saline) the last day. The BioDAQ food intake monitoring system (Research Diets, Inc) was used to monitor the food intake, and the food intake from the last GFP injection and asprosin were compared.

Mouse Body Composition—

Body composition was analyzed using an ECHO-MRI system (Echo medical systems, Texas) or DEXA scans where indicated. Lean mass, fat mass, and overall body weight were calculated using the manufacturer-provided software.

Recombinant Asprosin and GFP—

Human FBN1 (2732-2871 amino acids) cDNA was cloned and subsequently sub-cloned into a pET-22B vector for expression in Escherichia coli. The fusion protein that was expressed in E. coli is 146 amino acids long comprising of a six-amino-acid His tag on the N terminus and a 140-amino-acid wild-type asprosin. His-tagged GFP expressed in E. coli was obtained from Thermo Scientific as the control protein. The recombinant proteins were further purified using size exclusion columns and polymyxin B based endotoxin depletion columns (Detoxi-Gel™ Endotoxin Removing Gel by Thermo Scientific Inc.) with as many passages as required to bring the final endotoxin concentration equal to or below 2 EU/ml, and buffer exchanged into a PBS-Glycerol buffer or a 20 mM MOPS, pH 7.0, 300 mM NaCl, 150 mM Imidazole buffer. The purified proteins were subjected to SDS-PAGE analysis in order to determine the purity level. The His-GFP and His-asprosin proteins used in all recombinant protein experiments were >90% pure with endotoxin levels (determined using the Pierce™ LAL Chromogenic Endotoxin Quantitation Kit) as indicated (FIG. S7D) before and after passage through endotoxin depletion columns. Human aprosin with a C-terminal 6×His tag was produced by the UNC Protein Expression and Purification Core (UNC at Chapel Hill). Briefly the protein was expressed using the Expi293 transient transfection expression system (Gibco, ThermoFisher) according to manufactures included protocols. The media was collected, the cells cleared by centrifugation, and the resulting clarified media was sterile filtered and then concentrated and buffer exchanged into Ni-binding buffer (50 mM NaPo4 pH 7.4, 500 mM NaCl, 25 mM Imidazole) using a tangential flow filtration system (Millipore). Aprosin was purified from this solution via Ni affinity chromatography followed by size exclusion chromatography using an analytical Superdex75 column (GE Healthcare) equilibrated with PBS with 10% glycerol.

Sandwich ELISA and Western Blot—

For the asprosin sandwich ELISA, a mouse anti-asprosin monoclonal antibody against asprosin amino acids 106-134 (human profibrillin amino acids 2838-2865) was used as the capture antibody, and a goat anti-asprosin polyclonal antibody against asprosin amino acids 6-19 (human profibrillin amino acids 2737-2750) by Abnova was used as the detection antibody. An anti-goat secondary antibody linked to HRP was used to generate a signal. For the His-tag sandwich ELISA, the same procedure was used, except for the use of a goat anti-His polyclonal antibody (Abcam) as the detection antibody. EDTA plasma was used for plasma sandwich ELISAs. Plasma western blotting for asprosin was done using the same mouse monoclonal anti-asprosin antibody as for the ELISA. For the mammalian asprosin western blot, 40 μg mammalian asprosin was enzymatically deglycosylated using a protein deglycosylation mix (New England Biolabs). 20 μg glycosylated mammalian asprosin, 40 μg deglycosylated mammalian asprosin, and 20 μg bacterial asprosin were analyzed for molecular weight comparison.

Characterization of the Mouse Anti-Asprosin Monoclonal Antibody—

An in-vitro assay to test the potential of a mouse IgG monoclonal antibody (mAb) to neutralize asprosin was performed. To evaluate the mAb's neutralization potential, 10 nM asprosin was incubated with varying molar ratios of mAb for 1 hour on ice, and residual asprosin was measured using our sandwich ELISA. The in vitro IC50 was calculated using either the residual concentration, or absolute extinction values. Using guidelines from the UK NC3Rs (https://www.nc3rs.org.uk/mouse-decision-tree-blood-sampling retrieved on Feb. 1, 2017) to estimate the blood volume of a 50 g mouse, and setting the mAb molecular weight at 150 kDa, 44 μg mAb per 50 g mouse was determined to be sufficient to neutralize 50% of endogenous asprosin.

Epitope Mapping of the Mouse Anti-Asprosin Monoclonal Antibody—

Eleven octamer peptides with a two-amino-acid overlap covering the known immunization peptide used for developing the mouse anti-asprosin monoclonal antibody kkkelnqledkydkdylsgelgdnlkink (SEQ ID NO: 5) were printed in spots on glass slides by Raybiotech (Raybiotech, Norcross, Ga.) and incubated with the monoclonal antibody. The antibody was detected using a biotinylated anti-mouse antibody, followed by streptavidin conjugated with a fluorophore. Fluorescence of spots corresponding to specific octamer peptides was recorded. The results were reported in raw fluorescence (arbitrary numbers), where the highest number signifies strongest fluorescence and strongest antibody binding.

Asprosin Plasma Half-Life—

Asprosin expressed in HEK293 was labeled with biotin using a EZ-Link Sulfo-NHS-Biotin kit (Thermo Scientific), and excess biotin was removed using a Zeba Spin desalting column (Thermo Scientific). Final protein concentration was estimated using a BCA assay (Thermo Scientific). Approximately 30 ug/mouse of labeled asprosin was injected subcutaneously into C57Bl/6 mice. Blood was drawn before injection (baseline), and 30, 60, 120, and 360 minutes after injection, as well as after 24 and 48 hours. Biotinylated asprosin was detected using a custom designed sandwich ELISA. A plate was coated with the anti-asprosin monoclonal antibody (capture antibody), total plasma asprosin was bound, and only biotinylated asprosin was detected using Streptavidin-HRP.

Human and Mouse Adiponectin, Leptin, and Ghrelin—

Human adiponectin, mouse adiponectin, human leptin, mouse leptin, human ghrelin (total) and mouse ghrelin (total) ELISA kits (Millipore, Billerica, Mass.) were used to determine the concentrations of each plasma parameter in flash-frozen, previously unthawed plasma as per the manufacturer's instructions.

Statistical Methods—

All results are presented as mean±SEM. p values are calculated by unpaired Student's t test for all results, except where indicated by two-way ANOVA. *p<0.05, **p<0.01, and ***p<0.001.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
1. Agarwal, et al., Maturitas 76, 296-302 (2013).
2. Aponte, Y., Atasoy, D., and Sternson, S. M. (2011). AGRP neurons are sufficient to orchestrate feeding behavior rapidly and without training. Nat Neurosci 14, 351-355.
3. Aronoff, S. L., Berkowitz, K., and Shreiner, B. (2004). Glucose metabolism and regulation: beyond insulin and glucagon. Diabetes Spectrum.
4. Atasoy, D., Betley, J. N., Su, H. H., and Sternson, S. M. (2012). Deconstruction of a neural circuit for hunger. Nature 488, 172-177.
5. Bainbridge, et al., Genome Biol. 12, R68 (2011).
6. Behrens, O. K., and Bromer, W. W. (1958). Biochemistry of the protein hormones. Annual Review of Biochemistry 27, 57-100.
7. Berglund, E. D., Liu, C., Sohn, J.-W., Liu, T., Kim, M. H., Lee, C. E., Vianna, C. R., Williams, K. W., Xu, Y., and Elmquist, J. K. (2013). Serotonin 2C receptors in pro-opiomelanocortin neurons regulate energy and glucose homeostasis. J. Clin. Invest. 123, 5061-5070.
8. Bindlish, S., Presswala, L. S., and Schwartz, F. (2015). Lipodystrophy: Syndrome of severe insulin resistance. Postgrad Med 127, 511-516.
9. Butte, N. F., Wong, W. W., and Hopkinson, J. M. (2001). Energy requirements of lactating women derived from doubly labeled water and milk energy output. The Journal of Nutrition 131, 53-58.
10. Campbell, J. E., and Drucker, D. J. (2015). Islet α cells and glucagon-critical regulators of energy homeostasis. Nat Rev Endocrinol 11, 329-338.
11. Chopra, et al., Cell Metab. 13:35-43 (2011).
12. Chopra, et al., Science 322:1395-99 (2008).
13. Cui, X., Wang, Y., Tang, Y., Liu, Y., Zhao, L., Deng, J., Xu, G., Peng, X., Ju, S., Liu, G., et al. (2011). Seipin ablation in mice results in severe generalized lipodystrophy. Hum Mol Genet 20, 3022-3030.
14. Davis & Summers, Molecular Genetics and Metabolism 107, 635-647 (2012).
15. Denis, R. G. P., Joly-Amado, A., Webber, E., Langlet, F., Schaeffer, M., Padilla, S. L., Cansell, C., Dehouck, B., Castel, J., Delbès, A.-S., et al. (2015). Palatability Can Drive Feeding Independent of AgRP Neurons. Cell Metab. 22, 646-657.
16. Dumoulin, G., Hory, B., Nguyen, N. U., Henriet, M. T., Bresson, C., Regnard, J., and Saint-Hillier, Y. (1995). Acute oral calcium load decreases parathyroid secretion and suppresses tubular phosphate loss in long-term renal transplant recipients. Am. J. Nephrol. 15, 238-244.
17. Esposito, et al., Plastic Reconstruc Surg. 118:1048-1057 (2006).
18. Friedman, American Journal of Clinical Nutrition 89, 973S-979S (2009).
19. Gelato, et al., Clin Ther. 29(11):2269-88, 2007.
20. Goldblatt, et al., Am. J Med. Genet. A 155, 717-720 (2011).
21. Goldstein & Brown, Arterioscler. Thromb. Vasc. Biol. 29, 431-438 (2009).
22. Graul-Neumann, et al., Am. J. Med. Genet. A 152A, 2749-2755 (2010).
23. Guba & Harsanyi, Kiserletes orvostudomany 16:28-34 (1964).
24. Horn & Robinson, Am. J Med. Genet. A 155, 721-724 (2011).
25. Hou, Pediatrics and neonatology 50, 102-109 (2009).
26. Jaager, et al., PLoS ONE 7, e38833 (2012).
27. Jacquinet, et al., Eur J Med Genet 57, 230-234 (2014).
28. Jensen, et al., Proceedings of the National Academy of Sciences 111, 10155-10160 (2014).
29. Johnson, R. K., Driscoll, P., and Goran, M. I. (1996). Comparison of multiple-pass 24-hour recall estimates of energy intake with total energy expenditure determined by the doubly labeled water method in young children. Journal of the American Dietetic Association 96, 1140-1144.
30. Judge, D. P., and Dietz, H. C. (2005). Marfan's syndrome. The Lancet 366, 1965-1976.
31. Krashes, M. J., Koda, S., Ye, C., Rogan, S. C., Adams, A. C., Cusher, D. S., Maratos-Flier, E., Roth, B. L., and Lowell, B. B. (2011). Rapid, reversible activation of AgRP neurons drives feeding behavior in mice. J. Clin. Invest. 121, 1424-1428.
32. Kulstad, et al., Curr Opin Clin Nutr Metab Care. 10(4):488-93, 2007.
33. Liu, T., Kong, D., Shah, B. P., Ye, C., Koda, S., Saunders, A., Ding, J. B., Yang, Z., Sabatini, B. L., and Lowell, B. B. (2012). Fasting Activation of AgRP Neurons Requires NMDA Receptors and Involves Spinogenesis and Increased Excitatory Tone. Neuron 73, 511-522.
34. Liu, Y., Liu, H., Sauvey, C., Yao, L., Zarnowska, E. D., and Zhang, S.-C. (2013). Directed differentiation of forebrain GABA interneurons from human pluripotent stem cells. Nat Protoc 8, 1670-1679.
35. Loeys, et al., J. Med. Genet. 47, 476-485 (2010).
36. Lönnqvist, L., Reinhardt, D., Sakai, L., and Peltonen, L. (1998). Evidence for furin-type activity-mediated C-terminal processing of profibrillin-1 and interference in the processing by certain mutations. Hum Mol Genet 7, 2039-2044.
37. Lupski, et al., Genome Med 5, 57 (2013).
38. Luquet, S., Perez, F. A., Hnasko, T. S., and Palmiter, R. D. (2005). NPY/AgRP neurons are essential for feeding in adult mice but can be ablated in neonates. Science 310, 683-685.
39. Magnusson, I., Rothman, D. L., and Katz, L. D. (1992). Increased rate of gluconeogenesis in type II diabetes mellitus. A 13C nuclear magnetic resonance study. J. Clin. Invest. 90, 1323-1327.
40. Malik, et al., Nat Rev Endocrinol 9, 13-27 (2013).
41. Milewicz, et al., J. Clin. Invest. 95, 2373-2378 (1995).
42. Morgen & Sorensen, Nat Rev Endocrinol 10, 513-514 (2014).
43. Mueller, et al., World J Gastroentrol. 20(28):9361-73, 2014.
44. Nakajima, K.-I., Cui, Z., Li, C., Meister, J., Cui, Y., Fu, O., Smith, A. S., Jain, S., Lowell, B. B., Krashes, M. J., et al. (2016). Gs-coupled GPCR signalling in AgRP neurons triggers sustained increase in food intake. Nat Commun 7, 10268.
45. Nakazato, M., Murakami, N., Date, Y., Kojima, M., Matsuo, H., Kangawa, K., and Matsukura, S. (2001). A role for ghrelin in the central regulation of feeding. Nature 409, 194-198.
46. Nickel, W. (2003). The mystery of nonclassical protein secretion. European Journal of Biochemistry 270, 2109-2119.
47. Nolis, J Human Genet. 59, 16-23 (2013).
48. O'Neill, et al., Am. J. Med. Genet. A 143A, 1421-1430 (2007).
49. Oosting, et al., Ped Res. 72:362-9 (2012).
50. O'Reilly, et al., Cell 88, 277-285 (1997).
51. Passarge, E., Robinson, P. N., and Graul-Neumann, L. M. (2016). Marfanoid-progeroid-lipodystrophy syndrome: a newly recognized fibrillinopathy. Eur J Hum Genet 24, 1244-1247.
52. Pereira, L., Lee, S. Y., Gayraud, B., Andrikopoulos, K., Shapiro, S. D., Bunton, T., Biery, N.J., Dietz, H. C., Sakai, L. Y., and Ramirez, F. (1999). Pathogenetic sequence for aneurysm revealed in mice underexpressing fibrillin-1. Proceedings of the National Academy of Sciences 96, 3819-3823.
53. Pinto, S., Roseberry, A. G., Liu, H., Diano, S., Shanabrough, M., Cai, X., Friedman, J. M., and Horvath, T. L. (2004). Rapid rewiring of arcuate nucleus feeding circuits by leptin. Science 304, 110-115.
54. Pureza & Florea, Curr Heart Fail Rep 10, 307-314 (2013).
55. Pyeritz, Heart 95, 173-175 (2009).
56. Raghunath, et al., Journal of cell science 112 (Pt 7), 1093-1100 (1999).
57. Rajala, et al., Endocrin. 144:3765-73 (2003).
58. Reinhardt, et al., Ciba Foundation Symposium. 192:138-43, discussion 143-127 (1995).
59. Remington: The Science and Practice of Pharmacy, 21st Ed. Lippincott Williams and Wilkins, 2005.
60. Remington's Pharmaceutical Sciences, 18th Ed. Mack Printing Company, 1990.
61. Ren, H., Orozco, I. J., Su, Y., Suyama, S., Gutiérrez-Juarez, R., Horvath, T. L., Wardlaw, S. L., Plum, L., Arancio, O., and Accili, D. (2012). FoxO1 Target Gpr17 Activates AgRP Neurons to Regulate Food Intake. Cell 149, 1314-1326.
62. Ritty, et al., Journal of Biological Chemistry 274, 8933-8940 (1999).
63. Roberts, S. B. (1989). Use of the doubly labeled water method for measurement of energy expenditure, total body water, water intake, and metabolizable energy intake in humans and small animals. Can. J. Physiol. Pharmacol. 67, 1190-1198.
64. Rocha, et al., Nat Rev Cardiol 6:399-409 (2009).
65. Romere, C., Duerrschmid, C., Bournat, J., Constable, P., Jain, M., Xia, F., Saha, P. K., Del Solar, M., Zhu, B., York, B., et al. (2016). Asprosin, a Fasting-Induced Glucogenic Protein Hormone. Cell 165, 566-579.
66. Saha, P. K., Reddy, V. T., Konopleva, M., Andreeff, M., and Chan, L. (2010). The triterpenoid 2-cyano-3,12-dioxooleana-1,9-dien-28-oic-acid methyl ester has potent anti-diabetic effects in diet-induced diabetic mice and Lepr(db/db) mice. J. Biol. Chem. 285, 40581-40592.
67. Schoeller, D. A. (1988). Measurement of energy expenditure in free-living humans by using doubly labeled water. The Journal of Nutrition 118, 1278-1289.
68. Shoelson, et al., Gastroentrol. 132:2169-80 (2007).
69. Sohn, J.-W., Harris, L. E., Berglund, E. D., Liu, T., Vong, L., Lowell, B. B., Balthasar, N., Williams, K. W., and Elmquist, J. K. (2013). Melanocortin 4 Receptors Reciprocally Regulate Sympathetic and Parasympathetic Preganglionic Neurons. Cell 152, 612-619.
70. Spiegelman & Flier, Cell 104, 531-543 (2001).
71. Spiegelman & Flier, Cell 87, 377-389 (1996).
72. Takenouchi, et al., Am. J. Med. Genet. A 161, 3057-3062 (2013).
73. Tong, Q., Ye, C.-P., Jones, J. E., Elmquist, J. K., and Lowell, B. B. (2008). Synaptic release of GABA by AgRP neurons is required for normal regulation of energy balance. Nat Neurosci 11, 998-1000.
74. Trayhurn, et al., J Nutr. 136:1935S-39S (2006).
75. Trumbo, P., Schlicker, S., Yates, A. A., Poos, M., Food and Nutrition Board of the Institute of Medicine, The National Academies (2002). Dietary reference intakes for energy, carbohydrate, fiber, fat, fatty acids, cholesterol, protein and amino acids. Journal of the American Dietetic Association 102, 1621-1630.
76. Wallis, et al., Journal of cellular biochemistry 90, 641-652 (2003).
77. Xu, et al., J Clin Invest. 112:1821-30 (2003).
78. Yan, C., He, Y., Xu, Y., Shu, G., Wang, C., Yang, Y., Saito, K., Xu, P., Hinton, A. O., Yan, X., et al. (2016). Apolipoprotein A-IV Inhibits AgRP/NPY Neurons and Activates Pro-Opiomelanocortin Neurons in the Arcuate Nucleus. Neuroendocrinology 103, 476-488.
79. Zeyda & Stulnig, et al., Gerontol 55:379-86 (2009).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. An isolated antibody or antibody fragment that specifically binds a peptide consisting of SEQ ID NO:4.

2. The antibody or antibody fragment of claim 1, wherein the antibody is produced by hybridoma cell line deposited with the American Type Culture Collection under accession number ATCC PTA-123085.

3. The antibody or antibody fragment of claim 1, wherein the antibody is a humanized antibody.

4. The antibody or antibody fragment of claim 1, wherein the antibody is a single chain antibody.

5. The antibody or antibody fragment of claim 1, wherein the antibody is a nanobody.

6. The antibody or antibody fragment of claim 1, wherein the antibody is a humanized single chain antibody or nanobody.

7. The antibody or antibody fragment of claim 1, wherein the antibody is a bispecific antibody.

8. The antibody or antibody fragment of claim 1, wherein the antibody is a humanized bispecific antibody.

9. The antibody or antibody fragment of claim 1, wherein the antibody or antibody fragment is conjugated to a biologically active effector domain.

10. A hybridoma cell as deposited with the American Type Culture Collection under accession number ATCC PTA-123085.

11. A monoclonal antibody produced by a hybridoma deposited with the American Type Culture Collection under accession number ATCC PTA-123085.

12. A composition comprising the antibody of any one of claims 1-9.

13. Hybridoma cell line ATCC PTA-123085.

14. An antibody produced by the cell line of claim 13.

15. A method of measuring the level of asprosin in a sample from an individual, comprising the steps of:
a) contacting an antibody or antibody fragment that specifically binds a peptide consisting of SEQ ID NO:4 with a sample;
b) forming a complex between the antibody and asprosin from the sample; and
c) detecting the antibody/asprosin complex and determining the level of asprosin in the sample.

16. The method of claim 15, wherein the individual is suspected of having or is known to have insulin resistance, type II diabetes, or metabolic syndrome or is obese or overweight.

17. The method of claim 15 or 16, wherein the sample comprises plasma, blood, biopsy, saliva, semen, urine, hair, cerebrospinal fluid, cheek scrapings, or a combination thereof.

18. The method of any one of claims 15-17, wherein the antibody or antibody fragment is immobilized on a support.

19. The method of any one of claims 15-17, wherein the antibody/asprosin complex is immobilized on a support.

20. The method of any one of claims 15-19, wherein the antibody or antibody fragment is coupled to a detectable label.

21. The method of any one of claims 15-20, wherein the individual is identified as having or is at risk of developing insulin resistance, type II diabetes, or metabolic syndrome if the level of asprosin is greater than a reference level.

22. A method of treating insulin resistance, obesity, diabetes, and/or metabolic syndrome in an individual, comprising the step of providing an effective amount of the antibody or antibody fragment of any one of claim 1-9, 11, or 14 or the composition of claim 12 to the individual.

23. A method of inhibiting asprosin in an individual, comprising the step of providing an effective amount of the antibody of any one of claim 1-9, 11, or 14 or the composition of claim 7 to the individual.

24. The method of claim 23, wherein the individual has or is suspected of having insulin resistance, obesity, diabetes, and/or metabolic syndrome.

25. The method of claim 23 or 24, wherein the individual has a body mass index (BMI) of 30 or greater.

26. The method of claim 23, wherein the individual has a BMI of between 25 and 29.9.

27. Use of the antibody of any one of claim 1-9, 11, or 14 or the composition of claim 12 for the manufacture of a medicament for reducing asprosin levels in an individual.

28. The method of claim 27, wherein the individual has or is suspected of having insulin resistance, obesity, type II diabetes, and/or metabolic syndrome.

29. Use of the antibody of any one of claim 1-9, 11, or 14 or the composition of claim 12 for the manufacture of a medicament for treating insulin resistance, obesity, type II diabetes, and/or metabolic syndrome in an individual.


SEQUENCE LISTING (24 sequences)

SEQ ID NO: 1 - protein, 140 aa, Homo sapiens
STNETDASNIEDQSETEANVSLASWDVEKTAIFAFNISHVSNKVRILELLPALTTLTNHNRYLIESGNEDGFFKINQKEGISYLHFTKKKPVAGTYSLQISSTPLYKKKELNQLEDKYDKDYLSGELGDNLKMKIQVLLH

SEQ ID NO: 2 - protein, 147 aa, Homo sapiens
MHHHHHHSTNETDASNIEDQSETEANVSLASWDVEKTAIFAFNISHVSNKVRILELLPALTTLTNHNRYLIESGNEDGFFKINQKEGISYLHFTKKKPVAGTYSLQISSTPLYKKKELNQLEDKYDKDYLSGELGDNLKMKIQVLLH

SEQ ID NO: 3 - protein, 25 aa, Homo sapiens
ETEANVSLASWDVEKTAIFAFNISH

SEQ ID NO: 4 - protein, 28 aa, Homo sapiens
KKKELNQLEDKYDKDYLSGELGDNLKMK

SEQ ID NO: 5 - protein, 28 aa, Mus musculus
KKKELNQLEDKYDKDYLSGELGDNLKMK

SEQ ID NO: 6 - protein, 6 aa, Artificial Sequence
  Synthetic Peptide
RGRKRR

SEQ ID NO: 7 - protein, 171 aa, Homo sapiens
VSGEMDDNSLSPEACYECKINGYPKRGRKRRSTNETDASNIEDQSETEANVSLASWDVEKTAIFAFNISHVSNKVRILELLPALTTLTNHNRYLIESGNEDGFFKINQKEGISYLHFTKKKPVAGTYSLQISSTPLYKKKELNQLEDKYDKDYLSGELGDNLKMKIQVLLH

SEQ ID NO: 8 - protein, 36 aa, Homo sapiens
VSGEMDDNSLSPEACYECKINGYPKRGRKRRSTNET

SEQ ID NO: 9 - protein, 51 aa, Homo sapiens
VSGEMDDNSLSPEACYECKINGYPKRGRKRRSTNETDASNIELGQKRQKPM

SEQ ID NO: 10 - protein, 35 aa, Homo sapiens
VSGEMDDNSLSPEACYECDQWLPQTGQETEKHKRN

SEQ ID NO: 11 - protein, 29 aa, Homo sapiens
VSGEMDDNSLSPEACYECTGQETEKHKRN

SEQ ID NO: 12 - protein, 51 aa, Homo sapiens; identical to SEQ ID NO: 9

SEQ ID NO: 13 - protein, 33 aa, Homo sapiens
VSGEMDDNSLSPEACYECKINGYPKETEKHKRN

SEQ ID NO: 14 - protein, 51 aa, Homo sapiens; identical to SEQ ID NO: 9

SEQ ID NO: 15 - protein, 231 aa, Homo sapiens
QFSGGCQDINECGSAQAPCSYGCSNTEGGYLCGCPPGYFRIGQGHCVSGMGMGRGNPEPPVSGEMDDNSLSPEACYECKINGYPKRGRKRRSTNETDASNIEDQSETEANVSLASWDVEKTAIFAFNISHVSNKVRILELLPALTTLTNHNRYLIESGNEDGFFKINQKEGISYLHFTKKKPVAGTYSLQISSTPLYKKKELNQLEDKYDKDYLSGELGDNLKMKIQVLLH

SEQ ID NO: 16 - protein, 96 aa, Homo sapiens
QFSGGCQDINECGSAQAPCSYGCSNTEGGYLCGCPPGYFRIGQGHCVSGMGMGRGNPEPPVSGEMDDNSLSPEACYECKINGYPKRGRKRRSTNEN

SEQ ID NO: 17 - protein, 51 aa, Homo sapiens
QFSGGCQDINECGSAQAPCSYGCSNTEGGYLCGCPPGYFRIGQISLRQKPM

SEQ ID NO: 18 - protein, 95 aa, Homo sapiens
QFSGGCQDINECGSAQAPCSYGCSNTEGGYLCGCPPGYFRIGQGHCVSGMGMGRGNPEPPVSGEMDDNSLSPEACYECDQWLPQTGQETEKHKRN

SEQ ID NO: 19 - protein, 89 aa, Homo sapiens
QFSGGCQDINECGSAQAPCSYGCSNTEGGYLCGCPPGYFRIGQGHCVSGMGMGRGNPEPPVSGEMDDNSLSPEACYECTGQETEKHKRN

SEQ ID NO: 20 - protein, 51 aa, Homo sapiens; identical to SEQ ID NO: 17

SEQ ID NO: 21 - protein, 93 aa, Homo sapiens
QFSGGCQDINECGSAQAPCSYGCSNTEGGYLCGCPPGYFRIGQGHCVSGMGMGRGNPEPPVSGEMDDNSLSPEACYECKINGYPKETEKHKRN

SEQ ID NO: 22 - protein, 51 aa, Homo sapiens; identical to SEQ ID NO: 17

SEQ ID NO: 23 - protein, 140 aa, Homo sapiens; identical to SEQ ID NO: 1

SEQ ID NO: 24 - protein, 146 aa, Homo sapiens
HHHHHHSTNETDASNIEDQSETEANVSLASWDVEKTAIFAFNISHVSNKVRILELLPALTTLTNHNRYLIESGNEDGFFKINQKEGISYLHFTKKKPVAGTYSLQISSTPLYKKKELNQLEDKYDKDYLSGELGDNLKMKIQVLLH